(19) United States
(12) Patent Application Publication
(10) Pub. No.: US 2026/0152494 A1
(43) Pub. Date: Jun. 4, 2026

(54) CDK2 INHIBITORS AND METHODS OF USING THE SAME

(71) Applicant: Cedilla Therapeutics, Inc., Foxboro, MA (US)

(72) Inventors: Louise Clare KIRMAN, Foxboro, MA (US); Carl Eric SCHWARTZ, Foxboro, MA (US); Thomas P. BLAISDELL, Foxboro, MA (US)

(21) Appl. No.: 18/995,333

(22) PCT Filed: Jul. 28, 2023
(86) PCT No.: PCT/US2023/071256

§ 371 Date: Jan. 16, 2025

Related U.S. Application Data
(60) Provisional application No. 63/393,709, filed on Jul. 29, 2022.

(51) Int. Cl. C07D 401/12

(52) U.S. Cl. CPC C07D 417/14; A61K 31/397; A61K 31/407; A61K 31/4155; A61K 31/4245; A61K 31/427; A61K 31/438; A61K 31/444; A61K 31/4525; A61K 31/4535; A61K 31/454; A61K 31/497; A61K 31/501; A61K 31/5377; A61P 35/00; C07D 471/10; C07D 487/10; C07D 491/107; C07D 495/10

(57) ABSTRACT

The present disclosure provides compounds, compositions thereof, and methods of using the same for the inhibition of CDK2, and the treatment of CDK2 related diseases and disorders.

### CROSS REFERENCE TO RELATED APPLICATIONS

This application claims the benefit of priority to U.S. Provisional Application No. 63/393,709, filed Jul. 29, 2022, the entire contents of which is herein incorporated by reference.

### FIELD

The present disclosure relates generally to Cyclin-dependent kinase 2 (CDK2) inhibiting chemical compounds and uses thereof in the inhibition of the activity of CDK2. The disclosure also provides pharmaceutically acceptable compositions comprising compounds disclosed herein and methods of using said compounds and compositions in the treatment of various disorders related to CDK2 activity.

### BACKGROUND

Cell cycle dysregulation, including uncontrolled cell growth, impaired cell differentiation and abnormal apoptosis have been shown to be caused by over activity of Cyclin-dependent kinases (CDKs). CDKs are important serine/threonine protein kinases that become active when combined with a specific cyclin partner. There are various subtypes of CDKs, each having a different role during the cell cycle, with varying levels of activity during each of the phases. CDK1, CDK2, CDK4 and CDK6 have been found to be specifically important subtypes, where over activity of one or more of these subtypes may lead to dysregulation of the cell cycle and the development of a variety of cancers. The S phase of the cell cycle is responsible for DNA replication and is the phase where aberrant DNA replication may occur. The CDK2/cyclin E complex is required for the cell cycle transition from the G1 phase to the S phase and the CDK2/cyclin A complex is required for the cell cycle transition from the S phase to the G2 phase. Therefore, selective inhibition of the CDK2/cyclin E and/or CDK2/cyclin A complexes can prevent aberrant DNA replication and can be used to treat certain cancers.

Accordingly, there is a need for the development of compounds capable of inhibiting the activity of CDK2/cyclin complexes, and pharmaceutical compositions thereof, for the prevention, and treatment of CDK2 related diseases or disorders.

### SUMMARY

The present disclosure is based at least in part on the identification of compounds that bind and inhibit Cyclin-dependent kinase 2 (CDK2) and/or CDK2/cyclin complexes and methods of using the same to treat diseases associated with CDK2 activity. Disclosed herein is a compound according to Formula I or a pharmaceutically acceptable salt thereof:

In some embodiments, provided herein are compounds according to Formula I:

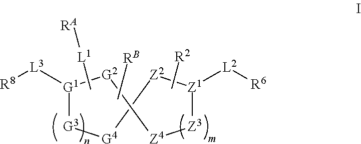

or a pharmaceutically acceptable salt thereof, wherein:
each variable is as defined and described herein.

Compounds of the present disclosure, and pharmaceutically acceptable compositions thereof, are useful for treating a variety of diseases, disorders or conditions, associated with CDK2 activity. Such diseases, disorders, or conditions include those described herein.

### DETAILED DESCRIPTION OF CERTAIN EMBODIMENTS

### 1. General Description of Compounds of the Disclosure

The present disclosure provides compounds capable of inhibiting Cyclin-dependent kinase 2 (CDK2) and/or CDK2/cyclin complexes.

In some embodiments, provided herein are compounds according to Formula I:

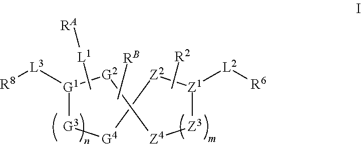

or a pharmaceutically acceptable salt thereof, wherein:
G¹ is N or CH;
each G², G³, and G⁴ is independently optionally substituted methylene, —O—, —S—, —N(R)—, —C(O)—, —S(O)—, or —S(O)₂—;
Z¹ is N or CH; and
each Z², Z³ and Z⁴ is independently optionally substituted methylene, —O—, —S—, —N(R)—, —C(O)—, —S(O)—, or —S(O)₂—;
R^{A} is an amide isostere,

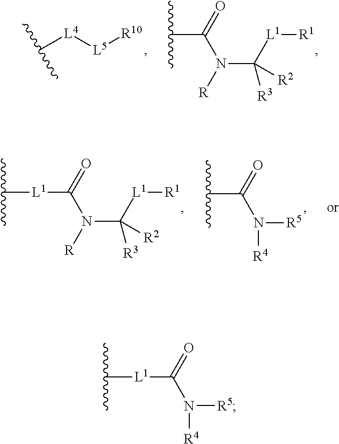

R^{B} is a hydrogen, an optionally substituted C₁₋₆ aliphatic group, or a halogen;
each L¹ is independently a covalent bond or a saturated or unsaturated, straight or branched, optionally substituted bivalent C₁₋₆ hydrocarbon chain, wherein 0-4 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
each of R² and R³ is independently hydrogen, an optionally substituted C₁₋₆ aliphatic group, —C₁₋₆ alkylene-OR, —C₁₋₃ alkylene-O—C₁₋₃ alkylene-R, —C(O)OR, or —C(O)NR₂; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R⁵ is hydrogen; or:
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R⁶ is hydrogen, —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁷ or R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, -L⁴-Cy, or Cy;
L¹ is a covalent bond, a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or, -Cy-, —NRC(O)NR—;
each L⁴ is independently a covalent bond, a saturated or unsaturated, straight or branched, optionally substituted bivalent C₁₋₄ hydrocarbon chain, wherein 0-2 methylene units of L⁴ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
L⁵ is a covalent bond or a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R⁸ is hydrogen, a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, -L⁴-Cy, or Cy;
R¹⁰ is hydrogen, —CN, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹;
each Cy is independently an optionally substituted cyclic group which may be bivalent selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), an 8-10 membered bicyclic aromatic carbocyclic ring, a 7-12 membered saturated or partially unsaturated bicyclic heterocyclic ring that is optionally bridged bicyclic (having 1-4 heteroatoms independently selected from nitrogen, oxygen, and sulfur), an 8-10 membered bicyclic heteroaromatic ring (having 1-5 heteroatoms independently selected from nitrogen, oxygen, and sulfur), and a 5-6 membered monocyclic heteroaromatic ring (having 1-4 heteroatoms independently selected from nitrogen, oxygen, and sulfur);
R^{z} is hydrogen, —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹; and
each R is independently hydrogen, halogen or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
an R group of R^{A} and one of Z¹, Z², Z³, Z⁴, G¹, G², G³, or G⁴ are taken together with their intervening atoms to form an optionally substituted 3-7 membered heterocyclic ring; and/or
an R group of R^{A} and one of R¹, R², or R³ are taken together with their intervening atoms to form a 5-7 membered heterocyclic ring; and/or
two R groups on the same atom are taken together with their intervening atoms to form an optionally substituted 3-7 membered saturated, partially unsaturated, or heteroaryl ring (having 0-3 heteroatoms independently selected from nitrogen, oxygen, and sulfur); or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
m is 0, 1, or 2; and
n is 0, 1, or 2.

Overexpression of CDK2 is associated with abnormal regulation of the cell-cycle. The cyclin E/CDK2 complex plays an important role in regulation of the G1/S transition, histone biosynthesis and centrosome duplication. Progressive phosphorylation of retinoblastoma (Rb) by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al., Nat. Rev. Drug. Discov. 2015; 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Mol. Cancer Ther. (2012) 11:1488-99; Herrera-Abreu et al., Cancer Res. (2016) 76: 2301-2313). Cyclin E amplification also reportedly contributes to trastuzumab resistance in HER2+ breast cancer. (Scaltriti et al., Proc Natl Acad Sci. (2011) 108: 3761-6). Cyclin E overexpression has also been reported to play a role in basal-like and triple negative breast cancer (TNBC), as well as inflammatory breast cancer. (Elsawaf & Sinn, Breast Care (2011) 6:273-278; Alexander et al., Oncotarget (2017) 8: 14897-14911).

Amplification or overexpression of cyclin E1 (CCNE1) is also associated with poor outcomes in ovarian, gastric, endometrial and other cancers. (Nakayama et al., Gene amplification CCNE1 is related to poor survival and potential therapeutic target in ovarian cancer, Cancer (2010) 116: 2621-34; Etemadmoghadam et al., Clin Cancer Res (2013) 19: 5960-71; Au-Yeung et al., Clin. Cancer Res. (2017) 23:1862-1874; Ayhan et al., Modern Pathology (2017) 30: 297-303; Ooi et al., Hum Pathol. (2017) 61: 58-67; Noske et al., Oncotarget (2017) 8: 14794-14805).

There remains a need in the art for CDK inhibitors, especially selective CDK2 inhibitors, which may be useful for the treatment of cancer or other proliferative diseases or conditions. In particular, CDK2 inhibitors may be useful in treating CCNE1 or CCNE2 amplified tumors.

### 2. Compounds and Definitions

Compounds of this present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. “Bicyclic” may refer to a “bridged bicyclic” or “spirocyclic” ring. As used herein, “bridged bicyclic” rings are to be understood to be a subset of, and falling within the scope of, “bicyclic ring”. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

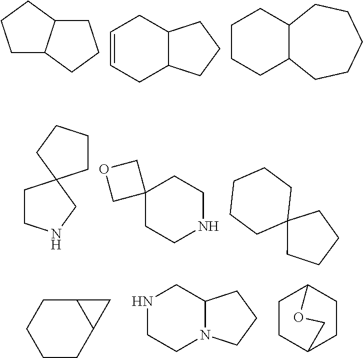

Exemplary bridged bicyclics, contemplated as falling under the scope of a “bicycle” or “bicyclic ring” include:

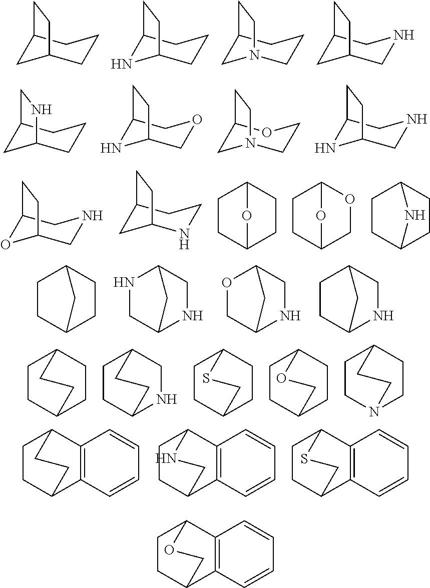

The term “Compound X” refers to 6-(1-benzyl-1H-pyrazole-4-carbonyl)-N-(3-(benzyloxy)-1-(methylamino)-1-oxobutan-2-yl)-2-(2,2-dimethylcyclopropane-1-carbonyl)-2,6-diazaspiro[3.4]octane-8-carboxamide. Compound X may also be depicted as

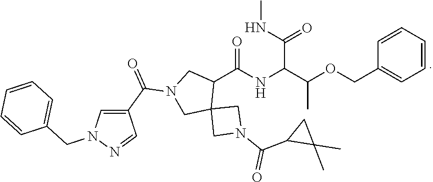 [SMILES: CNC(=O)C(NC(=O)C1CN(C(=O)c2cnn(Cc3ccccc3)c2)CC12CN(C(=O)C1CC1(C)C)C2)C(C)OCc1ccccc1]

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic or bicyclic. A heteroaryl ring may include one or more oxo (=O) or thioxo (=S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (=O) or thioxo (=S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₆R^{o}; —(CH₂)₀₋₆OR^{o}; —O(CH₂)₀₋₆R^{o}, —O—(CH₂)₀₋₆C(O)OR^{o}; —(CH₂)O—CH(OR^{o})₂; —(CH₂)₀₋₆SR^{o}; —(CH₂)₀₋₆Ph, which Ph may be substituted with R^{o}; —(CH₂)₀₋₄₆O(CH₂)₀₋₁Ph which Ph may be substituted with R^{o}; —CH═CHPh, which Ph may be substituted with R^{o}; —(CH₂)₀₋₆O(CH₂)₀₋₁-pyridyl which pyridyl may be substituted with R^{o}; —NO₂; —CN; —N₃; —(CH₂)₀₋₆N(R^{o})₂; —(CH₂)₀₋₆N(R^{o})C(O)R^{o}; —N(R^{o})C(S)R^{o}; —(CH₂)₀₋₆N(R^{o})C(O)NR^{o}₂; —N(R^{o})C(S)NR^{o}₂, —(CH₂)₀₋₆N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₆C(O)R^{o}; —C(S)R^{o}; —(CH₂)₀₋₆C(O)OR^{o}; —(CH₂)₀₋₆C(O)SR^{o}; —(CH₂)₀₋₆C(O)OSiR^{o}₃; —(CH₂)₀₋₆OC(O)R^{o}; —OC(O)(CH₂)₀₋₆SR^{o}, —(CH₂)₀₋₆SC(O)R^{o}; —(CH₂)₀₋₆C(O)NR^{o}₂; —C(S)NR^{o}₂; —C(S)SR^{o}; —SC(S)SR^{o}, —(CH₂)₀₋₆OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₆SSR^{o}; —(CH₂)₀₋₆S(O)₂R^{o}; —(CH₂)₀₋₆—S(O)₂OR^{o}; —(CH₂)₀₋₆OS(O)₂R^{o}; —S(O)₂NR^{o}₂; —(CH₂)₀₋₆S(O)R^{o}; —N(R^{o})S(O)₂NR^{o}₂; —N(R^{o})S(O)₂R^{o}; —N(OR^{o})R^{o}; —C(NH)NR^{o}₂, —P(O)₂R^{o}; —P(O)R^{o}₂, —P(O)(OR^{o})₂; —OP(O)(R^{o})OR^{o}; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5- to 6-membered heteroaryl ring), —CH₂-(5- to 6-membered saturated or partially unsaturated monocyclic heterocyclic ring [having 1-2 heteroatoms independently selected from nitrogen, oxygen, and sulfur]), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{o}, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below. An “optionally substituted” group may be substituted with one or more (i.e., from 1 to 6) substituents selected from the aforementioned monovalent substituents and one or more (i.e., from 1 to 6) suitable divalent substituents of ═O, ═S, ═NNR^{o}₂, ═NNHC(O)R^{o}, ═NNHC(O)OR^{o}, =NNHS(O)₂R^{o}, =NR^{o}, ═NOR^{o}, —O(C(R^{o}₂))₂₋₃O—, —(C(R^{o})₂)₂₋₆—, and —S(C(R^{o}₂))₂₋₃S—, wherein each of the one or more substituents is independently selected from said monovalent substituents and divalent substituents, and wherein said divalent substituents may be on a saturated carbon of an “optionally substituted” group.

Suitable monovalent substituents on R^{o} (or the ring formed by taking two independent occurrences of R^{o} together with their intervening atoms), wherein R^{o} may be substituted with one or more instances of said monovalent substituents (i.e., from 1 to 6) and suitable divalent substituents described at the end of this paragraph, and said monovalent substituents are each independently halogen, —(CH₂)₀₋₂R^{●}, -(haloR^{●}), —(CH₂)₀₋₂OH, —(CH₂)₀₋₂OR^{●}, —(CH₂)₀₋₂CH(OR)₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur). Suitable divalent substituents on a saturated carbon atom of R^{o} include ═O and =S.

Suitable divalent substituents on a saturated carbon atom of an “optionally substituted” group include the following: ═O, ═S, ═NNR*₂, ═NNHC(O)R*, ═NNHC(O)OR*, =NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(Rt)S(O)₂R^{†}; wherein each R^{†} is independently hydrogen, C₁₋₆ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of Rt, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

As used herein, the term “provided compound” or “compound of the present disclosure” refers to any genus, subgenus, and/or species set forth herein.

“One or more instances” or “one or more” as referencing substitutions, as used herein, refers to, for example, 1, 2, 3, 4, 5, 6, 7, etc. instances of substitution of functional groups, which may each be independently selected, on a chemical moiety to which “one or more” instances of substitution refers. It is to be understood that any “optionally substituted” moiety, may be substituted with “one or more” optional substituents each independently selected from those optional substituents as described herein.

“Amide isostere,” as used herein may refer to any chemical moiety with similar electronics or stereoelectronics to an amide moiety, examples include, but are not limited to, triazole, thiazole, oxadiazole, tetrazole, olefin, fluoroalkene, urea, ester, thioamide, phosphonamidate, sulfonamide, trifluoroethylamine, amidine, carbamate, and imidazole. In certain embodiments, the amide isostere is discussed in Kumari et al., J. Med. Chem. 2020, 63, 21, 12290-12358; Publication Date: Jul. 20, 2020, which is hereby incorporated by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

As used herein, the term “inhibitor” is defined as a compound that binds to and/or inhibits CDK2 with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM, when measured in an appropriate assay.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily or degratorily active metabolite or residue thereof.

As used herein, the term “inhibitorily active metabolite or residue thereof” means that a metabolite or residue thereof is also an inhibitor of a CDK2 protein, or a mutant thereof.

### 3. Description of Exemplary Embodiments

In certain embodiments, the present disclosure provides inhibitors of CDK2 activity.

In some embodiments, provided herein are compounds according to Formula I:

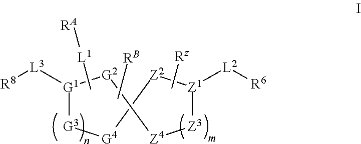

or a pharmaceutically acceptable salt thereof, wherein:
G¹ is N or CH;
each G², G³, and G⁴ is independently optionally substituted methylene, —O—, —S—, —N(R)—, —C(O)—, —S(O)—, or —S(O)₂—;
Z¹ is N or CH; and
each Z², Z³ and Z⁴ is independently optionally substituted methylene, —O—, —S—, —N(R)—, —C(O)—, —S(O)—, or —S(O)₂—;
R^{A} is an amide isostere,

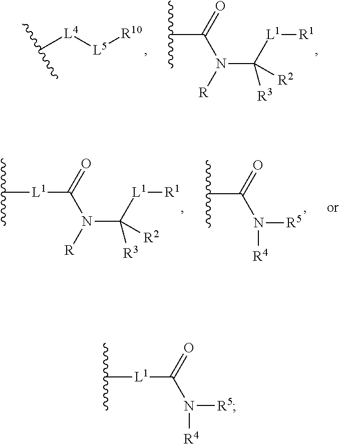

R^{B} is a hydrogen, an optionally substituted C₁₋₆ aliphatic group, or a halogen;
each L¹ is independently a covalent bond or a saturated or unsaturated, straight or branched, optionally substituted bivalent C₁₋₆ hydrocarbon chain, wherein 0-4 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
each of R² and R³ is independently hydrogen, an optionally substituted C₁₋₆ aliphatic group, —C₁₋₆ alkylene-OR, —C₁₋₃ alkylene-O—C₁₋₃ alkylene-R, —C(O)OR, or —C(O)NR₂; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R⁵ is hydrogen; or:
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R⁶ is hydrogen, —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁷ or R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, -L⁴-Cy, or Cy;
L³ is a covalent bond, a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or, -Cy-, —NRC(O)NR—;
each L⁴ is independently a covalent bond, a saturated or unsaturated, straight or branched, optionally substituted bivalent C₁₋₄ hydrocarbon chain, wherein 0-2 methylene units of L⁴ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
L⁵ is a covalent bond or a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R¹ is hydrogen, a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, -L⁴-Cy, or Cy;
R¹⁰ is hydrogen, —CN, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹;
each Cy is independently an optionally substituted cyclic group which may be bivalent selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), an 8-10 membered bicyclic aromatic carbocyclic ring, a 7-12 membered saturated or partially unsaturated bicyclic heterocyclic ring that is optionally bridged bicyclic (having 1-4 heteroatoms independently selected from nitrogen, oxygen, and sulfur), an 8-10 membered bicyclic heteroaromatic ring (having 1-5 heteroatoms independently selected from nitrogen, oxygen, and sulfur), and a 5-6 membered monocyclic heteroaromatic ring (having 1-4 heteroatoms independently selected from nitrogen, oxygen, and sulfur);
R^{z} is hydrogen, —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹; and
each R is independently hydrogen, halogen or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
an R group of R^{A} and one of Z¹, Z², Z³, Z⁴, G¹, G², G³, or G⁴ are taken together with their intervening atoms to form an optionally substituted 3-7 membered heterocyclic ring; and/or
an R group of R^{A} and one of R¹, R², or R³ are taken together with their intervening atoms to form a 5-7 membered heterocyclic ring; and/or
two R groups on the same atom are taken together with their intervening atoms to form an optionally substituted 3-7 membered saturated, partially unsaturated, or heteroaryl ring (having 0-3 heteroatoms independently selected from nitrogen, oxygen, and sulfur); or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
m is 0, 1, or 2; and
n is 0, 1, or 2.

In some embodiments, the compound is other than Compound X.

As described generally above, G¹ is N or CH. In some embodiments, G¹ is N. In some embodiments, G¹ is CH.

As described generally above, Z¹ is N or CH. In some embodiments, Z¹ is N. In some embodiments, Z¹ is CH.

In some embodiments, Z¹ is N and G¹ is N. In some embodiments, Z¹ is CH and G¹ is N.

As described generally above, each G², G³, and G⁴ is independently optionally substituted methylene, —O—, —S—, —N(R)—, —C(O)—, —S(O)—, or —S(O)₂—. In some embodiments, each G², G³, and G⁴ is methylene. In some embodiments, each G² is —O—, —S—, —N(R)—, —C(O)—, —S(O)—, or —S(O)₂— and G³ and G⁴ are methylene.

As described generally above, each Z², Z³ and Z⁴ is independently optionally substituted methylene, —O—, —S—, —N(R)—, —C(O)—, —S(O)—, or —S(O)₂—. In some embodiments, each Z², Z³ and Z⁴ is methylene.

As defined generally above, R^{A} is an amide isostere,

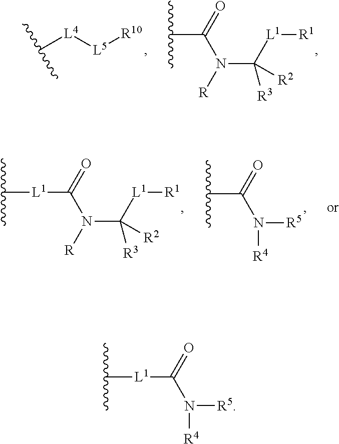

In some embodiments, R^{A} is

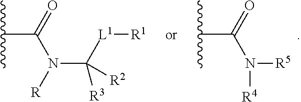

In some embodiments, R^{A} is

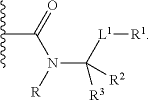

In some embodiments, R^{A} is

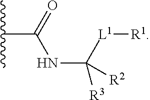

In some embodiments, R^{A} is

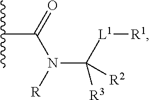

wherein the R group shown is an optionally substituted C₁₋₆ aliphatic group. In some embodiments, R^{A} is

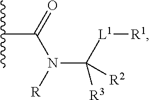

wherein the R group shown is an optionally substituted methyl group. In some embodiments, R^{A} is

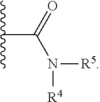

In some embodiments, R^{A} is

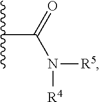

wherein R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur). In some embodiments, R^{A} is selected from those depicted in the compounds of Table 1, below.

As defined generally above, R^{B} is hydrogen, an optionally substituted C₁₋₆ aliphatic group, or a halogen. In some embodiments, R^{B} is hydrogen. In some embodiments, R^{B} is an optionally substituted C₁₋₆ aliphatic group. In some embodiments, R^{B} is a halogen. In some embodiments, R^{B} is a methyl group. In some embodiments, R^{B} is a fluoro group. In some embodiments, R^{B} is selected from those depicted in the compounds of Table 1, below.

As defined generally above, each L¹ is independently a covalent bond or a saturated or unsaturated, straight or branched, optionally substituted bivalent C₁₋₆ hydrocarbon chain, wherein 0-4 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—-Cy-, or —NRC(O)NR—. In some embodiments, L¹ is a covalent bond or a saturated or unsaturated, straight or branched, optionally substituted bivalent C₁₋₆ hydrocarbon chain, wherein 0-4 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L¹ is a covalent bond. In some embodiments, L¹ is a saturated or unsaturated, straight or branched, optionally substituted bivalent C₁₋₆ hydrocarbon chain, wherein 0-4 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O— or —NRC(O)NR—. In some embodiments, L¹ is a saturated or unsaturated, straight or branched, optionally substituted bivalent C₁₋₄ hydrocarbon chain, wherein 0-2 methylene units of L are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is a saturated or unsaturated, straight or branched, optionally substituted bivalent C₁₋₄ hydrocarbon chain. In some embodiments, L¹ is a saturated or unsaturated, straight or branched, optionally substituted bivalent C₁₋₄ hydrocarbon chain, wherein 1 or 2 methylene units of L¹ are replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is a saturated, straight or branched, optionally substituted bivalent C₁₋₄ hydrocarbon chain. In some embodiments, L¹ is a partially unsaturated, straight or branched, optionally substituted bivalent C₁₋₄ hydrocarbon chain. In some embodiments, L¹ is a saturated, straight, optionally substituted bivalent C₁₋₄ hydrocarbon chain, wherein 1-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is an optionally substituted straight or branched C₁₋₄ alkylene chain, wherein 1-2 methylene units of L¹ are independently replaced by —O—, —NR—, —S—, —C(O)O—, —C(O)—, —S(O)₂—, or —NRC(O)—. In some embodiments, L¹ is an optionally substituted straight or branched C₁₋₄ alkylene chain, wherein 1-2 methylene units of L¹ are independently replaced by —O—, —NR—, —C(O)O—, —C(O)—, or —NRC(O)—. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is an optionally substituted straight or branched C₁₋₄ alkylene chain, wherein 1-2 methylene units of L¹ are independently replaced by —O—. In some embodiments, L¹ is an optionally substituted straight or branched C₁₋₄ alkylene chain, wherein 1-2 methylene units of L¹ are independently replaced by —S—. In some embodiments, L¹ is an optionally substituted straight or branched C₁₋₄ alkylene chain, wherein 1-2 methylene units of L¹ are independently replaced by —S(O)₂—. In some embodiments, L¹ is an optionally substituted straight or branched C₁₋₄ alkylene chain, wherein 1-2 methylene units of L¹ are independently replaced by —NR—. In some embodiments, L¹ is an optionally substituted straight or branched C₁₋₄ alkylene chain, wherein 1-2 methylene units of L¹ are independently replaced by —C(O)O—. In some embodiments, L¹ is an optionally substituted straight or branched C₁₋₄ alkylene chain, wherein 1-2 methylene units of L¹ are independently replaced by —NRC(O)—. In some embodiments, L¹ is an unsubstituted straight chain C₁₋₄ alkynylene.

In some embodiments, L¹ is a covalent bond,

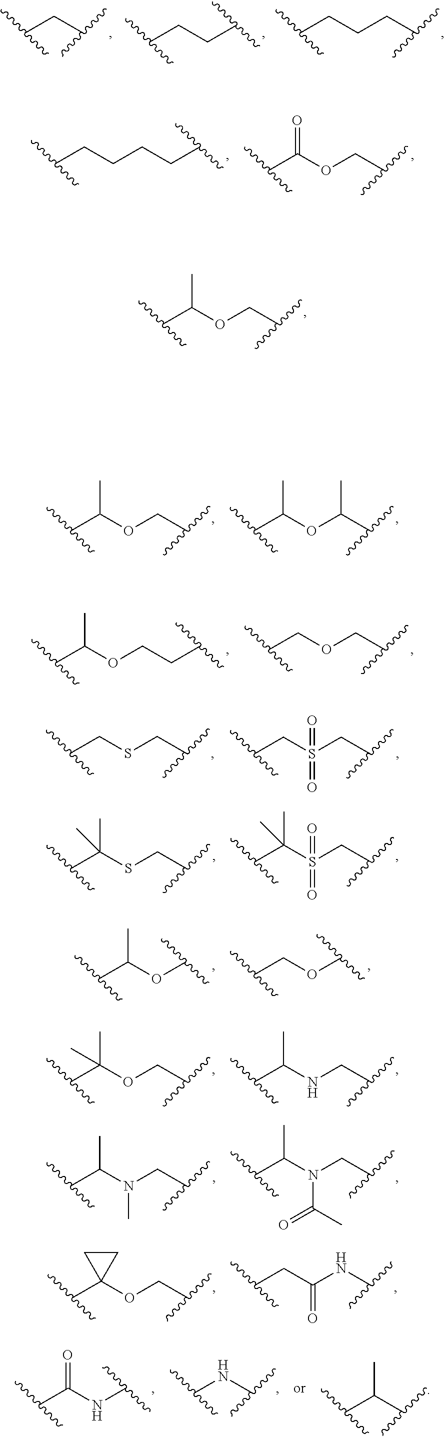

In some embodiments L¹ is

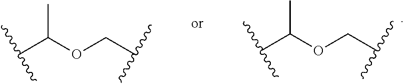

In some embodiments, L¹ is

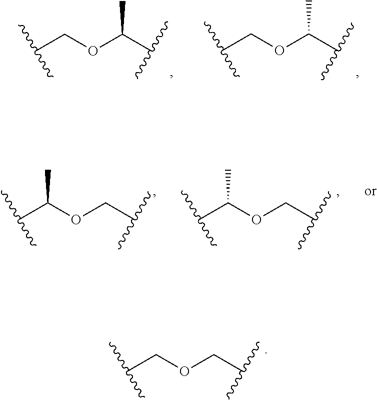

In some embodiments, L¹ is

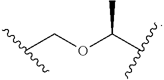 [SMILES: *CO[C@H](*)C]

In some embodiments, L¹ is

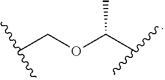 [SMILES: *CO[C@@H](*)C]

In some embodiments, L¹ is

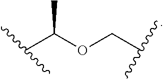 [SMILES: *CO[C@@H](*)C]

In some embodiments, L¹ is

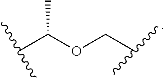 [SMILES: *CO[C@H](*)C]

In some embodiments, L¹ is

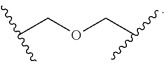 [SMILES: *COC*]

In some embodiments, L¹ is selected from those depicted in the compounds of Table 1, below.

As defined generally above, R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur).

In some embodiments, R¹ is hydrogen. In some embodiments, R¹ is an optionally substituted C₁₋₆ aliphatic group. In some embodiments, R¹ is methyl. In some embodiments, R¹ is ethyl. In some embodiments, R¹ is isopropyl.

In some embodiments, R¹ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R¹ is an optionally substituted cyclic group selected from a 3-8 membered saturated or partially unsaturated monocyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 3-8 membered saturated or partially unsaturated monocyclic heterocyclic ring (having 1-2 heteroatoms independently selected from nitrogen, oxygen, and sulfur). In some embodiments, R¹ is an optionally substituted 5-6 membered monocyclic heteroaromatic ring (having 1-4 heteroatoms independently selected from nitrogen, oxygen, and sulfur). In some embodiments, R¹ is an optionally substituted 8-10 membered bicyclic heteroaromatic ring (having 1-5 heteroatoms independently selected from nitrogen, oxygen, and sulfur). In some embodiments, R¹ is an optionally substituted cyclic group selected from phenyl, cyclohexyl, cyclopentyl, cyclobutyl, cyclopropyl, cycloheptyl, oxazolyl, pyridinyl, pyridazinyl, 1,3,4-oxadiazolyl, 1,2,3-triazolyl, pyrazolyl, and tetrahydropyranyl. In some embodiments, R¹ is optionally substituted phenyl. In some embodiments, R¹ is optionally substituted cyclohexyl.

In some embodiments, R¹ is an optionally substituted 7-12 membered saturated or partially unsaturated bicyclic heterocyclic ring that is optionally bridged bicyclic or spirocyclic (having 1-4 heteroatoms independently selected from nitrogen, oxygen, and sulfur). In some embodiments, R¹ is an optionally substituted a 7-12 membered saturated or partially unsaturated bridged bicyclic carbocyclic ring. In some embodiments, R¹ is an optionally substituted 7-12 membered bridge bicyclic carbocyclic ring or an optionally substituted 7-12 membered bridged bicyclic heterocyclic ring (having 1-4 heteroatoms independently selected from nitrogen, oxygen, and sulfur). In some embodiments, R¹ is optionally substituted oxabicyclo[2.2.2]octanyl. In some embodiments, R¹ is optionally substituted bicyclo[2.2.2]octanyl.

In some embodiments, R¹ is

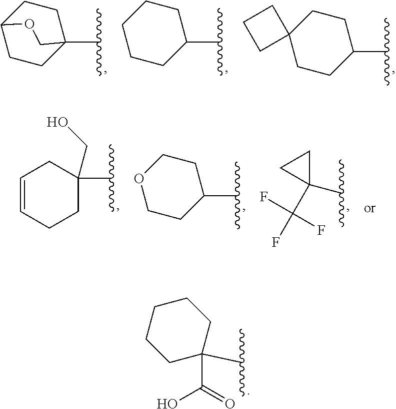

In some embodiments, R¹ is

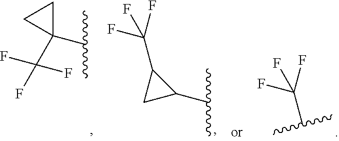

In some embodiments, R¹ is selected from those depicted in the compounds of Table 1, below.

As defined generally above, R² is hydrogen, an optionally substituted C₁₋₆ aliphatic group, —C₁₋₆ alkylene-OR, —C₁₋₃ alkylene-O—C₁₋₃ alkylene-R, —C(O)OR, —C(O)NR₂.

In some embodiments, R² is hydrogen, an optionally substituted C₁₋₆ aliphatic group, —C₁₋₆ alkylene-OR, —C₁₋₃ alkylene-O—C₁₋₃ alkylene-R, —C(O)OR, or —C(O)NR₂, and R³ is hydrogen. In some embodiments, R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur).

In some embodiments, R² is hydrogen, an optionally substituted C₁₋₆ aliphatic group, —C₁₋₆ alkylene-OR, —C₁₋₃ alkylene-O—C₁₋₃ alkylene-R, —C(O)OR, or —C(O)NR₂; and R³ is hydrogen. In some embodiments, R² is hydrogen, methyl, —CH₂OR, —CH₂OCH₂R, —C(O)OR, or —C(O)NR₂; and R³ is hydrogen. In some embodiments, R² is hydrogen. In some embodiments, R² is an optionally substituted C₁₋₆ aliphatic group. In some embodiments, R² is methyl. In some embodiments, R² is —C₁₋₆ alkylene-OR. In some embodiments, R² is —CH₂OR. In some embodiments, R² is —CH₂OCH₂R. In some embodiments, R² is —C(O)OR. In some embodiments, R² is —C(O)NR₂. In some embodiments, R² is —C(O)NR₂, wherein the two R groups, taken together with the intervening nitrogen atom, form an optionally substituted 4-7 membered saturated, partially unsaturated, or heteroaryl ring (having 0-3 heteroatoms, in addition to the nitrogen, independently selected from nitrogen, oxygen, and sulfur). In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur). In some embodiments, R² is —C(O)NR₂, wherein the two R groups, taken together with the intervening nitrogen atom, form an optionally substituted 4-7 membered saturated ring, selected from a piperidinyl, morpholinyl, piperazinyl, azetindinyl, pyrrolidinyl, azaspiro[3.3]heptanyl, and diazaspiro[3.3]heptanyl.

In some embodiments, R² is selected from those depicted in the compounds of Table 1, below.

As described generally above, R³ is hydrogen an optionally substituted C₁₋₆ aliphatic group, —C₁₋₆ alkylene-OR, —C₁₋₃ alkylene-O—C₁₋₃ alkylene-R, —C(O)OR, or —C(O)NR₂. In some embodiments, R³ is hydrogen.

In some embodiments, R³ is hydrogen and R² is a substituent in Table A:

TABLE A
Exemplary R² substituents

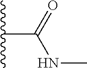 [SMILES: *C(=O)NC]

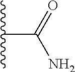 [SMILES: *C(N)=O]

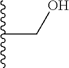 [SMILES: *CO]

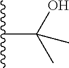 [SMILES: *C(C)(C)O]

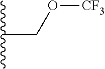 [SMILES: *COC(F)(F)F]

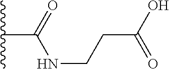 [SMILES: *C(=O)NCCC(=O)O]

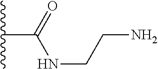 [SMILES: *C(=O)NCCN]

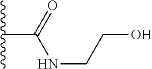 [SMILES: *C(=O)NCCO]

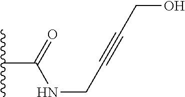 [SMILES: *C(=O)NCC#CCO]

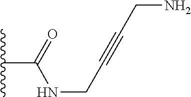 [SMILES: *C(=O)NCC#CCN]

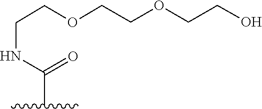 [SMILES: *C(=O)NCCOCCOCCO]

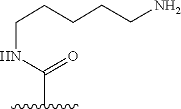 [SMILES: *C(=O)NCCCCCN]

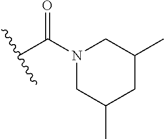 [SMILES: *C(=O)N1CC(C)CC(C)C1]

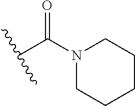 [SMILES: *C(=O)N1CCCCC1]

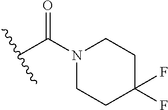 [SMILES: *C(=O)N1CCC(F)(F)CC1]

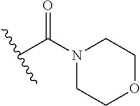 [SMILES: *C(=O)N1CCOCC1]

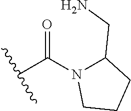 [SMILES: *C(=O)N1CCCC1CN]

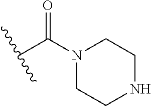 [SMILES: *C(=O)N1CCNCC1]

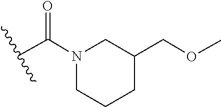 [SMILES: *C(=O)N1CCCC(COC)C1]

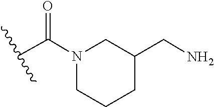 [SMILES: *C(=O)N1CCCC(CN)C1]

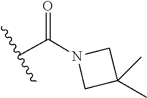 [SMILES: *C(=O)N1CC(C)(C)C1]

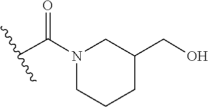 [SMILES: *C(=O)N1CCCC(CO)C1]

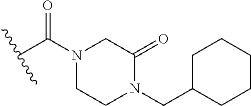 [SMILES: *C(=O)N1CCN(CC2CCCCC2)C(=O)C1]

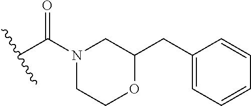 [SMILES: *C(=O)N1CCOC(Cc2ccccc2)C1]

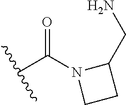 [SMILES: *C(=O)N1CCC1CN]

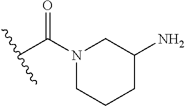 [SMILES: *C(=O)N1CCCC(N)C1]

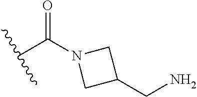 [SMILES: *C(=O)N1CC(CN)C1]

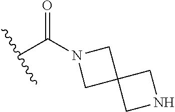 [SMILES: *C(=O)N1CC2(CNC2)C1]

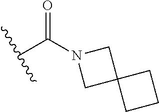 [SMILES: *C(=O)N1CC2(CCC2)C1]

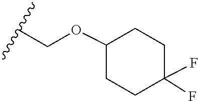 [SMILES: *COC1CCC(F)(F)CC1]

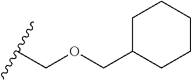 [SMILES: *COCC1CCCCC1]

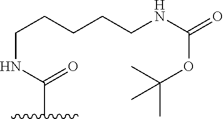 [SMILES: *C(=O)NCCCCCNC(=O)OC(C)(C)C]

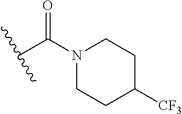 [SMILES: *C(=O)N1CCC(C(F)(F)F)CC1]

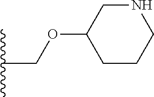 [SMILES: *COC1CCCNC1]

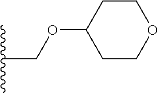 [SMILES: *COC1CCOCC1]

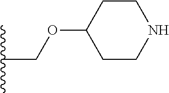 [SMILES: *COC1CCNCC1]

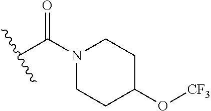 [SMILES: *C(=O)N1CCC(OC(F)(F)F)CC1]

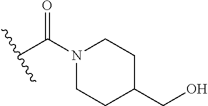 [SMILES: *C(=O)N1CCC(CO)CC1]

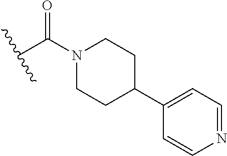 [SMILES: *C(=O)N1CCC(c2ccncc2)CC1]

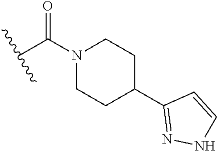 [SMILES: *C(=O)N1CCC(c2cc[nH]n2)CC1]

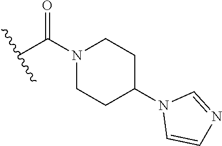 [SMILES: *C(=O)N1CCC(n2ccnc2)CC1]

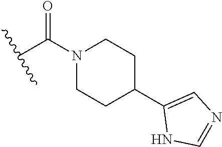 [SMILES: *C(=O)N1CCC(c2cnc[nH]2)CC1]

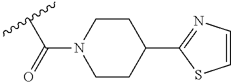 [SMILES: *C(=O)N1CCC(c2nccs2)CC1]

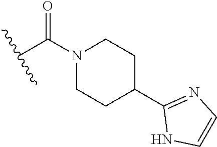 [SMILES: *C(=O)N1CCC(c2ncc[nH]2)CC1]

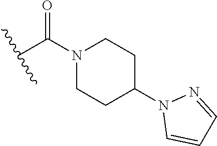 [SMILES: *C(=O)N1CCC(n2cccn2)CC1]

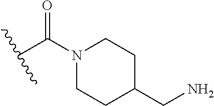 [SMILES: *C(=O)N1CCC(CN)CC1]

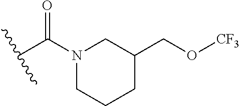 [SMILES: *C(=O)N1CCCC(COC(F)(F)F)C1]

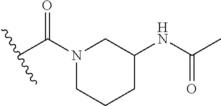 [SMILES: *C(=O)N1CCCC(NC(C)=O)C1]

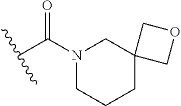 [SMILES: *C(=O)N1CCCC2(COC2)C1]

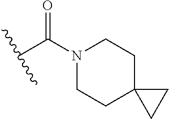 [SMILES: *C(=O)N1CCC2(CC1)CC2]

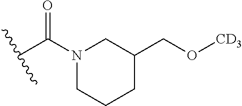 [SMILES: *C(=O)N1CCCC(COC([2H])([2H])[2H])C1]

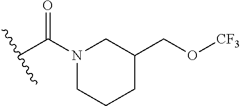 [SMILES: *C(=O)N1CCCC(COC(F)(F)F)C1]

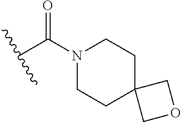 [SMILES: *C(=O)N1CCC2(CC1)COC2]

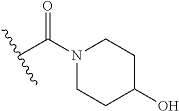 [SMILES: *C(=O)N1CCC(O)CC1]

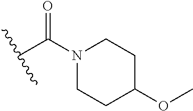 [SMILES: *C(=O)N1CCC(OC)CC1]

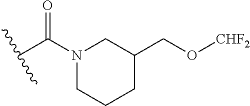 [SMILES: *C(=O)N1CCCC(COC(F)F)C1]

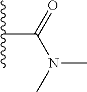 [SMILES: *C(=O)N(C)C]

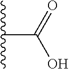 [SMILES: *C(=O)O]

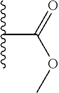 [SMILES: *C(=O)OC]

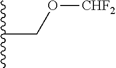 [SMILES: *COC(F)F]

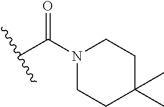 [SMILES: *C(=O)N1CCC(C)(C)CC1]

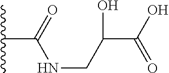 [SMILES: *C(=O)NCC(O)C(=O)O]

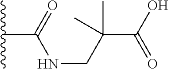 [SMILES: *C(=O)NCC(C)(C)C(=O)O]

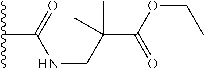 [SMILES: *C(=O)NCC(C)(C)C(=O)OCC]

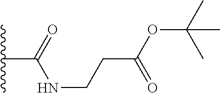 [SMILES: *C(=O)NCCC(=O)OC(C)(C)C]

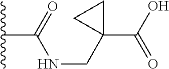 [SMILES: *C(=O)NCC1(C(=O)O)CC1]

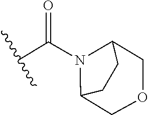 [SMILES: *C(=O)N1C2CCC1COC2]

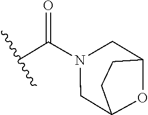 [SMILES: *C(=O)N1CC2CCC(C1)O2]

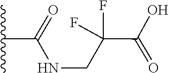 [SMILES: *C(=O)NCC(F)(F)C(=O)O]

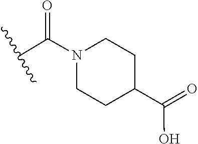 [SMILES: *C(=O)N1CCC(C(=O)O)CC1]

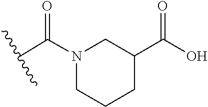 [SMILES: *C(=O)N1CCCC(C(=O)O)C1]

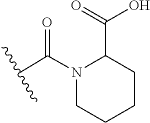 [SMILES: *C(=O)N1CCCCC1C(=O)O]

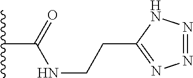 [SMILES: *C(=O)NCCc1nnn[nH]1]

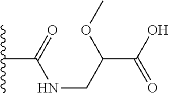 [SMILES: *C(=O)NCC(OC)C(=O)O]

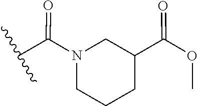 [SMILES: *C(=O)N1CCCC(C(=O)OC)C1]

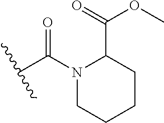 [SMILES: *C(=O)N1CCCCC1C(=O)OC]

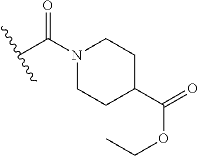 [SMILES: *C(=O)N1CCC(C(=O)OCC)CC1]

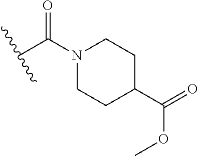 [SMILES: *C(=O)N1CCC(C(=O)OC)CC1]

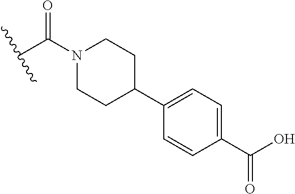 [SMILES: *C(=O)N1CCC(c2ccc(C(=O)O)cc2)CC1]

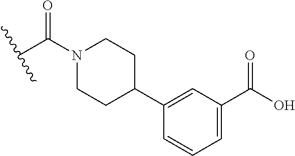 [SMILES: *C(=O)N1CCC(c2cccc(C(=O)O)c2)CC1]

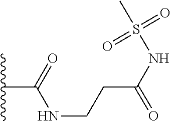 [SMILES: *C(=O)NCCC(=O)NS(C)(=O)=O]

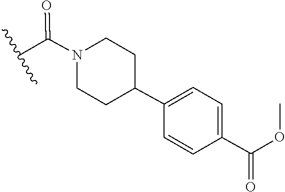 [SMILES: *C(=O)N1CCC(c2ccc(C(=O)OC)cc2)CC1]

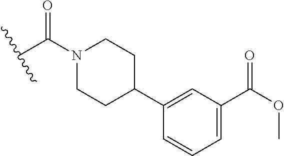 [SMILES: *C(=O)N1CCC(c2cccc(C(=O)OC)c2)CC1]

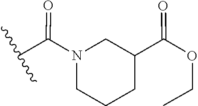 [SMILES: *C(=O)N1CCCC(C(=O)OCC)C1]

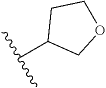 [SMILES: *C1CCOC1]

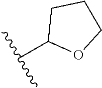 [SMILES: *C1CCCO1]

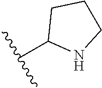 [SMILES: *C1CCCN1]

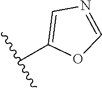 [SMILES: *c1cnco1]

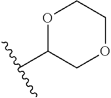 [SMILES: *C1COCCO1]

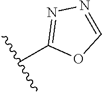 [SMILES: *c1nnco1]

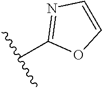 [SMILES: *c1ncco1]

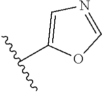 [SMILES: *c1cnco1]

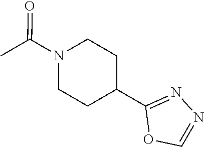

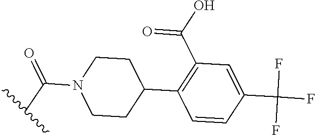 [SMILES: *C(=O)N1CCC(c2ccc(C(F)(F)F)cc2C(=O)O)CC1]

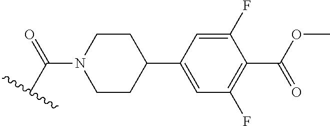 [SMILES: *C(=O)N1CCC(c2cc(F)c(C(=O)OC)c(F)c2)CC1]

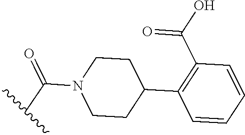 [SMILES: *C(=O)N1CCC(c2ccccc2C(=O)O)CC1]

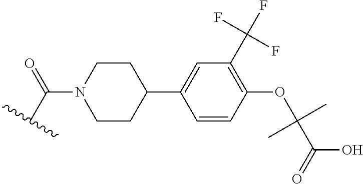 [SMILES: *C(=O)N1CCC(c2ccc(OC(C)(C)C(=O)O)c(C(F)(F)F)c2)CC1]

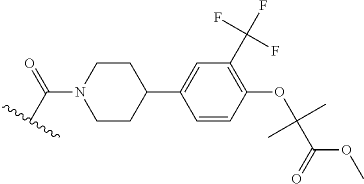 [SMILES: *C(=O)N1CCC(c2ccc(OC(C)(C)C(=O)OC)c(C(F)(F)F)c2)CC1]

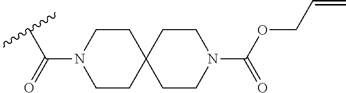 [SMILES: *C(=O)N1CCC2(CC1)CCN(C(=O)OCC=C)CC2]

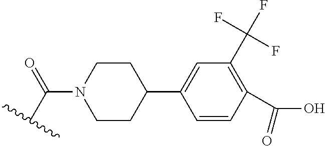 [SMILES: *C(=O)N1CCC(c2ccc(C(=O)O)c(C(F)(F)F)c2)CC1]

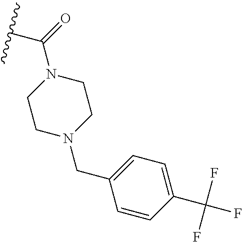 [SMILES: *C(=O)N1CCN(Cc2ccc(C(F)(F)F)cc2)CC1]

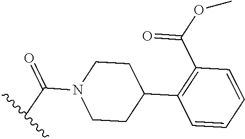 [SMILES: *C(=O)N1CCC(c2ccccc2C(=O)OC)CC1]

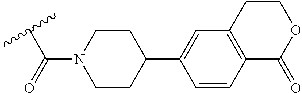 [SMILES: *C(=O)N1CCC(c2ccc3c(c2)CCOC3=O)CC1]

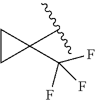 [SMILES: *C1(C(F)(F)F)CC1]

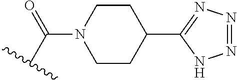 [SMILES: *C(=O)N1CCC(c2nnn[nH]2)CC1]

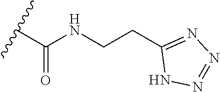 [SMILES: *C(=O)NCCc1nnn[nH]1]

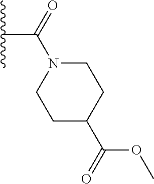 [SMILES: *C(=O)N1CCC(C(=O)OC)CC1]

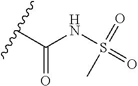 [SMILES: *C(=O)NS(C)(=O)=O]

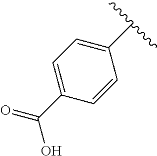 [SMILES: *c1ccc(C(=O)O)cc1]

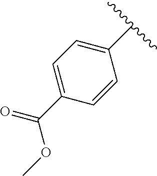 [SMILES: *c1ccc(C(=O)OC)cc1]

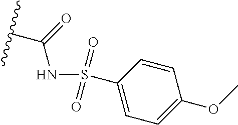 [SMILES: *C(=O)NS(=O)(=O)c1ccc(OC)cc1]

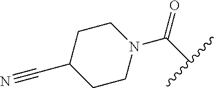 [SMILES: *C(=O)N1CCC(C#N)CC1]

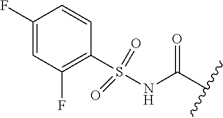 [SMILES: *C(=O)NS(=O)(=O)c1ccc(F)cc1F]

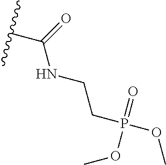 [SMILES: *C(=O)NCCP(=O)(OC)OC]

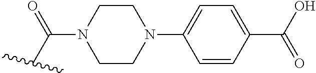 [SMILES: *C(=O)N1CCN(c2ccc(C(=O)O)cc2)CC1]

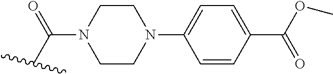 [SMILES: *C(=O)N1CCN(c2ccc(C(=O)OC)cc2)CC1]

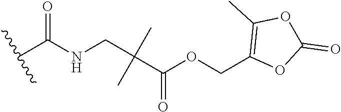 [SMILES: *C(=O)NCC(C)(C)C(=O)OCc1oc(=O)oc1C]

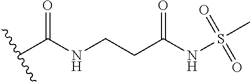 [SMILES: *C(=O)NCCC(=O)NS(C)(=O)=O]

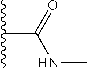 [SMILES: *C(=O)NC]

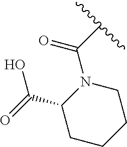 [SMILES: *C(=O)N1CCCC[C@@H]1C(=O)O]

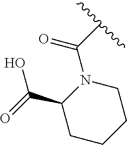 [SMILES: *C(=O)N1CCCC[C@H]1C(=O)O]

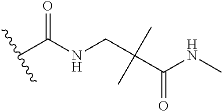 [SMILES: *C(=O)NCC(C)(C)C(=O)NC]

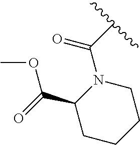 [SMILES: *C(=O)N1CCCC[C@H]1C(=O)OC]

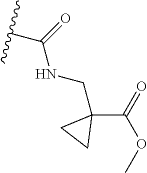 [SMILES: *C(=O)NCC1(C(=O)OC)CC1]

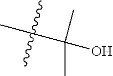 [SMILES: *C(C)(C)O]

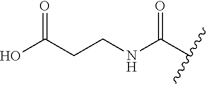 [SMILES: *C(=O)NCCC(=O)O]

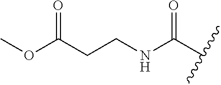 [SMILES: *C(=O)NCCC(=O)OC]

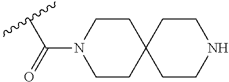 [SMILES: *C(=O)N1CCC2(CCNCC2)CC1]

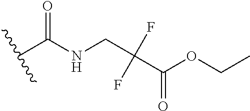 [SMILES: *C(=O)NCC(F)(F)C(=O)OCC]

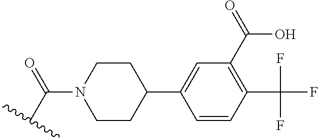 [SMILES: *C(=O)N1CCC(c2ccc(C(F)(F)F)c(C(=O)O)c2)CC1]

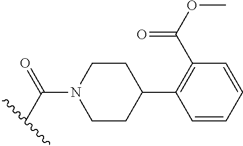 [SMILES: *C(=O)N1CCC(c2ccccc2C(=O)OC)CC1]

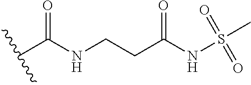 [SMILES: *C(=O)NCCC(=O)NS(C)(=O)=O]

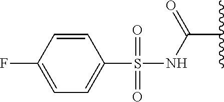 [SMILES: *C(=O)NS(=O)(=O)c1ccc(F)cc1]

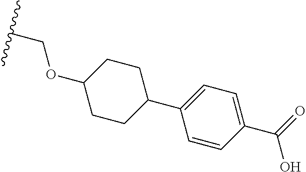 [SMILES: *COC1CCC(c2ccc(C(=O)O)cc2)CC1]

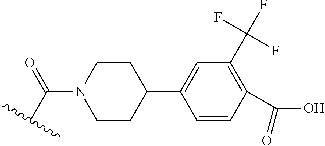 [SMILES: *C(=O)N1CCC(c2ccc(C(=O)O)c(C(F)(F)F)c2)CC1]

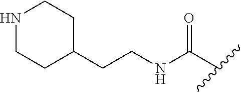 [SMILES: *C(=O)NCCC1CCNCC1]

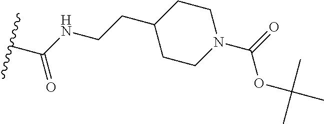 [SMILES: *C(=O)NCCC1CCN(C(=O)OC(C)(C)C)CC1]

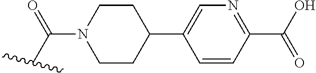 [SMILES: *C(=O)N1CCC(c2ccc(C(=O)O)nc2)CC1]

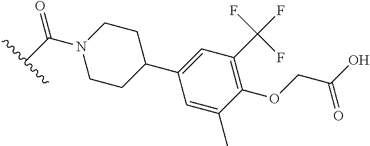 [SMILES: *C(=O)N1CCC(c2cc(C)c(OCC(=O)O)c(C(F)(F)F)c2)CC1]

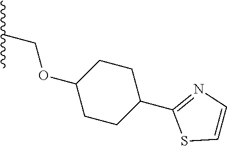 [SMILES: *COC1CCC(c2nccs2)CC1]

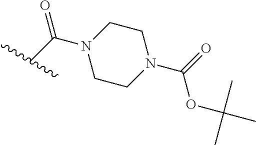 [SMILES: *C(=O)N1CCN(C(=O)OC(C)(C)C)CC1]

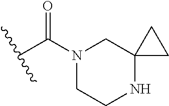 [SMILES: *C(=O)N1CCNC2(CC2)C1]

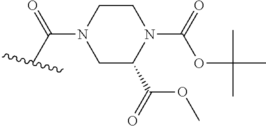 [SMILES: *C(=O)N1CCN(C(=O)OC(C)(C)C)[C@H](C(=O)OC)C1]

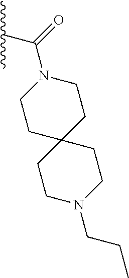 [SMILES: *C(=O)N1CCC2(CCN(CCC)CC2)CC1]

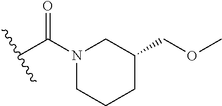 [SMILES: *C(=O)N1CCC[C@@H](COC)C1]

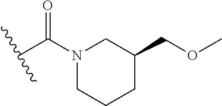 [SMILES: *C(=O)N1CCC[C@H](COC)C1]

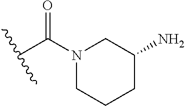 [SMILES: *C(=O)N1CCC[C@@H](N)C1]

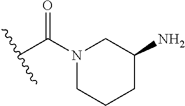 [SMILES: *C(=O)N1CCC[C@H](N)C1]

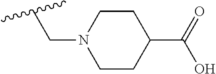 [SMILES: *CN1CCC(C(=O)O)CC1]

In some embodiments, R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur). In some embodiments, R² and R³ together with the intervening carbon atom form an optionally substituted 3-7 membered saturated or partially unsaturated carbocyclic ring. In some embodiments, R² and R³ together with the intervening carbon atom form an optionally substituted 3-7 membered saturated carbocyclic ring. In some embodiments, R² and R³ together with the intervening carbon atom form an optionally substituted 3-7 membered saturated or partially unsaturated heterocyclic ring (having 1-2 heteroatoms independently selected from nitrogen, oxygen, and sulfur). In some embodiments, R² and R³ together with the intervening carbon atom form an optionally substituted 3-7 membered saturated heterocyclic ring (having 1-2 heteroatoms independently selected from nitrogen, oxygen, and sulfur). In some embodiments, R² and R³ together with the intervening carbon atom form an optionally substituted oxetanyl, cyclopropyl, cyclobutyl, cyclopentyl, tetrahydropyranyl, piperidinyl, piperazinyl, morpholinyl, pyrrolidinyl, or 1,4-oxazepanyl. In some embodiments, R² and R³ form a cyclic group selected from those depicted in the compounds of Table 1, below.

As defined generally above, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), and R⁵ is hydrogen; or R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, independently selected from nitrogen, oxygen, and sulfur).

In some embodiments, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), and R⁵ is hydrogen. In some embodiments, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur). In some embodiments, R⁴ is an optionally substituted 3-8 membered saturated or partially unsaturated monocyclic carbocyclic ring. In some embodiments, R⁴ is an optionally substituted 7-12 membered saturated or partially unsaturated bicyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 3-8 membered saturated or partially unsaturated monocyclic heterocyclic ring (having 1-2 heteroatoms independently selected from nitrogen, oxygen, and sulfur). In some embodiments, R⁴ is an optionally substituted 5-6 membered monocyclic heteroaromatic ring (having 1-4 heteroatoms independently selected from nitrogen, oxygen, and sulfur). In some embodiments, R⁴ is an optionally substituted cyclic group selected from phenyl, piperidinyl, tetrahydropyranyl, 1,4-oxazepanyl, oxazolyl, cyclobutyl, cyclopentyl, or pyrrolidinyl. In some embodiments, R⁴ is selected from those depicted in the compounds of Table 1, below.

In some embodiments, R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur). In some embodiments, R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur). In some embodiments, R⁴ and R⁵ together with the intervening nitrogen atom form an optionally substituted heteroaryl ring (having 0-3 heteroatoms, in addition to the nitrogen, independently selected from nitrogen, oxygen, and sulfur).

In some embodiments, R⁴ and R⁵ together with the intervening nitrogen atom form an optionally substituted 6 membered saturated heterocyclic ring (having 0 or 1 additional nitrogen atoms, in addition to the intervening nitrogen). In some embodiments, R⁴ and R⁵ together with the intervening nitrogen atom form an optionally substituted 6 membered saturated heterocyclic ring.

In some embodiments, R⁴ and R⁵ together with the intervening nitrogen atom form an optionally substituted 6 membered saturated heterocyclic ring (having 1 additional nitrogen atom, in addition to the intervening nitrogen).

In some embodiments, R⁴ and R⁵ together with the intervening nitrogen atom form an optionally substituted cyclic group selected from piperindinyl, piperazinyl, morpholinyl, and pyrrolidinyl. In some embodiments, R⁴ and R⁵ together with the intervening nitrogen atom form a substituted cyclic group, wherein the cyclic group is substituted with one or more groups selected from —C₁₋₆ alkylene-phenyl, —O—C₁₋₆ alkylene-phenyl, —C₁₋₆ alkylene-cyclohexyl, —O—C₁₋₆ alkylene-cyclohexyl, —C₁₋₆ alkylene-COOH, —C₁₋₆ alkylene-C(O)O—(C₁₋₄alkyl), —C₁₋₆ alkylene-C(O)NHS(O)₂-(C₁₋₄alkyl). In some embodiments, R⁴ and R⁵ form a cyclic group selected from those depicted in the compounds of Table 1, below.

In some embodiments, R^{A} is a substituent of Table Bi, Bii, or Biii:

TABLE Bi
Exemplary R^{A} substituents

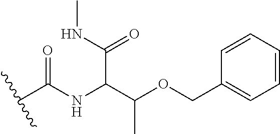 [SMILES: *C(=O)NC(C(=O)NC)C(C)OCc1ccccc1]

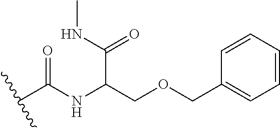 [SMILES: *C(=O)NC(COCc1ccccc1)C(=O)NC]

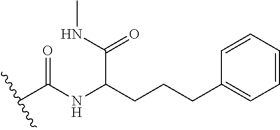 [SMILES: *C(=O)NC(CCCc1ccccc1)C(=O)NC]

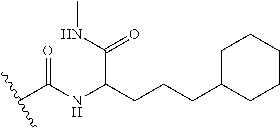 [SMILES: *C(=O)NC(CCCC1CCCCC1)C(=O)NC]

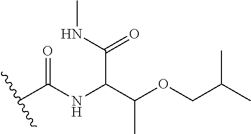 [SMILES: *C(=O)NC(C(=O)NC)C(C)OCC(C)C]

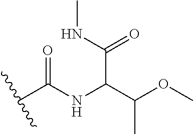 [SMILES: *C(=O)NC(C(=O)NC)C(C)OC]

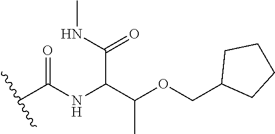 [SMILES: *C(=O)NC(C(=O)NC)C(C)OCC1CCCC1]

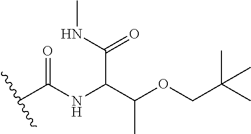 [SMILES: *C(=O)NC(C(=O)NC)C(C)OCC(C)(C)C]

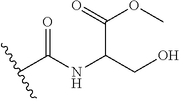 [SMILES: *C(=O)NC(CO)C(=O)OC]

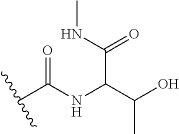 [SMILES: *C(=O)NC(C(=O)NC)C(C)O]

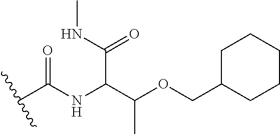 [SMILES: *C(=O)NC(C(=O)NC)C(C)OCC1CCCCC1]

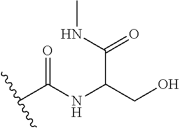 [SMILES: *C(=O)NC(CO)C(=O)NC]

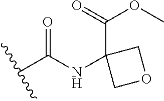 [SMILES: *C(=O)NC1(C(=O)OC)COC1]

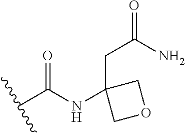 [SMILES: *C(=O)NC1(CC(N)=O)COC1]

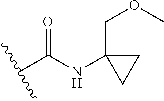 [SMILES: *C(=O)NC1(COC)CC1]

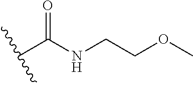 [SMILES: *C(=O)NCCOC]

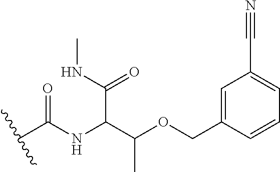 [SMILES: *C(=O)NC(C(=O)NC)C(C)OCc1cccc(C#N)c1]

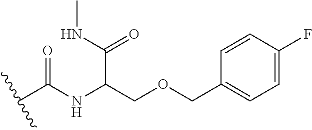 [SMILES: *C(=O)NC(COCc1ccc(F)cc1)C(=O)NC]

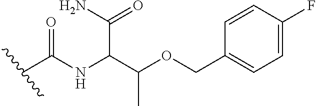 [SMILES: *C(=O)NC(C(N)=O)C(C)OCc1ccc(F)cc1]

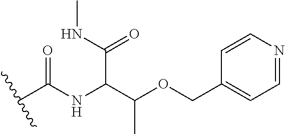 [SMILES: *C(=O)NC(C(=O)NC)C(C)OCc1ccncc1]

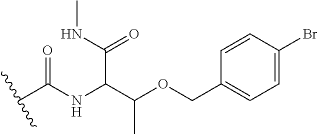 [SMILES: *C(=O)NC(C(=O)NC)C(C)OCc1ccc(Br)cc1]

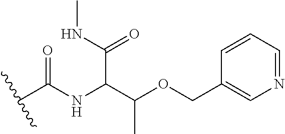 [SMILES: *C(=O)NC(C(=O)NC)C(C)OCc1cccnc1]

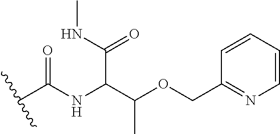 [SMILES: *C(=O)NC(C(=O)NC)C(C)OCc1ccccn1]

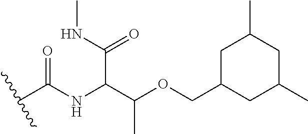 [SMILES: *C(=O)NC(C(=O)NC)C(C)OCC1CC(C)CC(C)C1]

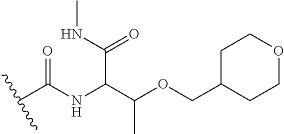 [SMILES: *C(=O)NC(C(=O)NC)C(C)OCC1CCOCC1]

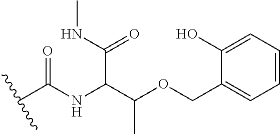 [SMILES: *C(=O)NC(C(=O)NC)C(C)OCc1ccccc1O]

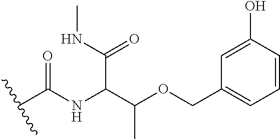 [SMILES: *C(=O)NC(C(=O)NC)C(C)OCc1cccc(O)c1]

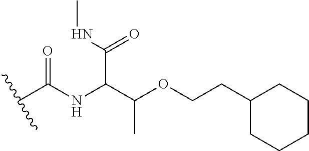 [SMILES: *C(=O)NC(C(=O)NC)C(C)OCCC1CCCCC1]

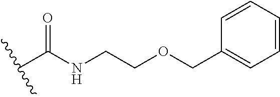 [SMILES: *C(=O)NCCOCc1ccccc1]

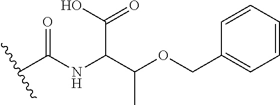 [SMILES: *C(=O)NC(C(=O)O)C(C)OCc1ccccc1]

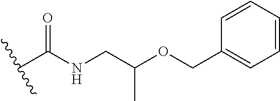 [SMILES: *C(=O)NCC(C)OCc1ccccc1]

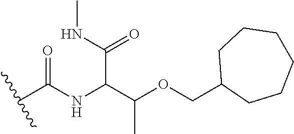 [SMILES: *C(=O)NC(C(=O)NC)C(C)OCC1CCCCCC1]

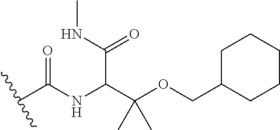 [SMILES: *C(=O)NC(C(=O)NC)C(C)(C)OCC1CCCCC1]

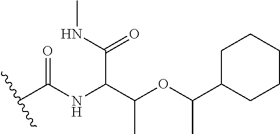 [SMILES: *C(=O)NC(C(=O)NC)C(C)OC(C)C1CCCCC1]

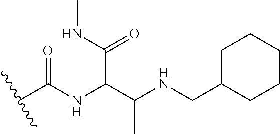 [SMILES: *C(=O)NC(C(=O)NC)C(C)NCC1CCCCC1]

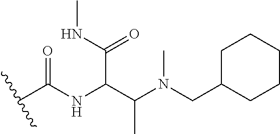 [SMILES: *C(=O)NC(C(=O)NC)C(C)N(C)CC1CCCCC1]

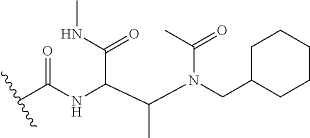 [SMILES: *C(=O)NC(C(=O)NC)C(C)N(CC1CCCCC1)C(C)=O]

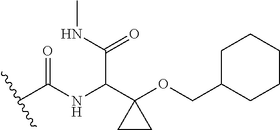 [SMILES: *C(=O)NC(C(=O)NC)C1(OCC2CCCCC2)CC1]

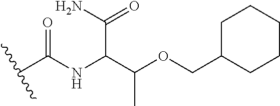 [SMILES: *C(=O)NC(C(N)=O)C(C)OCC1CCCCC1]

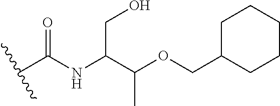 [SMILES: *C(=O)NC(CO)C(C)OCC1CCCCC1]

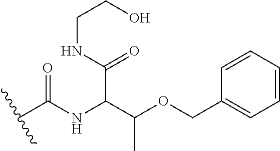 [SMILES: *C(=O)NC(C(=O)NCCO)C(C)OCc1ccccc1]

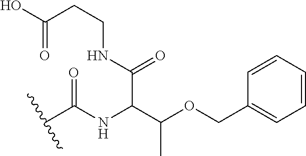 [SMILES: *C(=O)NC(C(=O)NCCC(=O)O)C(C)OCc1ccccc1]

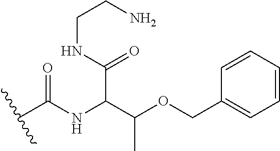 [SMILES: *C(=O)NC(C(=O)NCCN)C(C)OCc1ccccc1]

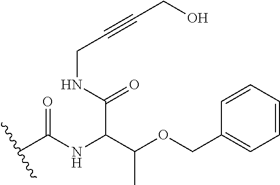 [SMILES: *C(=O)NC(C(=O)NCC#CCO)C(C)OCc1ccccc1]

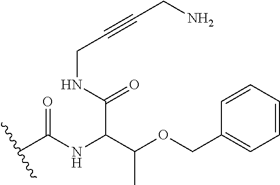 [SMILES: *C(=O)NC(C(=O)NCC#CCN)C(C)OCc1ccccc1]

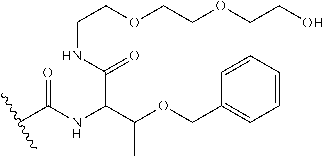 [SMILES: *C(=O)NC(C(=O)NCCOCCOCCO)C(C)OCc1ccccc1]

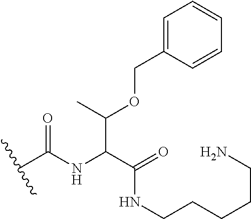 [SMILES: *C(=O)NC(C(=O)NCCCCCN)C(C)OCc1ccccc1]

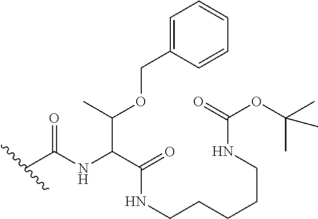 [SMILES: *C(=O)NC(C(=O)NCCCCCNC(=O)OC(C)(C)C)C(C)OCc1ccccc1]

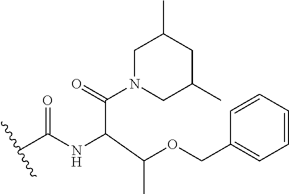 [SMILES: *C(=O)NC(C(=O)N1CC(C)CC(C)C1)C(C)OCc1ccccc1]

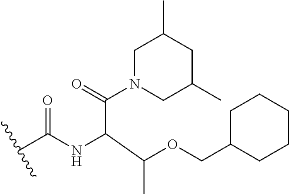 [SMILES: *C(=O)NC(C(=O)N1CC(C)CC(C)C1)C(C)OCC1CCCCC1]

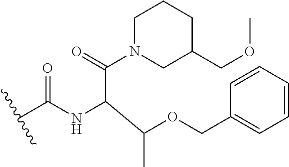 [SMILES: *C(=O)NC(C(=O)N1CCCC(COC)C1)C(C)OCc1ccccc1]

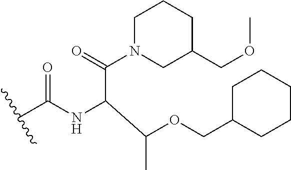 [SMILES: *C(=O)NC(C(=O)N1CCCC(COC)C1)C(C)OCC1CCCCC1]

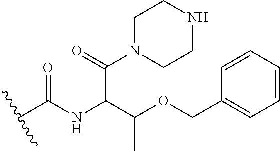 [SMILES: *C(=O)NC(C(=O)N1CCNCC1)C(C)OCc1ccccc1]

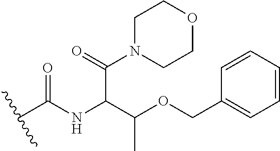 [SMILES: *C(=O)NC(C(=O)N1CCOCC1)C(C)OCc1ccccc1]

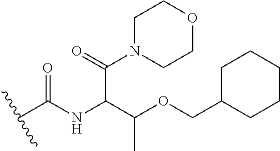 [SMILES: *C(=O)NC(C(=O)N1CCOCC1)C(C)OCC1CCCCC1]

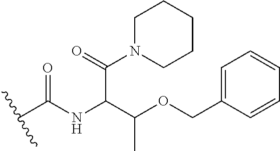 [SMILES: *C(=O)NC(C(=O)N1CCCCC1)C(C)OCc1ccccc1]

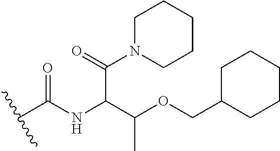 [SMILES: *C(=O)NC(C(=O)N1CCCCC1)C(C)OCC1CCCCC1]

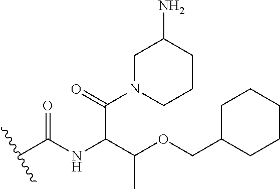 [SMILES: *C(=O)NC(C(=O)N1CCCC(N)C1)C(C)OCC1CCCCC1]

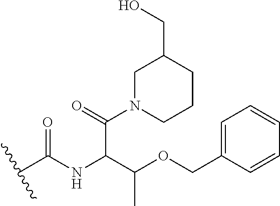 [SMILES: *C(=O)NC(C(=O)N1CCCC(CO)C1)C(C)OCc1ccccc1]

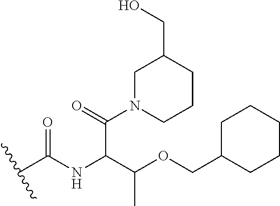 [SMILES: *C(=O)NC(C(=O)N1CCCC(CO)C1)C(C)OCC1CCCCC1]

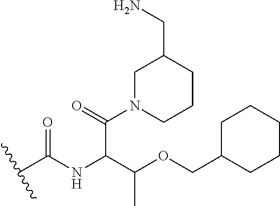 [SMILES: *C(=O)NC(C(=O)N1CCCC(CN)C1)C(C)OCC1CCCCC1]

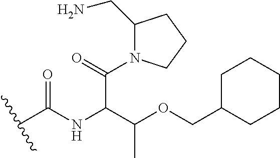 [SMILES: *C(=O)NC(C(=O)N1CCCC1CN)C(C)OCC1CCCCC1]

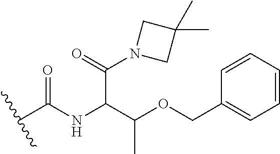

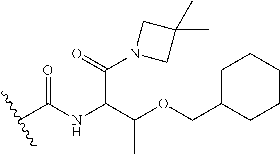 [SMILES: *C(=O)NC(C(=O)N1CC(C)(C)C1)C(C)OCC1CCCCC1]

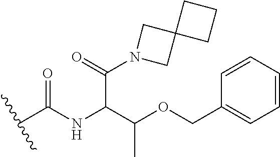 [SMILES: *C(=O)NC(C(=O)N1CC2(CCC2)C1)C(C)OCc1ccccc1]

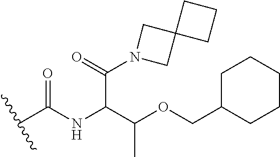 [SMILES: *C(=O)NC(C(=O)N1CC2(CCC2)C1)C(C)OCC1CCCCC1]

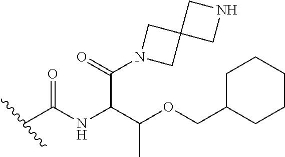 [SMILES: *C(=O)NC(C(=O)N1CC2(CNC2)C1)C(C)OCC1CCCCC1]

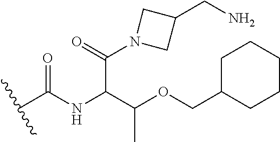 [SMILES: *C(=O)NC(C(=O)N1CC(CN)C1)C(C)OCC1CCCCC1]

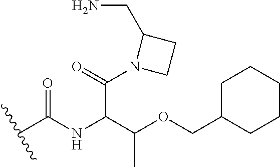 [SMILES: *C(=O)NC(C(=O)N1CCC1CN)C(C)OCC1CCCCC1]

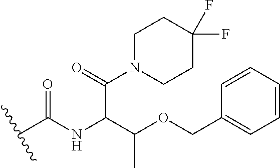 [SMILES: *C(=O)NC(C(=O)N1CCC(F)(F)CC1)C(C)OCc1ccccc1]

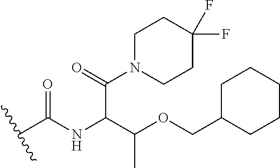 [SMILES: *C(=O)NC(C(=O)N1CCC(F)(F)CC1)C(C)OCC1CCCCC1]

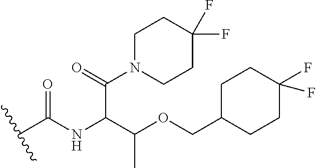 [SMILES: *C(=O)NC(C(=O)N1CCC(F)(F)CC1)C(C)OCC1CCC(F)(F)CC1]

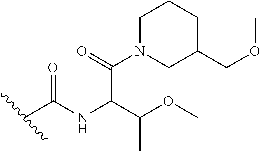 [SMILES: *C(=O)NC(C(=O)N1CCCC(COC)C1)C(C)OC]

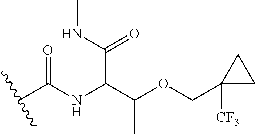 [SMILES: *C(=O)NC(C(=O)NC)C(C)OCC1(C(F)(F)F)CC1]

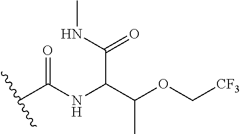 [SMILES: *C(=O)NC(C(=O)NC)C(C)OCC(F)(F)F]

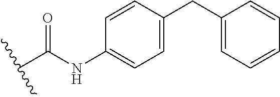 [SMILES: *C(=O)Nc1ccc(Cc2ccccc2)cc1]

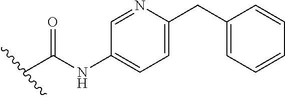 [SMILES: *C(=O)Nc1ccc(Cc2ccccc2)nc1]

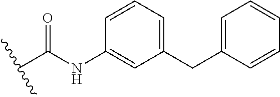 [SMILES: *C(=O)Nc1cccc(Cc2ccccc2)c1]

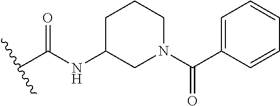 [SMILES: *C(=O)NC1CCCN(C(=O)c2ccccc2)C1]

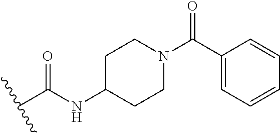 [SMILES: *C(=O)NC1CCN(C(=O)c2ccccc2)CC1]

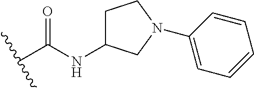 [SMILES: *C(=O)NC1CCN(c2ccccc2)C1]

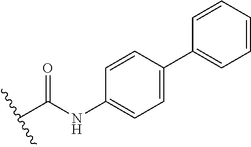 [SMILES: *C(=O)Nc1ccc(-c2ccccc2)cc1]

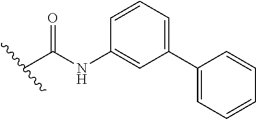 [SMILES: *C(=O)Nc1cccc(-c2ccccc2)c1]

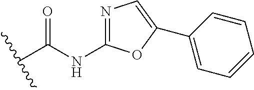 [SMILES: *C(=O)Nc1ncc(-c2ccccc2)o1]

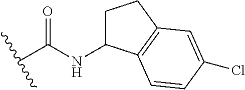 [SMILES: *C(=O)NC1CCc2cc(Cl)ccc21]

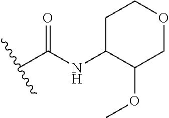 [SMILES: *C(=O)NC1CCOCC1OC]

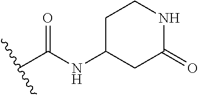 [SMILES: *C(=O)NC1CCNC(=O)C1]

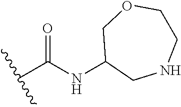 [SMILES: *C(=O)NC1CNCCOC1]

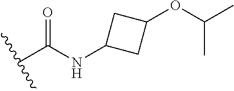 [SMILES: *C(=O)NC1CC(OC(C)C)C1]

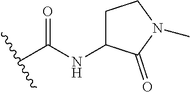 [SMILES: *C(=O)NC1CCN(C)C1=O]

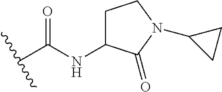 [SMILES: *C(=O)NC1CCN(C2CC2)C1=O]

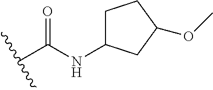 [SMILES: *C(=O)NC1CCC(OC)C1]

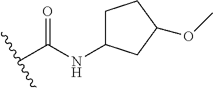 [SMILES: *C(=O)NC1CCC(OC)C1]

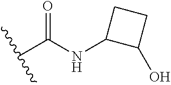 [SMILES: *C(=O)NC1CCC1O]

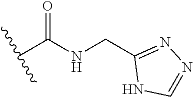 [SMILES: *C(=O)NCc1nnc[nH]1]

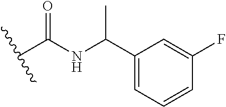 [SMILES: *C(=O)NC(C)c1cccc(F)c1]

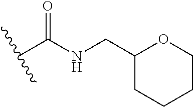 [SMILES: *C(=O)NCC1CCCCO1]

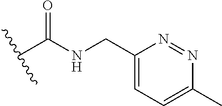 [SMILES: *C(=O)NCc1ccc(C)nn1]

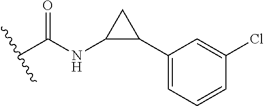 [SMILES: *C(=O)NC1CC1c1cccc(Cl)c1]

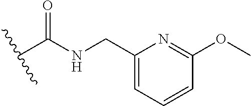 [SMILES: *C(=O)NCc1cccc(OC)n1]

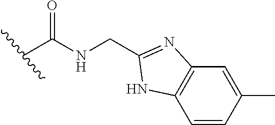 [SMILES: *C(=O)NCc1nc2cc(C)ccc2[nH]1]

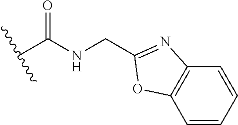 [SMILES: *C(=O)NCc1nc2ccccc2o1]

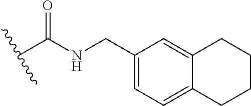 [SMILES: *C(=O)NCc1ccc2c(c1)CCCC2]

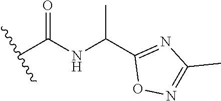 [SMILES: *C(=O)NC(C)c1nc(C)no1]

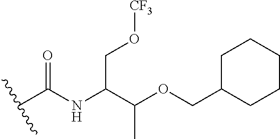 [SMILES: *C(=O)NC(COC(F)(F)F)C(C)OCC1CCCCC1]

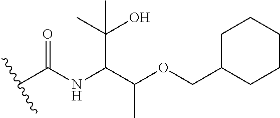 [SMILES: *C(=O)NC(C(C)OCC1CCCCC1)C(C)(C)O]

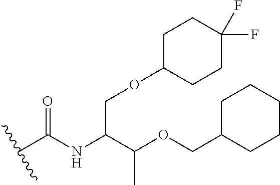 [SMILES: *C(=O)NC(COC1CCC(F)(F)CC1)C(C)OCC1CCCCC1]

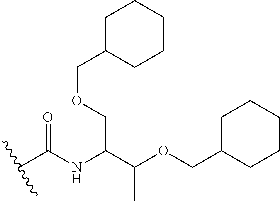 [SMILES: *C(=O)NC(COCC1CCCCC1)C(C)OCC1CCCCC1]

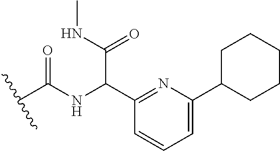 [SMILES: *C(=O)NC(C(=O)NC)c1cccc(C2CCCCC2)n1]

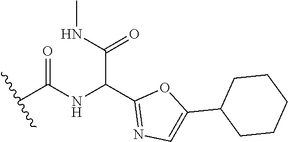 [SMILES: *C(=O)NC(C(=O)NC)c1ncc(C2CCCCC2)o1]

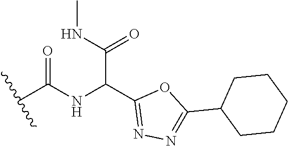 [SMILES: *C(=O)NC(C(=O)NC)c1nnc(C2CCCCC2)o1]

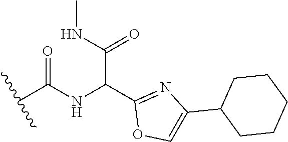 [SMILES: *C(=O)NC(C(=O)NC)c1nc(C2CCCCC2)co1]

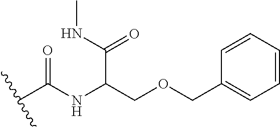 [SMILES: *C(=O)NC(COCc1ccccc1)C(=O)NC]

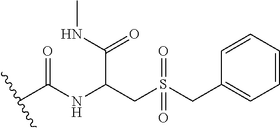 [SMILES: *C(=O)NC(CS(=O)(=O)Cc1ccccc1)C(=O)NC]

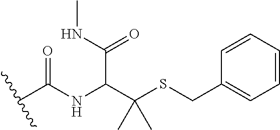 [SMILES: *C(=O)NC(C(=O)NC)C(C)(C)SCc1ccccc1]

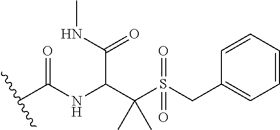 [SMILES: *C(=O)NC(C(=O)NC)C(C)(C)S(=O)(=O)Cc1ccccc1]

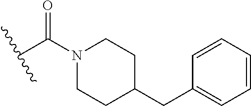 [SMILES: *C(=O)N1CCC(Cc2ccccc2)CC1]

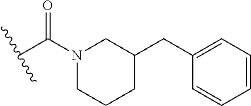 [SMILES: *C(=O)N1CCCC(Cc2ccccc2)C1]

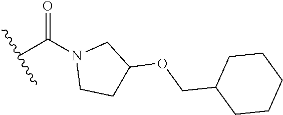 [SMILES: *C(=O)N1CCC(OCC2CCCCC2)C1]

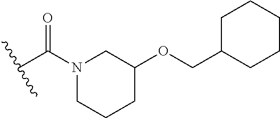 [SMILES: *C(=O)N1CCCC(OCC2CCCCC2)C1]

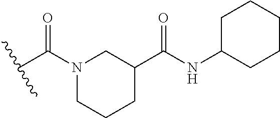 [SMILES: *C(=O)N1CCCC(C(=O)NC2CCCCC2)C1]

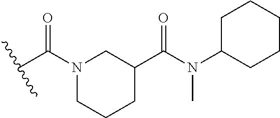 [SMILES: *C(=O)N1CCCC(C(=O)N(C)C2CCCCC2)C1]

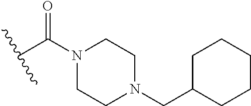 [SMILES: *C(=O)N1CCN(CC2CCCCC2)CC1]

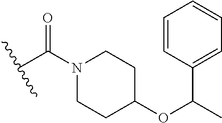 [SMILES: *C(=O)N1CCC(OC(C)c2ccccc2)CC1]

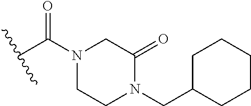 [SMILES: *C(=O)N1CCN(CC2CCCCC2)C(=O)C1]

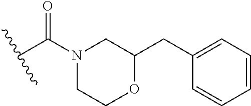 [SMILES: *C(=O)N1CCOC(Cc2ccccc2)C1]

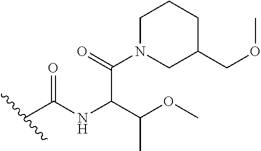 [SMILES: *C(=O)NC(C(=O)N1CCCC(COC)C1)C(C)OC]

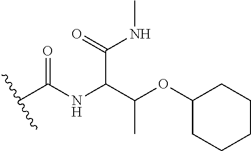 [SMILES: *C(=O)NC(C(=O)NC)C(C)OC1CCCCC1]

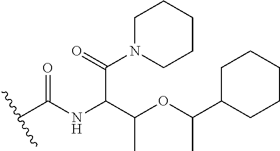 [SMILES: *C(=O)NC(C(=O)N1CCCCC1)C(C)OC(C)C1CCCCC1]

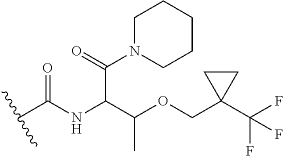 [SMILES: *C(=O)NC(C(=O)N1CCCCC1)C(C)OCC1(C(F)(F)F)CC1]

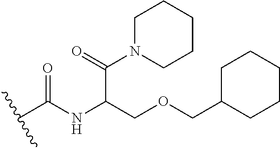 [SMILES: *C(=O)NC(COCC1CCCCC1)C(=O)N1CCCCC1]

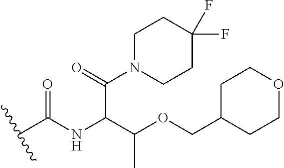 [SMILES: *C(=O)NC(C(=O)N1CCC(F)(F)CC1)C(C)OCC1CCOCC1]

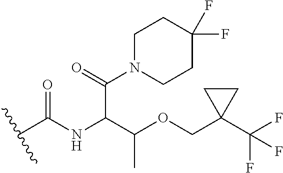 [SMILES: *C(=O)NC(C(=O)N1CCC(F)(F)CC1)C(C)OCC1(C(F)(F)F)CC1]

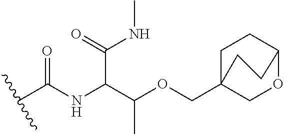 [SMILES: *C(=O)NC(C(=O)NC)C(C)OCC12CCC(CC1)OC2]

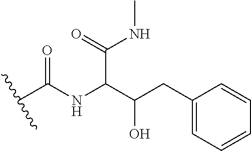 [SMILES: *C(=O)NC(C(=O)NC)C(O)Cc1ccccc1]

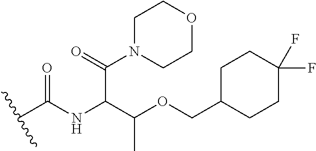 [SMILES: *C(=O)NC(C(=O)N1CCOCC1)C(C)OCC1CCC(F)(F)CC1]

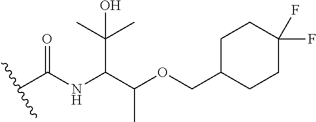 [SMILES: *C(=O)NC(C(C)OCC1CCC(F)(F)CC1)C(C)(C)O]

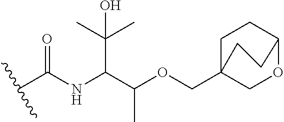 [SMILES: *C(=O)NC(C(C)OCC12CCC(CC1)OC2)C(C)(C)O]

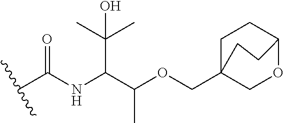 [SMILES: *C(=O)NC(C(C)OCC12CCC(CC1)OC2)C(C)(C)O]

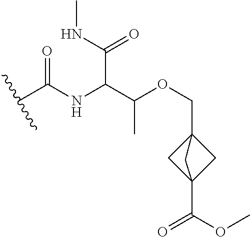 [SMILES: *C(=O)NC(C(=O)NC)C(C)OCC12CC(C(=O)OC)(C1)C2]

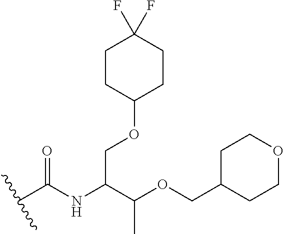 [SMILES: *C(=O)NC(COC1CCC(F)(F)CC1)C(C)OCC1CCOCC1]

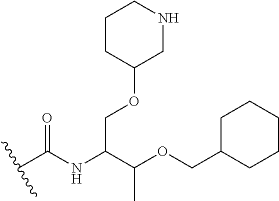 [SMILES: *C(=O)NC(COC1CCCNC1)C(C)OCC1CCCCC1]

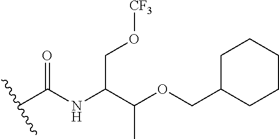 [SMILES: *C(=O)NC(COC(F)(F)F)C(C)OCC1CCCCC1]

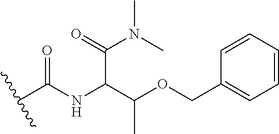 [SMILES: *C(=O)NC(C(=O)N(C)C)C(C)OCc1ccccc1]

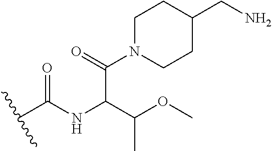 [SMILES: *C(=O)NC(C(=O)N1CCC(CN)CC1)C(C)OC]

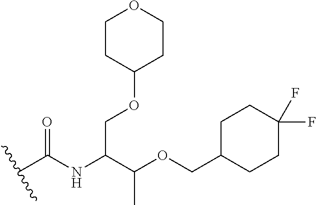 [SMILES: *C(=O)NC(COC1CCOCC1)C(C)OCC1CCC(F)(F)CC1]

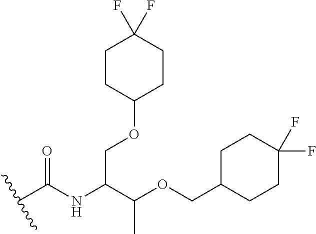 [SMILES: *C(=O)NC(COC1CCC(F)(F)CC1)C(C)OCC1CCC(F)(F)CC1]

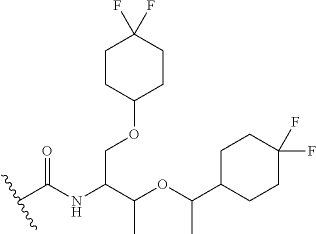 [SMILES: *C(=O)NC(COC1CCC(F)(F)CC1)C(C)OC(C)C1CCC(F)(F)CC1]

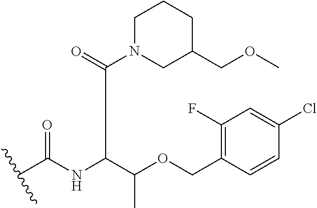 [SMILES: *C(=O)NC(C(=O)N1CCCC(COC)C1)C(C)OCc1ccc(Cl)cc1F]

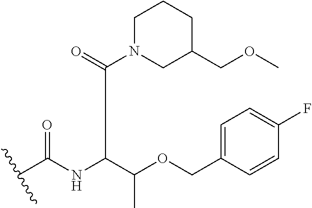 [SMILES: *C(=O)NC(C(=O)N1CCCC(COC)C1)C(C)OCc1ccc(F)cc1]

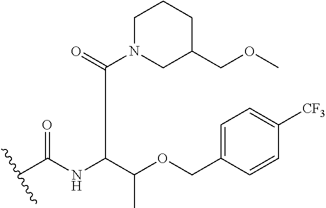 [SMILES: *C(=O)NC(C(=O)N1CCCC(COC)C1)C(C)OCc1ccc(C(F)(F)F)cc1]

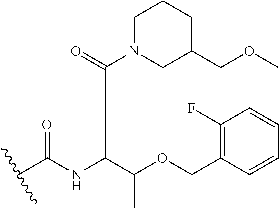 [SMILES: *C(=O)NC(C(=O)N1CCCC(COC)C1)C(C)OCc1ccccc1F]

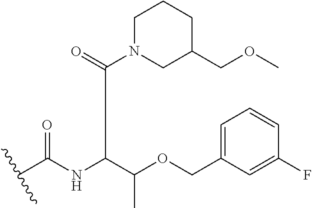 [SMILES: *C(=O)NC(C(=O)N1CCCC(COC)C1)C(C)OCc1cccc(F)c1]

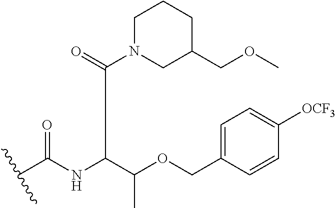 [SMILES: *C(=O)NC(C(=O)N1CCCC(COC)C1)C(C)OCc1ccc(OC(F)(F)F)cc1]

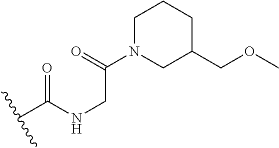 [SMILES: *C(=O)NCC(=O)N1CCCC(COC)C1]

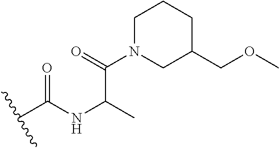 [SMILES: *C(=O)NC(C)C(=O)N1CCCC(COC)C1]

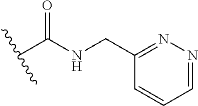 [SMILES: *C(=O)NCc1cccnn1]

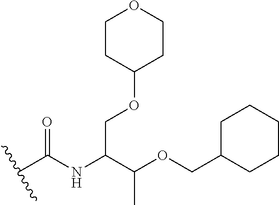 [SMILES: *C(=O)NC(COC1CCOCC1)C(C)OCC1CCCCC1]

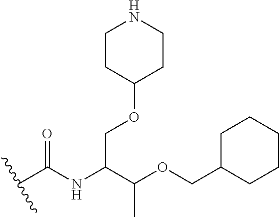 [SMILES: *C(=O)NC(COC1CCNCC1)C(C)OCC1CCCCC1]

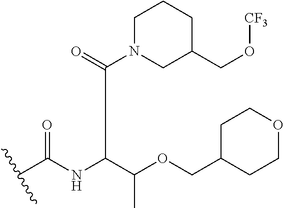 [SMILES: *C(=O)NC(C(=O)N1CCCC(COC(F)(F)F)C1)C(C)OCC1CCOCC1]

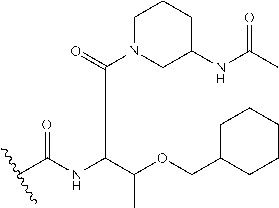 [SMILES: *C(=O)NC(C(=O)N1CCCC(NC(C)=O)C1)C(C)OCC1CCCCC1]

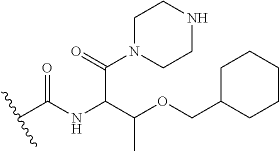 [SMILES: *C(=O)NC(C(=O)N1CCNCC1)C(C)OCC1CCCCC1]

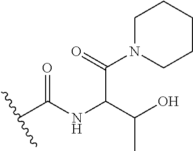 [SMILES: *C(=O)NC(C(=O)N1CCCCC1)C(C)O]

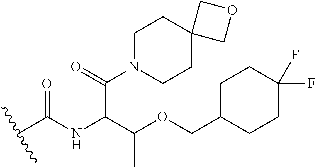 [SMILES: *C(=O)NC(C(=O)N1CCC2(CC1)COC2)C(C)OCC1CCC(F)(F)CC1]

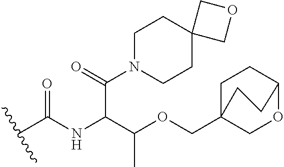 [SMILES: *C(=O)NC(C(=O)N1CCC2(CC1)COC2)C(C)OCC12CCC(CC1)OC2]

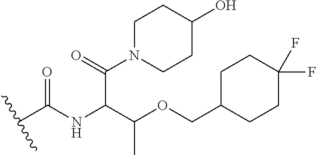 [SMILES: *C(=O)NC(C(=O)N1CCC(O)CC1)C(C)OCC1CCC(F)(F)CC1]

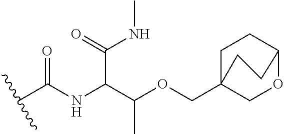 [SMILES: *C(=O)NC(C(=O)NC)C(C)OCC12CCC(CC1)OC2]

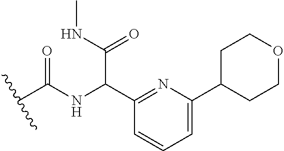 [SMILES: *C(=O)NC(C(=O)NC)c1cccc(C2CCOCC2)n1]

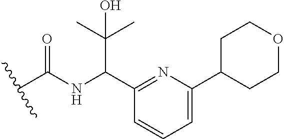 [SMILES: *C(=O)NC(c1cccc(C2CCOCC2)n1)C(C)(C)O]

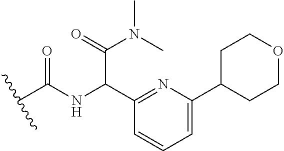 [SMILES: *C(=O)NC(C(=O)N(C)C)c1cccc(C2CCOCC2)n1]

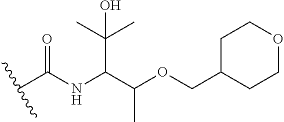 [SMILES: *C(=O)NC(C(C)OCC1CCOCC1)C(C)(C)O]

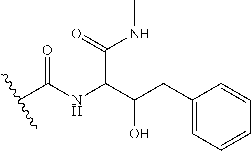 [SMILES: *C(=O)NC(C(=O)NC)C(O)Cc1ccccc1]

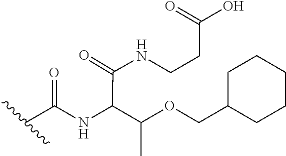 [SMILES: *C(=O)NC(C(=O)NCCC(=O)O)C(C)OCC1CCCCC1]

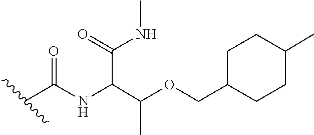 [SMILES: *C(=O)NC(C(=O)NC)C(C)OCC1CCC(C)CC1]

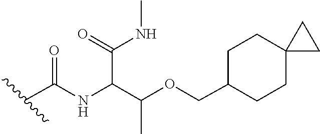 [SMILES: *C(=O)NC(C(=O)NC)C(C)OCC1CCC2(CC1)CC2]

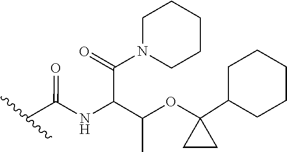 [SMILES: *C(=O)NC(C(=O)N1CCCCC1)C(C)OC1(C2CCCCC2)CC1]

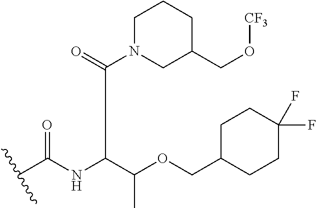 [SMILES: *C(=O)NC(C(=O)N1CCCC(COC(F)(F)F)C1)C(C)OCC1CCC(F)(F)CC1]

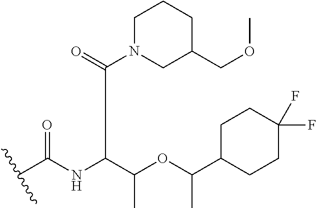 [SMILES: *C(=O)NC(C(=O)N1CCCC(COC)C1)C(C)OC(C)C1CCC(F)(F)CC1]

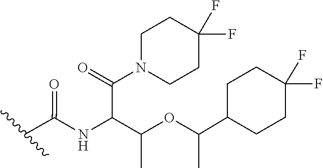 [SMILES: *C(=O)NC(C(=O)N1CCC(F)(F)CC1)C(C)OC(C)C1CCC(F)(F)CC1]

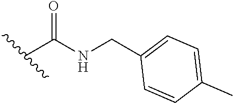 [SMILES: *C(=O)NCc1ccc(C)cc1]

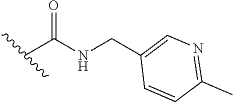 [SMILES: *C(=O)NCc1ccc(C)nc1]

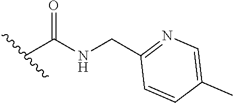 [SMILES: *C(=O)NCc1ccc(C)cn1]

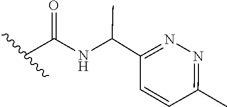 [SMILES: *C(=O)NC(C)c1ccc(C)nn1]

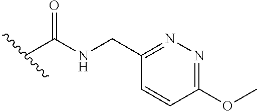 [SMILES: *C(=O)NCc1ccc(OC)nn1]

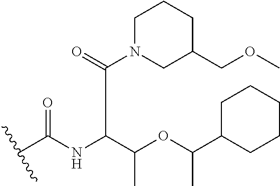 [SMILES: *C(=O)NC(C(=O)N1CCCC(COC)C1)C(C)OC(C)C1CCCCC1]

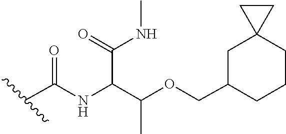 [SMILES: *C(=O)NC(C(=O)NC)C(C)OCC1CCCC2(CC2)C1]

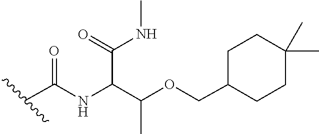 [SMILES: *C(=O)NC(C(=O)NC)C(C)OCC1CCC(C)(C)CC1]

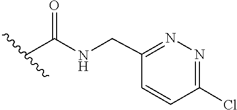 [SMILES: *C(=O)NCc1ccc(Cl)nn1]

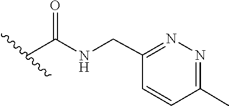 [SMILES: *C(=O)NCc1ccc(C)nn1]

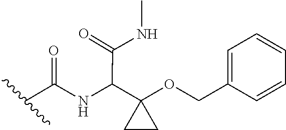 [SMILES: *C(=O)NC(C(=O)NC)C1(OCc2ccccc2)CC1]

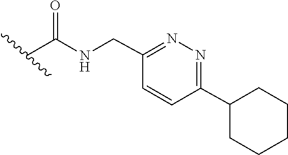 [SMILES: *C(=O)NCc1ccc(C2CCCCC2)nn1]

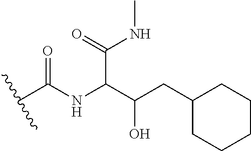 [SMILES: *C(=O)NC(C(=O)NC)C(O)CC1CCCCC1]

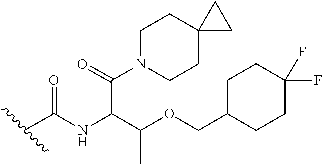 [SMILES: *C(=O)NC(C(=O)N1CCC2(CC1)CC2)C(C)OCC1CCC(F)(F)CC1]

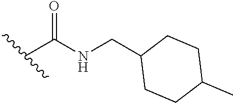 [SMILES: *C(=O)NCC1CCC(C)CC1]

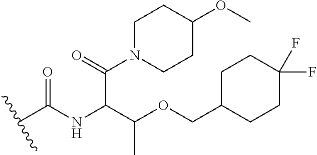 [SMILES: *C(=O)NC(C(=O)N1CCC(OC)CC1)C(C)OCC1CCC(F)(F)CC1]

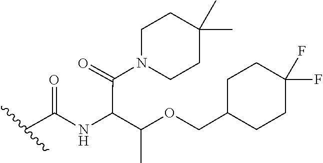 [SMILES: *C(=O)NC(C(=O)N1CCC(C)(C)CC1)C(C)OCC1CCC(F)(F)CC1]

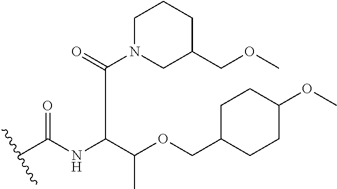 [SMILES: *C(=O)NC(C(=O)N1CCCC(COC)C1)C(C)OCC1CCC(OC)CC1]

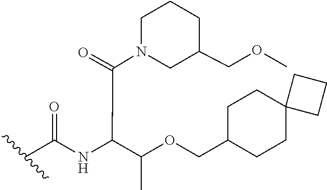 [SMILES: *C(=O)NC(C(=O)N1CCCC(COC)C1)C(C)OCC1CCC2(CCC2)CC1]

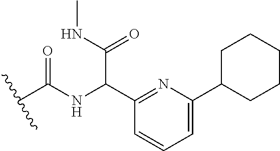 [SMILES: *C(=O)NC(C(=O)NC)c1cccc(C2CCCCC2)n1]

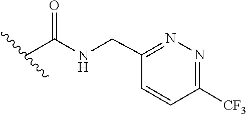 [SMILES: *C(=O)NCc1ccc(C(F)(F)F)nn1]

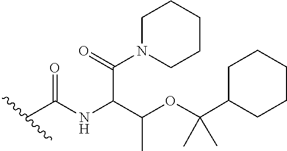 [SMILES: *C(=O)NC(C(=O)N1CCCCC1)C(C)OC(C)(C)C1CCCCC1]

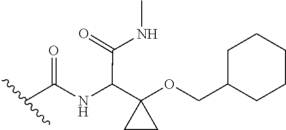 [SMILES: *C(=O)NC(C(=O)NC)C1(OCC2CCCCC2)CC1]

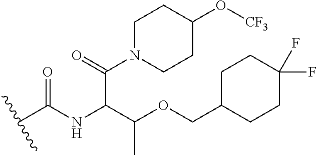 [SMILES: *C(=O)NC(C(=O)N1CCC(OC(F)(F)F)CC1)C(C)OCC1CCC(F)(F)CC1]

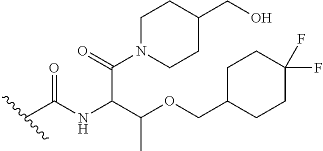 [SMILES: *C(=O)NC(C(=O)N1CCC(CO)CC1)C(C)OCC1CCC(F)(F)CC1]

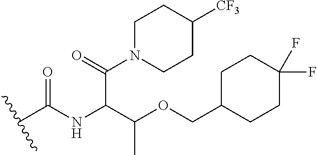 [SMILES: *C(=O)NC(C(=O)N1CCC(C(F)(F)F)CC1)C(C)OCC1CCC(F)(F)CC1]

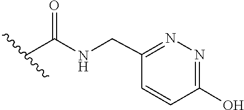 [SMILES: *C(=O)NCc1ccc(O)nn1]

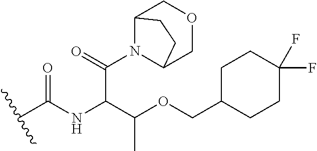 [SMILES: *C(=O)NC(C(=O)N1C2CCC1COC2)C(C)OCC1CCC(F)(F)CC1]

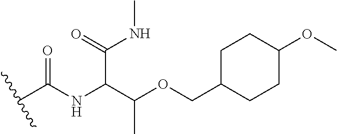 [SMILES: *C(=O)NC(C(=O)NC)C(C)OCC1CCC(OC)CC1]

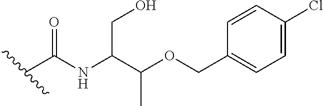 [SMILES: *C(=O)NC(CO)C(C)OCc1ccc(Cl)cc1]

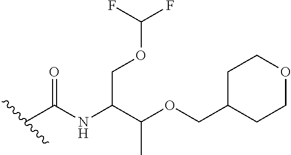 [SMILES: *C(=O)NC(COC(F)F)C(C)OCC1CCOCC1]

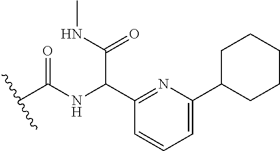 [SMILES: *C(=O)NC(C(=O)NC)c1cccc(C2CCCCC2)n1]

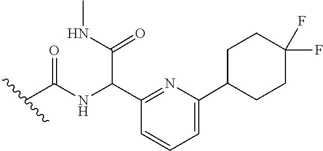 [SMILES: *C(=O)NC(C(=O)NC)c1cccc(C2CCC(F)(F)CC2)n1]

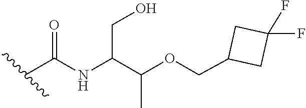 [SMILES: *C(=O)NC(CO)C(C)OCC1CC(F)(F)C1]

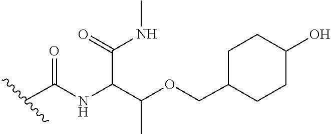 [SMILES: *C(=O)NC(C(=O)NC)C(C)OCC1CCC(O)CC1]

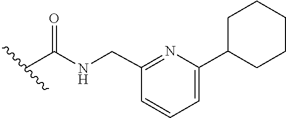 [SMILES: *C(=O)NCc1cccc(C2CCCCC2)n1]

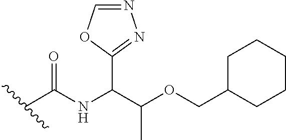 [SMILES: *C(=O)NC(c1nnco1)C(C)OCC1CCCCC1]

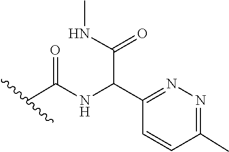 [SMILES: *C(=O)NC(C(=O)NC)c1ccc(C)nn1]

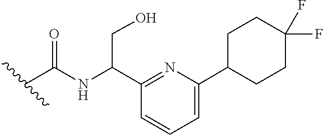 [SMILES: *C(=O)NC(CO)c1cccc(C2CCC(F)(F)CC2)n1]

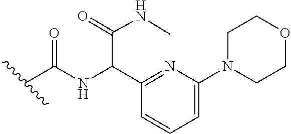 [SMILES: *C(=O)NC(C(=O)NC)c1cccc(N2CCOCC2)n1]

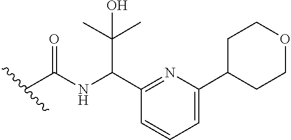 [SMILES: *C(=O)NC(c1cccc(C2CCOCC2)n1)C(C)(C)O]

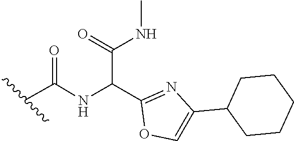 [SMILES: *C(=O)NC(C(=O)NC)c1nc(C2CCCCC2)co1]

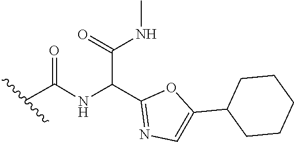 [SMILES: *C(=O)NC(C(=O)NC)c1ncc(C2CCCCC2)o1]

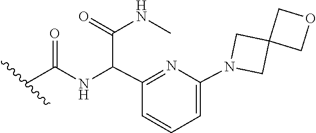 [SMILES: *C(=O)NC(C(=O)NC)c1cccc(N2CC3(COC3)C2)n1]

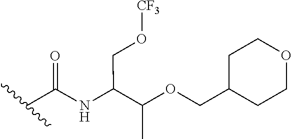 [SMILES: *C(=O)NC(COC(F)(F)F)C(C)OCC1CCOCC1]

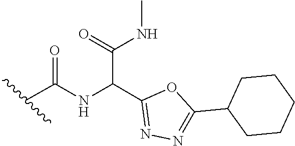 [SMILES: *C(=O)NC(C(=O)NC)c1nnc(C2CCCCC2)o1]

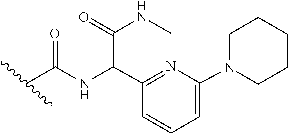 [SMILES: *C(=O)NC(C(=O)NC)c1cccc(N2CCCCC2)n1]

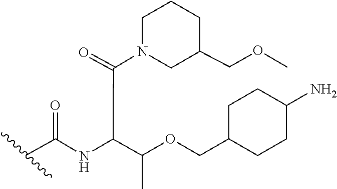 [SMILES: *C(=O)NC(C(=O)N1CCCC(COC)C1)C(C)OCC1CCC(N)CC1]

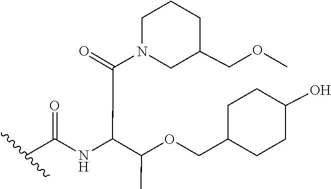 [SMILES: *C(=O)NC(C(=O)N1CCCC(COC)C1)C(C)OCC1CCC(O)CC1]

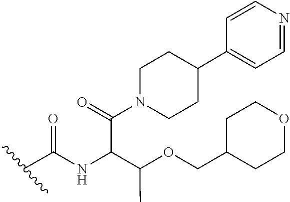 [SMILES: *C(=O)NC(C(=O)N1CCC(c2ccncc2)CC1)C(C)OCC1CCOCC1]

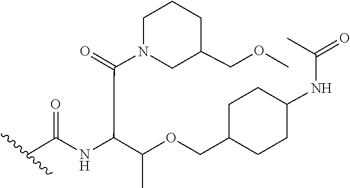 [SMILES: *C(=O)NC(C(=O)N1CCCC(COC)C1)C(C)OCC1CCC(NC(C)=O)CC1]

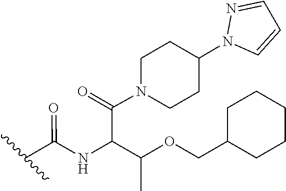 [SMILES: *C(=O)NC(C(=O)N1CCC(n2cccn2)CC1)C(C)OCC1CCCCC1]

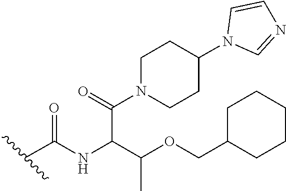 [SMILES: *C(=O)NC(C(=O)N1CCC(n2ccnc2)CC1)C(C)OCC1CCCCC1]

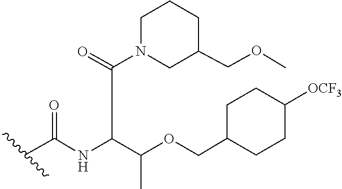 [SMILES: *C(=O)NC(C(=O)N1CCCC(COC)C1)C(C)OCC1CCC(OC(F)(F)F)CC1]

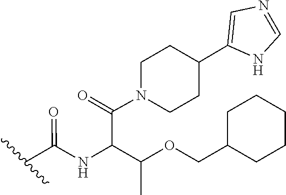 [SMILES: *C(=O)NC(C(=O)N1CCC(c2cnc[nH]2)CC1)C(C)OCC1CCCCC1]

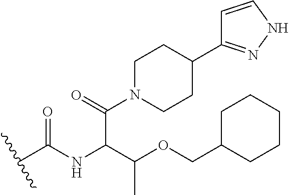 [SMILES: *C(=O)NC(C(=O)N1CCC(c2cc[nH]n2)CC1)C(C)OCC1CCCCC1]

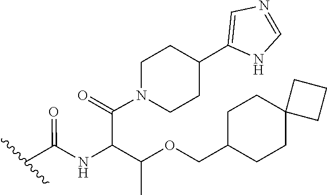 [SMILES: *C(=O)NC(C(=O)N1CCC(c2cnc[nH]2)CC1)C(C)OCC1CCC2(CCC2)CC1]

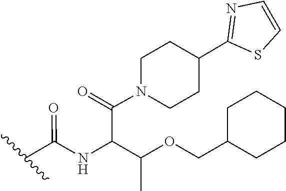 [SMILES: *C(=O)NC(C(=O)N1CCC(c2nccs2)CC1)C(C)OCC1CCCCC1]

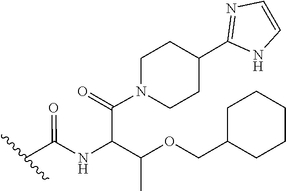 [SMILES: *C(=O)NC(C(=O)N1CCC(c2ncc[nH]2)CC1)C(C)OCC1CCCCC1]

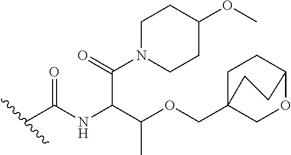 [SMILES: *C(=O)NC(C(=O)N1CCC(OC)CC1)C(C)OCC12CCC(CC1)OC2]

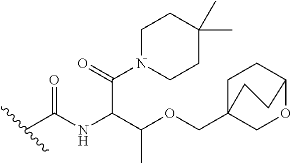 [SMILES: *C(=O)NC(C(=O)N1CCC(C)(C)CC1)C(C)OCC12CCC(CC1)OC2]

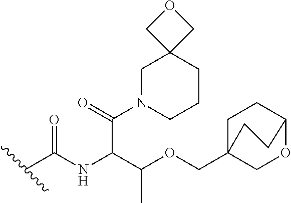 [SMILES: *C(=O)NC(C(=O)N1CCCC2(COC2)C1)C(C)OCC12CCC(CC1)OC2]

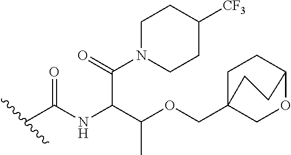 [SMILES: *C(=O)NC(C(=O)N1CCC(C(F)(F)F)CC1)C(C)OCC12CCC(CC1)OC2]

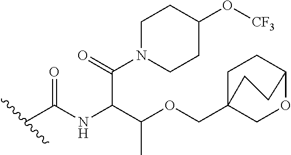 [SMILES: *C(=O)NC(C(=O)N1CCC(OC(F)(F)F)CC1)C(C)OCC12CCC(CC1)OC2]

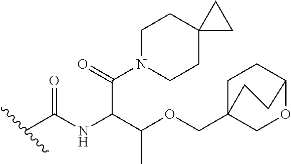 [SMILES: *C(=O)NC(C(=O)N1CCC2(CC1)CC2)C(C)OCC12CCC(CC1)OC2]

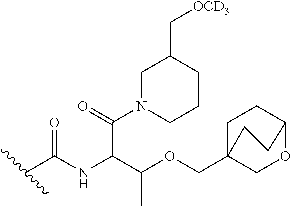

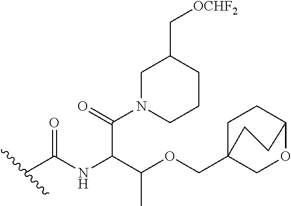 [SMILES: *C(=O)NC(C(=O)N1CCCC(COC(F)F)C1)C(C)OCC12CCC(CC1)OC2]

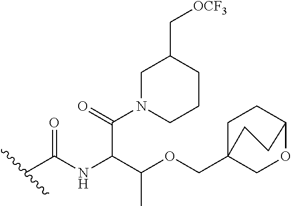 [SMILES: *C(=O)NC(C(=O)N1CCCC(COC(F)(F)F)C1)C(C)OCC12CCC(CC1)OC2]

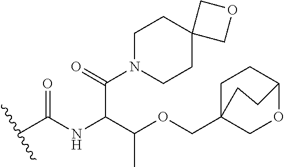 [SMILES: *C(=O)NC(C(=O)N1CCC2(CC1)COC2)C(C)OCC12CCC(CC1)OC2]

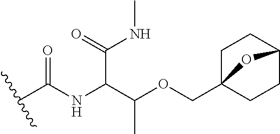 [SMILES: *C(=O)NC(C(=O)NC)C(C)OC[C@]12CC[C@H](CC1)O2]

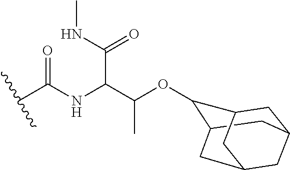 [SMILES: *C(=O)NC(C(=O)NC)C(C)OC1C2CC3CC(C2)CC1C3]

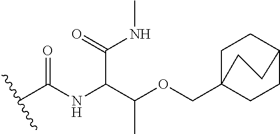 [SMILES: *C(=O)NC(C(=O)NC)C(C)OCC12CCC(CC1)CC2]

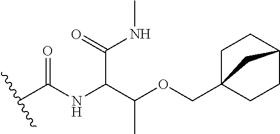 [SMILES: *C(=O)NC(C(=O)NC)C(C)OC[C@]12CC[C@H](CC1)C2]

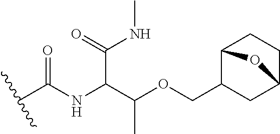 [SMILES: *C(=O)NC(C(=O)NC)C(C)OCC1C[C@H]2CC[C@@H]1O2]

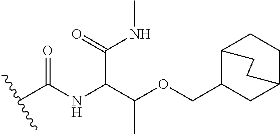 [SMILES: *C(=O)NC(C(=O)NC)C(C)OCC1CC2CCC1CC2]

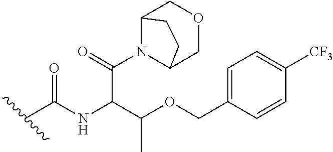 [SMILES: *C(=O)NC(C(=O)N1C2CCC1COC2)C(C)OCc1ccc(C(F)(F)F)cc1]

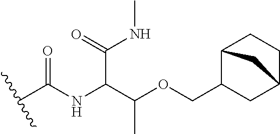 [SMILES: *C(=O)NC(C(=O)NC)C(C)OCC1C[C@H]2CC[C@@H]1C2]

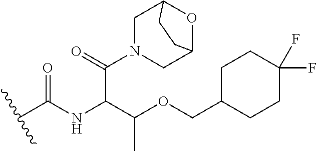 [SMILES: *C(=O)NC(C(=O)N1CC2CCC(C1)O2)C(C)OCC1CCC(F)(F)CC1]

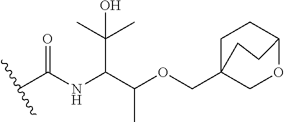 [SMILES: *C(=O)NC(C(C)OCC12CCC(CC1)OC2)C(C)(C)O]

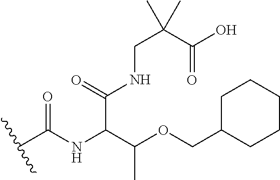 [SMILES: *C(=O)NC(C(=O)NCC(C)(C)C(=O)O)C(C)OCC1CCCCC1]

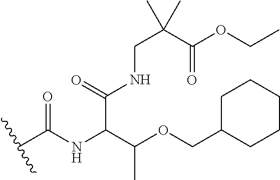 [SMILES: *C(=O)NC(C(=O)NCC(C)(C)C(=O)OCC)C(C)OCC1CCCCC1]

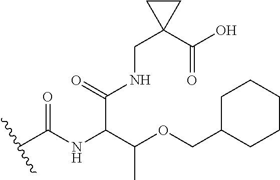 [SMILES: *C(=O)NC(C(=O)NCC1(C(=O)O)CC1)C(C)OCC1CCCCC1]

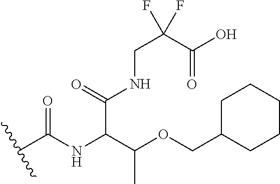 [SMILES: *C(=O)NC(C(=O)NCC(F)(F)C(=O)O)C(C)OCC1CCCCC1]

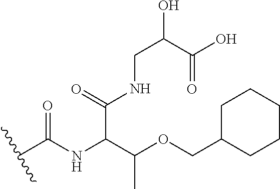 [SMILES: *C(=O)NC(C(=O)NCC(O)C(=O)O)C(C)OCC1CCCCC1]

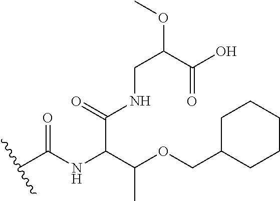 [SMILES: *C(=O)NC(C(=O)NCC(OC)C(=O)O)C(C)OCC1CCCCC1]

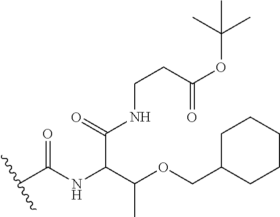 [SMILES: *C(=O)NC(C(=O)NCCC(=O)OC(C)(C)C)C(C)OCC1CCCCC1]

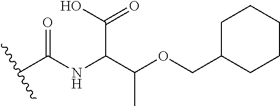 [SMILES: *C(=O)NC(C(=O)O)C(C)OCC1CCCCC1]

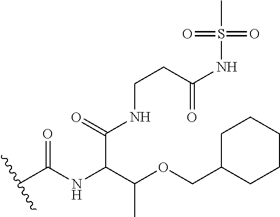 [SMILES: *C(=O)NC(C(=O)NCCC(=O)NS(C)(=O)=O)C(C)OCC1CCCCC1]

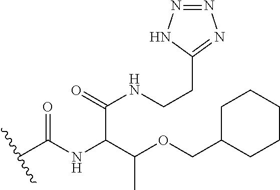 [SMILES: *C(=O)NC(C(=O)NCCc1nnn[nH]1)C(C)OCC1CCCCC1]

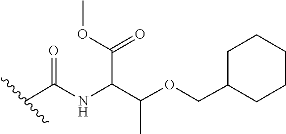 [SMILES: *C(=O)NC(C(=O)OC)C(C)OCC1CCCCC1]

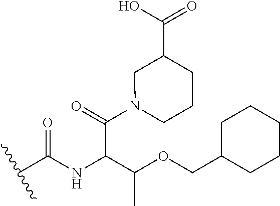 [SMILES: *C(=O)NC(C(=O)N1CCCC(C(=O)O)C1)C(C)OCC1CCCCC1]

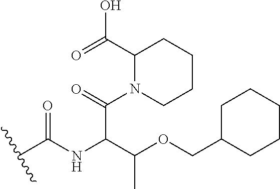 [SMILES: *C(=O)NC(C(=O)N1CCCCC1C(=O)O)C(C)OCC1CCCCC1]

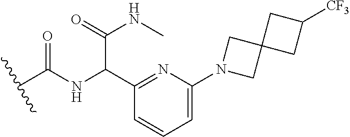 [SMILES: *C(=O)NC(C(=O)NC)c1cccc(N2CC3(CC(C(F)(F)F)C3)C2)n1]

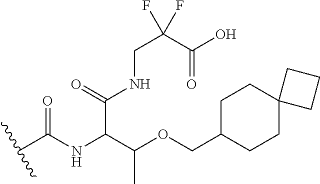 [SMILES: *C(=O)NC(C(=O)NCC(F)(F)C(=O)O)C(C)OCC1CCC2(CCC2)CC1]

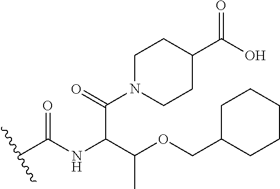 [SMILES: *C(=O)NC(C(=O)N1CCC(C(=O)O)CC1)C(C)OCC1CCCCC1]

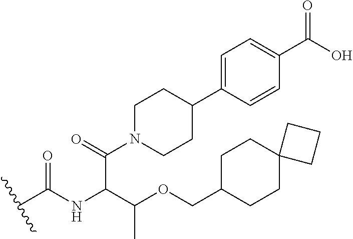 [SMILES: *C(=O)NC(C(=O)N1CCC(c2ccc(C(=O)O)cc2)CC1)C(C)OCC1CCC2(CCC2)CC1]

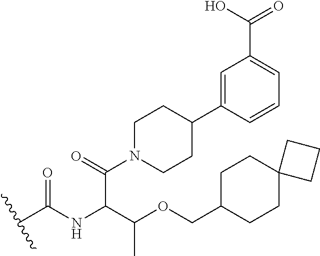 [SMILES: *C(=O)NC(C(=O)N1CCC(c2cccc(C(=O)O)c2)CC1)C(C)OCC1CCC2(CCC2)CC1]

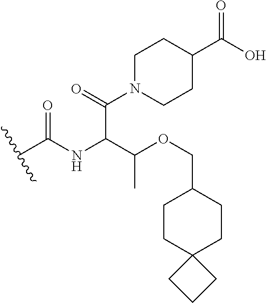 [SMILES: *C(=O)NC(C(=O)N1CCC(C(=O)O)CC1)C(C)OCC1CCC2(CCC2)CC1]

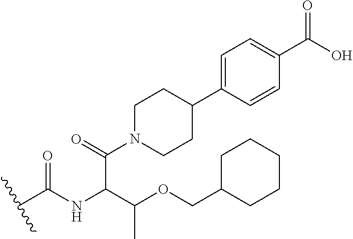 [SMILES: *C(=O)NC(C(=O)N1CCC(c2ccc(C(=O)O)cc2)CC1)C(C)OCC1CCCCC1]

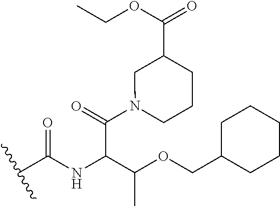 [SMILES: *C(=O)NC(C(=O)N1CCCC(C(=O)OCC)C1)C(C)OCC1CCCCC1]

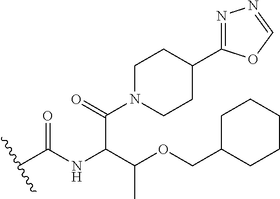 [SMILES: *C(=O)NC(C(=O)N1CCC(c2nnco2)CC1)C(C)OCC1CCCCC1]

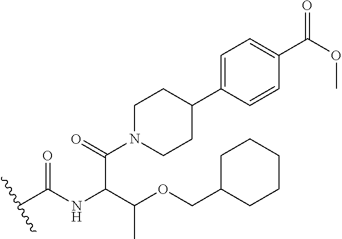 [SMILES: *C(=O)NC(C(=O)N1CCC(c2ccc(C(=O)OC)cc2)CC1)C(C)OCC1CCCCC1]

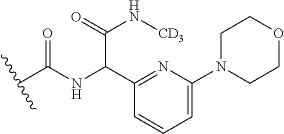 [SMILES: *C(=O)NC(C(=O)NC([2H])([2H])[2H])c1cccc(N2CCOCC2)n1]

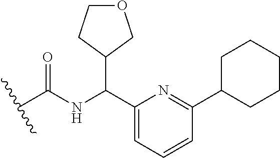 [SMILES: *C(=O)NC(c1cccc(C2CCCCC2)n1)C1CCOC1]

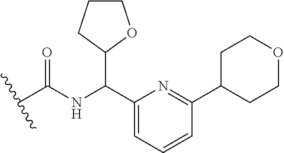 [SMILES: *C(=O)NC(c1cccc(C2CCOCC2)n1)C1CCCO1]

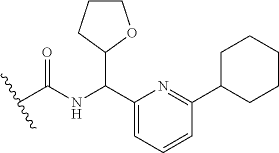 [SMILES: *C(=O)NC(c1cccc(C2CCCCC2)n1)C1CCCO1]

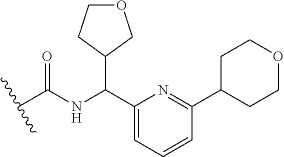 [SMILES: *C(=O)NC(c1cccc(C2CCOCC2)n1)C1CCOC1]

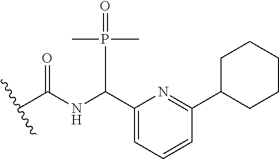 [SMILES: *C(=O)NC(c1cccc(C2CCCCC2)n1)P(C)(C)=O]

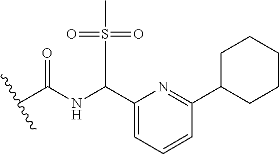 [SMILES: *C(=O)NC(c1cccc(C2CCCCC2)n1)S(C)(=O)=O]

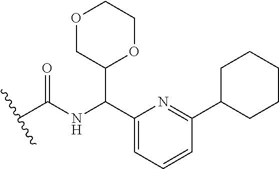 [SMILES: *C(=O)NC(c1cccc(C2CCCCC2)n1)C1COCCO1]

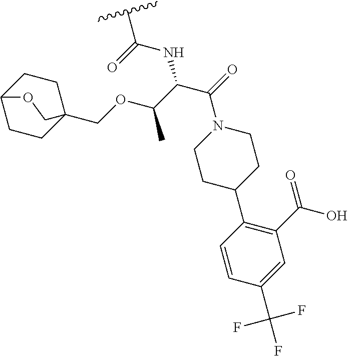 [SMILES: *C(=O)N[C@H](C(=O)N1CCC(c2ccc(C(F)(F)F)cc2C(=O)O)CC1)[C@@H](C)OCC12CCC(CC1)OC2]

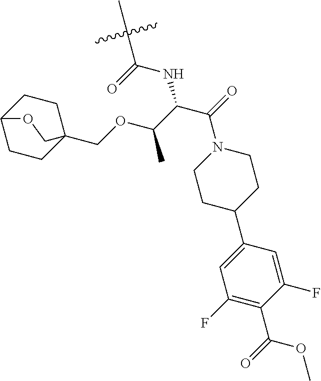 [SMILES: *C(=O)N[C@H](C(=O)N1CCC(c2cc(F)c(C(=O)OC)c(F)c2)CC1)[C@@H](C)OCC12CCC(CC1)OC2]

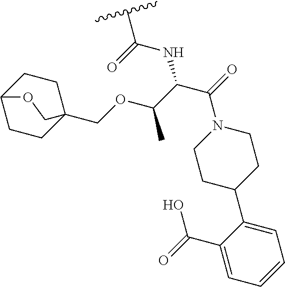 [SMILES: *C(=O)N[C@H](C(=O)N1CCC(c2ccccc2C(=O)O)CC1)[C@@H](C)OCC12CCC(CC1)OC2]

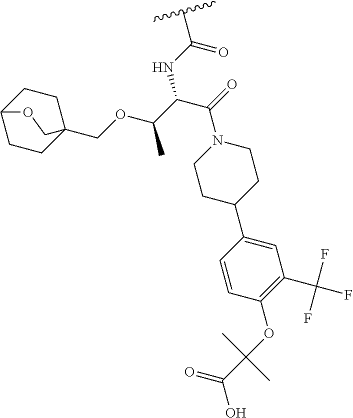 [SMILES: *C(=O)N[C@H](C(=O)N1CCC(c2ccc(OC(C)(C)C(=O)O)c(C(F)(F)F)c2)CC1)[C@@H](C)OCC12CCC(CC1)OC2]

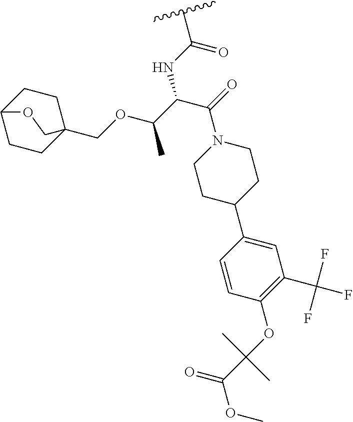 [SMILES: *C(=O)N[C@H](C(=O)N1CCC(c2ccc(OC(C)(C)C(=O)OC)c(C(F)(F)F)c2)CC1)[C@@H](C)OCC12CCC(CC1)OC2]

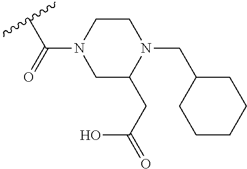 [SMILES: *C(=O)N1CCN(CC2CCCCC2)C(CC(=O)O)C1]

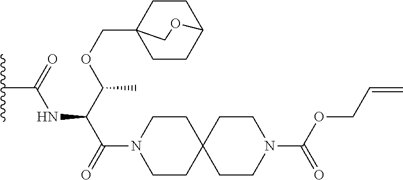 [SMILES: *C(=O)N[C@H](C(=O)N1CCC2(CCN(C(=O)OCC=C)CC2)CC1)[C@@H](C)OCC12CCC(CC1)OC2]

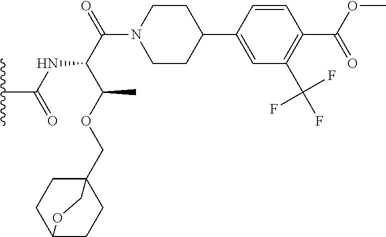 [SMILES: *C(=O)N[C@H](C(=O)N1CCC(c2ccc(C(=O)OC)c(C(F)(F)F)c2)CC1)[C@@H](C)OCC12CCC(CC1)OC2]

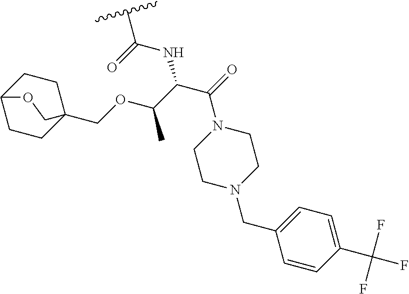 [SMILES: *C(=O)N[C@H](C(=O)N1CCN(Cc2ccc(C(F)(F)F)cc2)CC1)[C@@H](C)OCC12CCC(CC1)OC2]

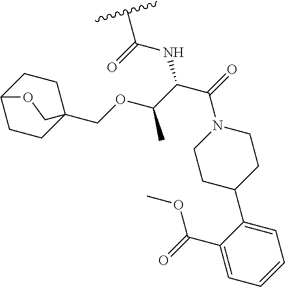 [SMILES: *C(=O)N[C@H](C(=O)N1CCC(c2ccccc2C(=O)OC)CC1)[C@@H](C)OCC12CCC(CC1)OC2]

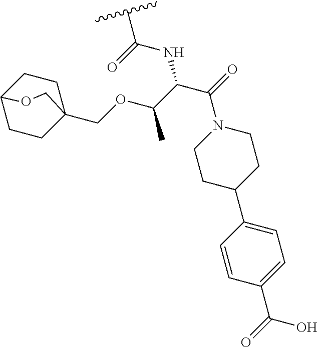 [SMILES: *C(=O)N[C@H](C(=O)N1CCC(c2ccc(C(=O)O)cc2)CC1)[C@@H](C)OCC12CCC(CC1)OC2]

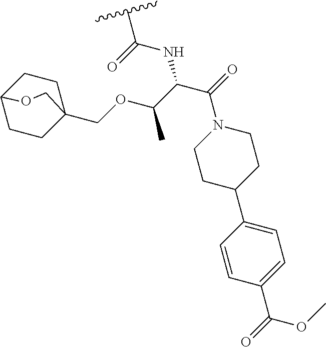 [SMILES: *C(=O)N[C@H](C(=O)N1CCC(c2ccc(C(=O)OC)cc2)CC1)[C@@H](C)OCC12CCC(CC1)OC2]

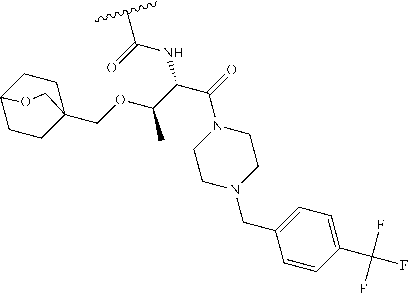 [SMILES: *C(=O)N[C@H](C(=O)N1CCN(Cc2ccc(C(F)(F)F)cc2)CC1)[C@@H](C)OCC12CCC(CC1)OC2]

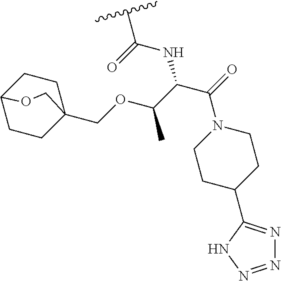 [SMILES: *C(=O)N[C@H](C(=O)N1CCC(c2nnn[nH]2)CC1)[C@@H](C)OCC12CCC(CC1)OC2]

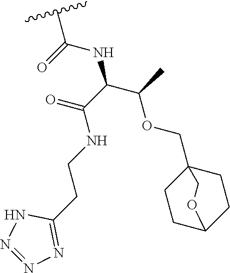 [SMILES: *C(=O)N[C@H](C(=O)NCCc1nnn[nH]1)[C@@H](C)OCC12CCC(CC1)OC2]

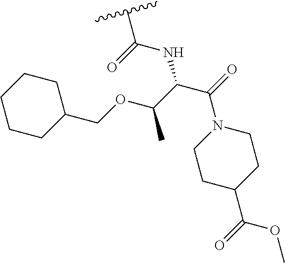 [SMILES: *C(=O)N[C@H](C(=O)N1CCC(C(=O)OC)CC1)[C@@H](C)OCC1CCCCC1]

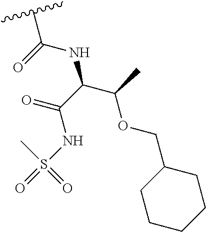 [SMILES: *C(=O)N[C@H](C(=O)NS(C)(=O)=O)[C@@H](C)OCC1CCCCC1]

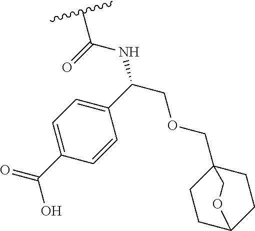 [SMILES: *C(=O)N[C@H](COCC12CCC(CC1)OC2)c1ccc(C(=O)O)cc1]

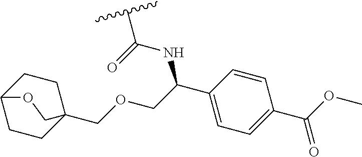 [SMILES: *C(=O)N[C@H](COCC12CCC(CC1)OC2)c1ccc(C(=O)OC)cc1]

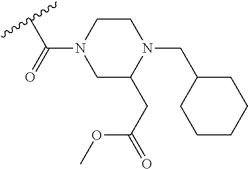 [SMILES: *C(=O)N1CCN(CC2CCCCC2)C(CC(=O)OC)C1]

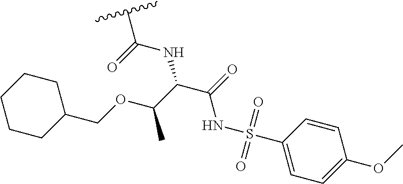 [SMILES: *C(=O)N[C@H](C(=O)NS(=O)(=O)c1ccc(OC)cc1)[C@@H](C)OCC1CCCCC1]

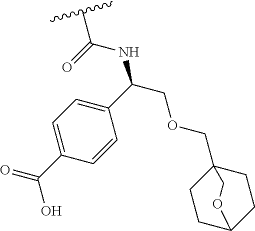 [SMILES: *C(=O)N[C@@H](COCC12CCC(CC1)OC2)c1ccc(C(=O)O)cc1]

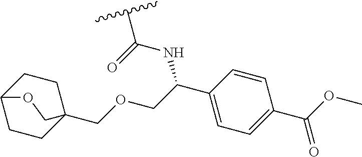 [SMILES: *C(=O)N[C@@H](COCC12CCC(CC1)OC2)c1ccc(C(=O)OC)cc1]

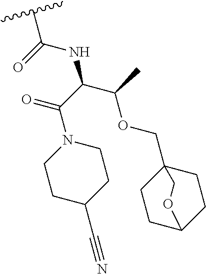 [SMILES: *C(=O)N[C@H](C(=O)N1CCC(C#N)CC1)[C@@H](C)OCC12CCC(CC1)OC2]

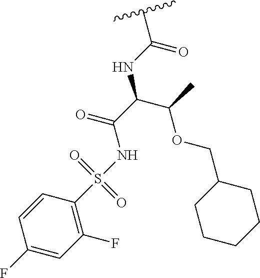 [SMILES: *C(=O)N[C@H](C(=O)NS(=O)(=O)c1ccc(F)cc1F)[C@@H](C)OCC1CCCCC1]

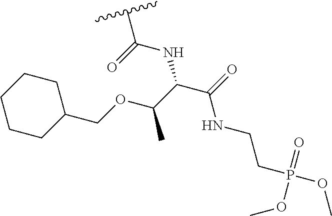 [SMILES: *C(=O)N[C@H](C(=O)NCCP(=O)(OC)OC)[C@@H](C)OCC1CCCCC1]

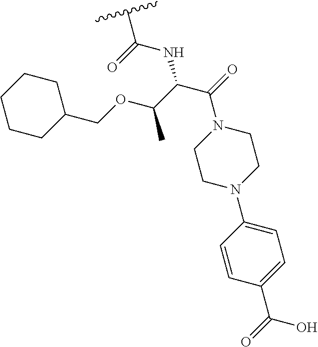 [SMILES: *C(=O)N[C@H](C(=O)N1CCN(c2ccc(C(=O)O)cc2)CC1)[C@@H](C)OCC1CCCCC1]

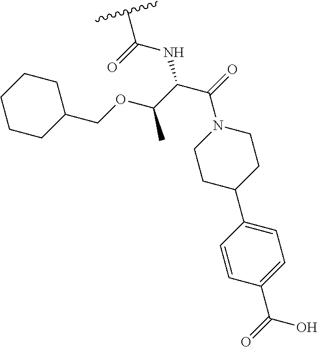 [SMILES: *C(=O)N[C@H](C(=O)N1CCC(c2ccc(C(=O)O)cc2)CC1)[C@@H](C)OCC1CCCCC1]

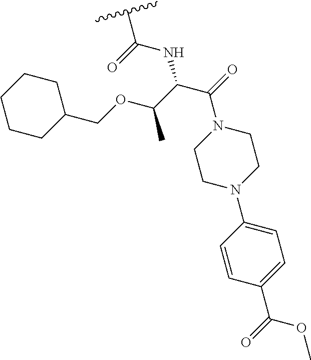 [SMILES: *C(=O)N[C@H](C(=O)N1CCN(c2ccc(C(=O)OC)cc2)CC1)[C@@H](C)OCC1CCCCC1]

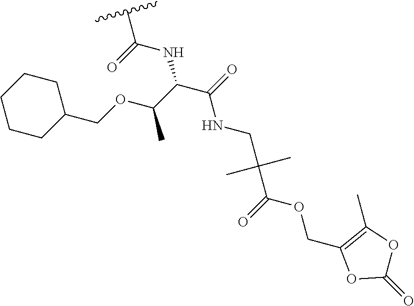 [SMILES: *C(=O)N[C@H](C(=O)NCC(C)(C)C(=O)OCc1oc(=O)oc1C)[C@@H](C)OCC1CCCCC1]

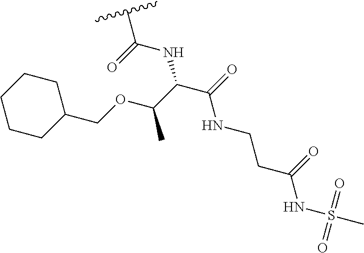 [SMILES: *C(=O)N[C@H](C(=O)NCCC(=O)NS(C)(=O)=O)[C@@H](C)OCC1CCCCC1]

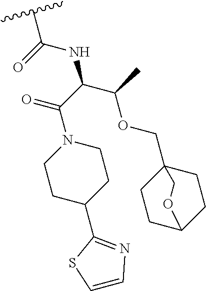 [SMILES: *C(=O)N[C@H](C(=O)N1CCC(c2nccs2)CC1)[C@@H](C)OCC12CCC(CC1)OC2]

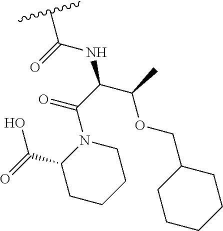 [SMILES: *C(=O)N[C@H](C(=O)N1CCCC[C@@H]1C(=O)O)[C@@H](C)OCC1CCCCC1]

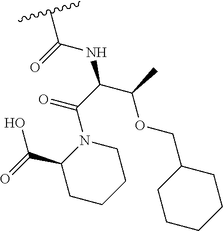 [SMILES: *C(=O)N[C@H](C(=O)N1CCCC[C@H]1C(=O)O)[C@@H](C)OCC1CCCCC1]

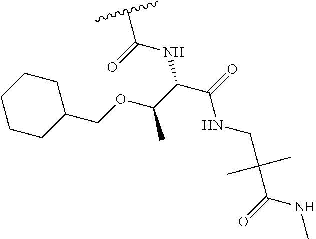 [SMILES: *C(=O)N[C@H](C(=O)NCC(C)(C)C(=O)NC)[C@@H](C)OCC1CCCCC1]

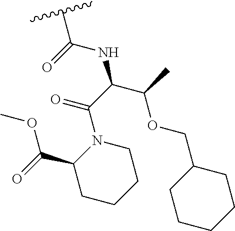 [SMILES: *C(=O)N[C@H](C(=O)N1CCCC[C@H]1C(=O)OC)[C@@H](C)OCC1CCCCC1]

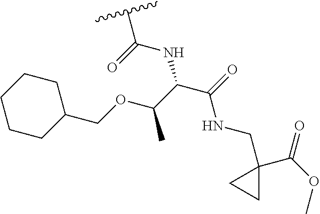 [SMILES: *C(=O)N[C@H](C(=O)NCC1(C(=O)OC)CC1)[C@@H](C)OCC1CCCCC1]

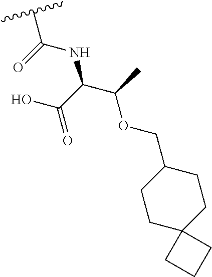 [SMILES: *C(=O)N[C@H](C(=O)O)[C@@H](C)OCC1CCC2(CCC2)CC1]

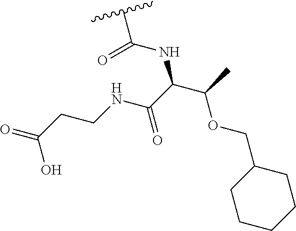 [SMILES: *C(=O)N[C@H](C(=O)NCCC(=O)O)[C@@H](C)OCC1CCCCC1]

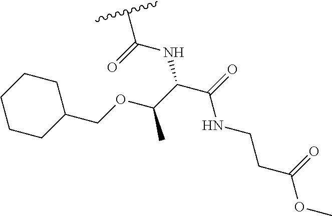 [SMILES: *C(=O)N[C@H](C(=O)NCCC(=O)OC)[C@@H](C)OCC1CCCCC1]

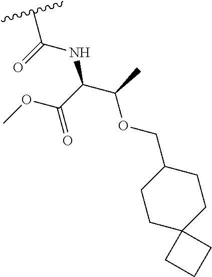 [SMILES: *C(=O)N[C@H](C(=O)OC)[C@@H](C)OCC1CCC2(CCC2)CC1]

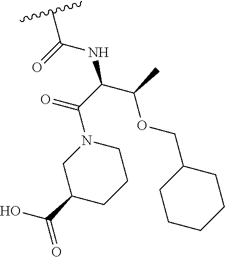 [SMILES: *C(=O)N[C@H](C(=O)N1CCC[C@@H](C(=O)O)C1)[C@@H](C)OCC1CCCCC1]

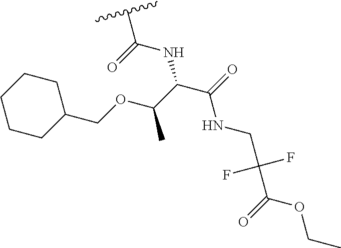 [SMILES: *C(=O)N[C@H](C(=O)NCC(F)(F)C(=O)OCC)[C@@H](C)OCC1CCCCC1]

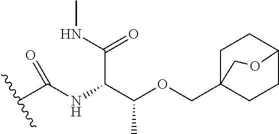 [SMILES: *C(=O)N[C@H](C(=O)NC)[C@@H](C)OCC12CCC(CC1)OC2]

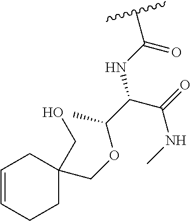 [SMILES: *C(=O)N[C@H](C(=O)NC)[C@@H](C)OCC1(CO)CC=CCC1]

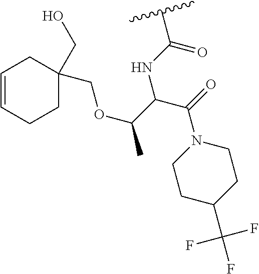 [SMILES: *C(=O)NC(C(=O)N1CCC(C(F)(F)F)CC1)[C@@H](C)OCC1(CO)CC=CCC1]

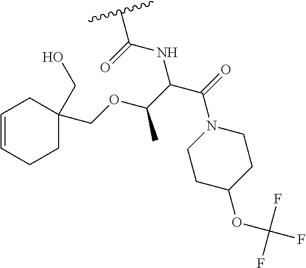 [SMILES: *C(=O)NC(C(=O)N1CCC(OC(F)(F)F)CC1)[C@@H](C)OCC1(CO)CC=CCC1]

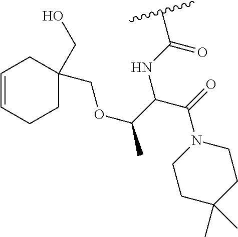 [SMILES: *C(=O)NC(C(=O)N1CCC(C)(C)CC1)[C@@H](C)OCC1(CO)CC=CCC1]

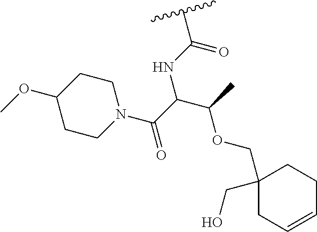 [SMILES: *C(=O)NC(C(=O)N1CCC(OC)CC1)[C@@H](C)OCC1(CO)CC=CCC1]

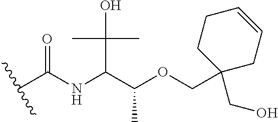 [SMILES: *C(=O)NC([C@@H](C)OCC1(CO)CC=CCC1)C(C)(C)O]

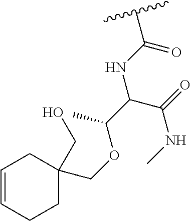 [SMILES: *C(=O)NC(C(=O)NC)[C@@H](C)OCC1(CO)CC=CCC1]

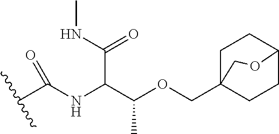 [SMILES: *C(=O)NC(C(=O)NC)[C@@H](C)OCC12CCC(CC1)OC2]

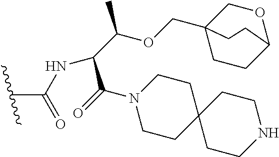 [SMILES: *C(=O)N[C@H](C(=O)N1CCC2(CCNCC2)CC1)[C@@H](C)OCC12CCC(CC1)OC2]

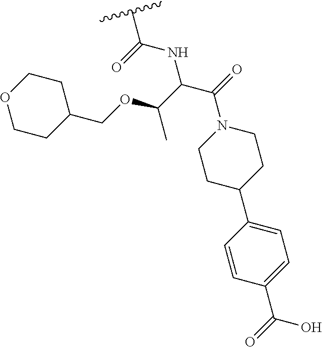 [SMILES: *C(=O)NC(C(=O)N1CCC(c2ccc(C(=O)O)cc2)CC1)[C@@H](C)OCC1CCOCC1]

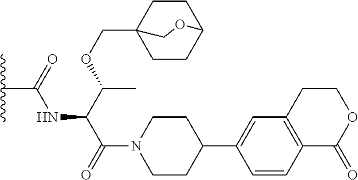 [SMILES: *C(=O)N[C@H](C(=O)N1CCC(c2ccc3c(c2)CCOC3=O)CC1)[C@@H](C)OCC12CCC(CC1)OC2]

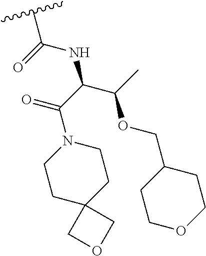 [SMILES: *C(=O)N[C@H](C(=O)N1CCC2(CC1)COC2)[C@@H](C)OCC1CCOCC1]

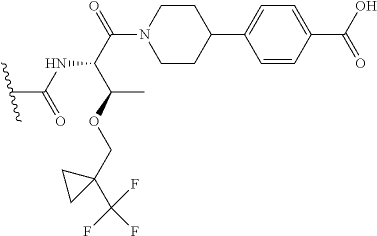 [SMILES: *C(=O)N[C@H](C(=O)N1CCC(c2ccc(C(=O)O)cc2)CC1)[C@@H](C)OCC1(C(F)(F)F)CC1]

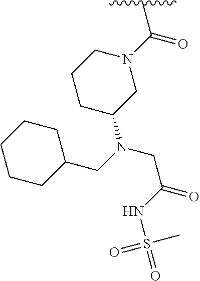 [SMILES: *C(=O)N1CCC[C@@H](N(CC(=O)NS(C)(=O)=O)CC2CCCCC2)C1]

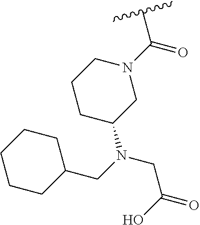 [SMILES: *C(=O)N1CCC[C@@H](N(CC(=O)O)CC2CCCCC2)C1]

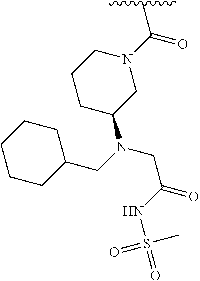 [SMILES: *C(=O)N1CCC[C@H](N(CC(=O)NS(C)(=O)=O)CC2CCCCC2)C1]

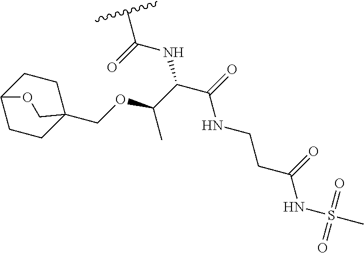 [SMILES: *C(=O)N[C@H](C(=O)NCCC(=O)NS(C)(=O)=O)[C@@H](C)OCC12CCC(CC1)OC2]

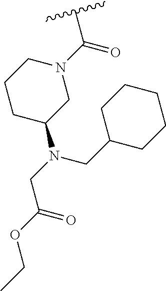 [SMILES: *C(=O)N1CCC[C@H](N(CC(=O)OCC)CC2CCCCC2)C1]

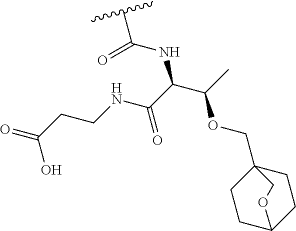 [SMILES: *C(=O)N[C@H](C(=O)NCCC(=O)O)[C@@H](C)OCC12CCC(CC1)OC2]

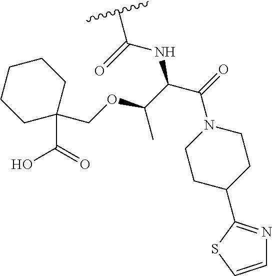 [SMILES: *C(=O)N[C@@H](C(=O)N1CCC(c2nccs2)CC1)[C@@H](C)OCC1(C(=O)O)CCCCC1]

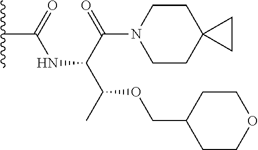 [SMILES: *C(=O)N[C@H](C(=O)N1CCC2(CC1)CC2)[C@@H](C)OCC1CCOCC1]

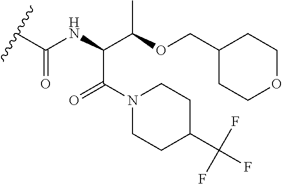 [SMILES: *C(=O)N[C@H](C(=O)N1CCC(C(F)(F)F)CC1)[C@@H](C)OCC1CCOCC1]

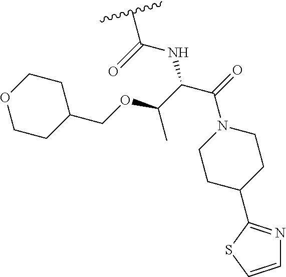 [SMILES: *C(=O)N[C@H](C(=O)N1CCC(c2nccs2)CC1)[C@@H](C)OCC1CCOCC1]

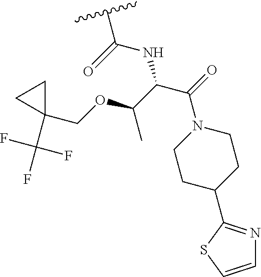 [SMILES: *C(=O)N[C@H](C(=O)N1CCC(c2nccs2)CC1)[C@@H](C)OCC1(C(F)(F)F)CC1]

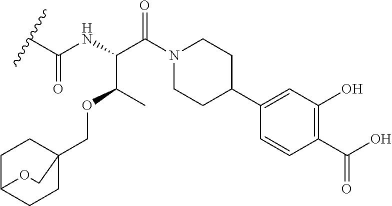 [SMILES: *C(=O)N[C@H](C(=O)N1CCC(c2ccc(C(=O)O)c(O)c2)CC1)[C@@H](C)OCC12CCC(CC1)OC2]

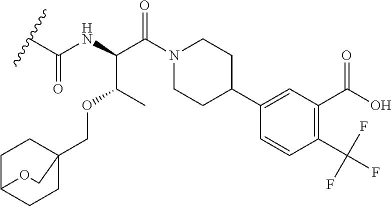 [SMILES: *C(=O)N[C@@H](C(=O)N1CCC(c2ccc(C(F)(F)F)c(C(=O)O)c2)CC1)[C@H](C)OCC12CCC(CC1)OC2]

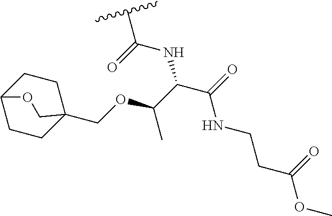 [SMILES: *C(=O)N[C@H](C(=O)NCCC(=O)OC)[C@@H](C)OCC12CCC(CC1)OC2]

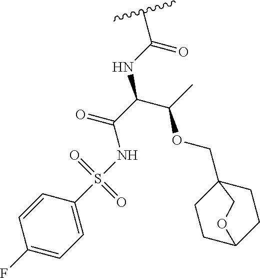 [SMILES: *C(=O)N[C@H](C(=O)NS(=O)(=O)c1ccc(F)cc1)[C@@H](C)OCC12CCC(CC1)OC2]

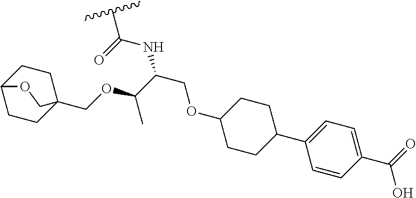 [SMILES: *C(=O)N[C@H](COC1CCC(c2ccc(C(=O)O)cc2)CC1)[C@@H](C)OCC12CCC(CC1)OC2]

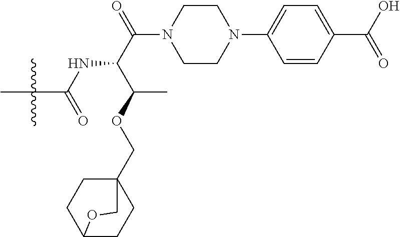 [SMILES: *C(=O)N[C@H](C(=O)N1CCN(c2ccc(C(=O)O)cc2)CC1)[C@@H](C)OCC12CCC(CC1)OC2]

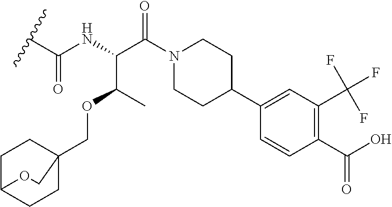 [SMILES: *C(=O)N[C@H](C(=O)N1CCC(c2ccc(C(=O)O)c(C(F)(F)F)c2)CC1)[C@@H](C)OCC12CCC(CC1)OC2]

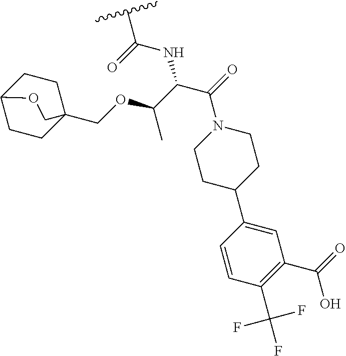 [SMILES: *C(=O)N[C@H](C(=O)N1CCC(c2ccc(C(F)(F)F)c(C(=O)O)c2)CC1)[C@@H](C)OCC12CCC(CC1)OC2]

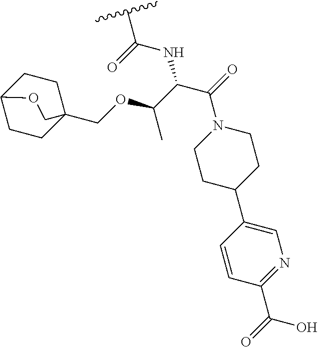 [SMILES: *C(=O)N[C@H](C(=O)N1CCC(c2ccc(C(=O)O)nc2)CC1)[C@@H](C)OCC12CCC(CC1)OC2]

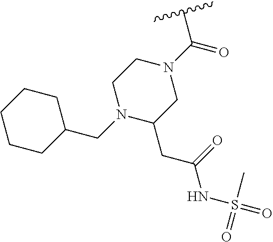 [SMILES: *C(=O)N1CCN(CC2CCCCC2)C(CC(=O)NS(C)(=O)=O)C1]

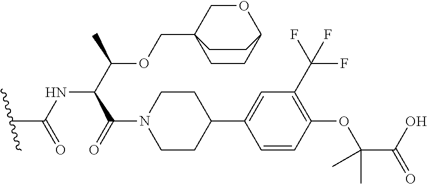 [SMILES: *C(=O)N[C@H](C(=O)N1CCC(c2ccc(OC(C)(C)C(=O)O)c(C(F)(F)F)c2)CC1)[C@@H](C)OCC12CCC(CC1)OC2]

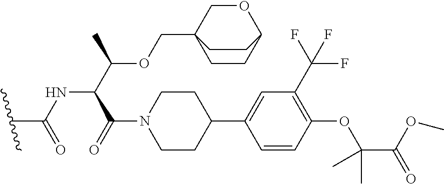 [SMILES: *C(=O)N[C@H](C(=O)N1CCC(c2ccc(OC(C)(C)C(=O)OC)c(C(F)(F)F)c2)CC1)[C@@H](C)OCC12CCC(CC1)OC2]

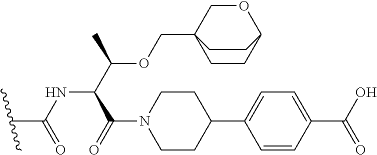 [SMILES: *C(=O)N[C@H](C(=O)N1CCC(c2ccc(C(=O)O)cc2)CC1)[C@@H](C)OCC12CCC(CC1)OC2]

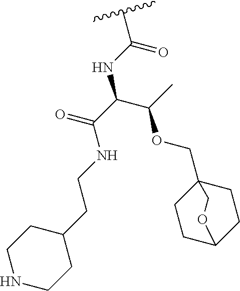 [SMILES: *C(=O)N[C@H](C(=O)NCCC1CCNCC1)[C@@H](C)OCC12CCC(CC1)OC2]

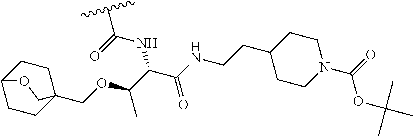 [SMILES: *C(=O)N[C@H](C(=O)NCCC1CCN(C(=O)OC(C)(C)C)CC1)[C@@H](C)OCC12CCC(CC1)OC2]

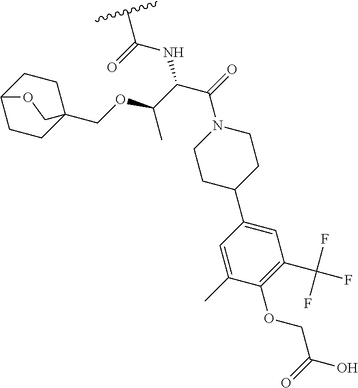 [SMILES: *C(=O)N[C@H](C(=O)N1CCC(c2cc(C)c(OCC(=O)O)c(C(F)(F)F)c2)CC1)[C@@H](C)OCC12CCC(CC1)OC2]

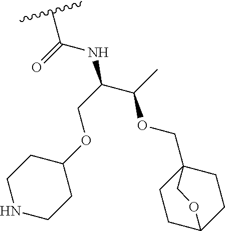 [SMILES: *C(=O)N[C@H](COC1CCNCC1)[C@@H](C)OCC12CCC(CC1)OC2]

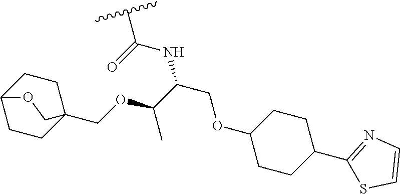 [SMILES: *C(=O)N[C@H](COC1CCC(c2nccs2)CC1)[C@@H](C)OCC12CCC(CC1)OC2]

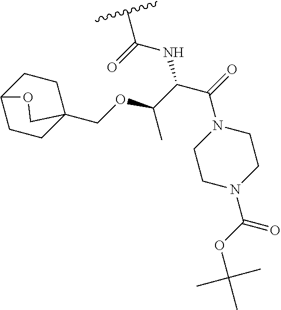 [SMILES: *C(=O)N[C@H](C(=O)N1CCN(C(=O)OC(C)(C)C)CC1)[C@@H](C)OCC12CCC(CC1)OC2]

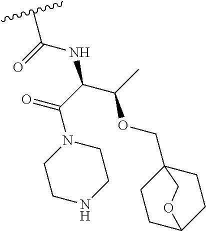 [SMILES: *C(=O)N[C@H](C(=O)N1CCNCC1)[C@@H](C)OCC12CCC(CC1)OC2]

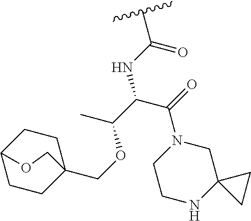 [SMILES: *C(=O)N[C@H](C(=O)N1CCNC2(CC2)C1)[C@@H](C)OCC12CCC(CC1)OC2]

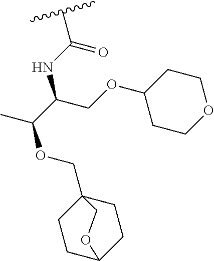 [SMILES: *C(=O)N[C@@H](COC1CCOCC1)[C@H](C)OCC12CCC(CC1)OC2]

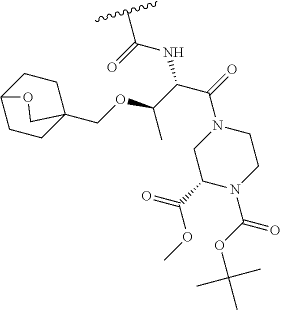 [SMILES: *C(=O)N[C@H](C(=O)N1CCN(C(=O)OC(C)(C)C)[C@H](C(=O)OC)C1)[C@@H](C)OCC12CCC(CC1)OC2]

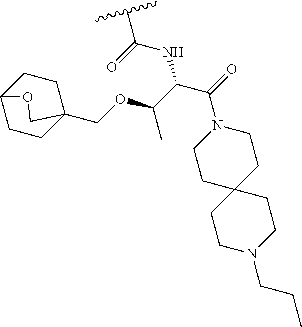 [SMILES: *C(=O)N[C@H](C(=O)N1CCC2(CCN(CCC)CC2)CC1)[C@@H](C)OCC12CCC(CC1)OC2]

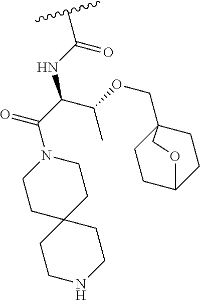 [SMILES: *C(=O)N[C@H](C(=O)N1CCC2(CCNCC2)CC1)[C@@H](C)OCC12CCC(CC1)OC2]

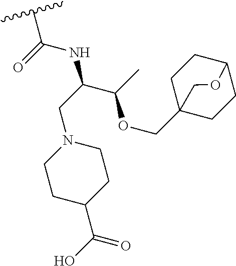 [SMILES: *C(=O)N[C@H](CN1CCC(C(=O)O)CC1)[C@@H](C)OCC12CCC(CC1)OC2]

TABLE Bii
Exemplary R^{A} substituents

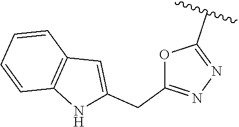 [SMILES: *c1nnc(Cc2cc3ccccc3[nH]2)o1]

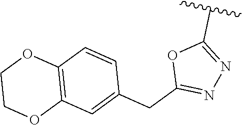 [SMILES: *c1nnc(Cc2ccc3c(c2)OCCO3)o1]

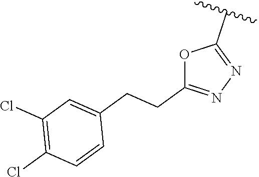 [SMILES: *c1nnc(CCc2ccc(Cl)c(Cl)c2)o1]

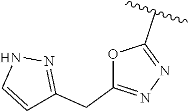 [SMILES: *c1nnc(Cc2cc[nH]n2)o1]

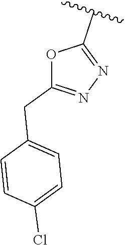 [SMILES: *c1nnc(Cc2ccc(Cl)cc2)o1]

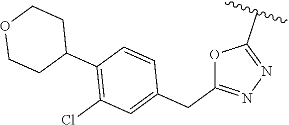 [SMILES: *c1nnc(Cc2ccc(C3CCOCC3)c(Cl)c2)o1]

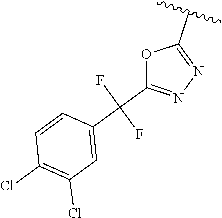 [SMILES: *c1nnc(C(F)(F)c2ccc(Cl)c(Cl)c2)o1]

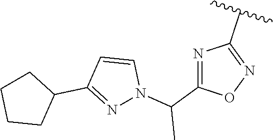 [SMILES: *c1noc(C(C)n2ccc(C3CCCC3)n2)n1]

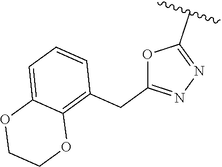 [SMILES: *c1nnc(Cc2cccc3c2OCCO3)o1]

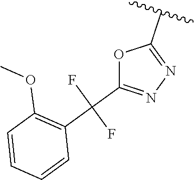 [SMILES: *c1nnc(C(F)(F)c2ccccc2OC)o1]

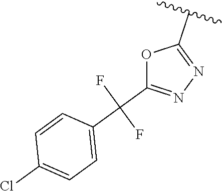 [SMILES: *c1nnc(C(F)(F)c2ccc(Cl)cc2)o1]

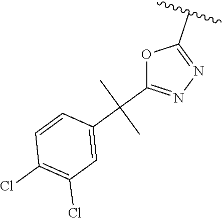 [SMILES: *c1nnc(C(C)(C)c2ccc(Cl)c(Cl)c2)o1]

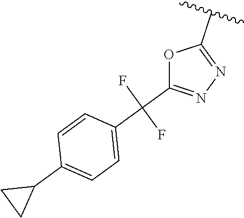 [SMILES: *c1nnc(C(F)(F)c2ccc(C3CC3)cc2)o1]

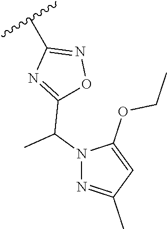 [SMILES: *c1noc(C(C)n2nc(C)cc2OCC)n1]

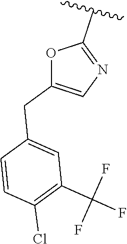 [SMILES: *c1ncc(Cc2ccc(Cl)c(C(F)(F)F)c2)o1]

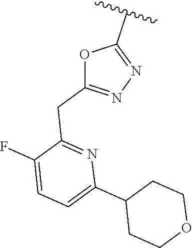 [SMILES: *c1nnc(Cc2nc(C3CCOCC3)ccc2F)o1]

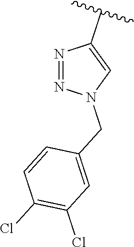 [SMILES: *c1cn(Cc2ccc(Cl)c(Cl)c2)nn1]

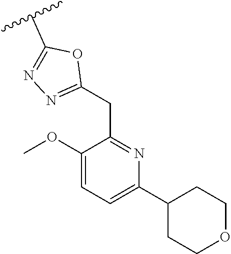 [SMILES: *c1nnc(Cc2nc(C3CCOCC3)ccc2OC)o1]

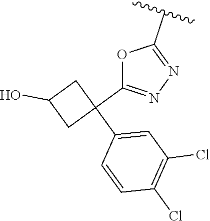 [SMILES: *c1nnc(C2(c3ccc(Cl)c(Cl)c3)CC(O)C2)o1]

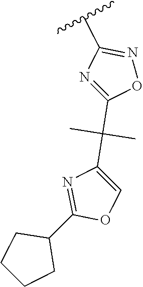 [SMILES: *c1noc(C(C)(C)c2coc(C3CCCC3)n2)n1]

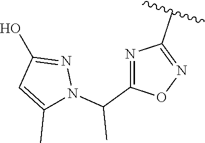 [SMILES: *c1noc(C(C)n2nc(O)cc2C)n1]

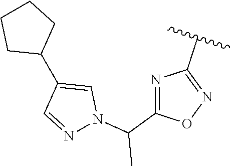 [SMILES: *c1noc(C(C)n2cc(C3CCCC3)cn2)n1]

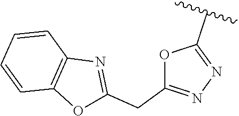 [SMILES: *c1nnc(Cc2nc3ccccc3o2)o1]

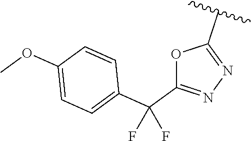 [SMILES: *c1nnc(C(F)(F)c2ccc(OC)cc2)o1]

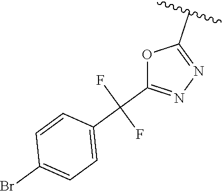 [SMILES: *c1nnc(C(F)(F)c2ccc(Br)cc2)o1]

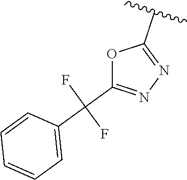 [SMILES: *c1nnc(C(F)(F)c2ccccc2)o1]

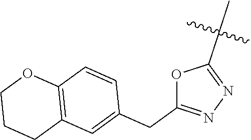 [SMILES: *c1nnc(Cc2ccc3c(c2)CCCO3)o1]

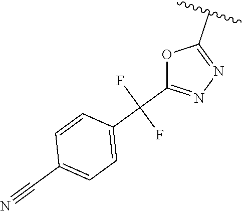 [SMILES: *c1nnc(C(F)(F)c2ccc(C#N)cc2)o1]

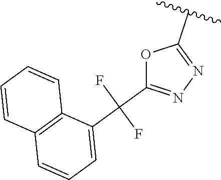 [SMILES: *c1nnc(C(F)(F)c2cccc3ccccc23)o1]

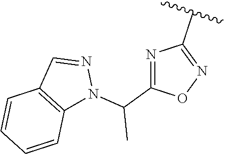 [SMILES: *c1noc(C(C)n2ncc3ccccc32)n1]

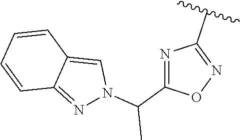 [SMILES: *c1noc(C(C)n2cc3ccccc3n2)n1]

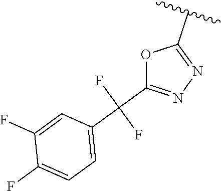 [SMILES: *c1nnc(C(F)(F)c2ccc(F)c(F)c2)o1]

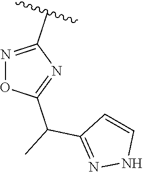 [SMILES: *c1noc(C(C)c2cc[nH]n2)n1]

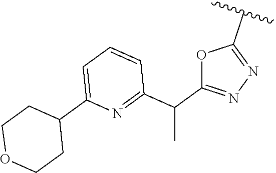 [SMILES: *c1nnc(C(C)c2cccc(C3CCOCC3)n2)o1]

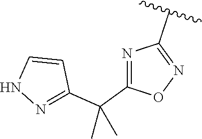 [SMILES: *c1noc(C(C)(C)c2cc[nH]n2)n1]

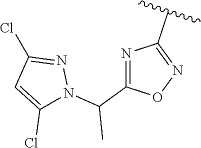 [SMILES: *c1noc(C(C)n2nc(Cl)cc2Cl)n1]

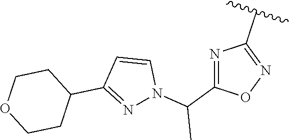 [SMILES: *c1noc(C(C)n2ccc(C3CCOCC3)n2)n1]

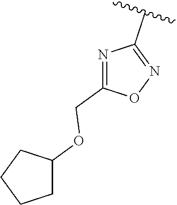 [SMILES: *c1noc(COC2CCCC2)n1]

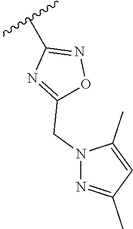 [SMILES: *c1noc(Cn2nc(C)cc2C)n1]

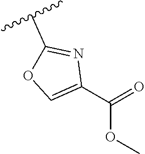 [SMILES: *c1nc(C(=O)OC)co1]

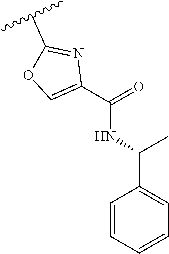 [SMILES: *c1nc(C(=O)N[C@H](C)c2ccccc2)co1]

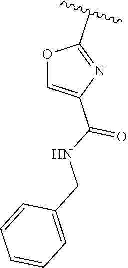 [SMILES: *c1nc(C(=O)NCc2ccccc2)co1]

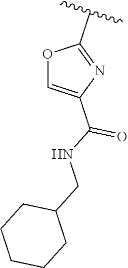 [SMILES: *c1nc(C(=O)NCC2CCCCC2)co1]

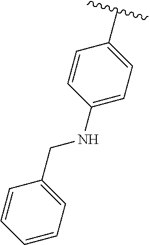 [SMILES: *c1ccc(NCc2ccccc2)cc1]

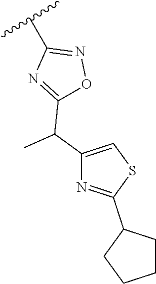 [SMILES: *c1noc(C(C)c2csc(C3CCCC3)n2)n1]

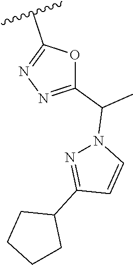 [SMILES: *c1nnc(C(C)n2ccc(C3CCCC3)n2)o1]

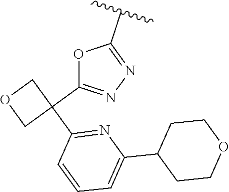 [SMILES: *c1nnc(C2(c3cccc(C4CCOCC4)n3)COC2)o1]

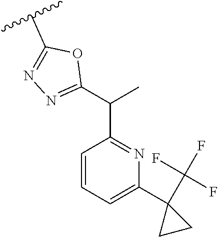 [SMILES: *c1nnc(C(C)c2cccc(C3(C(F)(F)F)CC3)n2)o1]

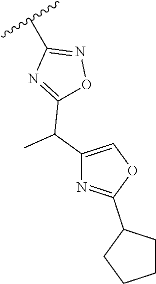 [SMILES: *c1noc(C(C)c2coc(C3CCCC3)n2)n1]

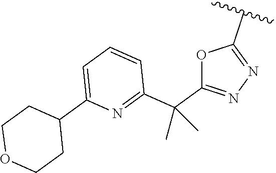 [SMILES: *c1nnc(C(C)(C)c2cccc(C3CCOCC3)n2)o1]

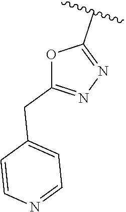 [SMILES: *c1nnc(Cc2ccncc2)o1]

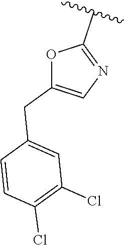 [SMILES: *c1ncc(Cc2ccc(Cl)c(Cl)c2)o1]

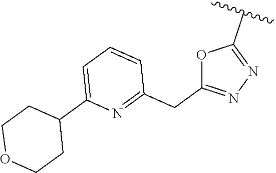 [SMILES: *c1nnc(Cc2cccc(C3CCOCC3)n2)o1]

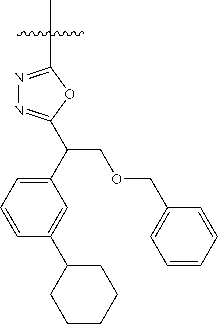 [SMILES: *c1nnc(C(COCc2ccccc2)c2cccc(C3CCCCC3)c2)o1]

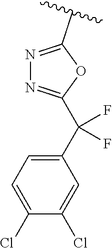 [SMILES: *c1nnc(C(F)(F)c2ccc(Cl)c(Cl)c2)o1]

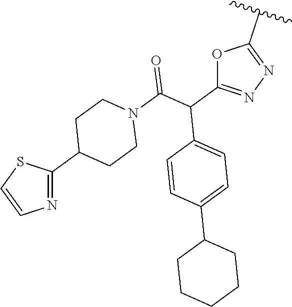 [SMILES: *c1nnc(C(C(=O)N2CCC(c3nccs3)CC2)c2ccc(C3CCCCC3)cc2)o1]

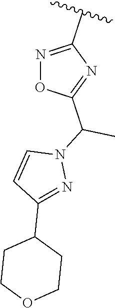 [SMILES: *c1noc(C(C)n2ccc(C3CCOCC3)n2)n1]

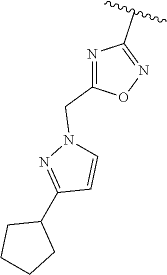 [SMILES: *c1noc(Cn2ccc(C3CCCC3)n2)n1]

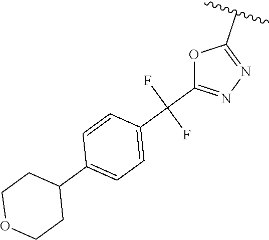 [SMILES: *c1nnc(C(F)(F)c2ccc(C3CCOCC3)cc2)o1]

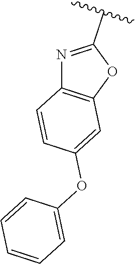 [SMILES: *c1nc2ccc(Oc3ccccc3)cc2o1]

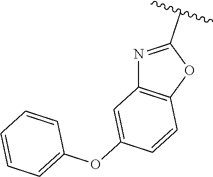 [SMILES: *c1nc2cc(Oc3ccccc3)ccc2o1]

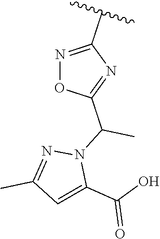 [SMILES: *c1noc(C(C)n2nc(C)cc2C(=O)O)n1]

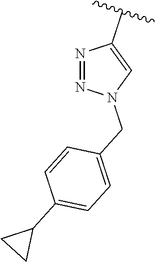 [SMILES: *c1cn(Cc2ccc(C3CC3)cc2)nn1]

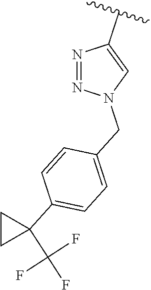 [SMILES: *c1cn(Cc2ccc(C3(C(F)(F)F)CC3)cc2)nn1]

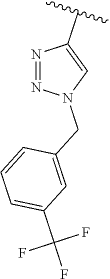 [SMILES: *c1cn(Cc2cccc(C(F)(F)F)c2)nn1]

TABLE Biii
Exemplary R^{A} substituents

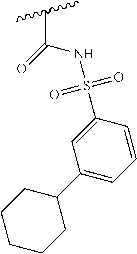 [SMILES: *C(=O)NS(=O)(=O)c1cccc(C2CCCCC2)c1]

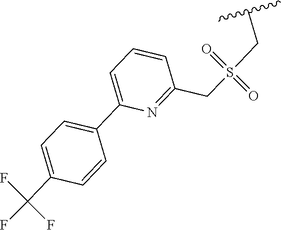 [SMILES: *CS(=O)(=O)Cc1cccc(-c2ccc(C(F)(F)F)cc2)n1]

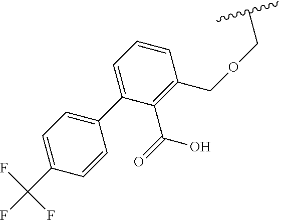 [SMILES: *COCc1cccc(-c2ccc(C(F)(F)F)cc2)c1C(=O)O]

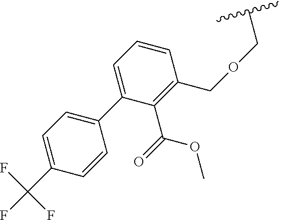 [SMILES: *COCc1cccc(-c2ccc(C(F)(F)F)cc2)c1C(=O)OC]

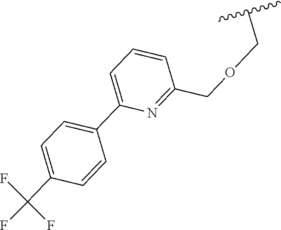 [SMILES: *COCc1cccc(-c2ccc(C(F)(F)F)cc2)n1]

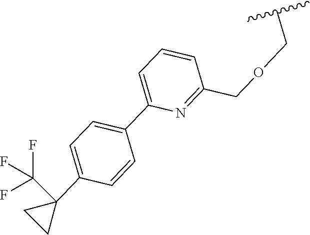 [SMILES: *COCc1cccc(-c2ccc(C3(C(F)(F)F)CC3)cc2)n1]

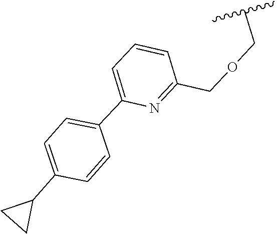 [SMILES: *COCc1cccc(-c2ccc(C3CC3)cc2)n1]

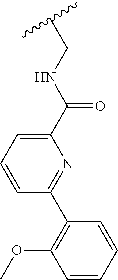 [SMILES: *CNC(=O)c1cccc(-c2ccccc2OC)n1]

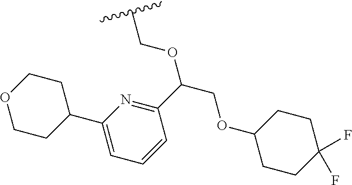 [SMILES: *COC(COC1CCC(F)(F)CC1)c1cccc(C2CCOCC2)n1]

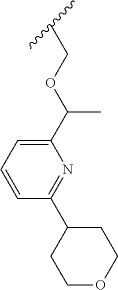 [SMILES: *COC(C)c1cccc(C2CCOCC2)n1]

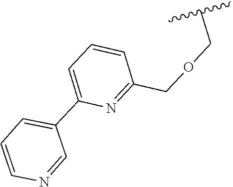 [SMILES: *COCc1cccc(-c2cccnc2)n1]

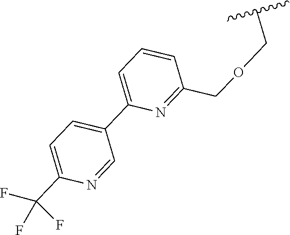 [SMILES: *COCc1cccc(-c2ccc(C(F)(F)F)nc2)n1]

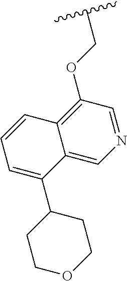 [SMILES: *COc1cncc2c(C3CCOCC3)cccc12]

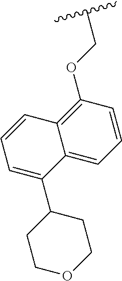 [SMILES: *COc1cccc2c(C3CCOCC3)cccc12]

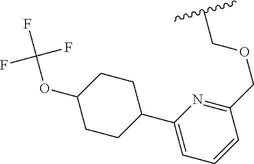 [SMILES: *COCc1cccc(C2CCC(OC(F)(F)F)CC2)n1]

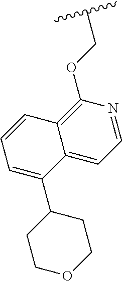 [SMILES: *COc1nccc2c(C3CCOCC3)cccc12]

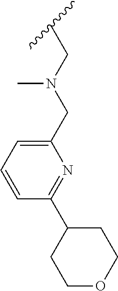 [SMILES: *CN(C)Cc1cccc(C2CCOCC2)n1]

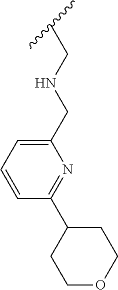 [SMILES: *CNCc1cccc(C2CCOCC2)n1]

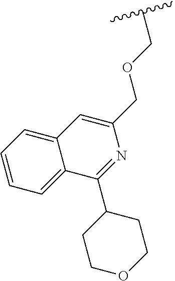 [SMILES: *COCc1cc2ccccc2c(C2CCOCC2)n1]

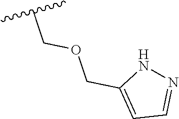 [SMILES: *COCc1ccn[nH]1]

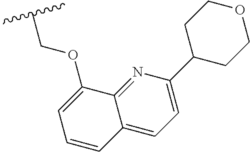 [SMILES: *COc1cccc2ccc(C3CCOCC3)nc12]

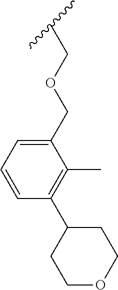 [SMILES: *COCc1cccc(C2CCOCC2)c1C]

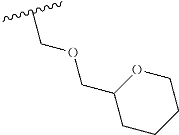 [SMILES: *COCC1CCCCO1]

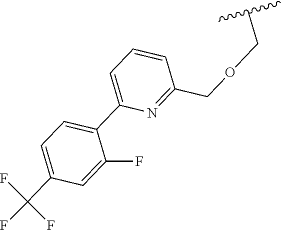 [SMILES: *COCc1cccc(-c2ccc(C(F)(F)F)cc2F)n1]

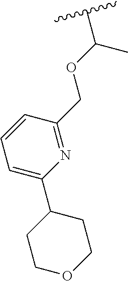 [SMILES: *C(C)OCc1cccc(C2CCOCC2)n1]

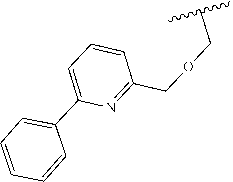 [SMILES: *COCc1cccc(-c2ccccc2)n1]

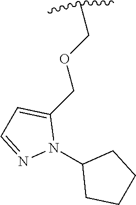 [SMILES: *COCc1ccnn1C1CCCC1]

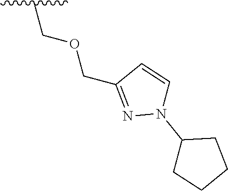 [SMILES: *COCc1ccn(C2CCCC2)n1]

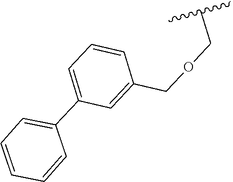 [SMILES: *COCc1cccc(-c2ccccc2)c1]

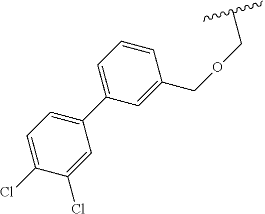 [SMILES: *COCc1cccc(-c2ccc(Cl)c(Cl)c2)c1]

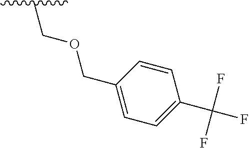 [SMILES: *COCc1ccc(C(F)(F)F)cc1]

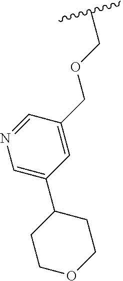 [SMILES: *COCc1cncc(C2CCOCC2)c1]

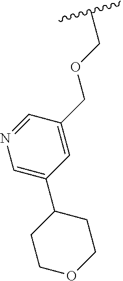 [SMILES: *COCc1cncc(C2CCOCC2)c1]

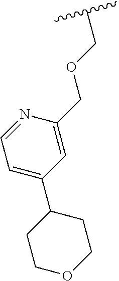 [SMILES: *COCc1cc(C2CCOCC2)ccn1]

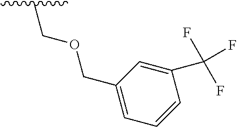 [SMILES: *COCc1cccc(C(F)(F)F)c1]

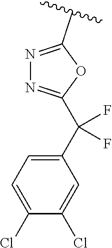 [SMILES: *c1nnc(C(F)(F)c2ccc(Cl)c(Cl)c2)o1]

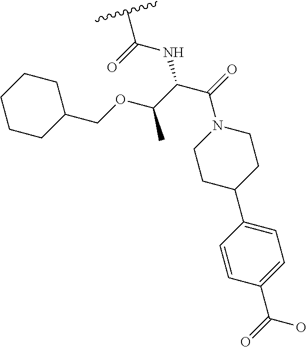 [SMILES: *C(=O)N[C@H](C(=O)N1CCC(c2ccc(C(=O)O)cc2)CC1)[C@@H](C)OCC1CCCCC1]

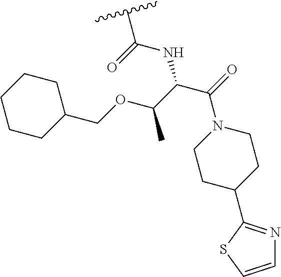 [SMILES: *C(=O)N[C@H](C(=O)N1CCC(c2nccs2)CC1)[C@@H](C)OCC1CCCCC1]

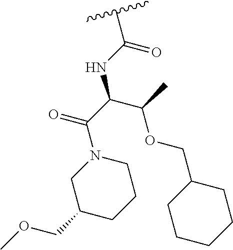 [SMILES: *C(=O)N[C@H](C(=O)N1CCC[C@H](COC)C1)[C@@H](C)OCC1CCCCC1]

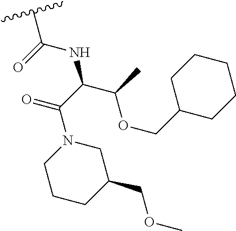 [SMILES: *C(=O)N[C@H](C(=O)N1CCC[C@H](COC)C1)[C@@H](C)OCC1CCCCC1]

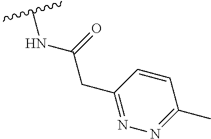 [SMILES: *NC(=O)Cc1ccc(C)nn1]

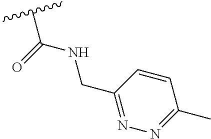 [SMILES: *C(=O)NCc1ccc(C)nn1]

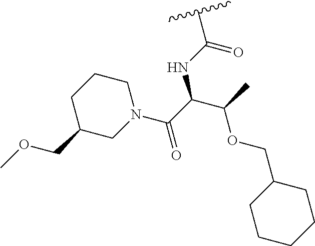 [SMILES: *C(=O)N[C@H](C(=O)N1CCC[C@H](COC)C1)[C@@H](C)OCC1CCCCC1]

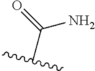 [SMILES: *C(N)=O]

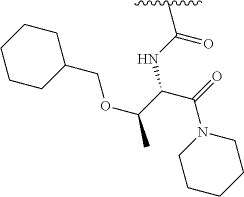 [SMILES: *C(=O)N[C@H](C(=O)N1CCCCC1)[C@@H](C)OCC1CCCCC1]

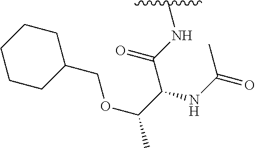 [SMILES: *NC(=O)[C@H](NC(C)=O)[C@H](C)OCC1CCCCC1]

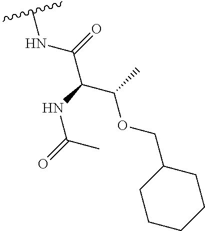 [SMILES: *NC(=O)[C@H](NC(C)=O)[C@H](C)OCC1CCCCC1]

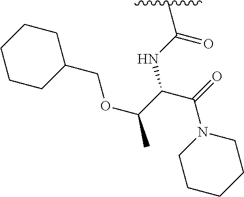 [SMILES: *C(=O)N[C@H](C(=O)N1CCCCC1)[C@@H](C)OCC1CCCCC1]

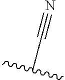 [SMILES: *C#N]

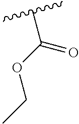 [SMILES: *C(=O)OCC]

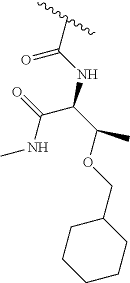 [SMILES: *C(=O)N[C@H](C(=O)NC)[C@@H](C)OCC1CCCCC1]

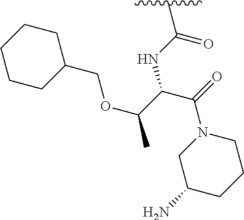 [SMILES: *C(=O)N[C@H](C(=O)N1CCC[C@H](N)C1)[C@@H](C)OCC1CCCCC1]

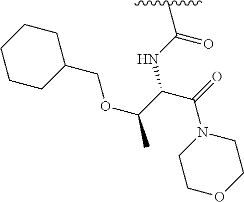 [SMILES: *C(=O)N[C@H](C(=O)N1CCOCC1)[C@@H](C)OCC1CCCCC1]

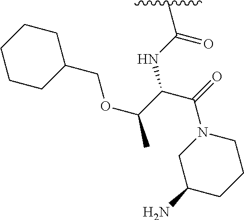 [SMILES: *C(=O)N[C@H](C(=O)N1CCC[C@@H](N)C1)[C@@H](C)OCC1CCCCC1]

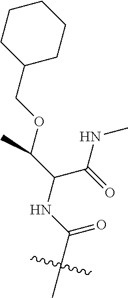 [SMILES: *C(=O)NC(C(=O)NC)[C@@H](C)OCC1CCCCC1]

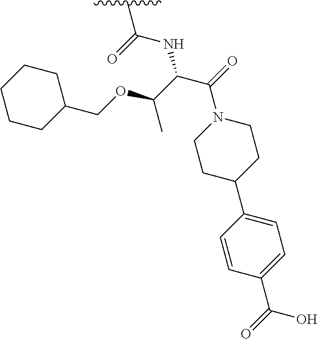 [SMILES: *C(=O)N[C@H](C(=O)N1CCC(c2ccc(C(=O)O)cc2)CC1)[C@@H](C)OCC1CCCCC1]

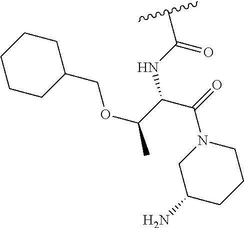 [SMILES: *C(=O)N[C@H](C(=O)N1CCC[C@H](N)C1)[C@@H](C)OCC1CCCCC1]

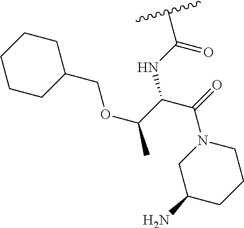 [SMILES: *C(=O)N[C@H](C(=O)N1CCC[C@@H](N)C1)[C@@H](C)OCC1CCCCC1]

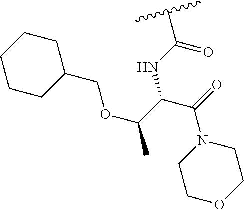 [SMILES: *C(=O)N[C@H](C(=O)N1CCOCC1)[C@@H](C)OCC1CCCCC1]

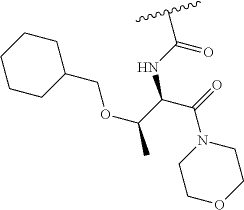 [SMILES: *C(=O)N[C@@H](C(=O)N1CCOCC1)[C@@H](C)OCC1CCCCC1]

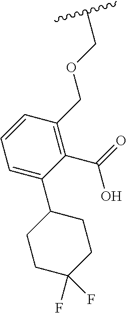 [SMILES: *COCc1cccc(C2CCC(F)(F)CC2)c1C(=O)O]

In some embodiments, R^{A} is

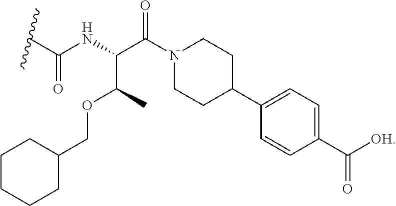 [SMILES: *C(=O)N[C@H](C(=O)N1CCC(c2ccc(C(=O)O)cc2)CC1)[C@@H](C)OCC1CCCCC1]

In some embodiments, R^{A} is

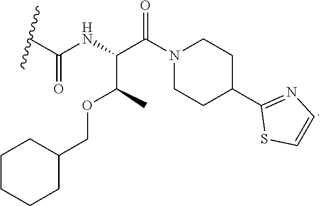 [SMILES: *C(=O)N[C@H](C(=O)N1CCC(c2nccs2)CC1)[C@@H](C)OCC1CCCCC1]

In some embodiments, R^{A} is

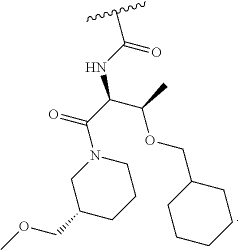 [SMILES: *C(=O)N[C@H](C(=O)N1CCC[C@H](COC)C1)[C@@H](C)OCC1CCCCC1]

In some embodiments, R^{A} is

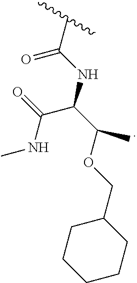 [SMILES: *C(=O)N[C@H](C(=O)NC)[C@@H](C)OCC1CCCCC1]

As defined generally above, L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—. In some embodiments, L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L² is a covalent bond

In some embodiments, L² is a saturated or unsaturated, straight or branched, optionally substituted bivalent C₁₋₄ hydrocarbon chain, wherein 0-2 methylene units of L² are independently replaced by —C(O)O—, —C(O)—, or —C(O)NR—. In some embodiments, L² is a C₁₋₄ alkylene chain, wherein 1-2 methylene units of L² are independently replaced by —C(O)O—, —C(O)—, or —C(O)NR—. In some embodiments, L² is C₁₋₄ alkylene chain, wherein 1 methylene unit of L² is replaced by —C(O)O—, —C(O)—, or —C(O)NR—. In some embodiments, L² is a saturated optionally substituted bivalent C₁₋₄ hydrocarbon chain. In some embodiments, L² is a saturated bivalent C₁₋₄ hydrocarbon chain, substituted on a single methylene unit by two substituents, which together with the intervening carbon atom form a 3-7 membered carbocyclic ring or heterocyclic ring (having 1-2 heteroatoms independently selected from nitrogen, oxygen, and sulfur). In some embodiments, L² is

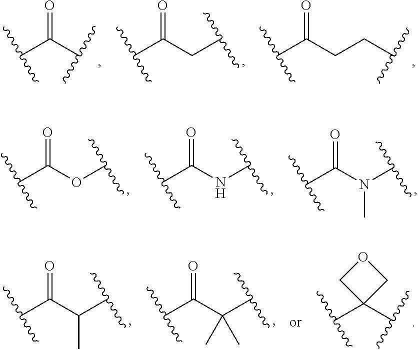

In some embodiments, L² is

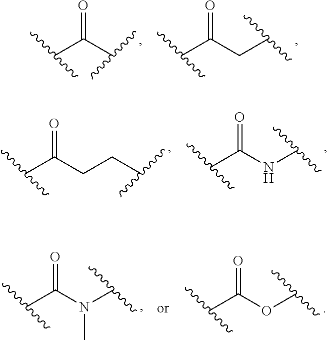

In some embodiments, L² is

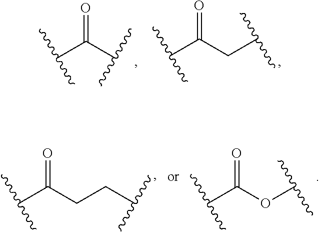

In some embodiments, L² is

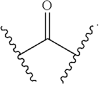 [SMILES: *C(*)=O]

In some embodiments, L² is selected from those depicted in the compounds of Table 1, below.

In some embodiments, L² is a saturated, straight or branched, optionally substituted bivalent C₁₋₄ hydrocarbon chain. In some embodiments, L² is methylene.

In some embodiments, L² is —S(O)₂—.

As defined generally above R⁶ is hydrogen, —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁷ or R⁷;

In some embodiments, R⁶ is hydrogen. In some embodiments, R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷.

In some embodiments, R⁶ is an optionally substituted C₁₋₆ aliphatic group. In some embodiments, R⁶ is an optionally substituted methyl, ethyl, isopropyl, or tert-butyl group.

In some embodiments, R⁶ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷. In some embodiments, R⁶ is a 3-8 membered saturated or partially unsaturated monocyclic carbocyclic ring, optionally substituted with one or more instances of R⁷. In some embodiments, R⁶ is a phenyl group, optionally substituted with one or more instances of R⁷. In some embodiments, R⁶ is a cyclic group selected from cyclopropyl, cyclobutyl, cyclohexyl and phenyl, wherein the cyclic group is optionally substituted with one or more instances of R⁷. In some embodiments, R⁶ is a cyclopropyl group, optionally substituted with one or more instances of R⁷. In some embodiments, R⁶ is a cyclopropyl group, optionally substituted with one instance of —CF₃. In some embodiments, R⁶ is selected from those depicted in the compounds of Table 1, below.

In some embodiments, R⁶ is a 3-8 membered saturated or partially unsaturated monocyclic heterocyclic ring (having 1-2 heteroatoms independently selected from nitrogen, oxygen, and sulfur), optionally substituted with one or more instances of R⁷. In some embodiments, R⁶ is tetrahydrofuranyl, optionally substituted with one or more instances of R⁷. In some embodiments, R⁶ is tetrahydropyranyl, optionally substituted with one or more instances of R⁷. In some embodiments, R⁶ is oxetanyl, optionally substituted with one or more instances of R⁷.

In some embodiments, R⁶ is a 5-6 membered monocyclic heteroaromatic ring (having 1-4 heteroatoms independently selected from nitrogen, oxygen, and sulfur), optionally substituted with one or more instances of R⁷. In some embodiments, R⁶ is furanyl, optionally substituted with one or more instances of R⁷. In some embodiments, R⁶ is pyrazolyl, optionally substituted with one or more instances of R⁷. In some embodiments, R⁶ is oxazolyl, optionally substituted with one or more instances of R⁷.

As defined generally above, each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy. In some embodiments, each instance of R⁷ is independently halogen, —OR, —CN, an optionally substituted C₁₋₆ aliphatic group, an optionally substituted C₁₋₆ aliphatic-Cy group, or Cy. In some embodiments, each instance of R⁷ is independently —F, methyl, ethyl, isopropyl, isobutyl, —CN, optionally substituted phenyl, optionally substituted benzyl, —CF₃, —CH₂OH, —CH₂OCH₃, —CH₂CH₂OCH₃, —CH₂CH₂F, cyclopropyl or —CH₂-(cyclopropyl). In some embodiments, each instance of R⁷ is independently a C₁₋₆ aliphatic group. In some embodiments, R⁷ is —CF₃.

In some embodiments, -L²-R⁶ is a substituent of Table C:

TABLE C
Exemplary —L²—R⁶ substituents

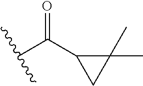 [SMILES: *C(=O)C1CC1(C)C]

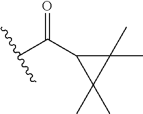 [SMILES: *C(=O)C1C(C)(C)C1(C)C]

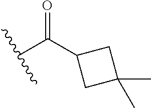 [SMILES: *C(=O)C1CC(C)(C)C1]

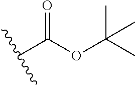 [SMILES: *C(=O)OC(C)(C)C]

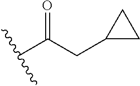 [SMILES: *C(=O)CC1CC1]

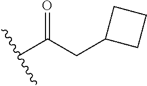 [SMILES: *C(=O)CC1CCC1]

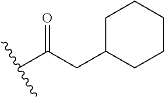 [SMILES: *C(=O)CC1CCCCC1]

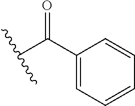 [SMILES: *C(=O)c1ccccc1]

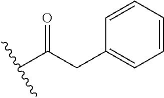 [SMILES: *C(=O)Cc1ccccc1]

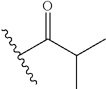 [SMILES: *C(=O)C(C)C]

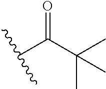 [SMILES: *C(=O)C(C)(C)C]

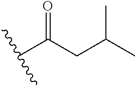 [SMILES: *C(=O)CC(C)C]

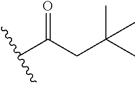 [SMILES: *C(=O)CC(C)(C)C]

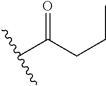 [SMILES: *C(=O)CCC]

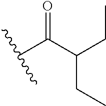 [SMILES: *C(=O)C(CC)CC]

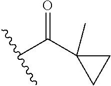 [SMILES: *C(=O)C1(C)CC1]

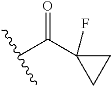 [SMILES: *C(=O)C1(F)CC1]

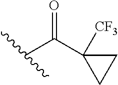 [SMILES: *C(=O)C1(C(F)(F)F)CC1]

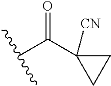 [SMILES: *C(=O)C1(C#N)CC1]

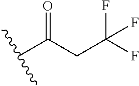 [SMILES: *C(=O)CC(F)(F)F]

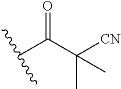 [SMILES: *C(=O)C(C)(C)C#N]

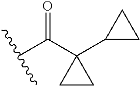 [SMILES: *C(=O)C1(C2CC2)CC1]

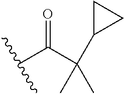 [SMILES: *C(=O)C(C)(C)C1CC1]

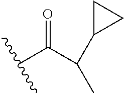 [SMILES: *C(=O)C(C)C1CC1]

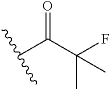 [SMILES: *C(=O)C(C)(C)F]

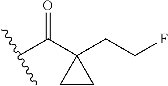 [SMILES: *C(=O)C1(CCF)CC1]

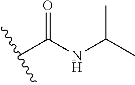 [SMILES: *C(=O)NC(C)C]

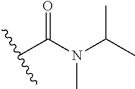 [SMILES: *C(=O)N(C)C(C)C]

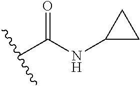 [SMILES: *C(=O)NC1CC1]

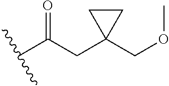 [SMILES: *C(=O)CC1(COC)CC1]

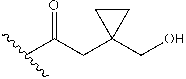 [SMILES: *C(=O)CC1(CO)CC1]

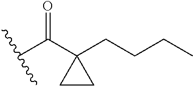 [SMILES: *C(=O)C1(CCCC)CC1]

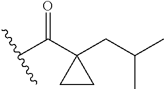 [SMILES: *C(=O)C1(CC(C)C)CC1]

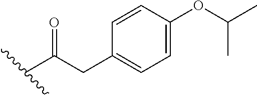 [SMILES: *C(=O)Cc1ccc(OC(C)C)cc1]

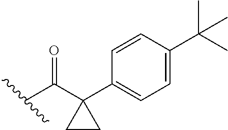 [SMILES: *C(=O)C1(c2ccc(C(C)(C)C)cc2)CC1]

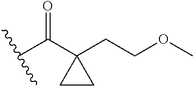 [SMILES: *C(=O)C1(CCOC)CC1]

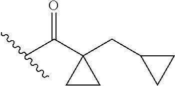 [SMILES: *C(=O)C1(CC2CC2)CC1]

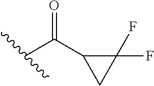 [SMILES: *C(=O)C1CC1(F)F]

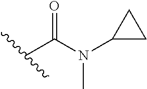 [SMILES: *C(=O)N(C)C1CC1]

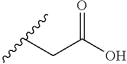 [SMILES: *CC(=O)O]

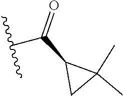 [SMILES: *C(=O)[C@@H]1CC1(C)C]

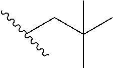 [SMILES: *CC(C)(C)C]

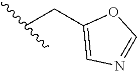 [SMILES: *Cc1cnco1]

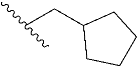 [SMILES: *CC1CCCC1]

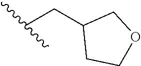 [SMILES: *CC1CCOC1]

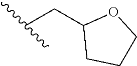 [SMILES: *CC1CCCO1]

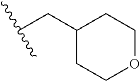 [SMILES: *CC1CCOCC1]

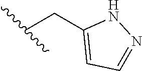 [SMILES: *Cc1ccn[nH]1]

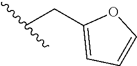 [SMILES: *Cc1ccco1]

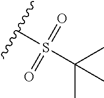 [SMILES: *S(=O)(=O)C(C)(C)C]

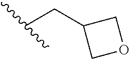 [SMILES: *CC1COC1]

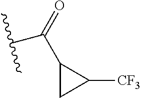 [SMILES: *C(=O)C1CC1C(F)(F)F]

—H

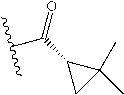 [SMILES: *C(=O)[C@H]1CC1(C)C]

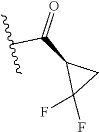 [SMILES: *C(=O)[C@H]1CC1(F)F]

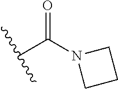 [SMILES: *C(=O)N1CCC1]

—CN
C(O)OC(CH₃)₃

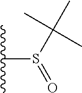 [SMILES: *S(=O)C(C)(C)C]

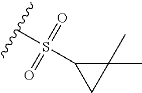 [SMILES: *S(=O)(=O)C1CC1(C)C]

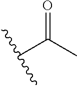 [SMILES: *C(C)=O]

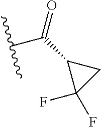 [SMILES: *C(=O)[C@@H]1CC1(F)F]

In some embodiments, -L²-R⁶ is

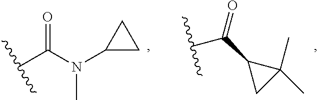

### —H, —CN,

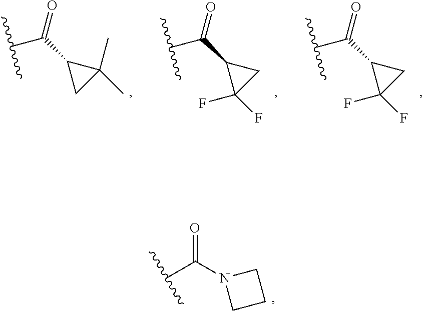

—C(O)OC(CH₃)₃,

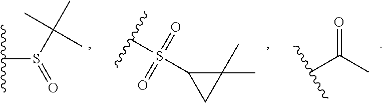

In some embodiments, -L²-R⁶ is

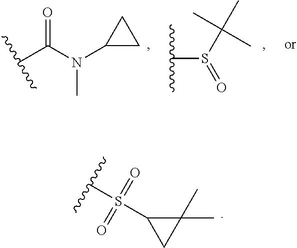

In some embodiments, -L²-R⁶ is

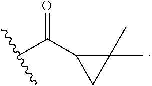 [SMILES: *C(=O)C1CC1(C)C]

In some embodiments, -L²-R¹ is

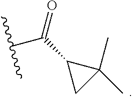 [SMILES: *C(=O)[C@H]1CC1(C)C]

In some embodiments, -L²-R⁶ is

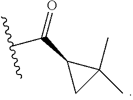 [SMILES: *C(=O)[C@@H]1CC1(C)C]

In some embodiments, -L²-R⁶ is

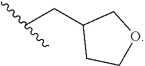 [SMILES: *CC1CCOC1]

In some embodiments, -L²-R⁶ is

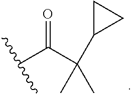 [SMILES: *C(=O)C(C)(C)C1CC1]

In some embodiments, -L²-R⁶ is

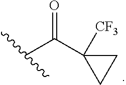 [SMILES: *C(=O)C1(C(F)(F)F)CC1]

As defined generally above, L³ is a covalent bond, a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or, -Cy-, —NRC(O)NR—. In some embodiments, L³ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L³ is a saturated or unsaturated, straight or branched, optionally substituted bivalent C₁₋₄ hydrocarbon chain, wherein 0-2 methylene units of L³ are independently replaced by —S(O)₂—, —C(O)NR—, or —C(O)—. In some embodiments, L³ is a C₁₋₄ alkylene chain, wherein 1-2 methylene units of L³ are independently replaced by —S(O)₂—, —C(O)NR—, or —C(O)—. In some embodiments, L³ is C₁₋₄ alkylene chain, wherein 1 methylene unit of L³ is replaced by —S(O)₂—, —C(O)NR—, or —C(O)—. In some embodiments, L³ is a saturated or unsaturated, straight or branched, optionally substituted bivalent C₁₋₄ alkylene chain, wherein 0-2 methylene units of L³ are independently replaced by —C(O)O—, or —C(O)—. In some embodiments, L³ is a C₁₋₄ alkylene chain, wherein 1-2 methylene units of L³ are independently replaced by —C(O)O—, or —C(O)—. In some embodiments, L³ is C₁₋₄ alkylene chain, wherein 1 methylene unit of L³ is replaced by —C(O)O—, or —C(O)—. In some embodiments, L³ is a saturated optionally substituted bivalent C₁₋₄ hydrocarbon chain. In some embodiments, L³ is a saturated bivalent C₁₋₄ hydrocarbon chain, substituted on a single methylene unit by two substituents, which together with the intervening carbon atom form a 3-7 membered carbocyclic ring or heterocyclic ring (having 1-2 heteroatoms independently selected from nitrogen, oxygen, and sulfur). In some embodiments, L³ is

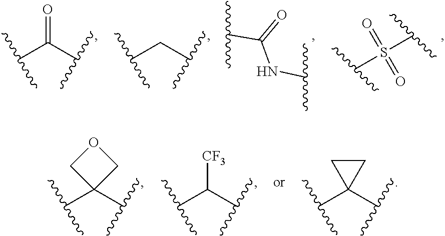

In some embodiments, L³ is

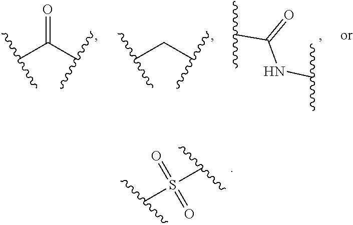

In some embodiments, L³ is

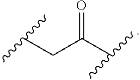 [SMILES: *CC(*)=O]

In some embodiments, L³ is

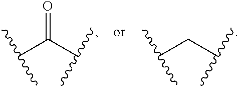

In some embodiments, L³ is

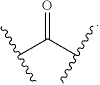 [SMILES: *C(*)=O]

In some embodiments, L³ is selected from those depicted in the compounds of Table 1, below.

As defined generally above, R⁸ is hydrogen, a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹.

In some embodiments, R⁸ is hydrogen. In some embodiments, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a cyclic group selected from a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a 3-8 membered saturated or partially unsaturated monocyclic heterocyclic ring (having 1-2 heteroatoms independently selected from nitrogen, oxygen, and sulfur), optionally substituted with one or more instances of R⁹. In some embodiments, R⁸ is a 5-6 membered monocyclic heteroaromatic ring (having 1-4 heteroatoms independently selected from nitrogen, oxygen, and sulfur), optionally substituted with one or more instances of R⁹. In some embodiments, R⁸ is an 8-10 membered bicyclic heteroaromatic ring (having 1-5 heteroatoms independently selected from nitrogen, oxygen, and sulfur), optionally substituted with one or more instances of R⁹. In some embodiments, R⁸ is a cyclic group selected from pyrazolyl, oxazolyl, thiazolyl, pyrrolidinyl, tetrahydropyranyl, pyridinyl, imidazolyl, indolyl, 1,2,4-triazolyl, 1,2,4-thiadiazolyl, piperidinyl, pyrazinyl, and indazolyl, wherein the cyclic group is optionally substituted with one or more instances of R⁹. In some embodiments, R⁸ is a pyrazolyl or thiazolyl group, optionally substituted with one or more instances of R⁹. In some embodiments, R⁸ is a pyrazolyl or thiazolyl group. In some embodiments, R⁸ is selected from those depicted in the compounds of Table 1, below.

As defined generally above, each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy.

In some embodiments, each instance of R⁹ is independently halogen, an optionally substituted C₁₋₆ aliphatic group, an optionally substituted C₁₋₆ aliphatic-Cy group, or Cy. In some embodiments, each instance of R⁹ is independently an optionally substituted C₁₋₆ aliphatic-Cy group, wherein the Cy is an optionally substituted group selected from phenyl, cyclohexyl, pyridinyl, piperidinyl, cyclopropyl, or tetrahydropyranyl. In some embodiments, R⁹ is a benzylic group. In some embodiments, each instance of R⁹ is independently halogen or an optionally substituted C₁₋₆ aliphatic group. In some embodiments, R⁹ is selected from those depicted in the compounds of Table 1, below.

In some embodiments, -L³-R¹ is a substituent of Table D:

TABLE D
Exemplary —L³—R⁸ substituents

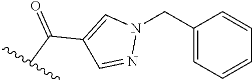 [SMILES: *C(=O)c1cnn(Cc2ccccc2)c1]

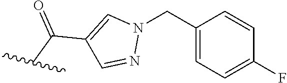 [SMILES: *C(=O)c1cnn(Cc2ccc(F)cc2)c1]

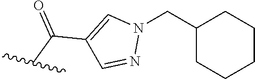 [SMILES: *C(=O)c1cnn(CC2CCCCC2)c1]

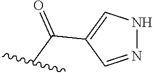 [SMILES: *C(=O)c1cn[nH]c1]

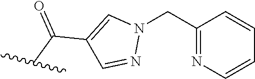 [SMILES: *C(=O)c1cnn(Cc2ccccn2)c1]

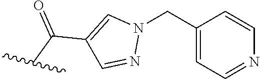 [SMILES: *C(=O)c1cnn(Cc2ccncc2)c1]

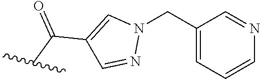 [SMILES: *C(=O)c1cnn(Cc2cccnc2)c1]

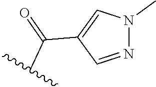 [SMILES: *C(=O)c1cnn(C)c1]

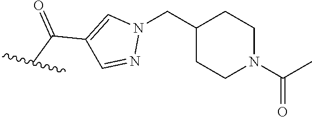 [SMILES: *C(=O)c1cnn(CC2CCN(C(C)=O)CC2)c1]

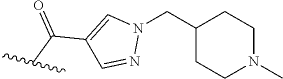 [SMILES: *C(=O)c1cnn(CC2CCN(C)CC2)c1]

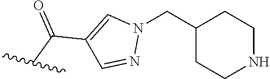 [SMILES: *C(=O)c1cnn(CC2CCNCC2)c1]

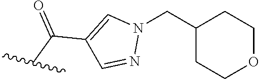 [SMILES: *C(=O)c1cnn(CC2CCOCC2)c1]

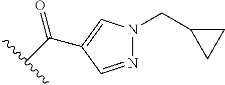 [SMILES: *C(=O)c1cnn(CC2CC2)c1]

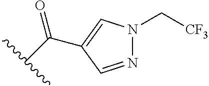 [SMILES: *C(=O)c1cnn(CC(F)(F)F)c1]

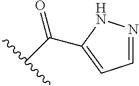 [SMILES: *C(=O)c1ccn[nH]1]

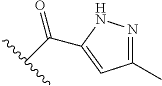 [SMILES: *C(=O)c1cc(C)n[nH]1]

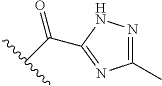 [SMILES: *C(=O)c1nc(C)n[nH]1]

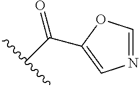 [SMILES: *C(=O)c1cnco1]

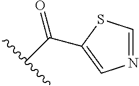 [SMILES: *C(=O)c1cncs1]

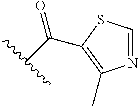 [SMILES: *C(=O)c1scnc1C]

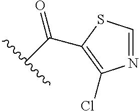 [SMILES: *C(=O)c1scnc1Cl]

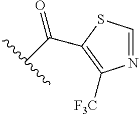 [SMILES: *C(=O)c1scnc1C(F)(F)F]

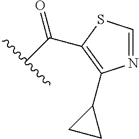 [SMILES: *C(=O)c1scnc1C1CC1]

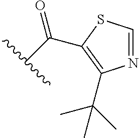 [SMILES: *C(=O)c1scnc1C(C)(C)C]

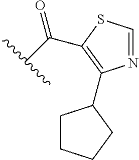 [SMILES: *C(=O)c1scnc1C1CCCC1]

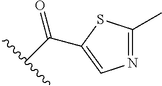 [SMILES: *C(=O)c1cnc(C)s1]

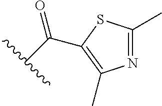 [SMILES: *C(=O)c1sc(C)nc1C]

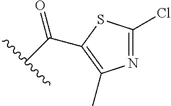 [SMILES: *C(=O)c1sc(Cl)nc1C]

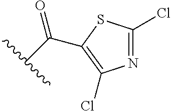 [SMILES: *C(=O)c1sc(Cl)nc1Cl]

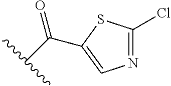 [SMILES: *C(=O)c1cnc(Cl)s1]

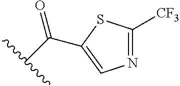 [SMILES: *C(=O)c1cnc(C(F)(F)F)s1]

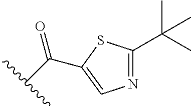 [SMILES: *C(=O)c1cnc(C(C)(C)C)s1]

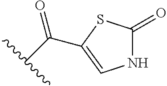 [SMILES: *C(=O)c1c[nH]c(=O)s1]

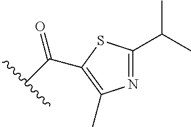 [SMILES: *C(=O)c1sc(C(C)C)nc1C]

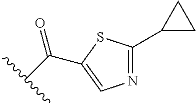 [SMILES: *C(=O)c1cnc(C2CC2)s1]

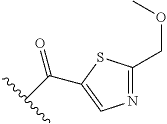 [SMILES: *C(=O)c1cnc(COC)s1]

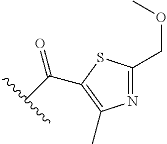 [SMILES: *C(=O)c1sc(COC)nc1C]

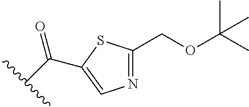 [SMILES: *C(=O)c1cnc(COC(C)(C)C)s1]

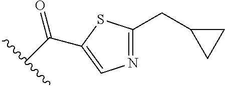 [SMILES: *C(=O)c1cnc(CC2CC2)s1]

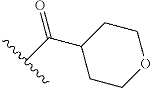 [SMILES: *C(=O)C1CCOCC1]

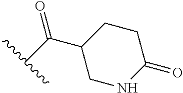 [SMILES: *C(=O)C1CCC(=O)NC1]

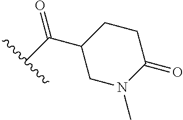 [SMILES: *C(=O)C1CCC(=O)N(C)C1]

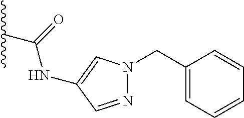 [SMILES: *C(=O)Nc1cnn(Cc2ccccc2)c1]

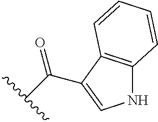 [SMILES: *C(=O)c1c[nH]c2ccccc12]

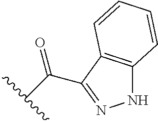 [SMILES: *C(=O)c1n[nH]c2ccccc12]

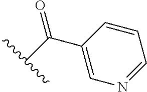 [SMILES: *C(=O)c1cccnc1]

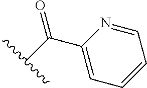 [SMILES: *C(=O)c1ccccn1]

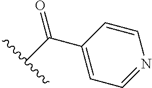 [SMILES: *C(=O)c1ccncc1]

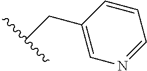 [SMILES: *Cc1cccnc1]

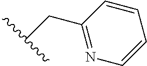 [SMILES: *Cc1ccccn1]

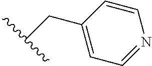 [SMILES: *Cc1ccncc1]

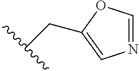 [SMILES: *Cc1cnco1]

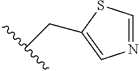 [SMILES: *Cc1cncs1]

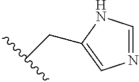 [SMILES: *Cc1cnc[nH]1]

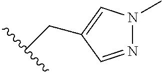 [SMILES: *Cc1cnn(C)c1]

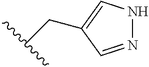 [SMILES: *Cc1cn[nH]c1]

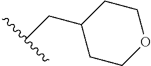 [SMILES: *CC1CCOCC1]

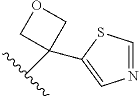 [SMILES: *C1(c2cncs2)COC1]

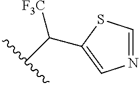 [SMILES: *C(c1cncs1)C(F)(F)F]

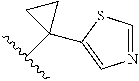 [SMILES: *C1(c2cncs2)CC1]

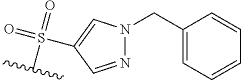 [SMILES: *S(=O)(=O)c1cnn(Cc2ccccc2)c1]

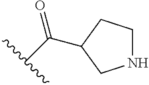 [SMILES: *C(=O)C1CCNC1]

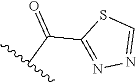 [SMILES: *C(=O)c1nncs1]

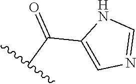 [SMILES: *C(=O)c1cnc[nH]1]

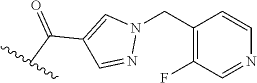 [SMILES: *C(=O)c1cnn(Cc2ccncc2F)c1]

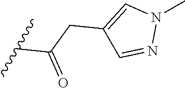 [SMILES: *C(=O)Cc1cnn(C)c1]

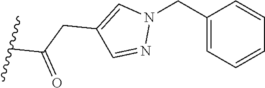 [SMILES: *C(=O)Cc1cnn(Cc2ccccc2)c1]

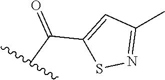 [SMILES: *C(=O)c1cc(C)ns1]

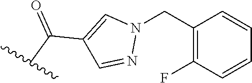 [SMILES: *C(=O)c1cnn(Cc2ccccc2F)c1]

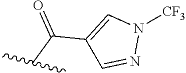 [SMILES: *C(=O)c1cnn(C(F)(F)F)c1]

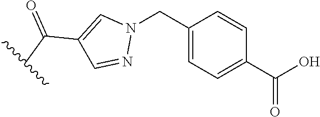 [SMILES: *C(=O)c1cnn(Cc2ccc(C(=O)O)cc2)c1]

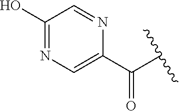 [SMILES: *C(=O)c1cnc(O)cn1]

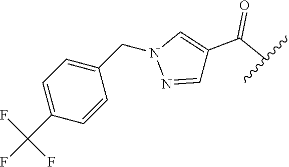 [SMILES: *C(=O)c1cnn(Cc2ccc(C(F)(F)F)cc2)c1]

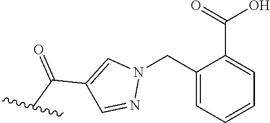 [SMILES: *C(=O)c1cnn(Cc2ccccc2C(=O)O)c1]

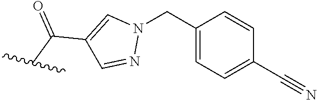 [SMILES: *C(=O)c1cnn(Cc2ccc(C#N)cc2)c1]

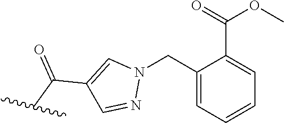 [SMILES: *C(=O)c1cnn(Cc2ccccc2C(=O)OC)c1]

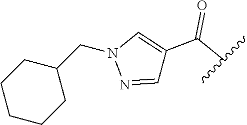 [SMILES: *C(=O)c1cnn(CC2CCCCC2)c1]

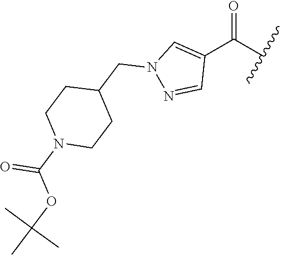 [SMILES: *C(=O)c1cnn(CC2CCN(C(=O)OC(C)(C)C)CC2)c1]

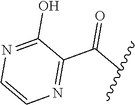 [SMILES: *C(=O)c1nccnc1O]

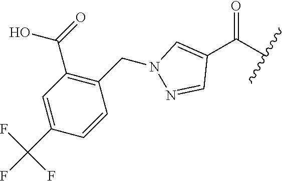 [SMILES: *C(=O)c1cnn(Cc2ccc(C(F)(F)F)cc2C(=O)O)c1]

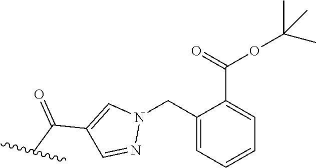 [SMILES: *C(=O)c1cnn(Cc2ccccc2C(=O)OC(C)(C)C)c1]

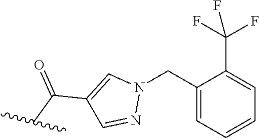 [SMILES: *C(=O)c1cnn(Cc2ccccc2C(F)(F)F)c1]

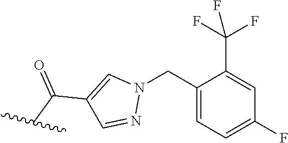 [SMILES: *C(=O)c1cnn(Cc2ccc(F)cc2C(F)(F)F)c1]

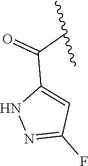 [SMILES: *C(=O)c1cc(F)n[nH]1]

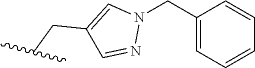 [SMILES: *Cc1cnn(Cc2ccccc2)c1]

—C(O)CH₃

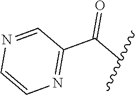 [SMILES: *C(=O)c1cnccn1]

—C(O)C(CH₃)₃

In some embodiments, -L³-R⁸ is

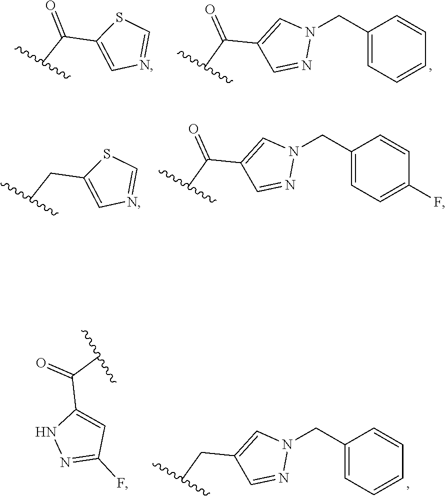 [SMILES: *C(=O)c1cncs1 ; *C(=O)c1cnn(Cc2ccccc2)c1 ; *Cc1cncs1 ; *C(=O)c1cnn(Cc2ccc(F)cc2)c1 ; *C(=O)c1cc(F)n[nH]1 ; *Cc1cnn(Cc2ccccc2)c1]

### —C(O)CH₃, or

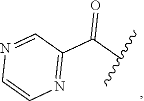 [SMILES: *C(=O)c1cnccn1]

—C(O)C(CH₃)₃.

In some embodiments, -L³-R¹ is

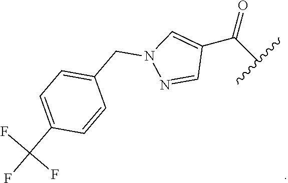 [SMILES: *C(=O)c1cnn(Cc2ccc(C(F)(F)F)cc2)c1]

In some embodiments, -L³-R⁸ is

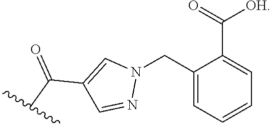 [SMILES: *C(=O)c1cnn(Cc2ccccc2C(=O)O)c1]

In some embodiments, -L³-R¹ is

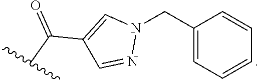 [SMILES: *C(=O)c1cnn(Cc2ccccc2)c1]

In some embodiments, -L³-R⁸ is

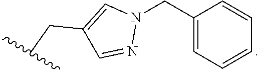 [SMILES: *Cc1cnn(Cc2ccccc2)c1]

In some embodiments, -L³-R⁸ is

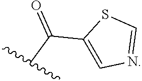 [SMILES: *C(=O)c1cncs1]

As generally described above, each L⁴ is independently a covalent bond, a saturated or unsaturated, straight or branched, optionally substituted bivalent C₁₋₄ hydrocarbon chain, wherein 0-2 methylene units of L⁴ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—-Cy-, or —NRC(O)NR—.

In some embodiments, L⁴ is an saturated or unsaturated, straight or branched, optionally substituted bivalent C₁₋₃ hydrocarbon chain, wherein 0-3 methylene units of L⁴ are independently replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L⁴ is an saturated or unsaturated, straight or branched, optionally substituted bivalent C₁₋₃ hydrocarbon chain, wherein 0-3 methylene units of L⁴ are independently replaced by —O—, —NR—, —S(O)₂—, or —C(O)—. In some embodiments, L⁴ is an saturated or unsaturated, straight or branched, optionally substituted bivalent C₁₋₃ hydrocarbon chain, wherein 1 methylene units of L⁴ is independently replaced by —O—. In some embodiments, L⁴ is an saturated or unsaturated, straight or branched, optionally substituted bivalent C₁₋₃ hydrocarbon chain, wherein 1 methylene units of L⁴ is independently replaced by —N—. In some embodiments, L⁴ is an saturated or unsaturated, straight or branched, optionally substituted bivalent C₁₋₃ hydrocarbon chain, wherein 1 methylene units of L⁴ is independently replaced by —N(CH₃)—. In some embodiments, L⁴ is an saturated or unsaturated, straight or branched, optionally substituted bivalent C₁₋₃ hydrocarbon chain, wherein 1 methylene units of L⁴ is independently replaced by —S(O)₂—.

In some embodiments, L⁴ (as read from left to right

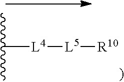

is —C(CH₃)H—O—CH₂—, —CH₂—O—C(CH₃)H—, —CH₂OCH₂—, —CH₂—NH—CH₂—, —CH₂—N(CH₃)—CH₂—, —C(O)NH—S(O)₂—, —CH₂—S(O)₂—CH₂—, —NHC(O)—, —CH₂O—, —OCH₂—, or

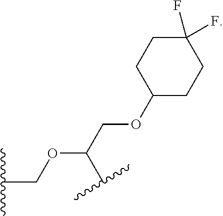 [SMILES: *COC(*)COC1CCC(F)(F)CC1]

In some embodiments, L⁴ is optionally substituted phenylene, an optionally substituted bivalent 5-6 membered monocyclic heteroarylene ring (having 1-4 heteroatoms independently selected from nitrogen, oxygen, and sulfur), or an optionally substituted bivalent 8-10 membered bicyclic heteroarylene ring (having 1-5 heteroatoms independently selected from nitrogen, oxygen, and sulfur).

In some embodiments, L⁴ is an optionally substituted phenylene. In some embodiments, L⁴ is an optionally substituted bivalent 5-6 membered monocyclic heteroarylene ring (having 1-4 heteroatoms independently selected from nitrogen, oxygen, and sulfur). In certain embodiments, L⁴ is an optionally substituted 5 membered monocyclic heteroarylene ring (having 1-4 heteroatoms independently selected from nitrogen, oxygen, and sulfur). In certain embodiments, L⁴ is an optionally substituted 6 membered monocyclic heteroarylene ring (having 1-4 heteroatoms independently selected from nitrogen, oxygen, and sulfur). In some embodiments, L⁴ is an optionally substituted bivalent 8-10 membered bicyclic heteroarylene ring (having 1-5 heteroatoms independently selected from nitrogen, oxygen, and sulfur).

In some embodiments, L⁴ is isoxazolylene, oxadiazolylene, 1,2,4-oxadiazolylene, oxazolylene, 1,3,4-oxadiazolylene, 4H-1,2,4-triazolylene, 1,2,3-triazolylene, phenylene, pyrrolylene, furanylene, thiopheneylene, pyridinylene, pyrazinylene, pyrimidinylene, pyridazinyl, thiadiazolylene, 1,3,4-thiadiazolylene, thiazolylene, isothiazolylene, or benzo[d]oxazolylene.

In some embodiments, L⁴ is a substituent of Table L4-a below, wherein the [symbol] on the left signifies the [symbol] in

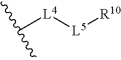

(i.e., the point of attachment of R^{A} to the 2,6-diazaspiro[3.4]octane moiety of Formula I) and the [symbol] on the right signifies the point of attachment of L⁴ onto L⁵. In some embodiments, L⁴ is selected rom those depicted in the compounds of Table 1, below. In some embodiments, L⁴ is selected from those depicted in Table L4-a.

TABLE L4-a
Exemplary L⁴ substituents

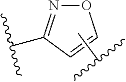

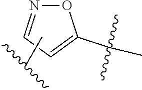

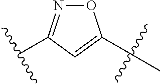 [SMILES: *c1cc(*)on1]

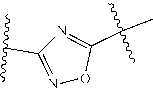 [SMILES: *c1noc(*)n1]

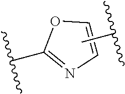

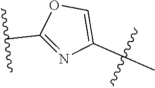 [SMILES: *c1coc(*)n1]

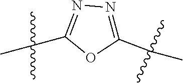 [SMILES: *c1nnc(*)o1]

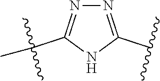 [SMILES: *c1nnc(*)[nH]1]

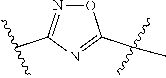 [SMILES: *c1noc(*)n1]

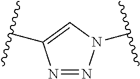 [SMILES: *c1cn(*)nn1]

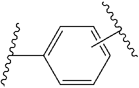

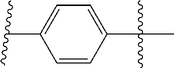 [SMILES: *c1ccc(*)cc1]

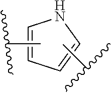

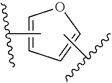

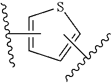

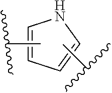

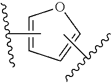

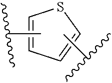

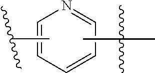

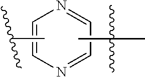

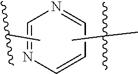

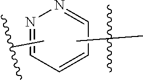

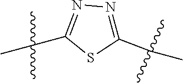 [SMILES: *c1nnc(*)s1]

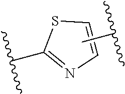

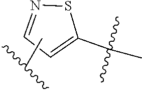

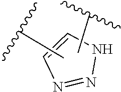

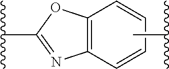

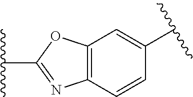 [SMILES: *c1ccc2nc(*)oc2c1]

As defined generally above, L⁵ is a covalent bond or a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—.

In some embodiments, L⁵ is a covalent bond or a saturated or unsaturated, straight or branched, optionally substituted bivalent C₁₋₂ hydrocarbon chain wherein a 1st methylene unit of L⁵ is replaced with a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic carbocyclene ring, phenyl, and a 3-8 membered saturated or partially unsaturated monocyclic heterocyclene ring (having 1-2 heteroatoms independently selected from nitrogen, oxygen, and sulfur), wherein the bivalent cyclic group is optionally substituted with one or more instances of R⁹; and wherein if L⁵ is a saturated or unsaturated, straight or branched, optionally substituted bivalent C₂ hydrocarbon chain wherein said 1^{st} methylene unit of L⁵ is replaced with said bivalent cyclic group, a 2^{nd} methylene unit of L⁵ is optionally replaced by replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L⁵ is a covalent bond. In some embodiments, L⁵ is an optionally substituted bivalent C₁₋₂ hydrocarbon chain. In some embodiments, L⁵ is a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic heterocyclene ring (having 1-2 heteroatoms independently selected from nitrogen, oxygen, and sulfur), wherein the bivalent cyclic group is optionally substituted with one or more instances of R⁹. In some embodiments, L⁵ is a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L⁵ is selected from the group consisting of —CH₂—, —C(CH₃)H—, —NH—, —C(O)—, —NH—, —CH₂CH₂—, —CF₂—, —C(CH₃)₂—, —CH₂O—, —OCH₂—, —C(O)O—CH₂—, —C(O)NH—, and

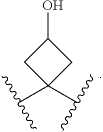 [SMILES: *C1(*)CC(O)C1]

In some embodiments, L⁵ is

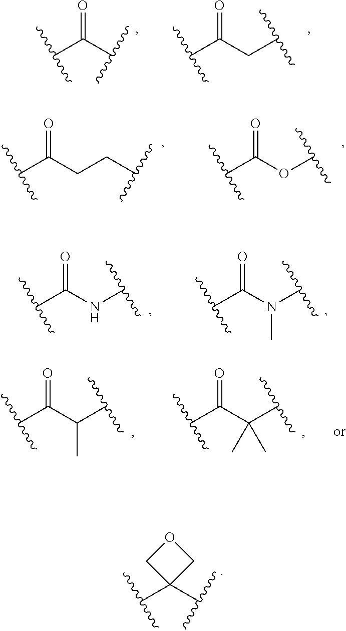

In some embodiments, L⁵ is

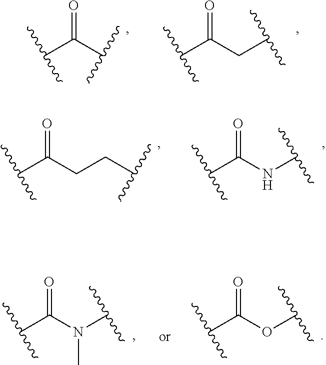

In some embodiments, L⁵ is

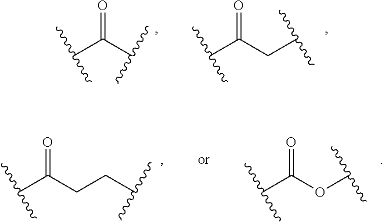

In some embodiments, L⁵ is

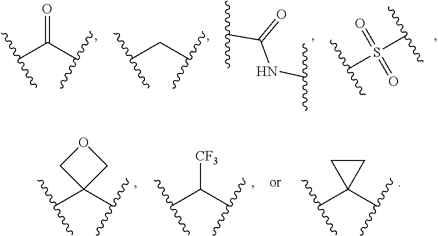

In some embodiments, L⁵ is

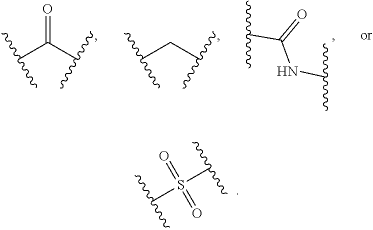

In some embodiments, L⁵ is

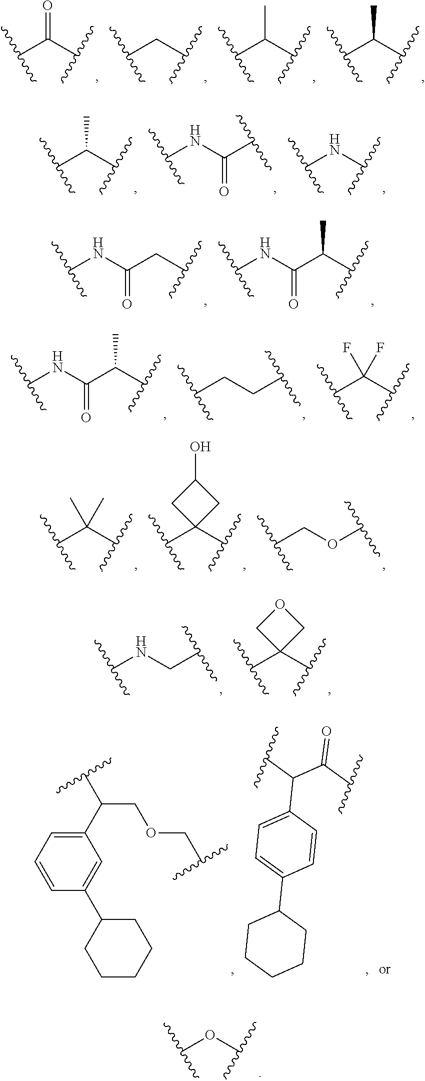

In some embodiments, L⁵ is a substituent depicted in the compounds of Table 1 below.

In some embodiments, the [symbol] on the left of L⁵ signifies the point of attachment to L⁴ and the [symbol] on the right of L⁵ signifies the point of attachment to R¹⁰.

In some embodiments, the L⁵ is a bivalent cyclic group substituted with 1 instance of R⁹. In some embodiments, L⁵ is a bivalent cyclic group substituted with 2 instances of R⁹. In some embodiments, L⁵ is a bivalent cyclic group substituted with 3 instances of R⁹. In some embodiments, L⁵ is a bivalent cyclic group substituted with 4 instances of R⁹. In some embodiments, L⁵ is a bivalent cyclic group substituted with 5 instances of R⁹.

In some embodiments L⁵ is selected from Table L5-a. In some embodiments the [symbol] on the left of the moiety in Table L5-a connects to L⁴ and the [symbol] on the right of the moiety connects to R¹⁰. In some embodiments the [symbol] on the right of the moiety in Table L5-a connects to L⁴ and the [symbol] on of the moiety connects to R¹⁰.

TABLE L5-a
Exemplary L⁵ Linkers

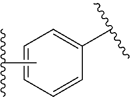

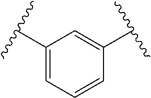 [SMILES: *c1cccc(*)c1]

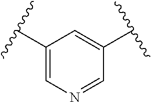 [SMILES: *c1cncc(*)c1]

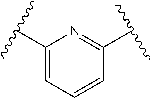 [SMILES: *c1cccc(*)n1]

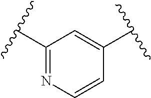 [SMILES: *c1ccnc(*)c1]

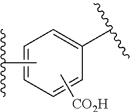

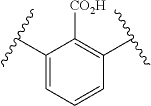 [SMILES: *c1cccc(*)c1C(=O)O]

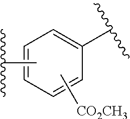

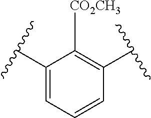 [SMILES: *c1cccc(*)c1C(=O)OC]

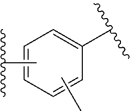

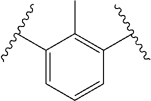 [SMILES: *c1cccc(*)c1C]

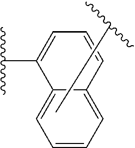

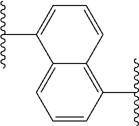 [SMILES: *c1cccc2c(*)cccc12]

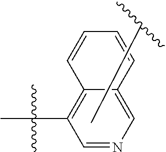

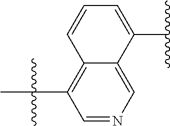 [SMILES: *c1cncc2c(*)cccc12]

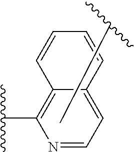

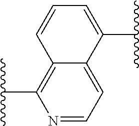 [SMILES: *c1cccc2c(*)nccc12]

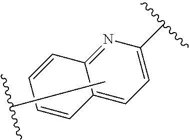

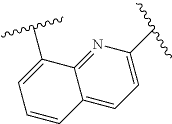 [SMILES: *c1ccc2cccc(*)c2n1]

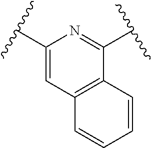 [SMILES: *c1cc2ccccc2c(*)n1]

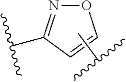

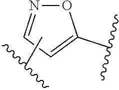

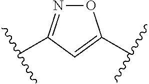 [SMILES: *c1cc(*)on1]

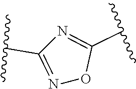 [SMILES: *c1noc(*)n1]

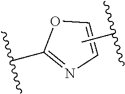

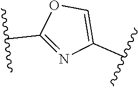 [SMILES: *c1coc(*)n1]

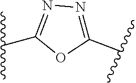 [SMILES: *c1nnc(*)o1]

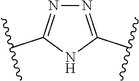 [SMILES: *c1nnc(*)[nH]1]

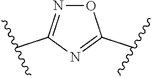 [SMILES: *c1noc(*)n1]

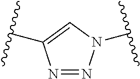 [SMILES: *c1cn(*)nn1]

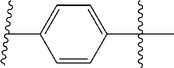 [SMILES: *c1ccc(*)cc1]

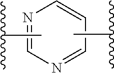

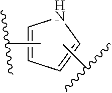

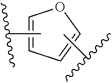

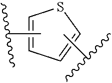

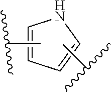

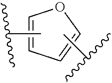

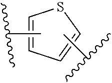

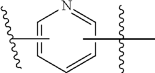

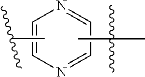

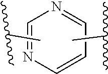

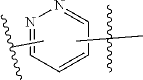

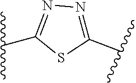 [SMILES: *c1nnc(*)s1]

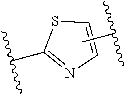

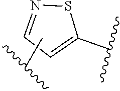

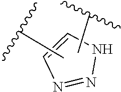

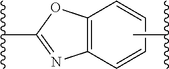

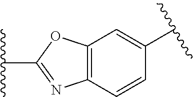 [SMILES: *c1ccc2nc(*)oc2c1]

As defined generally above, R¹⁰ is hydrogen, —CN, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹.

In some embodiments, R¹⁰ is hydrogen or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R¹⁰ is hydrogen. In some embodiments, R¹⁰ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R¹⁰ is a 3-8 membered saturated or partially unsaturated monocyclic carbocyclic ring. In some embodiments, R¹⁰ is a 7-12 membered saturated or partially unsaturated bicyclic carbocyclic ring. In some embodiments, R¹⁰ is phenyl. In some embodiments, R¹⁰ is an 8-10 membered bicyclic aromatic carbocyclic ring. In some embodiments, R¹⁰ is a 3-8 membered saturated or partially unsaturated monocyclic heterocyclic ring (having 1-3 heteroatoms independently selected from nitrogen, oxygen, and sulfur). In some embodiments, R¹⁰ is a 7-12 membered saturated or partially unsaturated bicyclic heterocyclic ring (having 1-4 heteroatoms independently selected from nitrogen, oxygen, and sulfur). In some embodiments, R¹⁰ is a 5-6 membered monocyclic heteroaromatic ring (having 1-4 heteroatoms independently selected from nitrogen, oxygen, and sulfur). In some embodiments, R¹⁰ is an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, the R¹⁰ is a cyclic group substituted with 1 instance of R⁹. In some embodiments, the R¹⁰ is a cyclic group substituted with 2 instances of R⁹. In some embodiments, the R¹⁰ is a cyclic group substituted with 3 instances of R⁹. In some embodiments, the R¹⁰ is a cyclic group substituted with 4 instances of R⁹. In some embodiments, the R¹⁰ is a cyclic group substituted with 5 instances of R⁹.

In some embodiments, R¹⁰ is selected from those depicted in the compounds of Table 1, below.

In some embodiments, R¹⁰ is a substituent of Table R10-a or R10-b.

TABLE R10-a
Exemplary R¹⁰ substituents

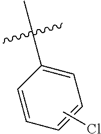

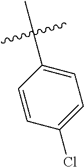 [SMILES: *c1ccc(Cl)cc1]

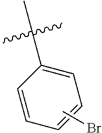

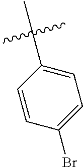 [SMILES: *c1ccc(Br)cc1]

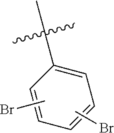

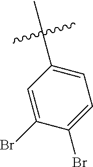 [SMILES: *c1ccc(Br)c(Br)c1]

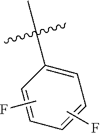

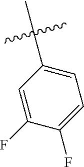 [SMILES: *c1ccc(F)c(F)c1]

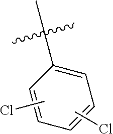

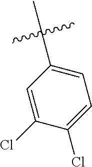 [SMILES: *c1ccc(Cl)c(Cl)c1]

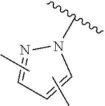

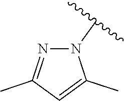

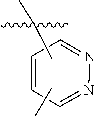

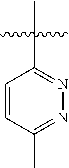 [SMILES: *c1ccc(C)nn1]

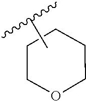

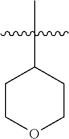 [SMILES: *C1CCOCC1]

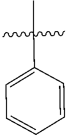 [SMILES: *c1ccccc1]

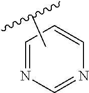

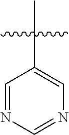 [SMILES: *c1cncnc1]

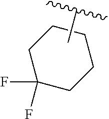

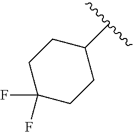 [SMILES: *C1CCC(F)(F)CC1]

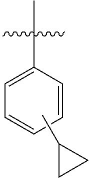

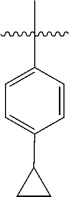 [SMILES: *c1ccc(C2CC2)cc1]

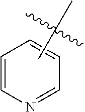

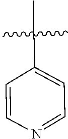 [SMILES: *c1ccncc1]

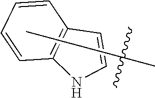

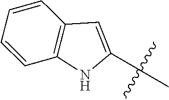 [SMILES: *c1cc2ccccc2[nH]1]

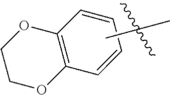

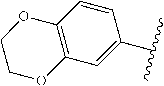 [SMILES: *c1ccc2c(c1)OCCO2]

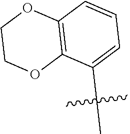 [SMILES: *c1cccc2c1OCCO2]

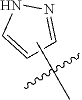

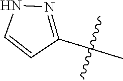 [SMILES: *c1cc[nH]n1]

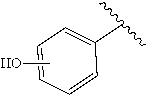

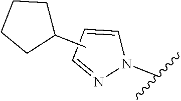

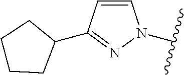 [SMILES: *n1ccc(C2CCCC2)n1]

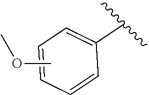

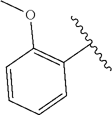 [SMILES: *c1ccccc1OC]

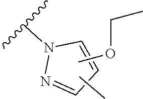

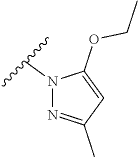 [SMILES: *n1nc(C)cc1OCC]

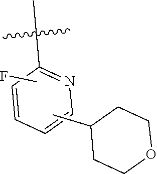

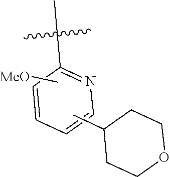

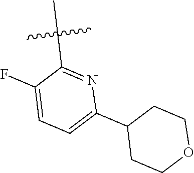 [SMILES: *c1nc(C2CCOCC2)ccc1F]

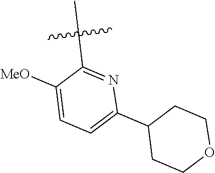 [SMILES: *c1nc(C2CCOCC2)ccc1OC]

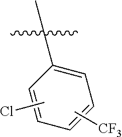

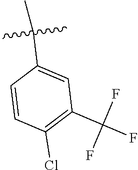 [SMILES: *c1ccc(Cl)c(C(F)(F)F)c1]

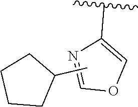

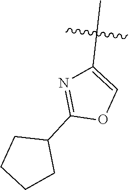 [SMILES: *c1coc(C2CCCC2)n1]

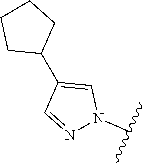 [SMILES: *n1cc(C2CCCC2)cn1]

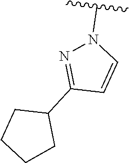 [SMILES: *n1ccc(C2CCCC2)n1]

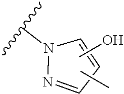

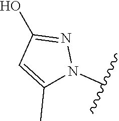 [SMILES: *n1nc(O)cc1C]

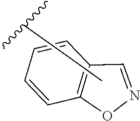

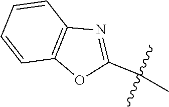 [SMILES: *c1nc2ccccc2o1]

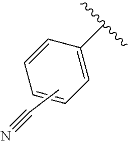

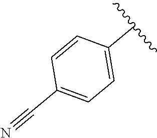 [SMILES: *c1ccc(C#N)cc1]

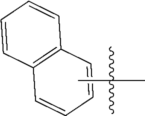

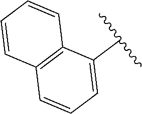 [SMILES: *c1cccc2ccccc12]

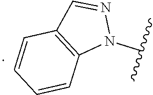 [SMILES: *n1ncc2ccccc21]

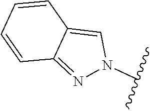 [SMILES: *n1cc2ccccc2n1]

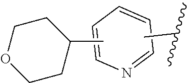

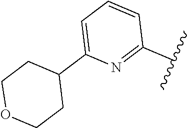 [SMILES: *c1cccc(C2CCOCC2)n1]

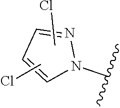

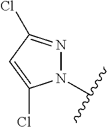 [SMILES: *n1nc(Cl)cc1Cl]

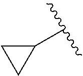 [SMILES: *C1CC1]

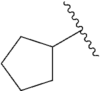 [SMILES: *C1CCCC1]

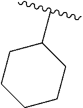 [SMILES: *C1CCCCC1]

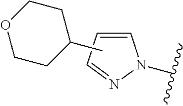

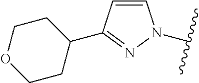 [SMILES: *n1ccc(C2CCOCC2)n1]

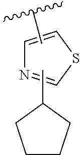

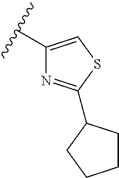 [SMILES: *c1csc(C2CCCC2)n1]

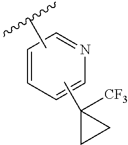

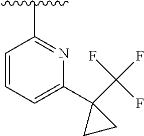 [SMILES: *c1cccc(C2(C(F)(F)F)CC2)n1]

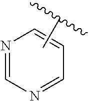

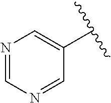 [SMILES: *c1cncnc1]

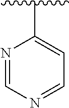 [SMILES: *c1ccncn1]

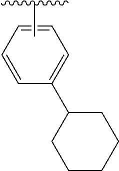

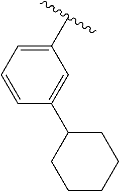 [SMILES: *c1cccc(C2CCCCC2)c1]

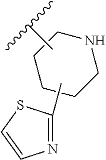

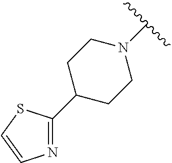 [SMILES: *N1CCC(c2nccs2)CC1]

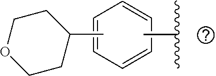

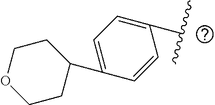 [SMILES: *c1ccc(C2CCOCC2)cc1]

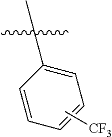

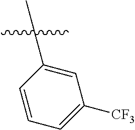 [SMILES: *c1cccc(C(F)(F)F)c1]

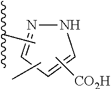

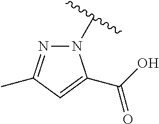 [SMILES: *n1nc(C)cc1C(=O)O]

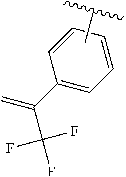

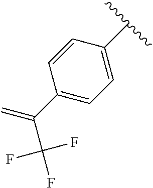 [SMILES: *c1ccc(C(=C)C(F)(F)F)cc1]

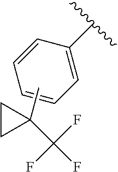

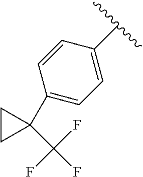 [SMILES: *c1ccc(C2(C(F)(F)F)CC2)cc1]

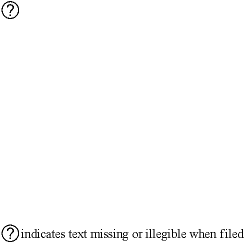

TABLE R10-b
Exemplary R¹⁰ groups

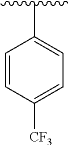 [SMILES: *c1ccc(C(F)(F)F)cc1]

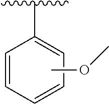

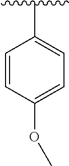 [SMILES: *c1ccc(OC)cc1]

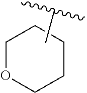

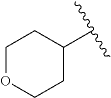 [SMILES: *C1CCOCC1]

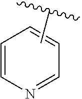

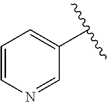 [SMILES: *c1cccnc1]

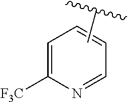

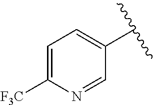 [SMILES: *c1ccc(C(F)(F)F)nc1]

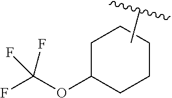

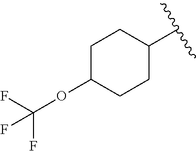 [SMILES: *C1CCC(OC(F)(F)F)CC1]

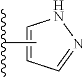

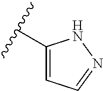 [SMILES: *c1ccn[nH]1]

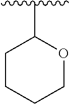 [SMILES: *C1CCCCO1]

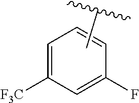

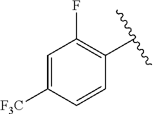 [SMILES: *c1ccc(C(F)(F)F)cc1F]

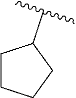 [SMILES: *C1CCCC1]

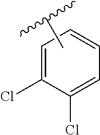

As described generally above, each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), an 8-10 membered bicyclic aromatic carbocyclic ring, a 7-12 membered saturated or partially unsaturated bicyclic heterocyclic ring that is optionally bridged bicyclic (having 1-4 heteroatoms independently selected from nitrogen, oxygen, and sulfur), an 8-10 membered bicyclic heteroaromatic ring (having 1-5 heteroatoms independently selected from nitrogen, oxygen, and sulfur), and a 5-6 membered monocyclic heteroaromatic ring (having 1-4 heteroatoms independently selected from nitrogen, oxygen, and sulfur).

In some embodiments, Cy is a 3-8 membered saturated or partially unsaturated monocyclic carbocyclic ring. In some embodiments, Cy is phenyl. In some embodiments, Cy is a 3-8 membered saturated or partially unsaturated monocyclic heterocyclic ring (having 1-2 heteroatoms independently selected from nitrogen, oxygen, and sulfur). In some embodiments, Cy is an 8-10 membered bicyclic aromatic carbocyclic ring an 8-10 membered bicyclic aromatic carbocyclic ring. In some embodiments, Cy is a 5-6 membered monocyclic heteroaromatic ring (having 1-4 heteroatoms independently selected from nitrogen, oxygen, and sulfur).

As described generally above, R^{Z} is hydrogen, —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹.

In some embodiments, R^{Z} is hydrogen. In some embodiments, R^{Z} is —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹.

In some embodiments, R^{Z} is —CN, an optionally substituted C₁₋₆ aliphatic group. In some embodiments, R^{Z} is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹.

As described generally above, each R is independently hydrogen, halogen or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
an R group of R^{A} and one of Z¹, Z², Z³, Z⁴, G¹, G², G³, or G⁴ are taken together with their intervening atoms to form an optionally substituted 3-7 membered heterocyclic ring; and/or
an R group of R^{A} and one of R¹, R², or R³ are taken together with their intervening atoms to form a 5-7 membered heterocyclic ring; and/or
two R groups on the same atom are taken together with their intervening atoms to form an optionally substituted 3-7 membered saturated, partially unsaturated, or heteroaryl ring (having 0-3 heteroatoms independently selected from nitrogen, oxygen, and sulfur); or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

In some embodiments, each R is hydrogen. In some embodiments, each R is independently halogen or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur). In some embodiments, each R is independently hydrogen, halogen or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur).

In some embodiments, an R group of R^{A} and one of Z¹, Z², Z³, Z⁴, G¹, G², G³, or G4 are taken together with their intervening atoms to form an optionally substituted 3-7 membered heterocyclic ring. In some embodiments, an R group of R^{A} and Z¹ are taken together with their intervening atoms to form an optionally substituted 3-7 membered heterocyclic ring. In some embodiments, an R group of R^{A} and Z² are taken together with their intervening atoms to form an optionally substituted 3-7 membered heterocyclic ring. In some embodiments, an R group of R^{A} and Z³ are taken together with their intervening atoms to form an optionally substituted 3-7 membered heterocyclic ring. In some embodiments, an R group of R^{A} and Z⁴ are taken together with their intervening atoms to form an optionally substituted 3-7 membered heterocyclic ring. In some embodiments, an R group of R^{A} and G¹ are taken together with their intervening atoms to form an optionally substituted 3-7 membered heterocyclic ring. In some embodiments, an R group of R^{A} and G² are taken together with their intervening atoms to form an optionally substituted 3-7 membered heterocyclic ring. In some embodiments, an R group of R^{A} and G³ are taken together with their intervening atoms to form an optionally substituted 3-7 membered heterocyclic ring. In some embodiments, an R group of R^{A} and G⁴ are taken together with their intervening atoms to form an optionally substituted 3-7 membered heterocyclic ring.

In some embodiments, an R group of R^{A} and R¹ are taken together with their intervening atoms to form a 5-7 membered heterocyclic ring. In some embodiments, an R group of R^{A} and R² are taken together with their intervening atoms to form a 5-7 membered heterocyclic ring. In some embodiments, an R group of R^{A} and R³ are taken together with their intervening atoms to form a 5-7 membered heterocyclic ring.

In some embodiments, two R groups on the same atom are taken together with their intervening atoms to form an optionally substituted 3-7 membered saturated, partially unsaturated, or heteroaryl ring (having 0-3 heteroatoms independently selected from nitrogen, oxygen, and sulfur); or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

In some embodiments, m is 0, 1, or 2. In some embodiments, m is 0. In some embodiments, m is 1. In some embodiments, m is 2. In some embodiments, n is 0. In some embodiments, n is 1.

In some embodiments, n is 2.

In some embodiments, the compound of Formula I is a compound of Formula IA, IA*, or IA**:

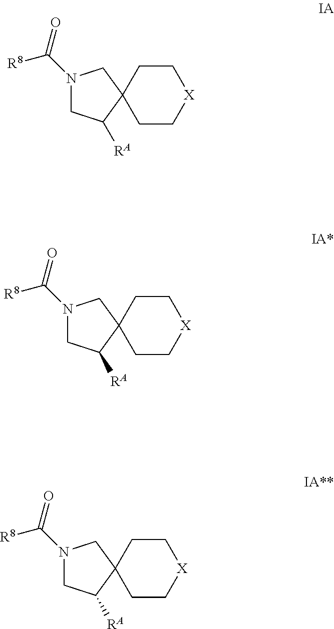

or a pharmaceutically acceptable salt thereof, wherein R⁸ and R^{A} and their constituent groups, are each as defined and described herein, and wherein X is selected from —O—, —N(R)—, —S(O)₂—, —S(O)—, and —C(S)—.

In some embodiments, the compound of Formula I is a compound of Formula IB⁰, IB⁰⁰, or IB⁰⁰⁰:

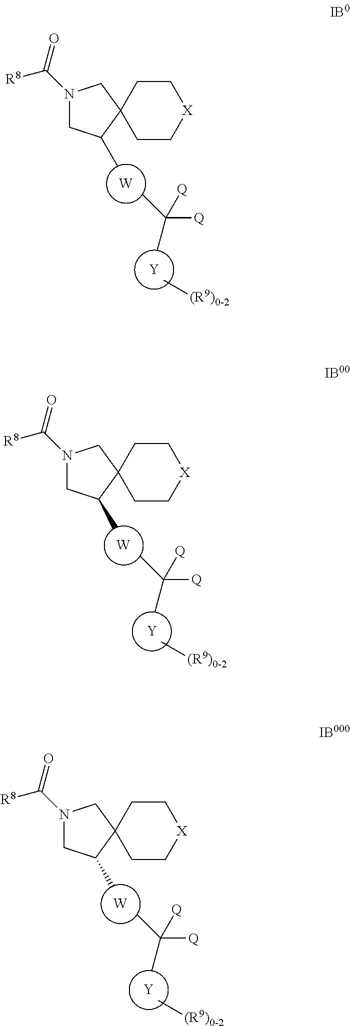

or a pharmaceutically acceptable salt thereof, wherein R⁸ and its constituent groups, are each as defined and described herein, and wherein
X is selected from —O—, —N(R)—, —S(O)₂—, —S(O)—, and —C(S)—;
each Q is independently hydrogen or halo,
Ring W is an optionally substituted 5-membered heteroarylene;
Ring Y is a 6-membered heteroaryl or phenyl; and
R⁹ is halo —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted C₁₋₆ aliphatic group.

In some embodiments, the compound of Formula I is a compound of Formula IB, IB*, or IB**:

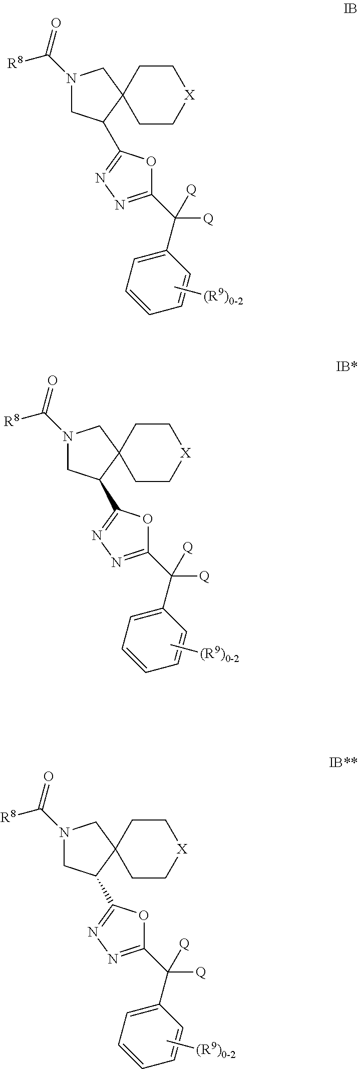

or a pharmaceutically acceptable salt thereof, wherein R⁸ and its constituent groups, are each as defined and described herein, and wherein
X is selected from —O—, —N(R)—, —S(O)₂—, —S(O)—, and —C(S)—;
each Q is independently hydrogen or halo; and
R⁹ is halo —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted C₁₋₆ aliphatic group.

In some embodiments, the compound of Formula I is a compound of Formula IC, IC*, IC** IC***, IC⁰, IC⁰⁰, IC⁰⁰⁰, IC⁰⁰⁰, IC^{†}, IC^{††}, IC^{†††}, or IC^{††††}:

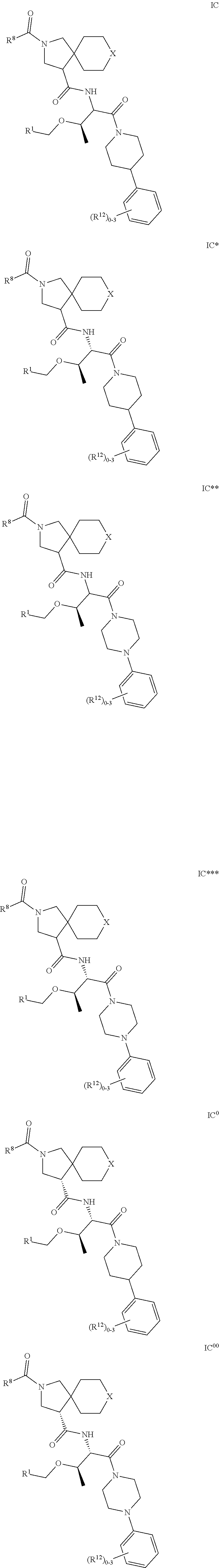

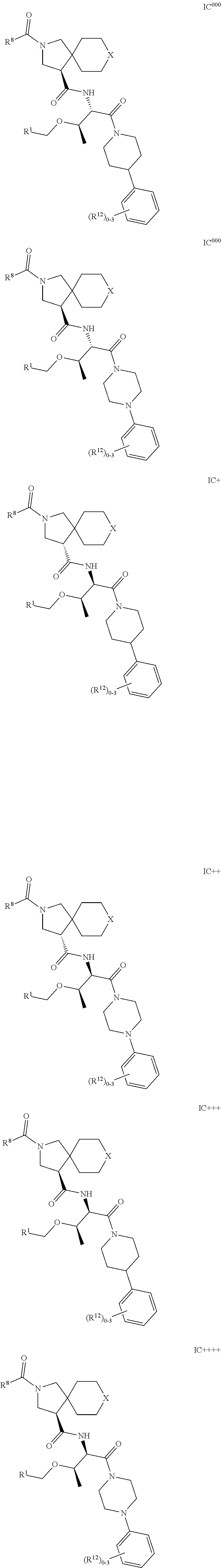

or a pharmaceutically acceptable salt thereof, wherein R¹, R⁸ and their constituent groups, are each as defined and described herein, and wherein
X is selected from —O—, —N(R)—, —S(O)₂—, —S(O)—, and —C(S)—;
R¹² has from 0 to 3 instances each independently selected from halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, and Cy. In some embodiments, R¹² has from 0 to 3 instances each independently selected from —F, —CF₃, —C(O)OH, —C(O)OCH₃, —C(O)OCH₂CH₃, —C(O)OCH₂CH₂CH₃, —C(O)OC(CH₃)₃, —OCH₂C(O)OH, —OCH₂C(O)OCH₃, —CH₃, —OC(CH₃)₂C(O)OH, —OC(CH₃)₂C(O)OCH₃.

In some embodiments, the compound of Formula I is a compound of any one of Formula IDa to IDr:

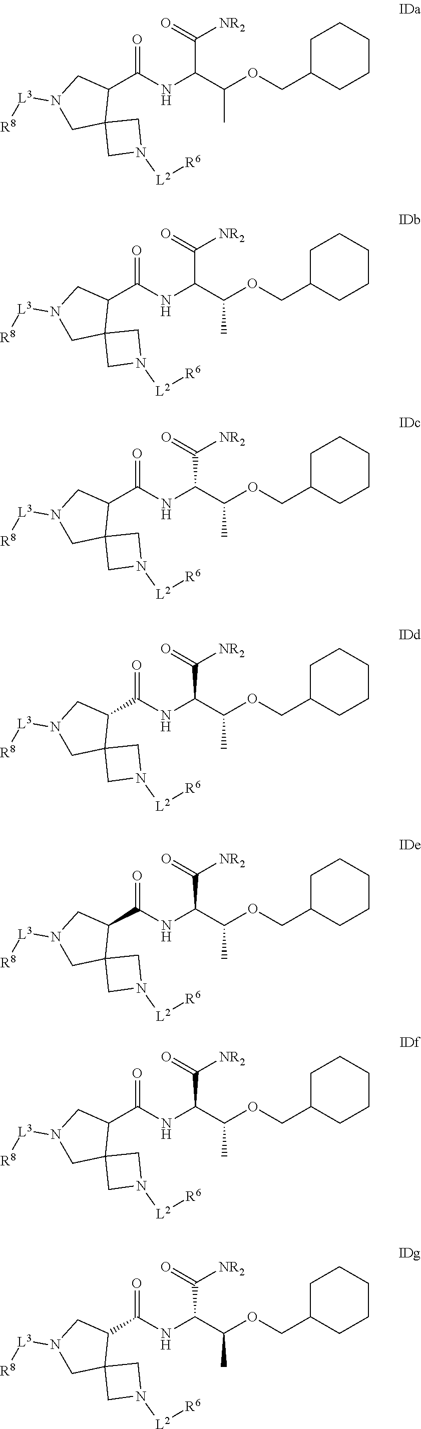

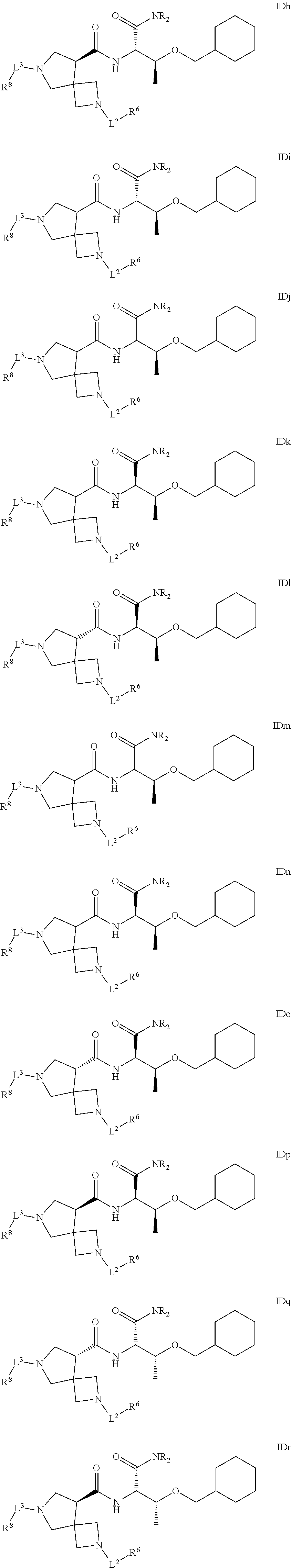

or a pharmaceutically acceptable salt thereof, wherein R⁶, R¹ their constituent groups, are each as defined and described herein, and wherein:
L¹ is an optionally substituted methylene or —C(O)—;
L² is optionally substituted methylene, —OC(O)—, —C(O)O—, —C(O)—, —S(O)₂—, —C(S)—, —NRS(O)₂—, —S(O)₂NR—, —NRC(O)—, or —C(O)NR—; and
each R is independently hydrogen or an optionally substituted C₁₋₆ aliphatic group; or
the two R groups on the same atom are taken together with their intervening atoms to form an optionally substituted 3-7 membered saturated or partially unsaturated ring (having 0-3 heteroatoms independently selected from nitrogen, oxygen, and sulfur) or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments the two R groups together with the nitrogen to which they are attached form an optionally substituted piperidyl group. In some embodiments the two R groups together with the nitrogen to which they are attached form a piperidyl group substituted with 5-6 membered heteroaryl or —CH₂O(C₁₋₃ alkyl).

In some embodiments, the compound of Formula I is a compound of any one of Formula

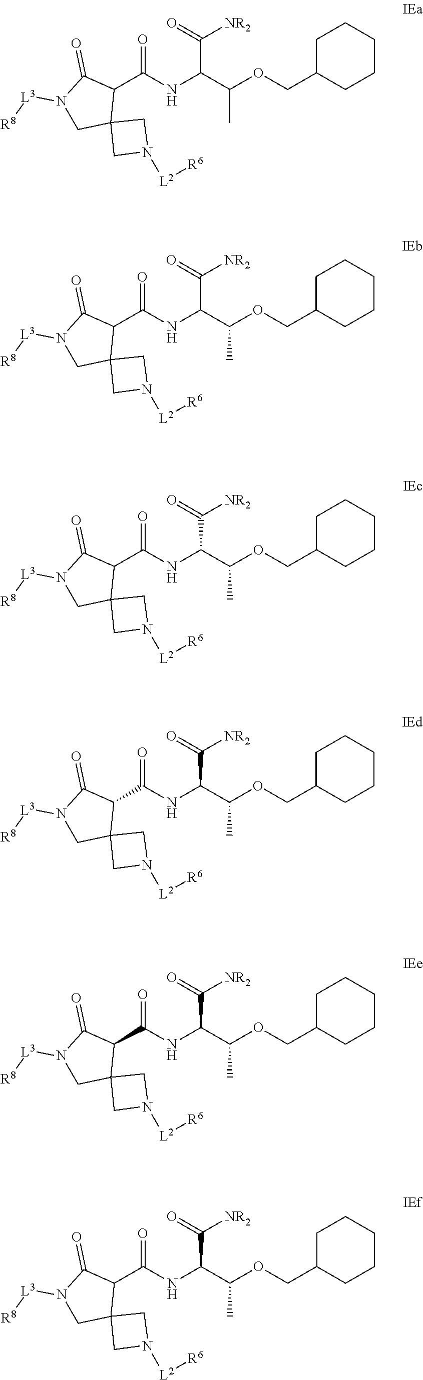

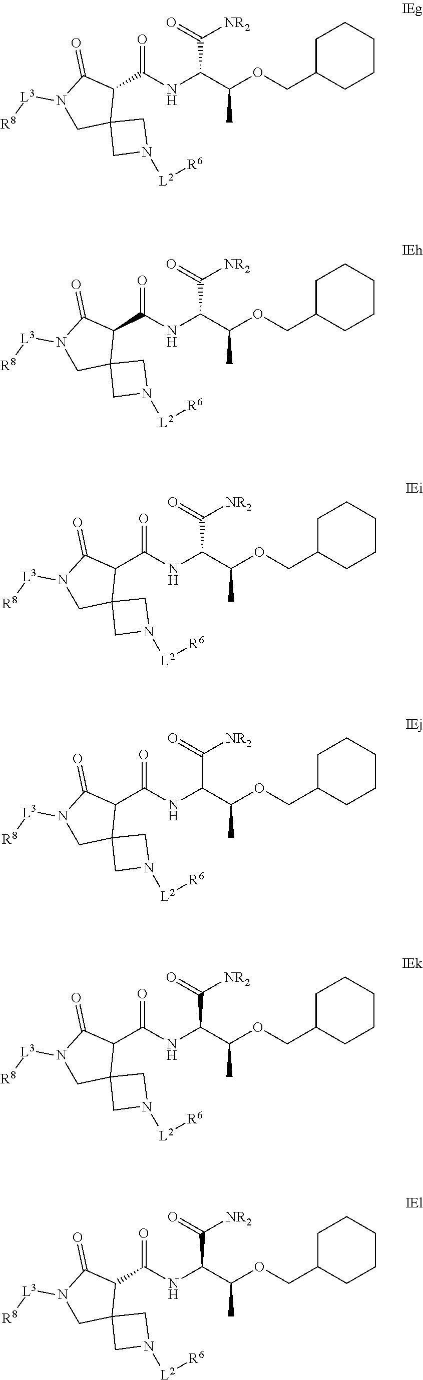

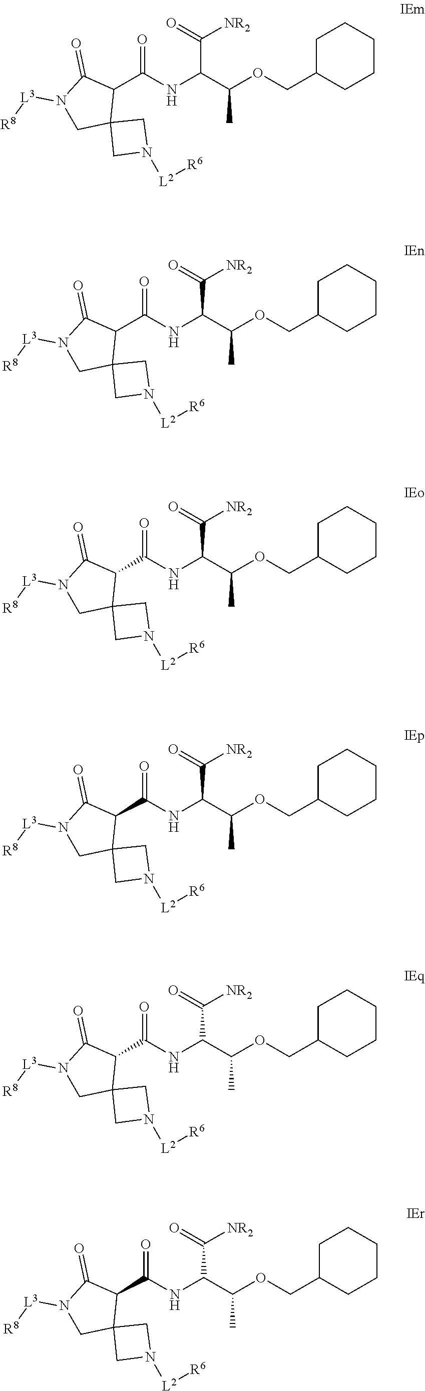

or a pharmaceutically acceptable salt thereof, wherein R⁶, R⁸ their constituent groups, are each as defined and described herein, and wherein:
L³ is an optionally substituted methylene or —C(O)—;
L² is optionally substituted methylene, —OC(O)—, —C(O)O—, —C(O)—, —S(O)₂—, —C(S)—, —NRS(O)₂—, —S(O)₂NR—, —NRC(O)—, or —C(O)NR—; and
each R is independently hydrogen or an optionally substituted C₁₋₆ aliphatic group; or
the two R groups on the same atom are taken together with their intervening atoms to form an optionally substituted 3-7 membered saturated or partially unsaturated ring (having 0-3 heteroatoms independently selected from nitrogen, oxygen, and sulfur) or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments the two R groups together with the nitrogen to which they are attached form an optionally substituted piperidyl group. In some embodiments the two R groups together with the nitrogen to which they are attached form a piperidyl group substituted with 5-6 membered heteroaryl or —CH₂O(C₁₋₃ alkyl).

In some embodiments, the compound of Formula I is a compound of any one of Formula IFa to IFi:

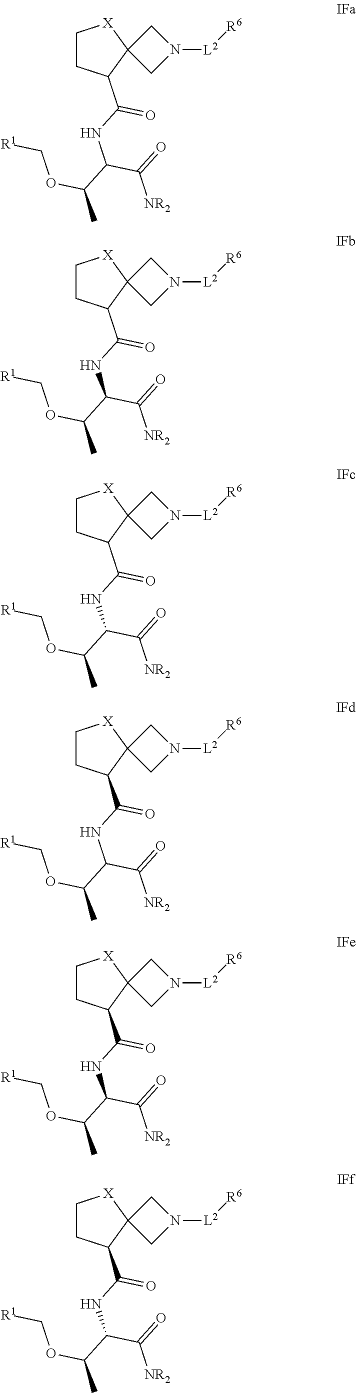

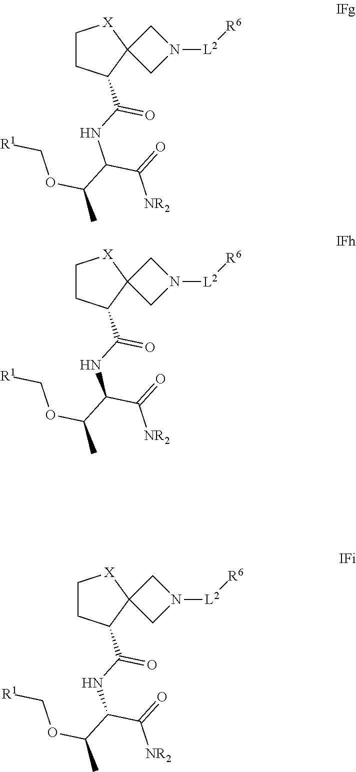

or a pharmaceutically acceptable salt thereof, wherein R⁶ and its constituent groups, are each as defined and described herein, and wherein:
X is selected from —O—, —N(R)—, —S(O)₂—, —S(O)—, —C(O)— and —C(S)—;
L² is optionally substituted methylene, —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—;
R¹ is an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
each R is independently hydrogen or an optionally substituted C₁₋₆ aliphatic group; or
the two R groups on the same atom are taken together with their intervening atoms to form an optionally substituted 3-7 membered saturated or partially unsaturated ring (having 0-3 heteroatoms independently selected from nitrogen, oxygen, and sulfur) or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments the two R groups together with the nitrogen to which they are attached form an optionally substituted piperidyl group. In some embodiments the two R groups together with the nitrogen to which they are attached form a piperidyl group substituted with 5-6 membered heteroaryl or —CH₂O(C₁₋₃ alkyl).

In some embodiments, the compound of Formula I is a compound of any one of Formula IF*a to IF*i:

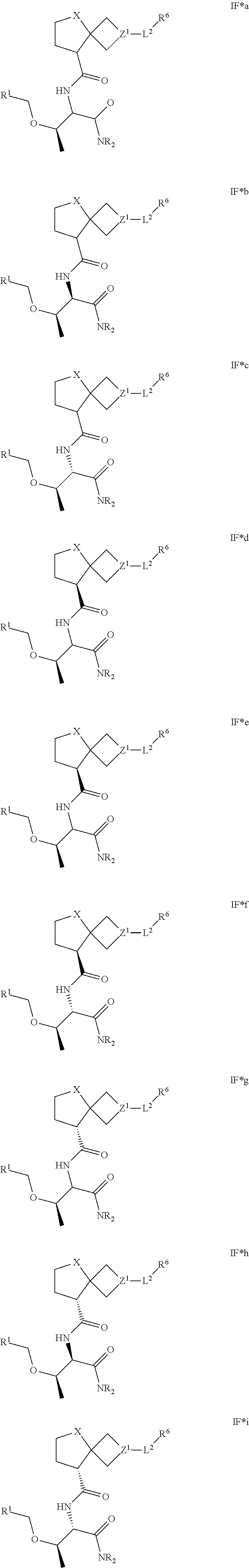

or a pharmaceutically acceptable salt thereof, wherein Z¹ and R⁶ and their constituent groups, are each as defined and described herein, and wherein:
X is selected from —O—, —N(R)—, —S(O)₂—, —S(O)—, —C(O)— and —C(S)—;
L² is optionally substituted methylene, —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—;
R¹ is an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
each R is independently hydrogen or an optionally substituted C₁₋₆ aliphatic group; or
the two R groups on the same atom are taken together with their intervening atoms to form an optionally substituted 3-7 membered saturated or partially unsaturated ring (having 0-3 heteroatoms independently selected from nitrogen, oxygen, and sulfur) or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments the two R groups together with the nitrogen to which they are attached form an optionally substituted piperidyl group. In some embodiments the two R groups together with the nitrogen to which they are attached form a piperidyl group substituted with 5-6 membered heteroaryl or —CH₂O(C₁₋₃ alkyl).

In some embodiments, the compound of Formula I is a compound of Formula IIA:

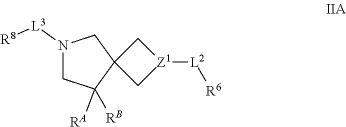

or a pharmaceutically acceptable salt thereof, wherein Z¹, R^{A}, R^{B} L², R⁶, L³ and R⁸, and their constituent groups, are each as defined and described herein. In some embodiments, Z¹, R^{A}, R^{B}, L², R⁶, L³ and R⁸, and their constituent groups, are each as defined and described in Formula I. In some embodiments, R^{A} is a substituent from Table Bi, Bii, or Biii. In some embodiments, -L²-R⁶ is a substituent from Table C. In some embodiments, -L³-R⁸ is a substituent from Table D. In some embodiments, R^{A} is a substituent from Table Bi, Bii, or Biii, and -L²-R⁶ is a substituent from Table C. In some embodiments, R^{A} is a substituent from Table Bi, Bii, or Biii, and -L³-R⁸ is a substituent from Table D. In some embodiments, -L²-R⁶ is a substituent from Table C, and -L³-R⁸ is a substituent from Table D. And in some embodiments, R^{A} is a substituent from Table Bi, Bii, or Biii, -L²-R⁶ is a substituent from Table C, and -L³-R⁸ is a substituent from Table D.

In some embodiments, the compound of Formula I is a compound of Formula IIB:

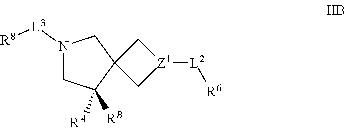

or a pharmaceutically acceptable salt thereof, wherein Z¹, R^{A}, R^{B}, L², R⁶, L³ and R⁸, and their constituent groups, are each as defined and described herein. In some embodiments, Z¹, R^{A}, R^{B}, L², R⁶, L³ and R⁸, and their constituent groups, are each as defined and described in Formula I. In some embodiments, R^{A} is a substituent from Table Bi, Bii, or Biii. In some embodiments, -L²-R⁶ is a substituent from Table C. In some embodiments, -L³-R⁸ is a substituent from Table D. In some embodiments, R^{A} is a substituent from Table Bi, Bii, or Biii, and -L²-R⁶ is a substituent from Table C. In some embodiments, R^{A} is a substituent from Table Bi, Bii, or Biii, and -L³-R¹ is a substituent from Table D. In some embodiments, -L²-R⁶ is a substituent from Table C, and -L³-R⁸ is a substituent from Table D. In some embodiments, R^{A} is a substituent from Table Bi, Bii, or Biii, -L²-R⁶ is a substituent from Table C, and -L³-R⁸ is a substituent from Table D.

In some embodiments, the compound of Formula I is a compound of Formula II:

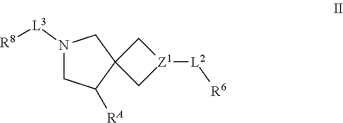

or a pharmaceutically acceptable salt thereof, wherein Z¹, R^{A}, L², R⁶, L³ and R⁸, and their constituent groups, are each as defined and described herein. In some embodiments, Z¹, R^{A}, L², R⁶, L³ and R⁸, and their constituent groups, are each as defined and described in Formula I. In some embodiments, R^{A} is a substituent from Table Bi, Bii, or Biii. In some embodiments, -L²-R⁶ is a substituent from Table C. In some embodiments, -L³-R⁸ is a substituent from Table D. In some embodiments, R^{A} is a substituent from Table Bi, Bii, or Biii, and -L²-R⁶ is a substituent from Table C. In some embodiments, R^{A} is a substituent from Table Bi, Bii, or Biii, and -L³-R⁸ is a substituent from Table D. In some embodiments, -L²-R⁶ is a substituent from Table C, and -L³-R⁸ is a substituent from Table D. And in some embodiments, R^{A} is a substituent from Table Bi, Bii, or Biii, -L²-R⁶ is a substituent from Table C, and -L³-R⁸ is a substituent from Table D.

In some embodiments, the compound of Formula I is a compound of Formula IIIa:

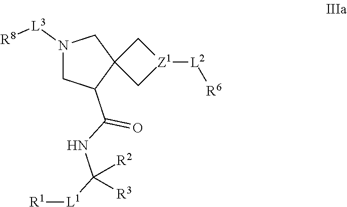

or a pharmaceutically acceptable salt thereof, wherein Z¹, L¹, R¹, R², R³, L², R⁶, L³ and R⁸, and their constituent groups, are each as defined and described herein. In some embodiments, R¹ is phenyl. In some embodiments, R¹ is cyclohexyl. In some embodiments, R¹ is

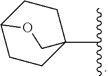 [SMILES: *C12CCC(CC1)OC2]

In some embodiments, R¹ is

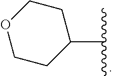 [SMILES: *C1CCOCC1]

In some embodiments, R¹ is

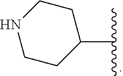 [SMILES: *C1CCNCC1]

In some embodiments, R¹ is

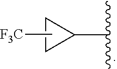

In some embodiments R¹ is —CF₃. In some embodiments, R² is a substituent from Table A. In some embodiments, R² is —C(O)NR₂, wherein the two R groups of —C(O)NR₂, taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₄alkyl), —O(C₁₋₄alkyl), C₁₋₄haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, L² is a methylene. In some embodiments, L³ is a methylene. In some embodiments, both L² and L³ are methylene. In some embodiments, L² is a —C(O)—. In some embodiments, L³ is a —C(O)—. In some embodiments, both L² and L³ are —C(O)—. In some embodiments, R^{A} is a substituent of Table Bi, Bii, or Biii. In some embodiments, -L²-R⁶ is

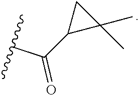 [SMILES: *C(=O)C1CC1(C)C]

In some embodiments, -L²-R⁶ is

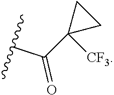 [SMILES: *C(=O)C1(C(F)(F)F)CC1]

In some embodiments, -L²-R⁶ is a substituent of Table C. In some embodiments, -L³-R¹ is a substituent from Table D. In some embodiments, -L³-R¹ is

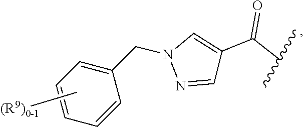

wherein R⁹ is —CF₃, —CN, —C(O)OH, —C(O)OCH₃, —C(O)OCH₂CH₃, —C(O)OC(CH₃)₃, or —C(O)OCH₂CH₂CH₃.

In some embodiments, the compound of Formula I is a compound of Formula IIIb:

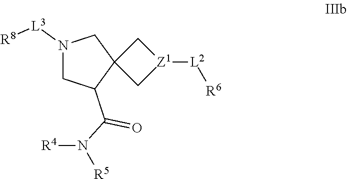

or a pharmaceutically acceptable salt thereof, wherein Z¹, R⁴, R⁵, L², R⁶, L³ and R⁸, and their constituent groups, are each as defined and described herein. In some embodiments, L² is a methylene. In some embodiments, L³ is a methylene. In some embodiments, both L² and L³ are methylene. In some embodiments, L² is —C(O)—. In some embodiments, L³ is —C(O)—. In some embodiments, both L² and L³ are —C(O)—. In some embodiments, -L²-R⁶ is a substituent from Table C. In some embodiments, -L²-R⁶ is

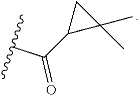 [SMILES: *C(=O)C1CC1(C)C]

In some embodiments, -L²-R⁶ is

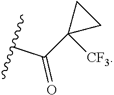 [SMILES: *C(=O)C1(C(F)(F)F)CC1]

In some embodiments, -L³-R⁸ is a substituent from Table D. In some embodiments, -L³-R¹ is

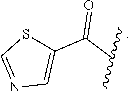 [SMILES: *C(=O)c1cncs1]

In some embodiments, R⁴ and R⁵, together, with the nitrogen to which they are attached, form an optionally substituted piperazine or an optionally substituted piperidine.

In some embodiments, the compound of Formula I is a compound of Formula IVa:

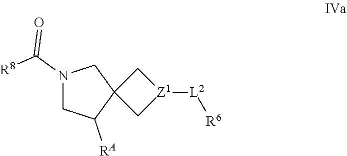

or a pharmaceutically acceptable salt thereof, wherein Z¹, R^{A}, L², R⁶, and R⁸, and their constituent groups, are each as defined and described herein. In some embodiments, R^{A} is a substituent from Table Bi, Bii, or Biii. In some embodiments, R^{A} is

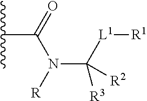

wherein R² is C(O)NR₂, wherein the two R groups of —C(O)NR₂, taken together with the intervening nitrogen atom, form a cyclic group selected from a 4-7 membered saturated heterocyclic ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, -L²-R⁶ is a substituent from Table C. In some embodiments, -L²-R⁶ is

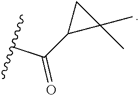 [SMILES: *C(=O)C1CC1(C)C]

In some embodiments, -L²-R⁶ is

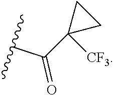 [SMILES: *C(=O)C1(C(F)(F)F)CC1]

In some embodiments, -L³-R⁸ is

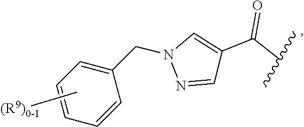

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

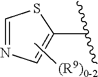

wherein R⁹ is methyl. In some embodiments, R¹ is

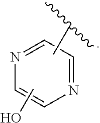

In some embodiments, the compound of Formula I is a compound of Formula IVb:

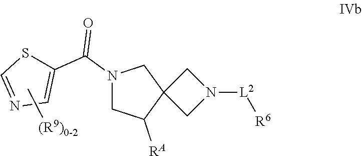

or a pharmaceutically acceptable salt thereof, wherein Z¹, R^{A}, L², R⁶, and R⁹, and their constituent groups, are each as defined and described herein. In some embodiments, the thiazolyl group is not substituted with R⁹. In some embodiments, R^{A} is a substituent from Table Bi, Bii, or Biii. In some embodiments, -L²-R⁶ is a substituent from Table C. In some embodiments, -L²-R⁶ is

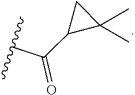 [SMILES: *C(=O)C1CC1(C)C]

In some embodiments, -L²-R⁶ is

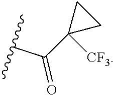 [SMILES: *C(=O)C1(C(F)(F)F)CC1]

In some embodiments, the compound of Formula I is a compound of Formula IVc:

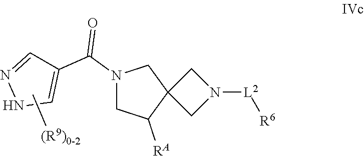

or a pharmaceutically acceptable salt thereof, wherein Z¹, R^{A}, L², R⁶, and R⁹, and their constituent groups, are each as defined and described herein. In some embodiments, the pyrazolyl group is not substituted with R⁹. In some embodiments, the pyrazolyl group is substituted with one instance of R⁹, wherein R⁹ is an optionally substituted benzyl group. In some embodiments, R^{A} is a substituent from Table Bi, Bii, or Biii. In some embodiments, -L²-R⁶ is a substituent from Table C. In some embodiments, -L²-R⁶ is

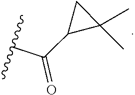 [SMILES: *C(=O)C1CC1(C)C]

In some embodiments, -L²-R⁶ is

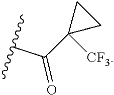 [SMILES: *C(=O)C1(C(F)(F)F)CC1]

In some embodiments, the compound of Formula I is a compound of Formula Va:

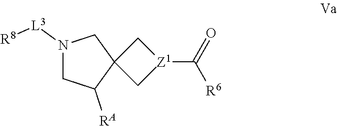

or a pharmaceutically acceptable salt thereof, wherein Z¹, R^{A}, R⁶, L³ and R¹, and their constituent groups, are each as defined and described herein. In some embodiments, R⁶ is an optionally substituted cyclopropyl group. In some embodiments, R^{A} is a substituent from Table Bi, Bii, or Biii. In some embodiments -L³-R⁸ is a substituent from Table D. In some embodiments, -L³-R⁸ is

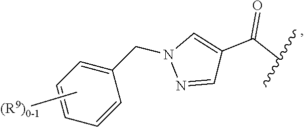

wherein R⁹ is —CF₃, —CN, —C(O)OH, —C(O)OCH₃, —C(O)OCH₂CH₃, —C(O)OC(CH₃)₃, or —C(O)OCH₂CH₂CH₃.

In some embodiments, the compound of Formula I is a compound of Formula Vb:

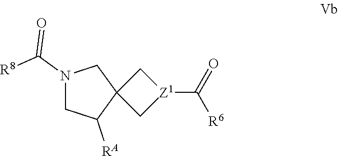

or a pharmaceutically acceptable salt thereof, wherein Z¹, R^{A}, R⁶, and R⁸, and their constituent groups, are each as defined and described herein. In some embodiments, R⁶ is an optionally substituted cyclopropyl group. In some embodiments, R^{A} is a substituent from Table Bi, Bii, or Biii. In some embodiments, R⁶ is

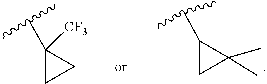

In some embodiments, R⁸ is

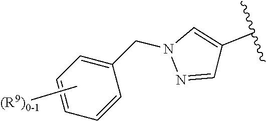

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

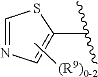

wherein R⁹ is methyl. In some embodiments, R¹ is

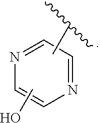

In some embodiments, the compound of Formula I is a compound of Formula VIa:

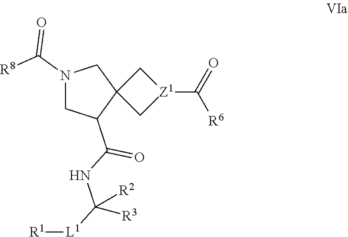

or a pharmaceutically acceptable salt thereof, wherein Z¹, L¹, R¹, R², R³, R⁶, and R⁸, and their constituent groups, are each as defined and described herein. In some embodiments, L¹ is

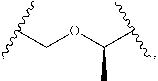 [SMILES: *CO[C@@H](*)C]

wherein the [symbol] on the left connects to R¹. In some embodiments, R⁶ is an optionally substituted cyclopropyl group. In some embodiments, L¹ is

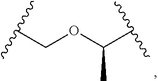 [SMILES: *CO[C@@H](*)C]

wherein the [symbol] on the left connects to R¹, wherein R¹ is

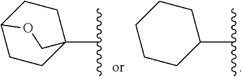

In some embodiments, R¹ is phenyl. In some embodiments, R¹ is cyclohexyl. In some embodiments, R¹ is

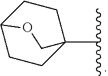 [SMILES: *C12CCC(CC1)OC2]

In some embodiments, R¹ is

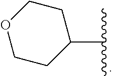 [SMILES: *C1CCOCC1]

In some embodiments, R¹ is

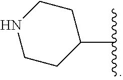 [SMILES: *C1CCNCC1]

In some embodiments, R¹ is

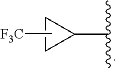

In some embodiments, R¹ is —CF₃. In some embodiments, R² is a substituent from Table A. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, R² is —C(O)NR₂, wherein the two R groups of —C(O)NR₂, taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

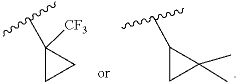

In some embodiments, R⁸ is

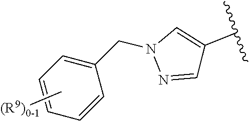

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

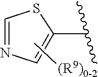

wherein R⁹ is methyl. In some embodiments, R⁸ is

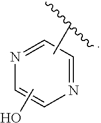

In some embodiments, the compound of Formula I is a compound of Formula VIb:

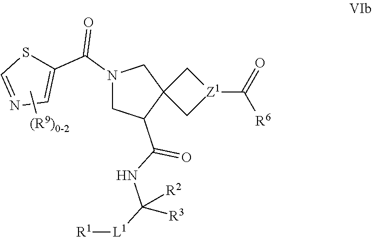

or a pharmaceutically acceptable salt thereof, wherein Z¹, L¹, R¹, R², R³, R⁶, and R⁹, and their constituent groups, are each as defined and described herein. In some embodiments, R¹ is phenyl.

In some embodiments, R¹ is cyclohexyl. In some embodiments, R¹ is

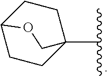 [SMILES: *C12CCC(CC1)OC2]

In some embodiments, R¹ is

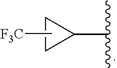

In some embodiments, R¹ is —CF₃. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

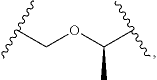 [SMILES: *CO[C@@H](*)C]

wherein the [symbol] on the left connects to R¹. In some embodiments, L¹ is

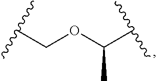 [SMILES: *CO[C@@H](*)C]

wherein the [symbol] on the left connects to R¹, wherein R¹ is

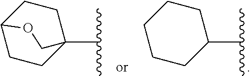 [SMILES: *C12CCC(CC1)OC2 ; *C1CCCCC1]

In some embodiments, R² is a substituent from Table A. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, the thiazolyl group is not substituted with R⁹. In some embodiments, R² is C(O)NR₂, wherein the two R groups, —C(O)NR₂, taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments R is an optionally substituted cyclopropyl group. In some embodiments, R⁶ is

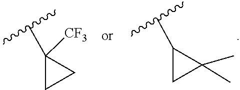

In some embodiments, the compound of Formula I is a compound of Formula VIc:

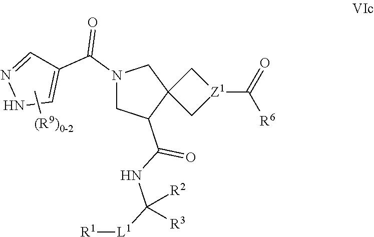

or a pharmaceutically acceptable salt thereof, wherein Z¹, L¹, R¹, R², R³, R⁶, and R⁹, and their constituent groups, are each as defined and described herein. In some embodiments, R¹ is phenyl. In some embodiments, R¹ is

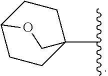 [SMILES: *C12CCC(CC1)OC2]

In some embodiments, R¹ is cyclohexyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

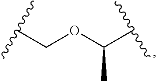 [SMILES: *CO[C@@H](*)C]

wherein the [symbol] on the left connects to R¹. In some embodiments, L¹ is

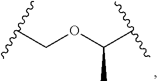 [SMILES: *CO[C@@H](*)C]

wherein the [symbol] on the left connects to R¹, wherein R¹ is

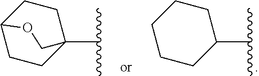 [SMILES: *C12CCC(CC1)OC2 ; *C1CCCCC1]

In some embodiments, R² is a substituent from Table A. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, the pyrazolyl group is not substituted with R⁹. In some embodiments, the pyrazolyl group is substituted with one instance of R⁹, wherein R⁹ is a benzyl group. In some embodiments, R² is —C(O)NR₂, wherein the two R groups of —C(O)NR₂, taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

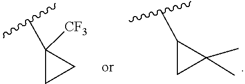

In some embodiments, the compound of Formula I is a compound of Formula VId.

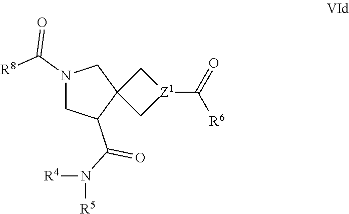

or a pharmaceutically acceptable salt thereof, wherein Z¹, R⁴, R⁵, R⁶, and R⁸, and their constituent groups, are each as defined and described herein. In some embodiments, R⁶ is an optionally substituted cyclopropyl group. In some embodiments, R⁶ is

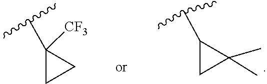

In some embodiments, R⁸ is

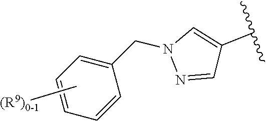

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

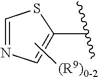

wherein R⁹ is methyl. In some embodiments, R⁸ is

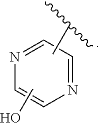

In some embodiments, R⁴ and R⁵, together, with the nitrogen to which they are attached, form an optionally substituted piperazine or an optionally substituted piperidine.

In some embodiments, the compound of Formula I is a compound of Formula VIe:

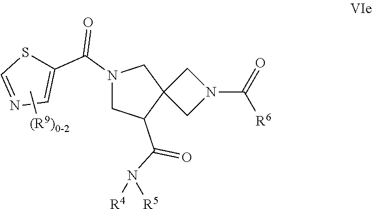

or a pharmaceutically acceptable salt thereof, wherein Z¹, R⁴, R⁵, R⁶, and R⁹, and their constituent groups, are each as defined and described herein. In some embodiments, R⁶ is an optionally substituted cyclopropyl group. In some embodiments, R⁶ is

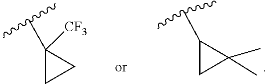

In some embodiments, R⁹ is methyl. In some embodiments, the thiazolyl group is not substituted with R⁹. In some embodiments, R⁴ and R⁵, together, with the nitrogen to which they are attached, form an optionally substituted piperazine or an optionally substituted piperidine.

In some embodiments, the compound of Formula I is a compound of Formula VIf:

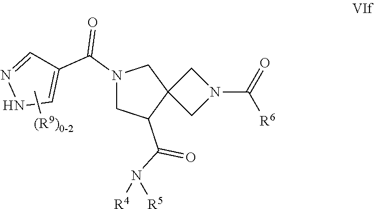

or a pharmaceutically acceptable salt thereof, wherein Z¹, R⁴, R⁵, R⁶, and R⁹, and their constituent groups, are each as defined and described herein. In some embodiments, R⁶ is an optionally substituted cyclopropyl group. In some embodiments, the pyrazolyl group is not substituted with R⁹. In some embodiments, R⁶ is

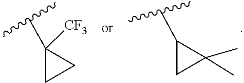

In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with CF₃, —CN, —C(O)OH, —C(O)OCH₃, —C(O)OCH₂CH₃, —C(O)OC(CH₃)₃, or —C(O)OCH₂CH₂CH₃. In some embodiments, R⁴ and R⁵, together, with the nitrogen to which they are attached, form an optionally substituted piperazine or an optionally substituted piperidine.

In some embodiments, the compound of Formula I is a compound of Formula VIIa:

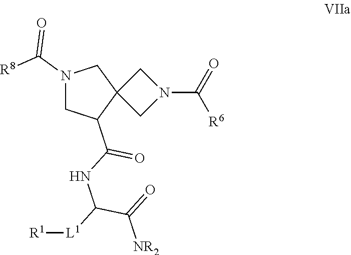

or a pharmaceutically acceptable salt thereof, wherein Z¹, L¹, R¹, R², R⁶, and R⁸, and their constituent groups, are each as defined and described herein. In some embodiments, R¹ is phenyl. In some embodiments, R¹ is

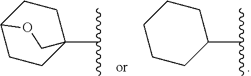 [SMILES: *C12CCC(CC1)OC2 ; *C1CCCCC1]

In some embodiments, R¹ is

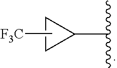

In some embodiments, R¹ is —CF₃. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

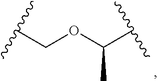 [SMILES: *CO[C@@H](*)C]

wherein the [symbol] on the left connects to R¹. In some embodiments, L¹ is

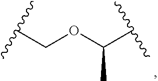 [SMILES: *CO[C@@H](*)C]

wherein the [symbol] on the left connects to R¹, wherein R¹ is

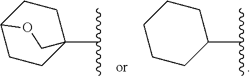 [SMILES: *C12CCC(CC1)OC2 ; *C1CCCCC1]

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁸ is

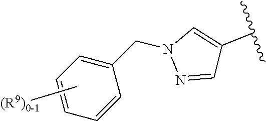

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R¹ is

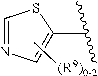

wherein R⁹ is methyl. In some embodiments, R⁸ is

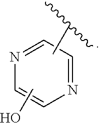

In some embodiments, R⁶ is an optionally substituted cyclopropyl group. In some embodiments, R⁶ is

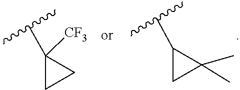

In some embodiments, the compound of Formula I is a compound of Formula VIIb:

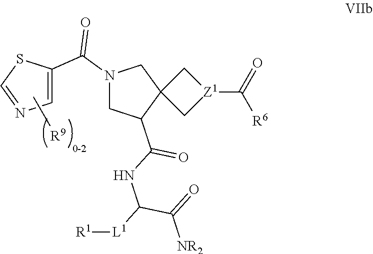

or a pharmaceutically acceptable salt thereof, wherein Z¹, L¹, R¹, R², R⁶, and R⁹, and their constituent groups, are each as defined and described herein. In some embodiments, R¹ is phenyl. In some embodiments, R¹ is cyclohexyl. In some embodiments, R¹ is

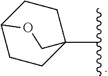 [SMILES: *C12CCC(CC1)OC2]

In some embodiments, R¹ is

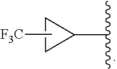

In some embodiments, R¹ is —CF₃. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

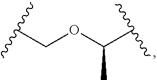 [SMILES: *CO[C@@H](*)C]

wherein the [symbol] on the left connects to R¹. In some embodiments, R⁶ is an optionally substituted cyclopropyl group. In some embodiments, L¹ is

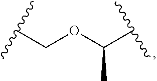 [SMILES: *CO[C@@H](*)C]

wherein the [symbol] on the left connects to R¹, wherein R¹ is

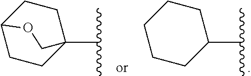 [SMILES: *C12CCC(CC1)OC2 ; *C1CCCCC1]

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

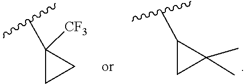

In some embodiments, the thiazolyl group is not substituted with R⁹.

In some embodiments, the compound of Formula I is a compound of Formula VIIc:

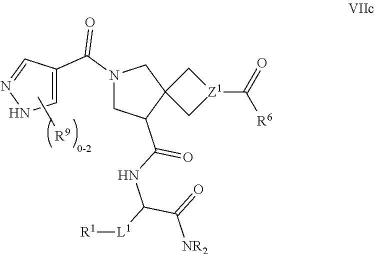

or a pharmaceutically acceptable salt thereof, wherein Z¹, L¹, R¹, R², R⁶, and R⁹, and their constituent groups, are each as defined and described herein. In some embodiments, R¹ is phenyl. In some embodiments, R¹ is cyclohexyl. In some embodiments, R¹ is

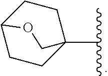 [SMILES: *C12CCC(CC1)OC2]

In some embodiments, R¹ is

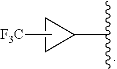

In some embodiments, R¹ is —CF₃. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

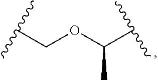 [SMILES: *CO[C@@H](*)C]

wherein the [symbol] on the left connects to R¹. In some embodiments, L¹ is

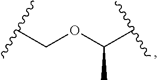 [SMILES: *CO[C@@H](*)C]

wherein the [symbol] on the left connects to R¹, wherein R¹ is

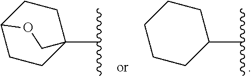 [SMILES: *C12CCC(CC1)OC2 ; *C1CCCCC1]

In embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

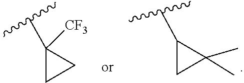

In some embodiments, the pyrazolyl group is not substituted with R⁹. In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with CF₃, —CN, —C(O)OH, —C(O)OCH₃, —C(O)OCH₂CH₃, —C(O)OC(CH₃)₃, or —C(O)OCH₂CH₂CH₃.

In some embodiments, the compound of Formula I is a compound of Formula VIIIa:

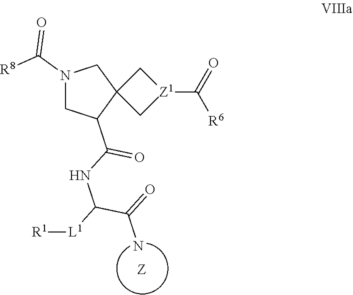

or a pharmaceutically acceptable salt thereof, wherein Z¹, L¹, R¹, R⁶, and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

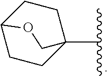 [SMILES: *C12CCC(CC1)OC2]

In some embodiments, L¹ is an optionally substituted straight or branched C₁₋₄ alkylene chain, wherein 1-2 methylene units of L¹ are independently replaced by —O—, —NR—, —C(O)O—, or —NRC(O)—In some embodiments, L¹ is

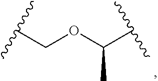 [SMILES: *CO[C@@H](*)C]

wherein the [symbol] on the left connects to R¹. In some embodiments, L¹ is

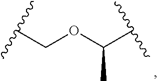 [SMILES: *CO[C@@H](*)C]

wherein the [symbol] on the left connects to R¹, wherein R¹ is

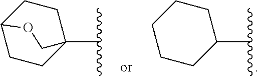 [SMILES: *C12CCC(CC1)OC2 ; *C1CCCCC1]

In some embodiments, R¹ is

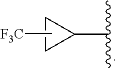

In some embodiments, R¹ is —CF₃. In some embodiments, cyclic moiety Z with the intervening nitrogen atom, forms a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁸ is

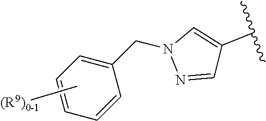

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

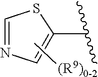

wherein R⁹ is methyl. In some embodiments, R⁸ is

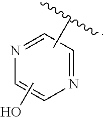

In some embodiments, R⁶ is

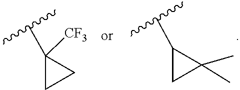

In some embodiments, R⁶ is an optionally substituted cyclopropyl group. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula VIIIb:

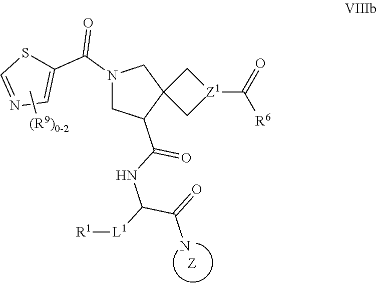

or a pharmaceutically acceptable salt thereof, wherein Z¹, L¹, R¹, and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is

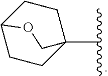 [SMILES: *C12CCC(CC1)OC2]

In some embodiments, R¹ is

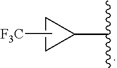

in some embodiments, R¹ is —CF₃. In some embodiments, R¹ is cyclohexyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

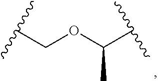 [SMILES: *CO[C@@H](*)C]

wherein the [symbol] on the left connects to R¹. In some embodiments, L¹ is

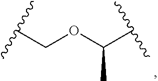 [SMILES: *CO[C@@H](*)C]

wherein the [symbol] on the left connects to R¹, wherein R¹ is

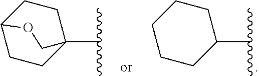 [SMILES: *C12CCC(CC1)OC2 ; *C1CCCCC1]

In some embodiments, the cyclic moiety Z taken together with the intervening nitrogen atom, forms a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

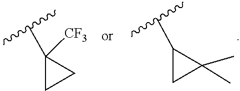

In some embodiments, the thiazolyl group is not substituted with R⁹. In some embodiments, the thiazolyl group is substituted with 0-1 R⁹ instances which are methyl. In some embodiments, Z, or in any of the aforementioned embodiments of this paragraph, is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiment formula VIIIc:

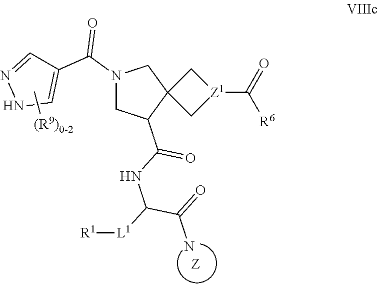

or a pharmaceutically acceptable salt thereof, wherein Z¹, L¹, R¹, R⁶, and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

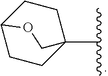 [SMILES: *C12CCC(CC1)OC2]

In some embodiments, L¹ is an optionally substituted straight or branched C₁₋₄ alkylene chain, wherein 1-2 methylene units of L¹ are independently replaced by —O—, —NR—, —C(O)O—, or —NRC(O)—In some embodiments, L¹ is

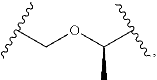 [SMILES: *CO[C@@H](*)C]

wherein the [symbol] on the left connects to R¹. In some embodiments, L¹ is

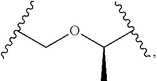 [SMILES: *CO[C@@H](*)C]

wherein the [symbol] on the left connects to R¹, wherein R¹ is

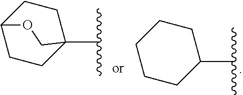

In some embodiments, R¹ is

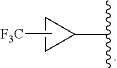

In some embodiments, R¹ is —CF₃. In some embodiments, the cyclic moiety Z taken together with the intervening nitrogen atom, forms a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

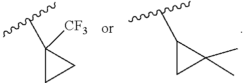

In some embodiments, the pyrazolyl group is not substituted with R⁹. In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with CF₃, —CN, —C(O)OH, —C(O)OCH₃, —C(O)OCH₂CH₃, or —C(O)OCH₂CH₂CH₃. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXa:

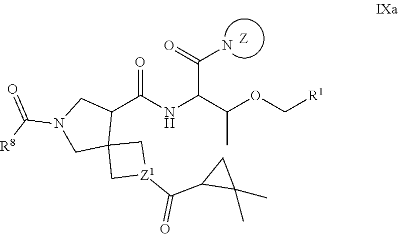

or a pharmaceutically acceptable salt thereof, wherein Z¹, R¹ and R⁸, and their constituent groups, are each as defined and described herein and wherein T is N or CH, and cyclic moiety Z is an optionally substituted cyclic group formed from two R groups, as defined and described herein. In some embodiments, R¹ is phenyl. In some embodiments, R¹ is

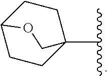 [SMILES: *C12CCC(CC1)OC2]

In some embodiments, R¹ is

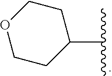 [SMILES: *C1CCOCC1]

In some embodiments, R¹ is

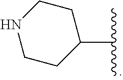 [SMILES: *C1CCNCC1]

In some embodiments, R¹ is cyclohexyl. In some embodiments, R¹ is

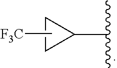

In some embodiments, R¹ is —CF₃. In some embodiments, R⁸ is

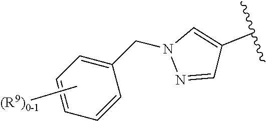

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. It some embodiments, R⁸ is

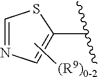

wherein R⁹ is methyl. In some embodiments, R⁸ is

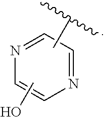

In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXa*:

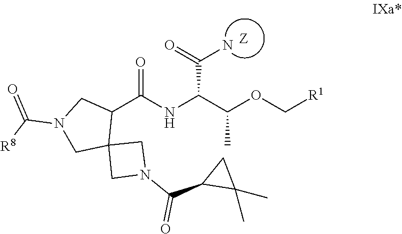

or a pharmaceutically acceptable salt thereof, wherein Z¹, R¹ and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

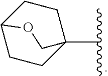 [SMILES: *C12CCC(CC1)OC2]

In some embodiments, R¹ is

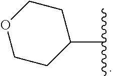 [SMILES: *C1CCOCC1]

In some embodiments, R¹ is

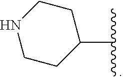 [SMILES: *C1CCNCC1]

In some embodiments, R¹ is

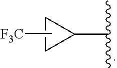

In some embodiments, R¹ is —CF₃. In some embodiments, R⁸ is

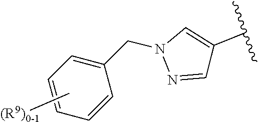

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

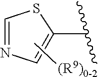

wherein R⁹ is methyl. In some embodiments, R¹ is

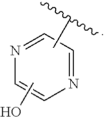

In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXa**:

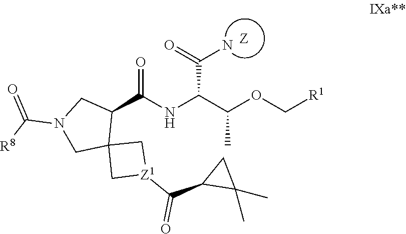

or a pharmaceutically acceptable salt thereof, wherein Z¹, R¹ and R⁸, and their constituent groups, are each as defined and described herein and cyclic moiety Z is an optionally substituted cyclic group formed from two R groups, as defined and described herein. In some embodiments, R¹ is phenyl. In some embodiments, R¹ is cyclohexyl. In some embodiments, R¹ is

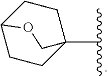 [SMILES: *C12CCC(CC1)OC2]

In some embodiments, R¹ is

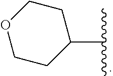 [SMILES: *C1CCOCC1]

In some embodiments, R¹ is

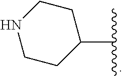 [SMILES: *C1CCNCC1]

In some embodiments, R¹ is

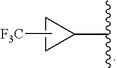

In some embodiments, R¹ is —CF₃. In some embodiments, R⁸ is

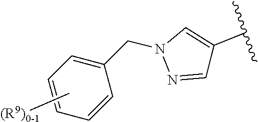

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R¹ is

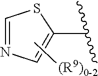

wherein R⁹ is methyl. In some embodiments, R⁸ is

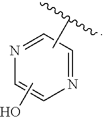

In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXb:

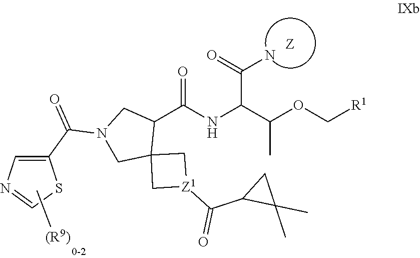

or a pharmaceutically acceptable salt thereof, wherein Z¹, R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

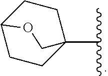 [SMILES: *C12CCC(CC1)OC2]

In some embodiments, R¹ is

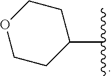 [SMILES: *C1CCOCC1]

In some embodiments, R¹ is

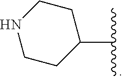 [SMILES: *C1CCNCC1]

In some embodiments, R¹ is

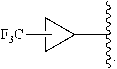

In some embodiments, R¹ is —CF₃. In some embodiments, the thiazolyl group is not substituted with R⁹. In some embodiments, the thiazolyl group is substituted with one instance of R⁹, wherein R⁹ is a benzyl group. In some embodiments, the thiazolyl group is substituted with one or two instances of R⁹, which are methyl groups. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXb*:

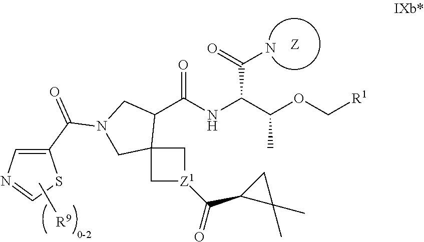

or a pharmaceutically acceptable salt thereof, wherein Z¹, R¹ and R⁹, and their constituent groups, are each as defined and described herein and cyclic moiety Z is an optionally substituted cyclic group formed from two R groups, as defined and described herein. In some embodiments, R¹ is phenyl. In some embodiments, R¹ is cyclohexyl. In some embodiments, R¹ is

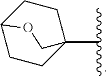 [SMILES: *C12CCC(CC1)OC2]

In some embodiments, R¹ is

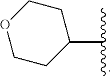 [SMILES: *C1CCOCC1]

In some embodiments, R¹ is

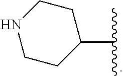 [SMILES: *C1CCNCC1]

In some embodiments, R¹ is

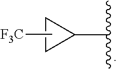

In some embodiments, R¹ is —CF₃. In some embodiments, the thiazolyl group is not substituted with R⁹. In some embodiments, the thiazolyl group is substituted with one instance of R⁹, wherein R⁹ is a benzyl group. In some embodiments, the thiazolyl group is substituted with one or two instances of R⁹, which are methyl groups. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXb**:

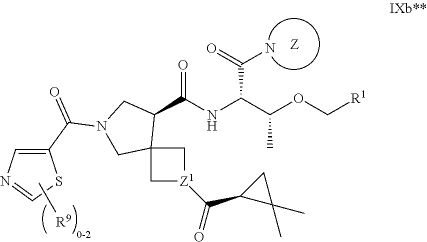

or a pharmaceutically acceptable salt thereof, wherein Z¹, R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

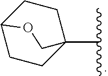 [SMILES: *C12CCC(CC1)OC2]

In some embodiments, R¹ is

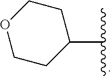 [SMILES: *C1CCOCC1]

In some embodiments, R¹ is

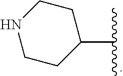 [SMILES: *C1CCNCC1]

In some embodiments, R¹ is

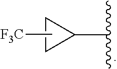

In some embodiments, R¹ is —CF₃. In some embodiments, the thiazolyl group is not substituted with R⁹. In some embodiments, the thiazolyl group is substituted with one instance of R⁹, wherein R⁹ is a benzyl group. In some embodiments, the thiazolyl group is substituted with one or two instances of R⁹, which are methyl groups. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXc:

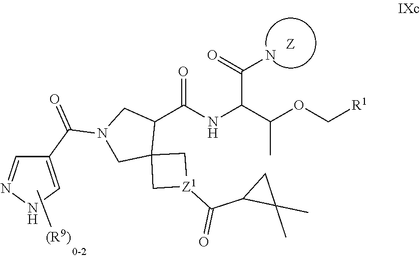

or a pharmaceutically acceptable salt thereof, wherein Z¹, R¹ and R⁹, and their constituent groups, are each as defined and described herein and cyclic moiety Z is an optionally substituted cyclic group formed from two R groups, as defined and described herein. In some embodiments, R¹ is phenyl. In some embodiments, R¹ is cyclohexyl. In some embodiments, R¹ is

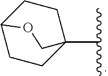 [SMILES: *C12CCC(CC1)OC2]

In some embodiments, R¹ is

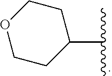 [SMILES: *C1CCOCC1]

In some embodiments, R¹ is

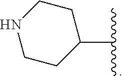 [SMILES: *C1CCNCC1]

In some embodiments, R¹ is

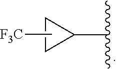

In some embodiments, R¹ is —CF₃. In some embodiments, the pyrazolyl group is not substituted with R⁹. In some embodiments, the pyrazolyl group is substituted with one instance of R⁹, wherein R⁹ is a benzyl group. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXc*:

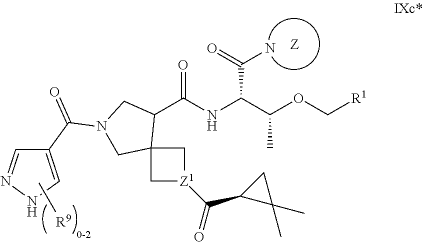

or a pharmaceutically acceptable salt thereof, wherein Z¹, R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

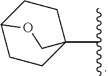 [SMILES: *C12CCC(CC1)OC2]

In some embodiments, R¹ is

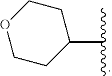 [SMILES: *C1CCOCC1]

In some embodiments, R¹ is

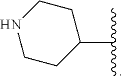 [SMILES: *C1CCNCC1]

In some embodiments, R¹ is

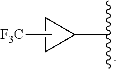

In some embodiments, R¹ is —CF₃. In some embodiments, the pyrazolyl group is not substituted with R⁹. In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with —CF₃, —CN, —C(O)OH, —C(O)OCH₃, —C(O)OCH₂CH₃, or —C(O)OCH₂CH₂CH₃. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula IXc**:

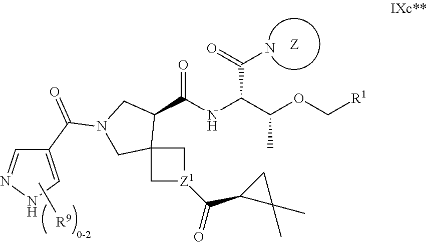

or a pharmaceutically acceptable salt thereof, wherein Z¹, R¹ and R⁹, and their constituent groups, are each as defined and described herein and cyclic moiety Z is an optionally substituted cyclic group formed from two R groups, as defined and described herein. In some embodiments, R¹ is phenyl. In some embodiments, R¹ is

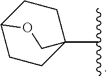 [SMILES: *C12CCC(CC1)OC2]

n some embodiments, R¹ is

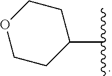 [SMILES: *C1CCOCC1]

In some embodiments, R¹ is

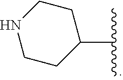 [SMILES: *C1CCNCC1]

In some embodiments, R¹ is

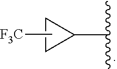

In some embodiments, R¹ is —CF₃. In some embodiments, R¹ is cyclohexyl. In some embodiments, the pyrazolyl group is not substituted with R⁹. In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with —CF₃, —CN, —C(O)OH, —C(O)OCH₃, —C(O)OCH₂CH₃, or —C(O)OCH₂CH₂CH₃. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula Xa:

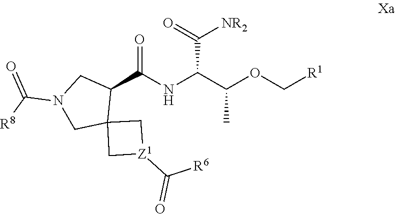

or a pharmaceutically acceptable salt thereof, wherein Z¹, R¹, R⁶, and R⁸, and their constituent groups, are each as defined and described herein. In some embodiments, R¹ is phenyl. In some embodiments, R¹ is cyclohexyl. In some embodiments, R¹ is

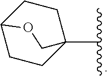 [SMILES: *C12CCC(CC1)OC2]

In some embodiments, R¹ is

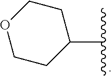 [SMILES: *C1CCOCC1]

In some embodiments, R¹ is

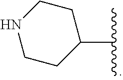 [SMILES: *C1CCNCC1]

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

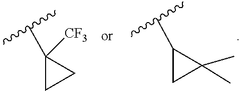

In some embodiments, R⁸ is

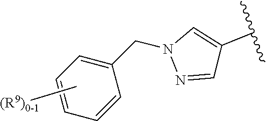

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R¹ is

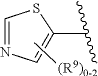

wherein R⁹ is methyl. In some embodiments, R⁸ is

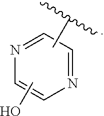

In some embodiments, the compound of Formula I is a compound of Formula Xb:

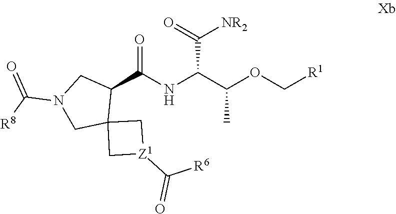

or a pharmaceutically acceptable salt thereof, wherein Z¹, R¹, R⁶, and R⁸, and their constituent groups, are each as defined and described herein. In some embodiments, R¹ is phenyl. In some embodiments, R¹ is cyclohexyl. In some embodiments, R¹ is

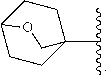 [SMILES: *C12CCC(CC1)OC2]

In some embodiments, R¹ is

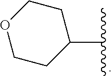 [SMILES: *C1CCOCC1]

In some embodiments, R¹ is

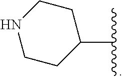 [SMILES: *C1CCNCC1]

In some embodiments, R⁶ is an optionally substituted cyclopropyl group. In some embodiments, R⁶ is

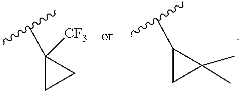

In some embodiments, R⁸ is

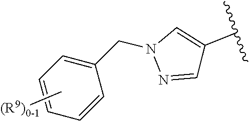

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

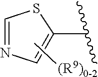

wherein R⁹ is methyl. In some embodiments, R⁸ is

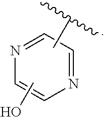

In some embodiments, the compound of Formula I is a compound of Formula Xc:

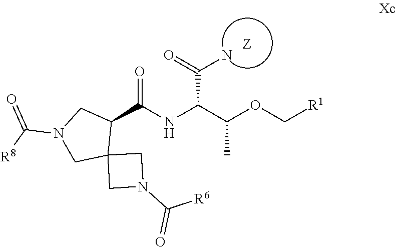

or a pharmaceutically acceptable salt thereof, wherein Z¹, ring Z, R¹, R⁶, and R⁸, and their constituent groups, are each as defined and described herein. In some embodiments, R¹ is phenyl. In some embodiments, R¹ is cyclohexyl. In some embodiments, R¹ is

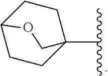 [SMILES: *C12CCC(CC1)OC2]

In some embodiments, R¹ is

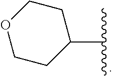 [SMILES: *C1CCOCC1]

In some embodiments, R¹ is

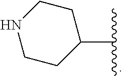 [SMILES: *C1CCNCC1]

In some embodiments, R⁶ is an optionally substituted cyclopropyl group. In some embodiments, R⁶ is

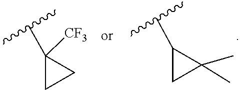

In some embodiments, R⁸ is

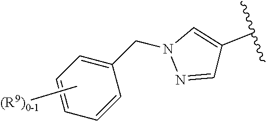

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

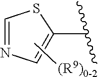

wherein R⁹ is methyl. In some embodiments, R⁸ is

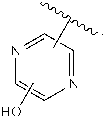

In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula XIa:

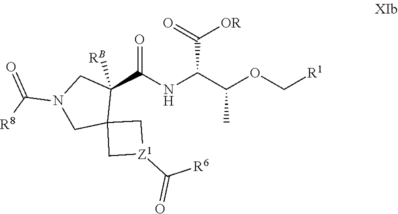

or a pharmaceutically acceptable salt thereof, wherein Z¹, R^{B}, R¹, R⁶, and R⁸, and their constituent groups, are each as defined and described herein. In some embodiments, R¹ is phenyl. In some embodiments, R¹ is cyclohexyl. In some embodiments, R¹ is

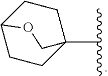 [SMILES: *C12CCC(CC1)OC2]

In some embodiments, R¹ is

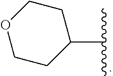 [SMILES: *C1CCOCC1]

In some embodiments, R¹ is

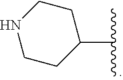 [SMILES: *C1CCNCC1]

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

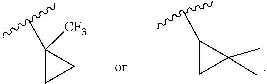

In some embodiments, R⁸ is

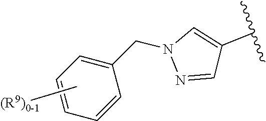

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R¹ is

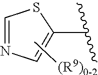

wherein R⁹ is methyl. In some embodiments, R⁸ is

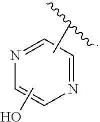

In some embodiments, the compound of Formula I is a compound of Formula XIb:

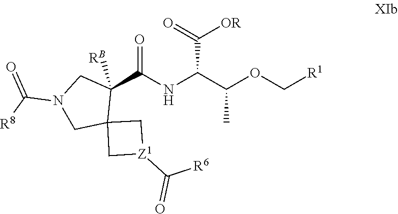

or a pharmaceutically acceptable salt thereof, wherein Z¹, R^{B}, R¹, R⁶, and R⁸, and their constituent groups, are each as defined and described herein. In some embodiments, R¹ is phenyl. In some embodiments, R¹ is cyclohexyl. In some embodiments, R¹ is

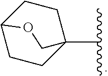 [SMILES: *C12CCC(CC1)OC2]

In some embodiments, R¹ is

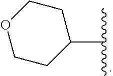 [SMILES: *C1CCOCC1]

In some embodiments, R¹ is

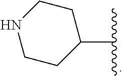 [SMILES: *C1CCNCC1]

In some embodiments, R⁶ is an optionally substituted cyclopropyl group. In some embodiments, R⁶ is

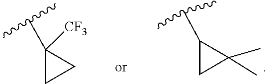

In some embodiments, R⁸ is

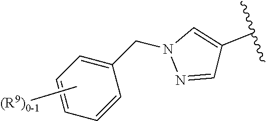

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

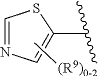

wherein R⁹ is methyl. In some embodiments, R⁸ is

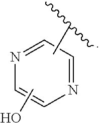

In some embodiments, the compound of Formula I is a compound of Formula XIc:

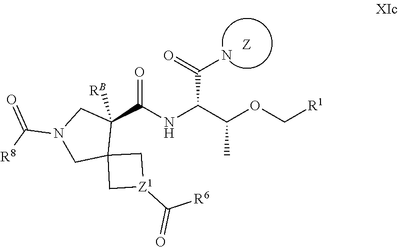

or a pharmaceutically acceptable salt thereof, wherein Z¹, ring Z, R^{B}, R¹, R⁶, and R⁸, and their constituent groups, are each as defined and described herein. In some embodiments, R¹ is phenyl. In some embodiments, R¹ is cyclohexyl. In some embodiments, R¹ is

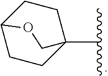 [SMILES: *C12CCC(CC1)OC2]

In some embodiments, R¹ is

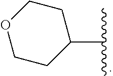 [SMILES: *C1CCOCC1]

In some embodiments, R¹ is

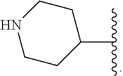 [SMILES: *C1CCNCC1]

In some embodiments, R⁶ is an optionally substituted cyclopropyl group. In some embodiments, R⁶ is

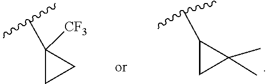

In some embodiments, R⁸ is

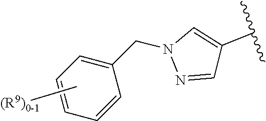

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

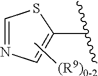

wherein R⁹ is methyl. In some embodiments, R⁸ is

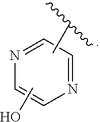

In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I is a compound of Formula XIIa or XIIb:

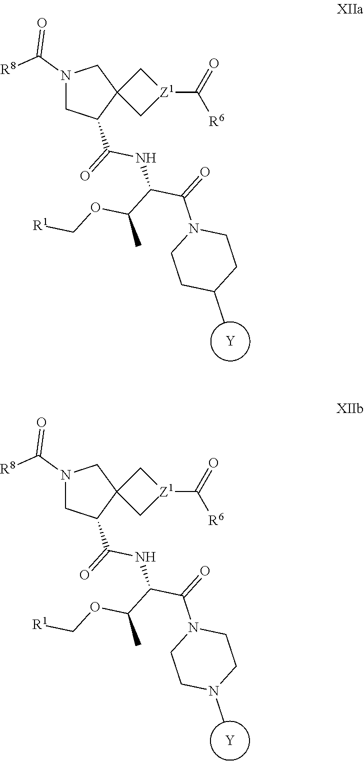

or a pharmaceutically acceptable salt thereof, wherein Z¹, R¹, R⁶, and R⁸, and their constituent groups, are each as defined and described herein, and wherein

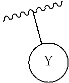

is —CH₂—Cy or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R¹ is phenyl. In some embodiments, R¹ is cyclohexyl. In some embodiments, R¹ is

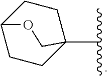 [SMILES: *C12CCC(CC1)OC2]

In some embodiments, R¹ is

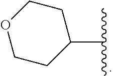 [SMILES: *C1CCOCC1]

In some embodiments, R¹ is

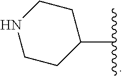 [SMILES: *C1CCNCC1]

In some embodiments, R⁶ is an optionally substituted cyclopropyl group. In some embodiments, R⁶ is or

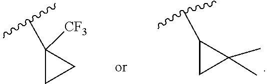

In some embodiments, R⁸ is

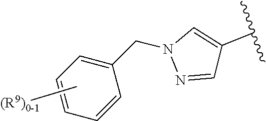

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R¹ is

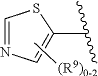

wherein R⁹ is methyl. In some embodiments, R⁸ is

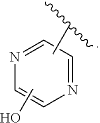

In some embodiments, the compound of Formula I is a compound of Formula XIIIa, XIIIb, XIIIc, or XIIId:

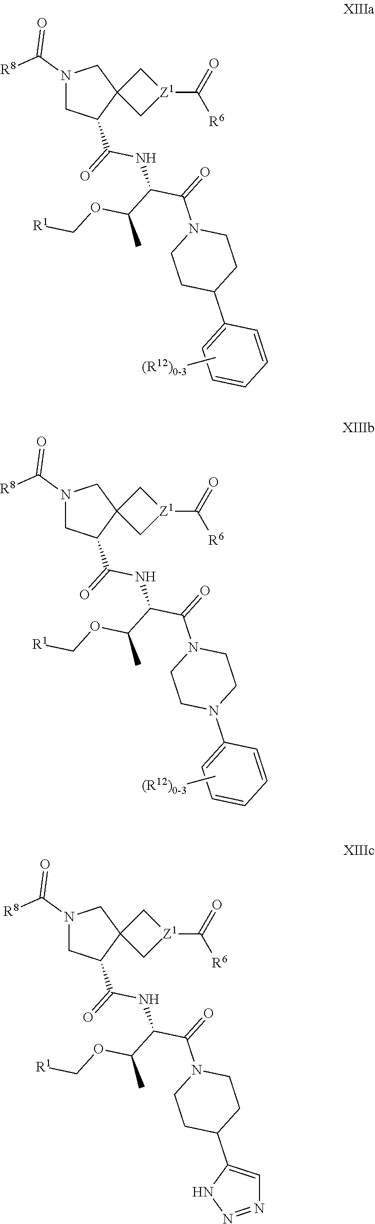

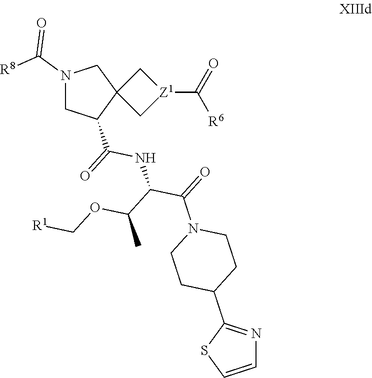

or a pharmaceutically acceptable salt thereof, wherein Z¹, R¹, R⁶, and R⁸, and their constituent groups, are each as defined and described herein, and wherein
R¹² has from 0 to 3 instances each independently selected from halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, and Cy. In some embodiments, R¹² has from 0 to 3 instances each independently selected from —F, —CF₃, —C(O)OH, —C(O)OCH₃, —C(O)OCH₂CH₃, —C(O)OCH₂CH₂CH₃, —C(O)OC(CH₃)₃, —OCH₂C(O)OH, —OCH₂C(O)OCH₃, —CH₃, —OC(CH₃)₂C(O)OH, —OC(CH₃)₂C(O)OCH₃. In some embodiments, R¹ is cyclohexyl. In some embodiments, R¹ is

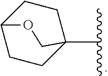 [SMILES: *C12CCC(CC1)OC2]

In some embodiments, R¹ is

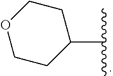 [SMILES: *C1CCOCC1]

In some embodiments, R¹ is

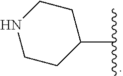 [SMILES: *C1CCNCC1]

In some embodiments, R⁶ is an optionally substituted cyclopropyl group. In some embodiments, R⁶ is

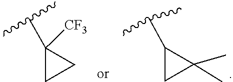

In some embodiments, R⁸ is

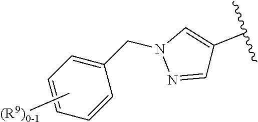

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

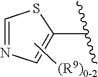

wherein R⁹ is methyl. In some embodiments, R⁸ is

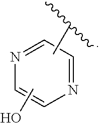

In some embodiments, the compound of Formula I is a compound of Formula XIV:

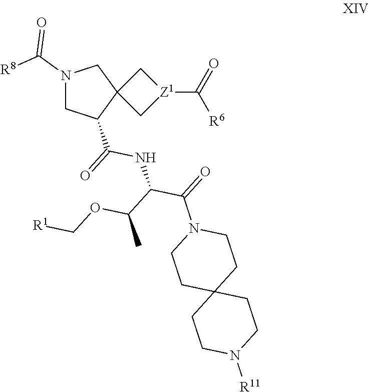

or a pharmaceutically acceptable salt thereof, wherein Z¹, R¹, R⁶, and R⁸, and their constituent groups, are each as defined and described herein, and wherein R¹¹ is hydrogen, —S(O)₂R, —S(O)₂NR₂, —S(O)R, —S(O)NR₂, —C(O)R, —C(O)OR, C(O)NR₂, —C(O)N(R)OR, an optionally substituted C₁₋₆ aliphatic group, an optionally substituted C₁₋₆ aliphatic-Cy group, or Cy. In some embodiments, R¹¹ is hydrogen, —C(O)OR, or an optionally substituted C₁₋₆ aliphatic group. In some embodiments, R¹¹ is cyclohexyl. In some embodiments, R¹ is

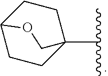 [SMILES: *C12CCC(CC1)OC2]

In some embodiments, R¹ is

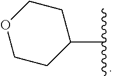 [SMILES: *C1CCOCC1]

In some embodiments, R¹ is

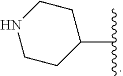 [SMILES: *C1CCNCC1]

In some embodiments, R⁶ is an optionally substituted cyclopropyl group. In some embodiments, R⁶ is

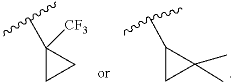

In some embodiments, R⁸ is

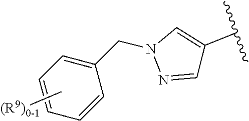

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R¹ is

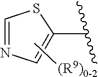

wherein R⁹ is methyl. In some embodiments, R⁸ is

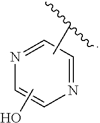

Exemplary compounds of the present disclosure are set forth in Table 1, below.

TABLE 1
Exemplary Compounds

| Cmpd | |
|---|---|
| # | Structure |
| I-1 | 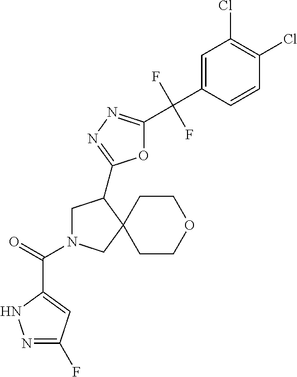 [SMILES: O=C(c1cc(F)n[nH]1)N1CC(c2nnc(C(F)(F)c3ccc(Cl)c(Cl)c3)o2)C2(CCOCC2)C1] |
| I-2 | 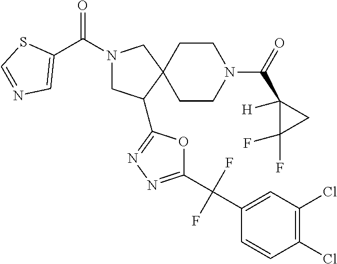 [SMILES: O=C(c1cncs1)N1CC(c2nnc(C(F)(F)c3ccc(Cl)c(Cl)c3)o2)C2(CCN(C(=O)[C@H]3CC3(F)F)CC2)C1] |
| I-3 | 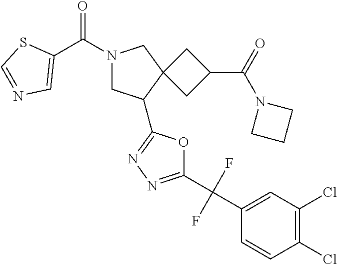 [SMILES: O=C(c1cncs1)N1CC(c2nnc(C(F)(F)c3ccc(Cl)c(Cl)c3)o2)C2(CC(C(=O)N3CCC3)C2)C1] |
| I-4 | 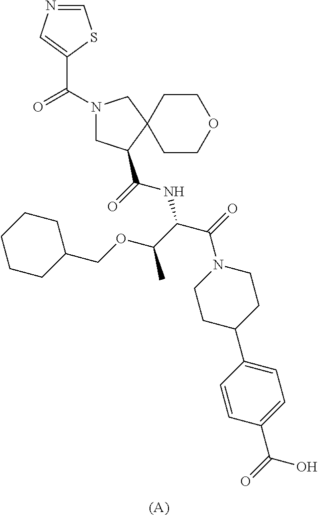 [SMILES: C[C@@H](OCC1CCCCC1)[C@H](NC(=O)[C@H]1CN(C(=O)c2cncs2)CC12CCOCC2)C(=O)N1CCC(c2ccc(C(=O)O)cc2)CC1 ; "(A)"] |
| | 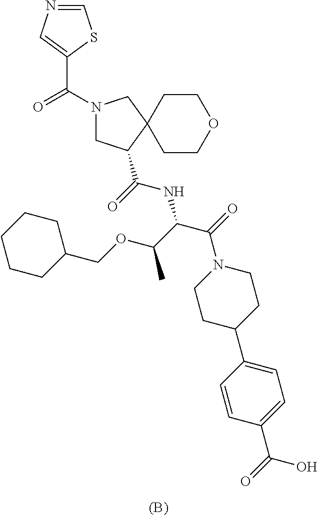 [SMILES: C[C@@H](OCC1CCCCC1)[C@H](NC(=O)[C@@H]1CN(C(=O)c2cncs2)CC12CCOCC2)C(=O)N1CCC(c2ccc(C(=O)O)cc2)CC1 ; "(B)"] |
| I-5 | 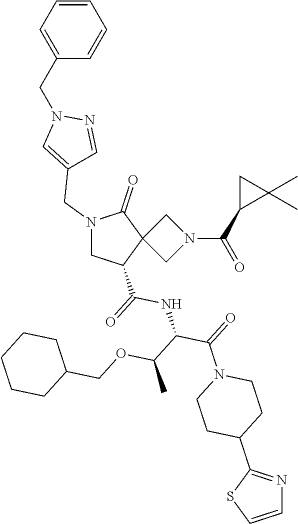 [SMILES: C[C@@H](OCC1CCCCC1)[C@H](NC(=O)[C@@H]1CN(Cc2cnn(Cc3ccccc3)c2)C(=O)C12CN(C(=O)[C@H]1CC1(C)C)C2)C(=O)N1CCC(c2nccs2)CC1] |
| I-6 | 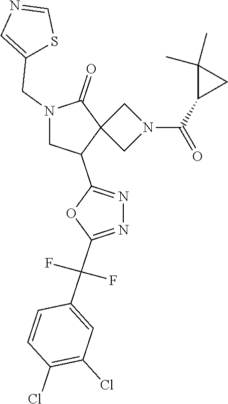 [SMILES: CC1(C)C[C@@H]1C(=O)N1CC2(C1)C(=O)N(Cc1cncs1)CC2c1nnc(C(F)(F)c2ccc(Cl)c(Cl)c2)o1] |
| I-7 | 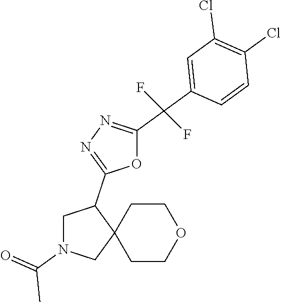 [SMILES: CC(=O)N1CC(c2nnc(C(F)(F)c3ccc(Cl)c(Cl)c3)o2)C2(CCOCC2)C1] |
| I-8 | 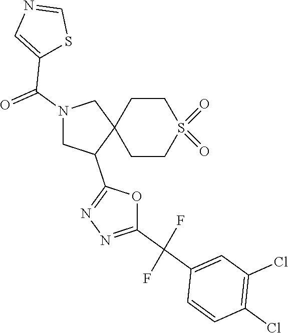 [SMILES: O=C(c1cncs1)N1CC(c2nnc(C(F)(F)c3ccc(Cl)c(Cl)c3)o2)C2(CCS(=O)(=O)CC2)C1] |
| I-9 | 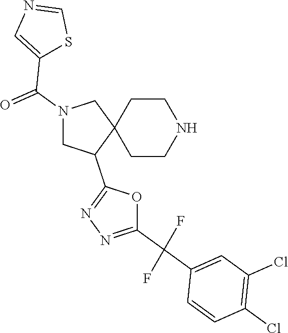 [SMILES: O=C(c1cncs1)N1CC(c2nnc(C(F)(F)c3ccc(Cl)c(Cl)c3)o2)C2(CCNCC2)C1] |
| I-10 | 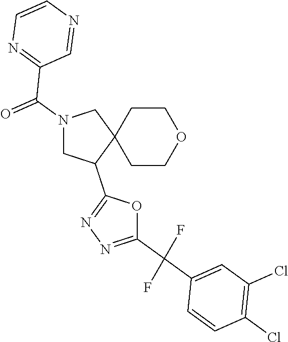 [SMILES: O=C(c1cnccn1)N1CC(c2nnc(C(F)(F)c3ccc(Cl)c(Cl)c3)o2)C2(CCOCC2)C1] |
| I-11 | 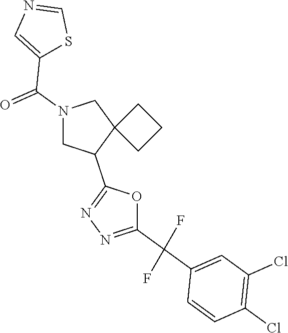 [SMILES: O=C(c1cncs1)N1CC(c2nnc(C(F)(F)c3ccc(Cl)c(Cl)c3)o2)C2(CCC2)C1] |
| I-12 | 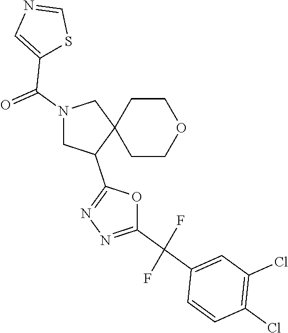 [SMILES: O=C(c1cncs1)N1CC(c2nnc(C(F)(F)c3ccc(Cl)c(Cl)c3)o2)C2(CCOCC2)C1] |
| I-13 | 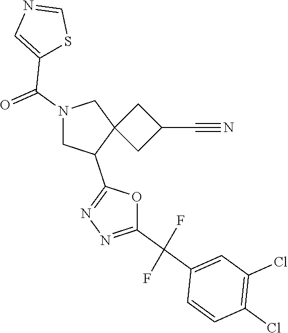 [SMILES: N#CC1CC2(C1)CN(C(=O)c1cncs1)CC2c1nnc(C(F)(F)c2ccc(Cl)c(Cl)c2)o1] |
| I-14 | 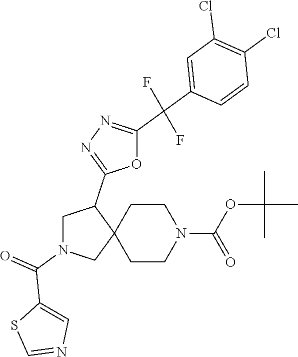 [SMILES: CC(C)(C)OC(=O)N1CCC2(CC1)CN(C(=O)c1cncs1)CC2c1nnc(C(F)(F)c2ccc(Cl)c(Cl)c2)o1] |
| I-15 | 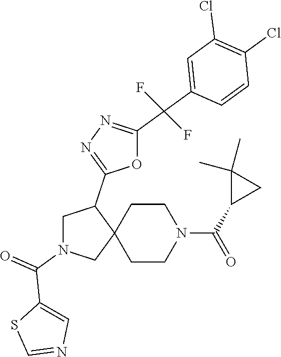 [SMILES: CC1(C)C[C@@H]1C(=O)N1CCC2(CC1)CN(C(=O)c1cncs1)CC2c1nnc(C(F)(F)c2ccc(Cl)c(Cl)c2)o1] |
| I-16 | 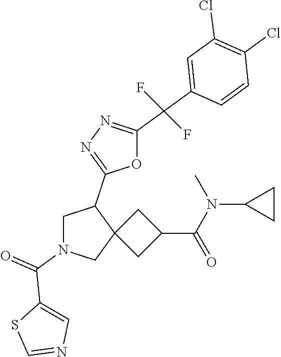 [SMILES: CN(C(=O)C1CC2(C1)CN(C(=O)c1cncs1)CC2c1nnc(C(F)(F)c2ccc(Cl)c(Cl)c2)o1)C1CC1] |
| I-17 | 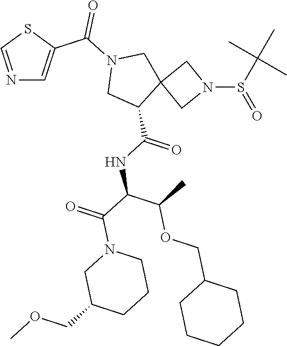 [SMILES: COC[C@H]1CCCN(C(=O)[C@@H](NC(=O)[C@@H]2CN(C(=O)c3cncs3)CC23CN(S(=O)C(C)(C)C)C3)[C@@H](C)OCC2CCCCC2)C1] |
| I-18 | 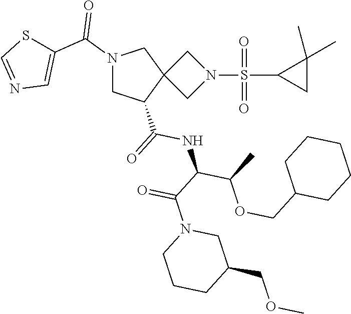 [SMILES: COC[C@H]1CCCN(C(=O)[C@@H](NC(=O)[C@@H]2CN(C(=O)c3cncs3)CC23CN(S(=O)(=O)C2CC2(C)C)C3)[C@@H](C)OCC2CCCCC2)C1] |
| I-19 | 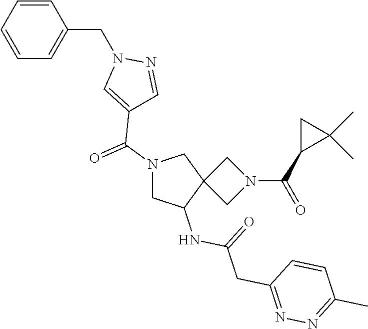 [SMILES: Cc1ccc(CC(=O)NC2CN(C(=O)c3cnn(Cc4ccccc4)c3)CC23CN(C(=O)[C@H]2CC2(C)C)C3)nn1] |
| I-20 | 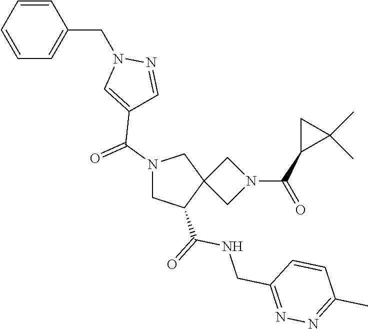 [SMILES: Cc1ccc(CNC(=O)[C@@H]2CN(C(=O)c3cnn(Cc4ccccc4)c3)CC23CN(C(=O)[C@H]2CC2(C)C)C3)nn1] |
| I-21 | 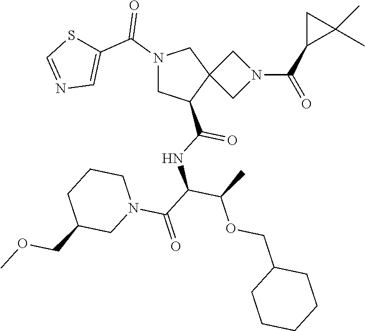 |
| I-22 | 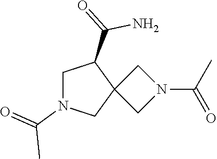 [SMILES: CC(=O)N1C[C@@H](C(N)=O)C2(C1)CN(C(C)=O)C2] |
| I-23 | 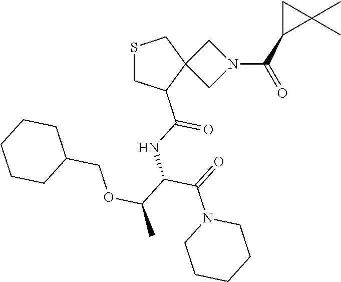 [SMILES: C[C@@H](OCC1CCCCC1)[C@H](NC(=O)C1CSCC12CN(C(=O)[C@H]1CC1(C)C)C2)C(=O)N1CCCCC1] |
| I-24 | 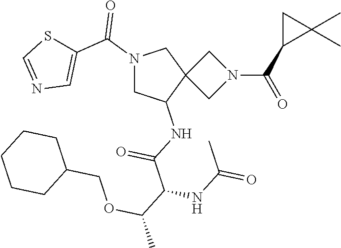 [SMILES: CC(=O)N[C@@H](C(=O)NC1CN(C(=O)c2cncs2)CC12CN(C(=O)[C@H]1CC1(C)C)C2)[C@H](C)OCC1CCCCC1] |
| I-25 | 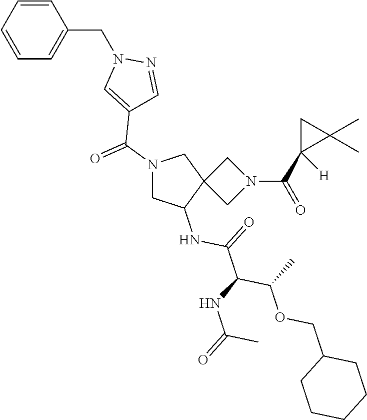 [SMILES: CC(=O)N[C@@H](C(=O)NC1CN(C(=O)c2cnn(Cc3ccccc3)c2)CC12CN(C(=O)[C@H]1CC1(C)C)C2)[C@H](C)OCC1CCCCC1] |
| I-26 | 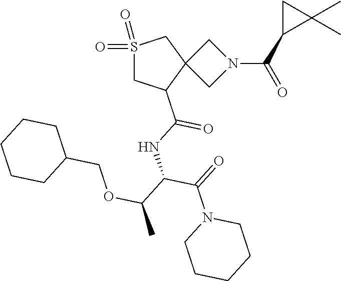 [SMILES: C[C@@H](OCC1CCCCC1)[C@H](NC(=O)C1CS(=O)(=O)CC12CN(C(=O)[C@H]1CC1(C)C)C2)C(=O)N1CCCCC1] |
| I-27 | 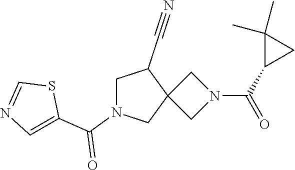 [SMILES: CC1(C)C[C@@H]1C(=O)N1CC2(CN(C(=O)c3cncs3)CC2C#N)C1] |
| I-28 | 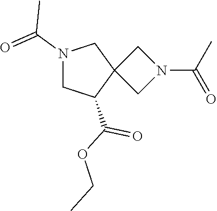 [SMILES: CCOC(=O)[C@@H]1CN(C(C)=O)CC12CN(C(C)=O)C2] |
| I-29 | 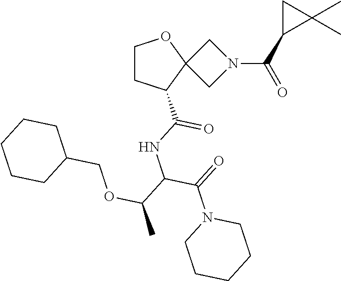 [SMILES: C[C@@H](OCC1CCCCC1)C(NC(=O)[C@@H]1CCOC12CN(C(=O)[C@H]1CC1(C)C)C2)C(=O)N1CCCCC1] |
| | (A) |
| | 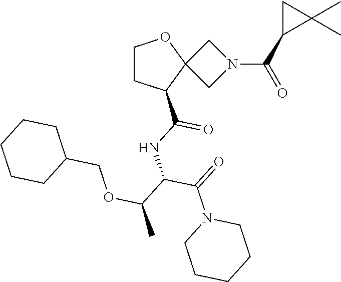 [SMILES: C[C@@H](OCC1CCCCC1)[C@H](NC(=O)[C@H]1CCOC12CN(C(=O)[C@H]1CC1(C)C)C2)C(=O)N1CCCCC1] |
| | (B) |
| I-30 | 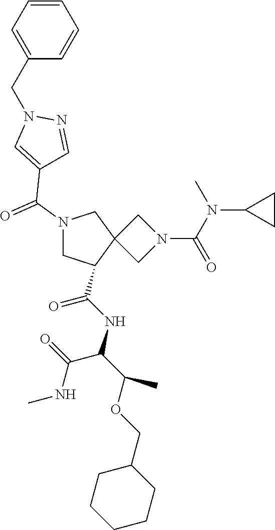 |
| I-31 | 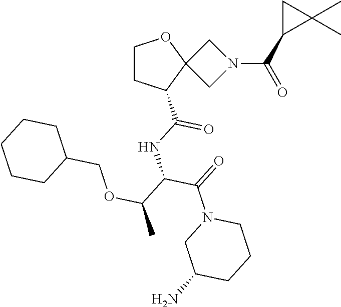 [SMILES: C[C@@H](OCC1CCCCC1)[C@H](NC(=O)[C@@H]1CCOC12CN(C(=O)[C@H]1CC1(C)C)C2)C(=O)N1CCC[C@H](N)C1] |
| | (A) |
| | 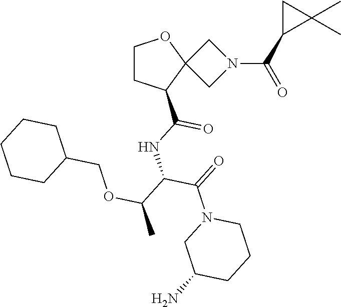 [SMILES: C[C@@H](OCC1CCCCC1)[C@H](NC(=O)[C@H]1CCOC12CN(C(=O)[C@H]1CC1(C)C)C2)C(=O)N1CCC[C@H](N)C1] |
| | (B) |
| I-32 | 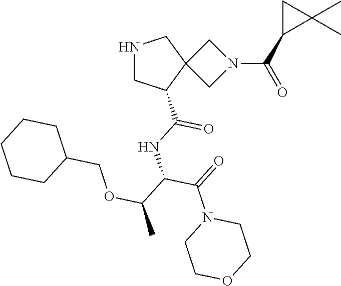 [SMILES: C[C@@H](OCC1CCCCC1)[C@H](NC(=O)[C@@H]1CNCC12CN(C(=O)[C@H]1CC1(C)C)C2)C(=O)N1CCOCC1] |
| I-33 | 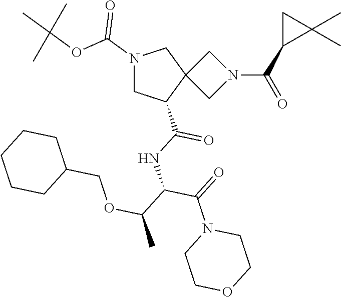 [SMILES: C[C@@H](OCC1CCCCC1)[C@H](NC(=O)[C@@H]1CN(C(=O)OC(C)(C)C)CC12CN(C(=O)[C@H]1CC1(C)C)C2)C(=O)N1CCOCC1] |
| I-34 | 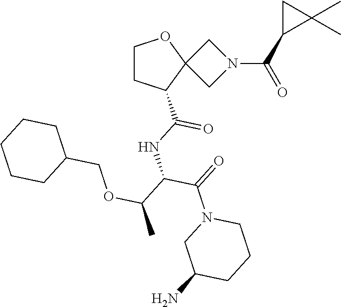 [SMILES: C[C@@H](OCC1CCCCC1)[C@H](NC(=O)[C@@H]1CCOC12CN(C(=O)[C@H]1CC1(C)C)C2)C(=O)N1CCC[C@@H](N)C1] |
| | (A) |
| | 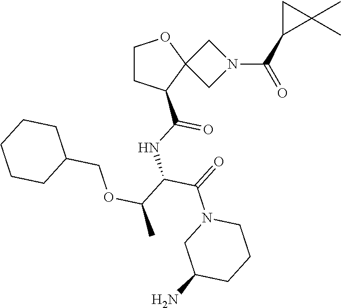 [SMILES: C[C@@H](OCC1CCCCC1)[C@H](NC(=O)[C@H]1CCOC12CN(C(=O)[C@H]1CC1(C)C)C2)C(=O)N1CCC[C@@H](N)C1] |
| | (B) |
| I-35 | 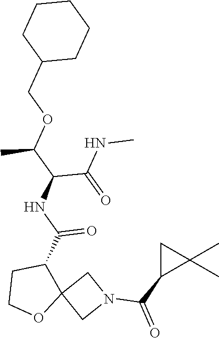 [SMILES: CNC(=O)[C@@H](NC(=O)[C@H]1CCOC12CN(C(=O)[C@H]1CC1(C)C)C2)[C@@H](C)OCC1CCCCC1] |
| | (A) |
| | 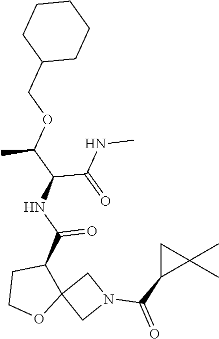 [SMILES: CNC(=O)[C@@H](NC(=O)[C@@H]1CCOC12CN(C(=O)[C@H]1CC1(C)C)C2)[C@@H](C)OCC1CCCCC1] |
| | (B) |
| I-36 | 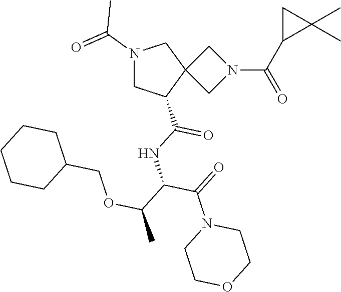 [SMILES: CC(=O)N1C[C@@H](C(=O)N[C@H](C(=O)N2CCOCC2)[C@@H](C)OCC2CCCCC2)C2(C1)CN(C(=O)C1CC1(C)C)C2] |
| I-37 | 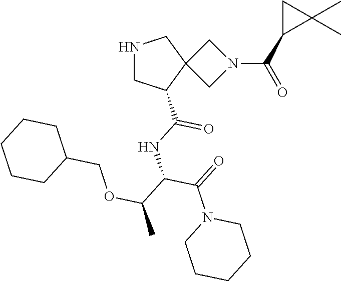 [SMILES: C[C@@H](OCC1CCCCC1)[C@H](NC(=O)[C@@H]1CNCC12CN(C(=O)[C@H]1CC1(C)C)C2)C(=O)N1CCCCC1] |
| I-38 | 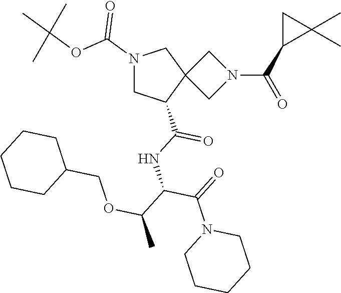 [SMILES: C[C@@H](OCC1CCCCC1)[C@H](NC(=O)[C@@H]1CN(C(=O)OC(C)(C)C)CC12CN(C(=O)[C@H]1CC1(C)C)C2)C(=O)N1CCCCC1] |
| I-39 | 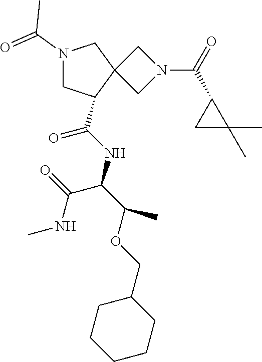 [SMILES: CNC(=O)[C@@H](NC(=O)[C@@H]1CN(C(C)=O)CC12CN(C(=O)[C@H]1CC1(C)C)C2)[C@@H](C)OCC1CCCCC1] |
| I-40 | 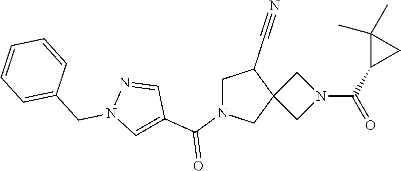 [SMILES: CC1(C)C[C@@H]1C(=O)N1CC2(CN(C(=O)c3cnn(Cc4ccccc4)c3)CC2C#N)C1] |
| I-41 | 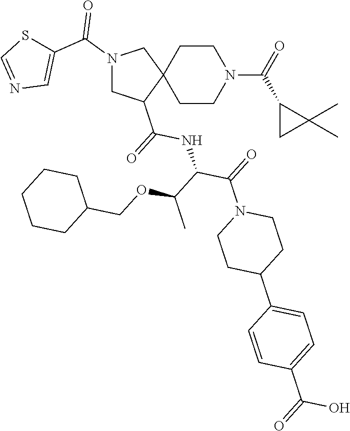 [SMILES: C[C@@H](OCC1CCCCC1)[C@H](NC(=O)C1CN(C(=O)c2cncs2)CC12CCN(C(=O)[C@H]1CC1(C)C)CC2)C(=O)N1CCC(c2ccc(C(=O)O)cc2)CC1] |
| I-42 | 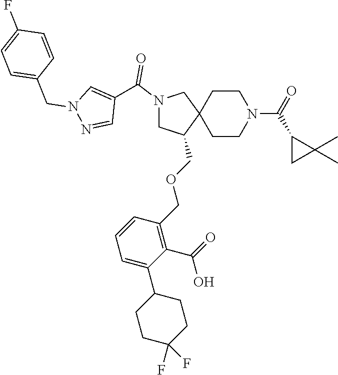 [SMILES: CC1(C)C[C@@H]1C(=O)N1CCC2(CC1)CN(C(=O)c1cnn(Cc3ccc(F)cc3)c1)C[C@H]2COCc1cccc(C2CCC(F)(F)CC2)c1C(=O)O] |
| I-43 | 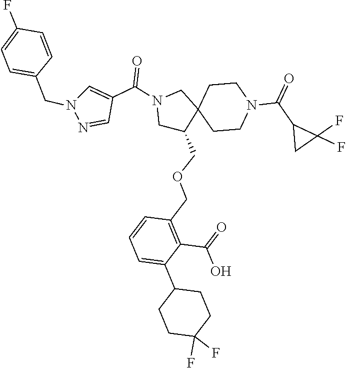 [SMILES: O=C(O)c1c(COC[C@@H]2CN(C(=O)c3cnn(Cc4ccc(F)cc4)c3)CC23CCN(C(=O)C2CC2(F)F)CC3)cccc1C1CCC(F)(F)CC1] |
| I-44 | 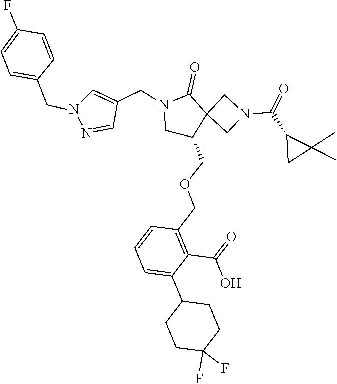 [SMILES: CC1(C)C[C@@H]1C(=O)N1CC2(C1)C(=O)N(Cc1cnn(Cc3ccc(F)cc3)c1)C[C@H]2COCc1cccc(C2CCC(F)(F)CC2)c1C(=O)O] |

In some embodiments, the present disclosure provides a compound set forth in Table 1, above, or a pharmaceutically acceptable salt thereof. In some embodiments, the disclosure provides a compound set forth in Table 1, above, or a pharmaceutically acceptable salt thereof, and any enantiomers, diastereomers, or conformation isomers thereof. The present disclosure contemplates any and all enantiomers, diastereomers and conformation isomers of a compound shown herein.

In some embodiments, the present disclosure provides a pharmaceutical composition comprising a compound of the present disclosure, or a pharmaceutically acceptable salt thereof, together with a pharmaceutically acceptable carrier, excipient, vehicle, adjuvant or diluent. In some embodiments, the present disclosure provides a pharmaceutical composition comprising a compound set forth in Table 1 above, or a pharmaceutically acceptable salt thereof, together with a pharmaceutically acceptable carrier, excipient, vehicle, adjuvant or diluent. In some embodiments, the pharmaceutical composition further comprises an additional therapeutic agent.

In some embodiments, the present disclosure provides a complex comprising a CDK2 protein and a compound of the present disclosure.

In some embodiments, the present disclosure provides a method of inhibiting the activity of a cyclin-dependent kinase (CDK). In some embodiments, the method comprises contacting a compound of the present disclosure with a CDK. In some embodiments, the compound and the CDK are contacted in vivo. In some embodiments, the compound and the CDK are contacted in vitro. In some embodiments, the CDK is selected from CDK1, CDK2, CDK3, CDK4, CDK5, CDK6, CDK7, CDK8, CDK9, CDK1O, CDK11, CDK12 and CDK13. In some embodiments, the CDK is CDK2. In some embodiments, the CDK is CDK3. In some embodiments, the CDK is CDK4. In some embodiments, the CDK is CDK6. In some embodiments, the method inhibits the activity of both CDK2 and CDK3. In some embodiments, the method inhibits the activity of CDK2 and one or both of CDK4 and CDK6.

In some embodiments, the compounds of the present disclosure inhibit the activity of one or more CDKs selected from CDK1, CDK2, CDK3, CDK4, CDK5, CDK6, CDK7, CDK8, CDK9, CDK10, CDK11, CDK12 and CDK13. In some embodiments, the compounds of the present disclosure inhibit CDK2. In some embodiments, the compounds of the present disclosure inhibit CDK3. In some embodiments, the compounds of the present disclosure inhibit CDK4. In some embodiments, the compounds of the present disclosure inhibit CDK5. In some embodiments, the compounds of the present disclosure inhibit CDK6. In some embodiments, the compounds of the present disclosure are CDK2/3 inhibitors. In some embodiments, the compounds of the present disclosure are CDK2/4/6 inhibitors.

In some embodiments, the present disclosure provides compounds that selectively inhibit CDK2 over other cyclin-dependent kinases (CDKs). In some embodiments, the compounds of the present disclosure selectively inhibit CDK2 over one or more other CDKs, selected from CDK1, CDK3, CDK4, CDK5, CDK6, CDK7, CDK8, CDK9, CDK10, CDK11, CDK12 and CDK13. In some embodiments, the compounds of the present disclosure selectively inhibit CDK2 over CDK4.

In some embodiments, the compounds of the present disclosure selectively inhibit CDK2 over CDK6. In some embodiments, the compounds of the present disclosure selectively inhibit CDK2 over CDK4 and CDK6.

In some embodiments, the present disclosure provides compounds that selectively inhibit CDK2/cyclin E complexes over other CDK complexes.

### 4. General Methods of Providing the Present Compounds

The compounds of this disclosure may be prepared or isolated in general by synthetic and/or semi-synthetic methods known to those skilled in the art for analogous compounds and by methods described in detail in the Examples, herein.

In the Schemes below, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

Compounds of the present disclosure, including those of Formula I and the compounds of Table 1, can generally be prepared according the methods described below and from adapted procedures of the Examples. Reagents and conditions can be modified and substituted using knowledge common to one of ordinary skill in the art, and techniques for similar compounds presented in the Examples, as needed, in order to arrive at the compounds of the present disclosure.

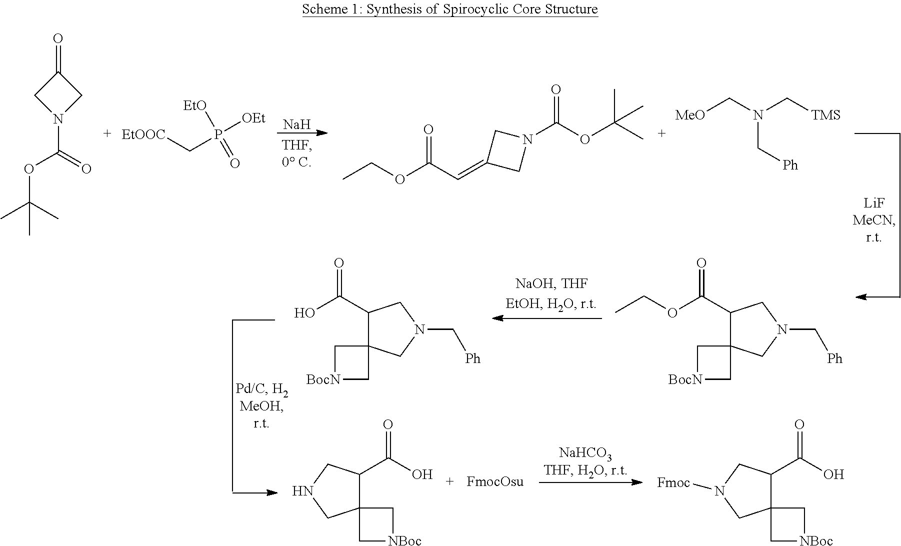

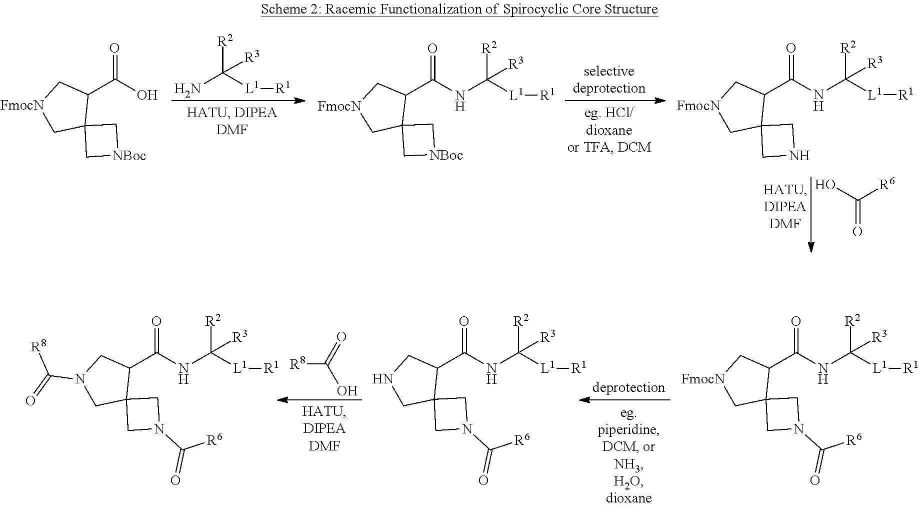

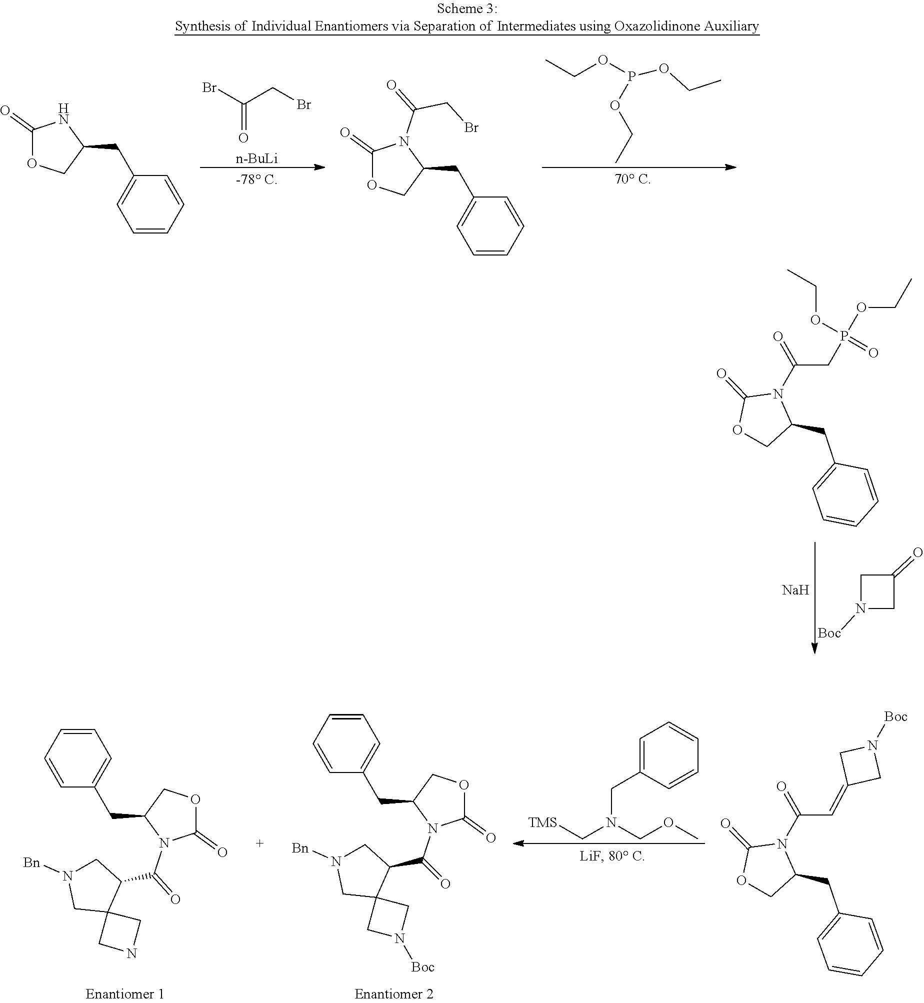

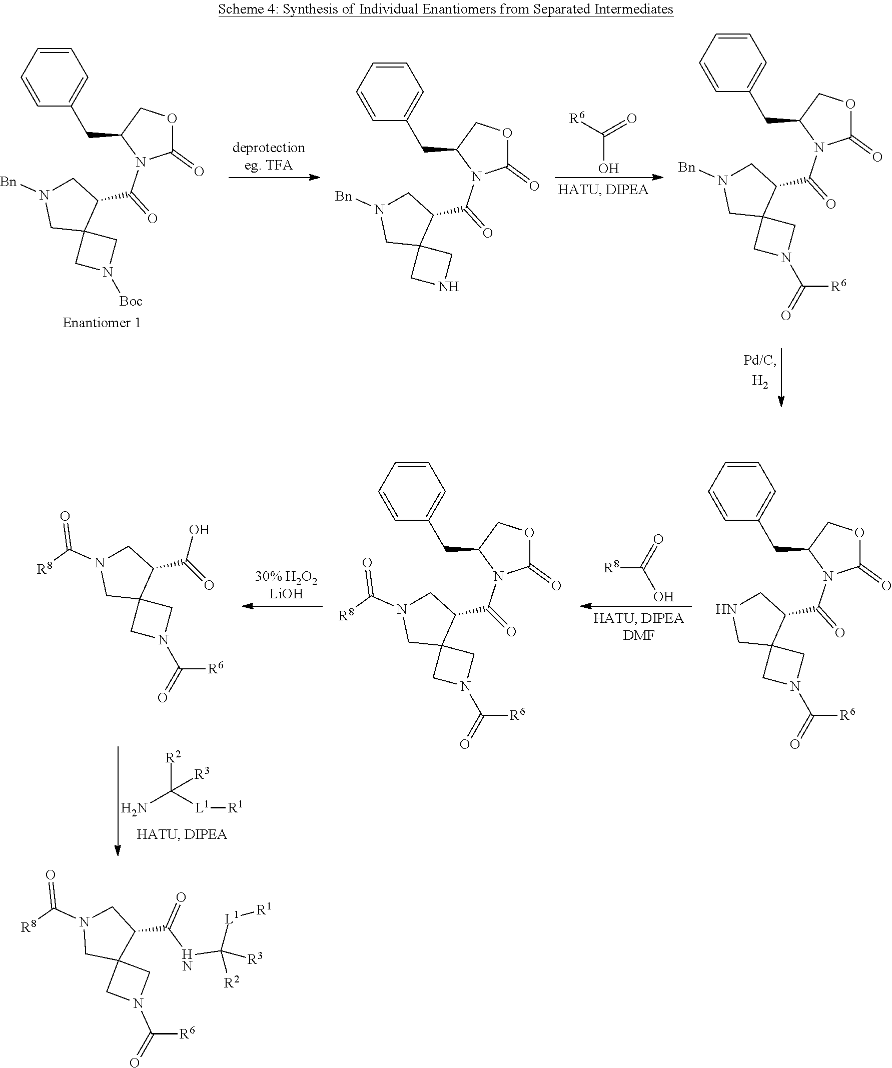

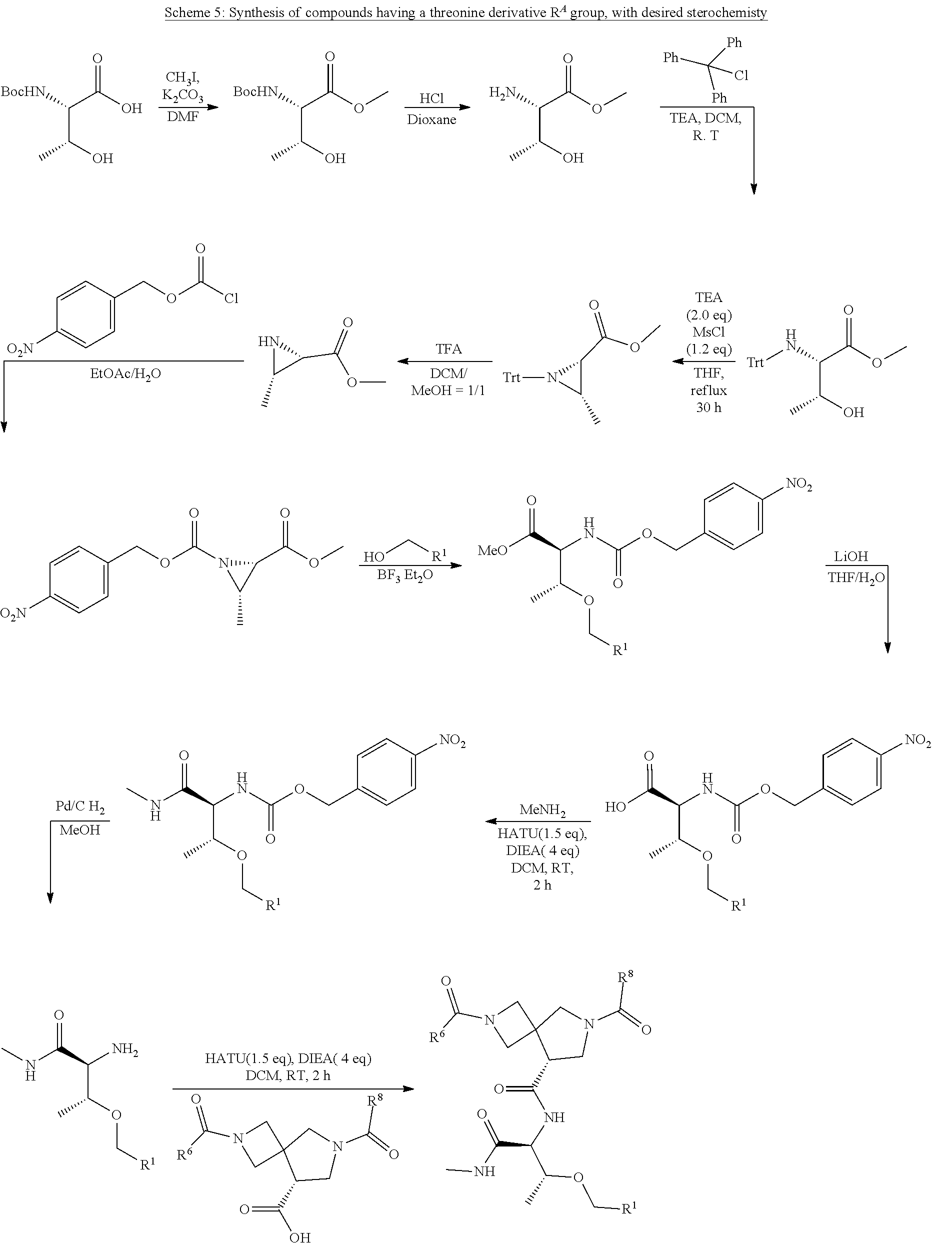

### 5. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably inhibit a CDK2 protein, or a mutant thereof, in a biological sample or in a patient. In certain embodiments, the amount of compound in compositions of this disclosure is such that it is effective to measurably inhibit a CDK2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered subcutaneously, orally, intraperitoneally or intravenously. In some embodiments, the compositions are administered orally. In some embodiments, the compositions are administered intraperitoneally. In some embodiments, the compositions are administered intravenously. In some embodiments, the compositions are administered subcutaneously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food.

In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the modulation of the activity CDK2. In some embodiments, the compounds and compositions described herein are CDK2 inhibitors.

In some embodiments, the compounds and compositions of the present disclosure are useful for treating diseases and disorders associated with CDK2 activity, including, but not limited to cancers, myeloproliferative disorders, autoimmune disorders, inflammatory disorders, viral infections, fibrotic disorders, and neurodegenerative disorders.

In some embodiments, the disclosure provides a method of inhibiting the activity of a CDK2, the method comprising contacting a compound of the present disclosure, or a pharmaceutically acceptable salt thereof with the CDK2. In some embodiments, the contacting takes place in vitro. In some embodiments, the contacting takes place in vivo.

In some embodiments, the disclosure provides a method of treating, preventing or lessening the severity of a disease or disorder associated with CDK2 activity in a patient, including, but not limited to cancers, myeloproliferative disorders, autoimmune disorders, inflammatory disorders, fibrotic disorders, and neurodegenerative disorders, said method comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the treatment of a disease or disorder associated with CDK2 activity.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the manufacture of a medicament for treating a disease or disorder associated with CDK2 activity.

In some embodiments, the disease or disorder associated with CDK2 activity is a CDK2-mediated disease or disorder. In some embodiments, the disease or disorder associated with CDK2 activity is a disease or disorder caused by CDK2 over-activity.

In some embodiments, the disease or disorder associated with CDK2 activity is cancer.

In some embodiments, the cancer is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

In some embodiments, the cancer is characterized by amplification or overexpression of CCNE1 and/or CCNE2.

In some embodiments, the cancer is breast cancer. In some embodiments, the breast cancer is a breast cancer selected from ER-positive/HR-positive breast cancer, HER2-negative breast cancer, ER-positive/HR-positive breast cancer, HER2-positive breast cancer, triple negative breast cancer (TNBC), inflammatory breast cancer, endocrine resistant breast cancer, trastuzumab resistant breast cancer, breast cancer with primary or acquired resistance to CDK4/CDK6 inhibition, advanced breast cancer and metastatic breast cancer. In some embodiments the breast cancer is characterized by amplification or overexpression of CCNE1 and/or CCNE2.

In some embodiments, the cancer is ovarian cancer. In some embodiments, the ovarian cancer is high-grade serous ovarian cancer (HGSOC). In some embodiments the ovarian cancer is characterized by amplification or overexpression of CCNE1 and/or CCNE2.

In some embodiments, the cancer is bladder cancer. In some embodiments, the bladder cancer is characterized by amplification or overexpression of CCNE1 and/or CCNE2.

In some embodiments, the cancer is uterine cancer. In some embodiments, the uterine cancer is characterized by amplification or overexpression of CCNE1 and/or CCNE2.

In some embodiments, the cancer is prostate cancer. In some embodiments, the prostate cancer is characterized by amplification or overexpression of CCNE1 and/or CCNE2.

In some embodiments, the cancer is lung cancer. In some embodiments, the lung cancer is a lung cancer selected from non-small cell lung cancer, small cell lung cancer, squamous cell carcinoma, adenocarcinoma, and mesothelioma. In some embodiments, the lung cancer is characterized by amplification or overexpression of CCNE1 and/or CCNE2. In some embodiments, the lung cancer is CCNE1 amplified squamous cell carcinoma or CCNE1 amplified adenocarcinoma.

In some embodiments, the cancer is head and neck cancer. In some embodiments, the head and neck cancer is characterized by amplification or overexpression of CCNE1 and/or CCNE2.

In some embodiments, the cancer is colorectal cancer. In some embodiments, the colorectal cancer is characterized by amplification or overexpression of CCNE1 and/or CCNE2.

In some embodiments, the cancer is kidney cancer. In some embodiments, the kidney cancer is renal cell carcinoma (RCC). In some embodiments, the kidney cancer is characterized by amplification or overexpression of CCNE1 and/or CCNE2.

In some embodiments, the cancer is liver cancer. In some embodiments, the liver cancer is hepatocellular carcinoma (HCC). In some embodiments, the liver cancer is characterized by amplification or overexpression of CCNE1 and/or CCNE2.

In some embodiments, the cancer is pancreatic cancer. In some embodiments, the pancreatic cancer is characterized by amplification or overexpression of CCNE1 and/or CCNE2.

In some embodiments, the cancer is stomach cancer. In some embodiments, the stomach cancer is characterized by amplification or overexpression of CCNE1 and/or CCNE2.

In some embodiments, the cancer is melanoma. In some embodiments, the melanoma is characterized by amplification or overexpression of CCNE1 and/or CCNE2. CDK2 expression is regulated by essential melanocytic transcription factor MITF. It has been found that CDK2 depletion suppresses the growth of melanoma (Du et al., Cancer Cell. 2004 December; 6(6): 565-576)

In some embodiments, the cancer is thyroid cancer. In some embodiments, the thyroid cancer is characterized by amplification or overexpression of CCNE1 and/or CCNE2.

In some embodiments, the disease or disorder associated with CDK2 activity is a myeloproliferative disorder.

In some embodiments, the disease or disorder associated with CDK2 activity is a neurodegenerative disease or disorder. In some embodiments, the neurodegenerative disease or disorder is Alzheimer's disease (AD). It has been reported that neuronal cell death in subjects suffering from AD is preceded by cell cycle events. Inhibition of one or more CDKs can inhibit cell cycle events and therefore stave off neuronal cell death (Yang et al., J Neurosci. 2003 Apr. 1; 23(7):2557-2563).

In some embodiments, the disease or disorder associated with CDK2 activity is a liver disease.

In some embodiments, the disease or disorder associated with CDK2 activity is liver fibrosis. It has been reported that CCNE1 knockout mice do not develop liver fibrosis upon exposure to pro-fibrotic toxin CCl₄, suggesting that liver fibrosis can be treated via administration of a CDK2 inhibitor (Nevzorova, et al., Hepatology. 2012 September; 56(3): 1140-1149.)

In some embodiments, the disease or disorder associated with CDK2 activity is Cushing disease. Pituitary cyclin E/E2F1 signaling is a molecular mechanism underlying neuroendocrine regulation of the hypothalamic-pituitary-adrenal axis, and therefore provides a subcellular therapeutic target for CDK2 inhibitors of pituitary ACTH-dependent hypercortisolism, also known as Cushing disease (Liu, et al., J Clin Endocrinol Metab. 2015 July; 100(7): 2557-2564).

In some embodiments, the disease or disorder associated with CDK2 activity is a kidney disease.

In some embodiments, the disease or disorder associated with CDK2 activity is polycystic kidney disease. It has been reported that CDK2/CDK5 inhibitor roscovitine yields effective arrest of cystic kidney disease in mouse models of polycystic kidney disease (Bukanov, et al., Nature. 2006 Dec. 14; 444(7121):949-52).

In some embodiments, the disease or disorder associated with CDK2 activity is an autoimmune disorder. CDK2 ablation has been shown to promote immune tolerance by supporting the function of regulatory T cells (Chunder et al., J Immunol. 2012 Dec. 15; 189(12):5659-66).

In some embodiments, the disease or disorder associated with CDK2 activity is an inflammatory disorder. Cyclin E ablation has been shown to attenuate hepatitis in mice, while p27 knockout mice display exacerbation of renal inflammation (Ehedego et al., Oncogene. 2018 June; 37(25):3329-3339; Ophascharoensuk et al., Nat Med. 1998 May; 4(5):575-80). In some embodiments, the inflammatory disorder is hepatitis.

Tn some embodiments, the compounds and compositions of the present disclosure are useful as male contraceptives. Based on the finding that male CDK2 knockout mice are sterile, CDK2 inhibitors have been studied as possible male contraceptives (Faber, et al., Biol Reprod. 2020 August; 103(2): 357-367). In some embodiments, the present disclosure provides a method of reducing male fertility comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

In some embodiments, the compounds and compositions of the present disclosure are useful for treating diseases and disorders associated with CDK5 activity, including, but not limited to cancers, myeloproliferative disorders, autoimmune disorders, inflammatory disorders, viral infections, fibrotic disorders, and neurodegenerative disorders. In some embodiments, the compounds and compositions of the present disclosure are useful for treating neurodegenerative disorders associated with CDK5 activity.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents that the compounds of the present disclosure may also be combined with include, without limitation: endocrine therapeutic agents, chemotherapeutic agents and other CDK inhibitory compounds.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of an endocrine therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional CDK inhibitory compounds. In some embodiments, the one or more additional CDK inhibitory compounds are CDK4, or CDK4/CDK6 inhibitors. In some embodiments, the one or more additional CDK inhibitory compounds are CDK4, CDK6, CDK7 or CDK4/CDK6 inhibitors. In some embodiments, the one or more additional CDK inhibitory compounds are CDK4 inhibitors. In some embodiments, the one or more additional CDK inhibitory compounds are CDK6 inhibitors.

In some embodiments, the one or more additional CDK inhibitory compounds are CDK7 inhibitors. In some embodiments, the one or more additional CDK inhibitory compounds are CDK4/CDK6 inhibitors.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of a chemotherapeutic agent. In some embodiments, the chemotherapeutic agent is a taxane. In some embodiments, the chemotherapeutic agent is a platinum agent. In some embodiments, the chemotherapeutic agent is trastuzumab.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition the present disclosure are administered as a multiple dosage regimen within greater than 24 hours apart.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### EXAMPLES

As depicted in the Examples below, in certain exemplary embodiments, compounds are prepared according to the general procedures provided herein. It will be appreciated that, although the general methods depict the synthesis of certain compounds of the present disclosure, the general methods, and other methods known to one of ordinary skill in the art, can be applied to all compounds and subclasses and species of each of these compounds, as described herein.

### Example 1: Synthesis Procedures

### Synthesis of (S)-6-(1-benzyl-1H-pyrazole-4-carbonyl)-N8-((2S,3R)-3-(cyclohexylmethoxy)-1-(methylamino)-1-oxobutan-2-yl)-N2-cyclopropyl-N2-methyl-2,6-diazaspiro[3.4]octane-2,8-dicarboxamide (I-30)

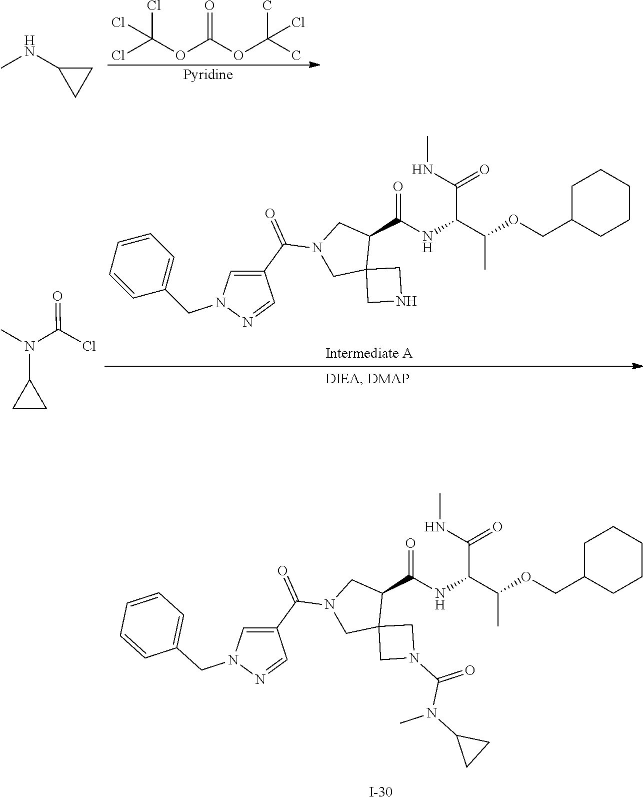

The intermediate A used in the 3^{rd} step of the above reaction scheme (the spirocyclic intermediate) was synthesized as follows, I-30 was synthesized according the procedure for I-294 from a prior application, which is reiterated below and shown in the Scheme with intermediate A:

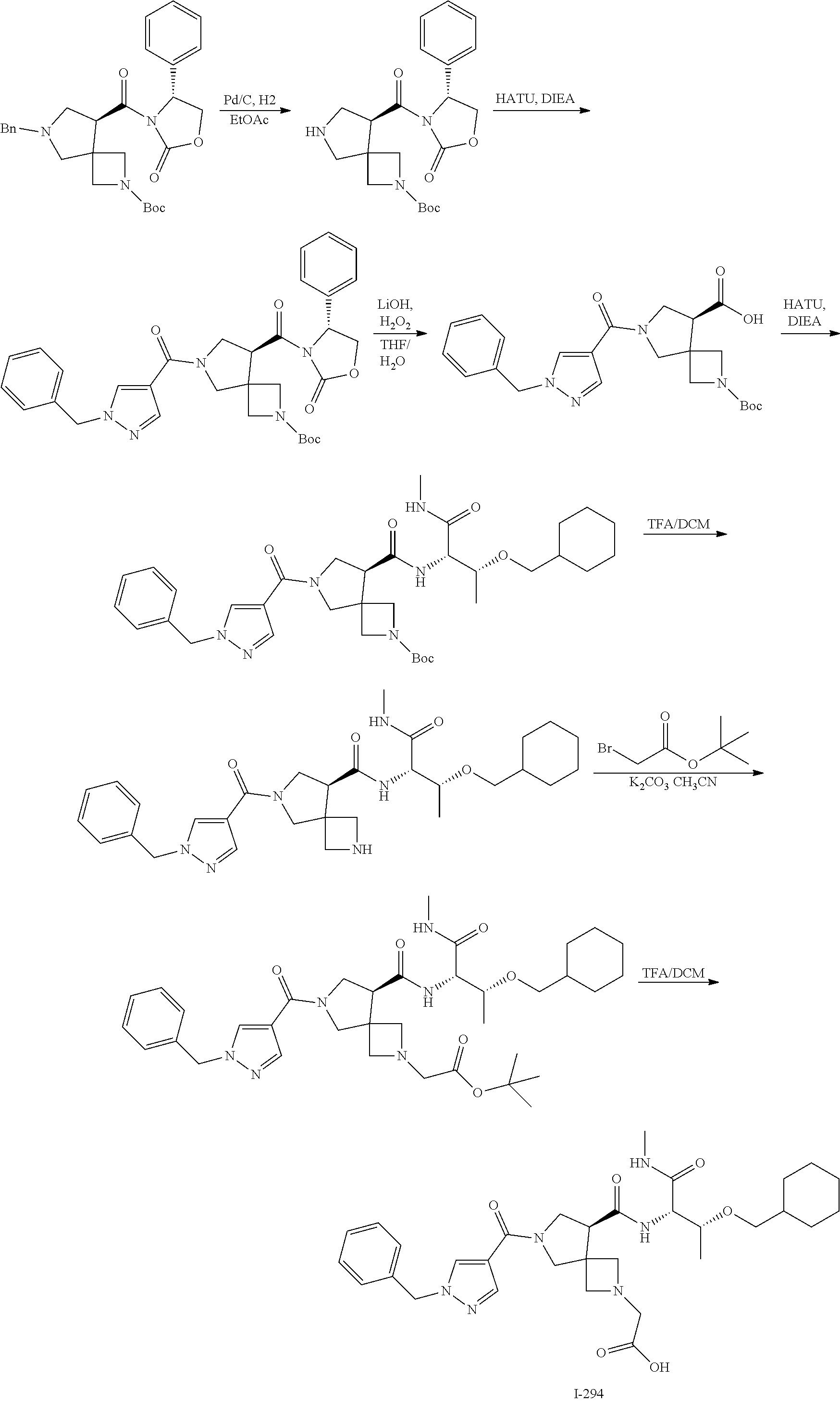

Step 1: To a solution of tert-butyl (S)-6-benzyl-8-((R)-2-oxo-4-phenyloxazolidine-3-carbonyl)-2,6-diazaspiro[3.4]octane-2-carboxylate (Intermediate 11 synthesized as shown below) (1 g, 2.0 mmol) in EtOAc (10 mL) was added 10% Pd/C (300 mg). The reaction mixture was stirred under a H₂ atmosphere for 48 h. The mixture was filtered and concentrated to afford crude tert-butyl (S)-8-((R)-2-oxo-4-phenyloxazolidine-3-carbonyl)-2,6-diazaspiro[3.4]octane-2-carboxylate (800 mg, 100%) which was used directly in the next step without further purification. LCMS m/z=402.2 [M+H]⁺.

Step 2: To a solution of 1-benzyl-1H-pyrazole-4-carboxylic acid (2.2 g, 11.0 mmol) in DCM (40 mL) was added HATU (4.79 g, 12.6 mmol) and the mixture was stirred at room temperature for 30 min. tert-butyl (S)-8-((R)-2-oxo-4-phenyloxazolidine-3-carbonyl)-2,6-diazaspiro[3.4]octane-2-carboxylate (4.2 g, 10.5 mmol) and DIPEA (2.03 g, 15.75 mmol) were added and the reaction stirred at room temperature for another 2 h. The mixture was diluted with water (100 mL) and extracted with EtOAc (150 mL×3). The combined organic layers were washed with brine, dried over Na₂SO₄, filtered and concentrated. The residue obtained was purified by column chromatography on silica gel (eluent: DCM:MeOH=30:1) to afford tert-butyl (S)-6-(1-benzyl-1H-pyrazole-4-carbonyl)-8-((R)-2-oxo-4-phenyloxazolidine-3-carbonyl)-2,6-diazaspiro[3.4]octane-2-carboxylate (4.7 g, 77%) as a yellow solid. LCMS m/z=586.3 [M+H]⁺.

Step 3: To a solution of tert-butyl (S)-6-(1-benzyl-1H-pyrazole-4-carbonyl)-8-((R)-2-oxo-4-phenyloxazolidine-3-carbonyl)-2,6-diazaspiro[3.4]octane-2-carboxylate (100 mg, 0.17 mmol) in a mixture of THF and H₂O (3 mL/0.25 mL) at 0° C. was added a solution of lithium hydroxide monohydrate (10 mg, 0.43 mmol) in water (0.25 mL) and 30% H₂O₂ (12 mg, 0.34 mmol) in water (0.25 mL). The reaction mixture was stirred at 0° C. for 1 h then diluted with water (15 mL) and extracted with EtOAc (30 mL). The aqueous layer was collected and acidified with 1M HCl to pH ˜2 and extracted with EtOAc (80 mL×3). The combined organic layers were washed with brine, dried over Na₂SO₄, filtered and concentrated to afford crude (S)-6-(1-benzyl-1H-pyrazole-4-carbonyl)-2-(tert-butoxycarbonyl)-2,6-diazaspiro[3.4]octane-8-carboxylic acid (50 mg, 66%) as a white solid which was used directly in the next step. LCMS m/z=441.2 [M+H]⁺.

Step 4: To a solution of (S)-6-(1-benzyl-1H-pyrazole-4-carbonyl)-2-(tert-butoxycarbonyl)-2,6-diazaspiro[3.4]octane-8-carboxylic acid (100 mg, 0.227 mmol) in DCM (2 mL) was added HATU (103 mg, 0.272 mmol) and the mixture was stirred at room temperature for 30 min. (2S,3R)-2-amino-3-(cyclohexylmethoxy)-N-methylbutanamide hydrochloride (52 mg, 0.227 mmol) and DIPEA (117 mg, 0.908 mmol) were added and the reaction stirred at room temperature for another 2 h. The mixture was diluted with water (30 mL) and extracted with EtOAc (50 mL×3). The combined organic layers were washed with brine, dried over Na₂SO₄, filtered and concentrated. The residue obtained was purified by column chromatography on silica gel (eluent: DCM:MeOH=30:1) to afford tert-butyl (S)-6-(1-benzyl-1H-pyrazole-4-carbonyl)-8-(((2S,3R)-3-(cyclohexylmethoxy)-1-(methylamino)-1-oxobutan-2-yl)carbamoyl)-2,6-diazaspiro[3.4]octane-2-carboxylate (114 mg, 78%) as a yellow solid. LCMS m/z=651.4 [M+H]⁺.

Step 5 (synthesis of intermediate A): To a solution of (S)-6-(1-benzyl-1H-pyrazole-4-carbonyl)-8-(((2S,3R)-3-(cyclohexylmethoxy)-1-(methylamino)-1-oxobutan-2-yl)carbamoyl)-2,6-diazaspiro[3.4]octane-2-carboxylate (50 mg, 0.077 mmol) in DCM (2 mL) was added TFA (1 mL). The reaction mixture was stirred at room temperature for 1 h. The solvent was removed under vacuum to afford (S)-6-(1-benzyl-1H-pyrazole-4-carbonyl)-N-((2S,3R)-3-(cyclohexylmethoxy)-1-(methylamino)-1-oxobutan-2-yl)-2,6-diazaspiro[3.4]octane-8-carboxamide (42 mg, 100%) which was used directly in the next step. LCMS m/z=551.3 [M+H]⁺.

Steps 6-7 are for exemplary purposes relating to the synthesis of I-30.

Step 6: To a solution of (S)-6-(1-benzyl-1H-pyrazole-4-carbonyl)-N-((2S,3R)-3-(cyclohexylmethoxy)-1-(methylamino)-1-oxobutan-2-yl)-2,6-diazaspiro[3.4]octane-8-carboxamide (85 mg, 1.54 mmol) in CH₃CN (2 mL) was added tert-butyl 2-bromoacetate (30 mg, 1.54 mmol) and K₂CO₃ (85 mg, 6.17 mmol). The mixture was stirred at room temperature overnight. The mixture was diluted with water (20 mL) and extracted with EtOAc (50 mL×2). The combined organic layers were washed with brine, dried over Na₂SO₄, filtered and concentrated. The residue obtained was purified by prep-TLC (eluent: DCM/MeOH=30:1) to afford tert-butyl 2-((S)-6-(1-benzyl-1H-pyrazole-4-carbonyl)-8-(((2S,3R)-3-(cyclohexylmethoxy)-1-(methylamino)-1-oxobutan-2-yl)carbamoyl)-2,6-diazaspiro[3.4]octan-2-yl)acetate (25 mg, 24%) as a colorless oil. LCMS m/z=665.50 [M+H]⁺.

Step 7: To a solution of tert-butyl 2-((S)-6-(1-benzyl-1H-pyrazole-4-carbonyl)-8-(((2S,3R)-3-(cyclohexylmethoxy)-1-(methylamino)-1-oxobutan-2-yl)carbamoyl)-2,6-diazaspiro[3.4]octan-2-yl)acetate (25 mg, 0.37 mmol) in DCM (0.6 mL) was added TFA (0.2 mL). The mixture was stirred at room temperature for 2 h. The solvent was removed under vacuum. The residue was purified by prep-HPLC to afford 2-((S)-6-(1-benzyl-1H-pyrazole-4-carbonyl)-8-(((2S,3R)-3-(cyclohexylmethoxy)-1-(methylamino)-1-oxobutan-2-yl)carbamoyl)-2,6-diazaspiro[3.4]octan-2-yl)acetic acid (I-294) (11 mg, 50%) as a white solid. LCMS m/z=609.4 [M+H]⁺; ¹H NMR (400 MHz, DMSO-d6) δ 8.36-8.28 (m, 1H), 8.15-8.06 (m, 1H), 7.83-7.74 (m, 2H), 7.38-7.23 (m, 5H), 5.38-5.33 (m, 2H), 4.27-4.20 (m, 1H), 4.06-3.99 (m, 1H), 3.92-3.44 (m, 11H), 3.38-3.32 (m, 3H), 3.26-3.21 (m, 1H), 3.14-3.09 (m, 1H), 2.60-2.55 (m, 3H), 1.70-1.58 (m, 5H), 1.49-1.38 (m, 1H), 1.20-1.04 (m, 6H), 0.89-0.79 (m, 2H).

### Intermediate 11: tert-butyl (S)-6-benzyl-8-((R)-2-oxo-4-phenyloxazolidine-3-carbonyl)-2,6-diazaspiro[3.4]octane-2-carboxylate & Intermediate 12: tert-butyl (R)-6-benzyl-8-((R)-2-oxo-4-phenyloxazolidine-3-carbonyl)-2,6-diazaspiro[3.4]octane-2-carboxylate

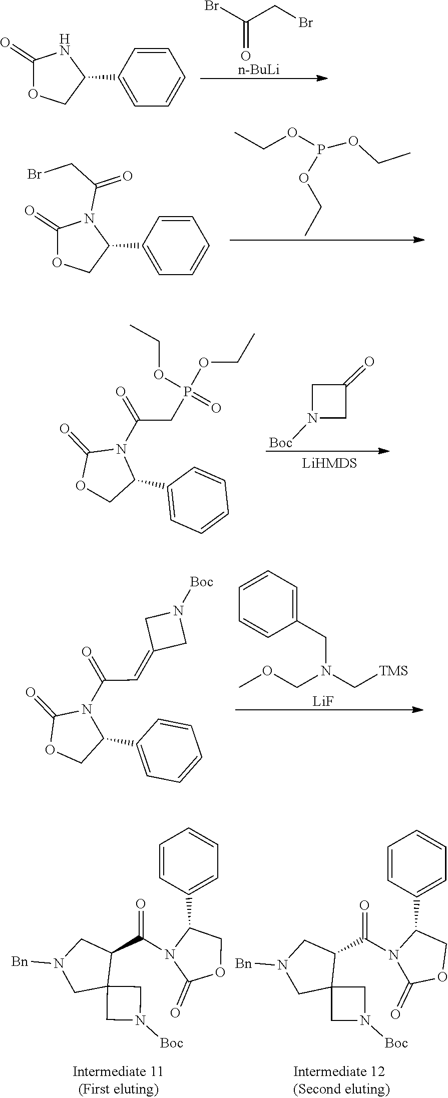

Step 1: To a solution of (R)-4-phenyloxazolidin-2-one (10 g, 61 mmol) in anhydrous THF (100 mL) at −78° C. under a N₂ atmosphere was added n-BuLi (2.5 M in Hexanes, 27 mL, 67 mmol) dropwise. The reaction mixture was stirred at −78° C. for 0.5 h then 2-bromoacetyl bromide (5.6 mL, 64 mmol) was added. The reaction was allowed to warm to room temperature and stirred for another 2 h. The mixture was diluted with EtOAc (100 mL), quenched with sat. NH₄Cl (100 mL), extracted with EtOAc (100 mL×2), dried over Na₂SO₄, filtered and concentrated. The residue was purified by column chromatography on silica gel (eluent: Pet. Ether:EtOAc=10:1 to 3:1) to afford (R)-3-(2-bromoacetyl)-4-phenyloxazolidin-2-one (9 g, 52%) as a yellow oil. ¹H NMR (400 MHz, DMSO-d6) δ 7.42-7.37 (m, 2H), 7.36-7.30 (m, 3H), 5.52-5.46 (m, 1H), 4.83-4.75 (m, 2H), 4.56-4.50 (m, 1H), 4.24-4.18 (m, 1H).

Step 2: A mixture of (R)-3-(2-bromoacetyl)-4-phenyloxazolidin-2-one (10 g, 35 mmol) in triethyl phosphite (29 g, 175 mmol) was heated at 50° C. for 18 h. The excess triethyl phosphite was removed under vacuum at 70° C. to afford diethyl (R)-(2-oxo-2-(2-oxo-4-phenyloxazolidin-3-yl)ethyl)phosphonate (12 g crude, 99%) as a yellow oil. LCMS m/z=342.1 [M+H]⁺; ¹H NMR (400 MHz, CDCl₃) δ 7.41-7.29 (m, 5H), 5.55-5.48 (m, 1H), 4.78-4.70 (m, 1H), 4.20-4.14 (m, 1H), 4.03-3.96 (m, 5H), 3.60-3.48 (m, 1H), 1.27-1.15 (in, 8H).

Step 3: To a solution of diethyl (R)-(2-oxo-2-(2-oxo-4-phenyloxazolidin-3-yl)ethyl)phosphonate (10 g, 29 mmol) in anhydrous THF (100 mL) at 0° C. under N₂ atmosphere was added LiHMDS (1.0 M in THF, 29 mL, 29 mmol) dropwise. The reaction mixture was stirred at 0° C. for 30 min then tert-butyl 3-oxoazetidine-1-carboxylate (21.68 g, 127 mmol) was added. The reaction was warmed to room temperature and stirred for 1 h. The reaction was diluted with EtOAc (200 mL), and the organic layer was washed with sat. NH₄Cl (100 mL), dried over Na₂SO₄, filtered and concentrated. The residue was purified by column chromatography on silica gel (eluent: Pet. Ether:EtOAc=10:1) to afford tert-butyl (R)-3-(2-oxo-2-(2-oxo-4-phenyloxazolidin-3-yl)ethylidene)azetidine-1-carboxylate (10 g, 95%) as a yellow solid. LCMS m/z=492.0 [M+H]⁺; ¹H NMR (400 MHz, DMSO-d₆) δ 7.41-7.36 (m, 2H), 7.34-7.28 (m, 3H), 7.20-7.15 (m, 1H), 5.53-5.46 (m, 1H), 4.79-4.73 (m, 1H), 4.61 (s, 4H), 4.21-4.14 (m, 1H), 1.37 (s, 9H).

Step 4: A mixture of tert-butyl (R)-3-(2-oxo-2-(2-oxo-4-phenyloxazolidin-3-yl)ethylidene)azetidine-1-carboxylate (10 g, 27.90 mmol), N-benzyl-1-methoxy-N-((trimethylsilyl)methyl)methanamine (8.61 g, 36.27 mmol) and LiF (2.17 g, 83.71 mmol) in acetonitrile (100 mL) was heated at 80° C. for 16 h. After cooling to room temperature, the mixture was diluted with water (100 mL) and extracted with EtOAc (100 mL×3). The combined organic layers were washed with brine, dried over Na₂SO₄, filtered and concentrated. The mixture was purified by column chromatography on silica gel (eluent: Pet. Ether:EtOAc=6:1) to afford tert-butyl (S)-6-benzyl-8-((R)-2-oxo-4-phenyloxazolidine-3-carbonyl)-2,6-diazaspiro[3.4]octane-2-carboxylate (Intermediate 11) (8 g, 58%) as a yellow solid as the first eluting isomer. LCMS m/z=492 [M+H]⁺; ¹H NMR (400 MHz, DMSO-d₆) δ 7.41-7.22 (m, 10H), 5.49-5.43 (m, 1H), 4.75-4.69 (m, 1H), 4.23-4.11 (m, 2H), 3.93-3.85 (m, 1H), 3.79 (s, 1H), 3.65-3.58 (m, 2H), 3.56-3.51 (m, 2H), 3.24-3.15 (m, 1H), 2.94-2.87 (m, 1H), 2.57-2.52 (m, 1H), 2.35-2.27 (m, 1H), 1.36 (s, 9H). Further elution provided tert-butyl (R)-6-benzyl-8-((R)-2-oxo-4-phenyloxazolidine-3-carbonyl)-2,6-diazaspiro[3.4]octane-2-carboxylate (Intermediate 12) (4 g, 29%). LCMS m/z=492 [M+H]⁺; ¹H NMR (400 MHz, DMSO-d₆) δ 7.37-7.24 (m, 10H), 5.47-5.41 (m, 1H), 4.76-4.69 (m, 1H), 4.35-4.28 (m, 1H), 4.21-4.14 (m, 1H), 4.00-3.92 (m, 1H), 3.66-3.60 (m, 1H), 3.59-3.55 (m, 2H), 3.36 (s, 2H), 3.19-3.15 (m, 1H), 3.06-2.98 (m, 1H), 2.94-2.88 (m, 1H), 2.55 (s, 1H), 1.36 (s, 9H).

(S)-6-(1-benzyl-1H-pyrazole-4-carbonyl)-N8-((2S,3R)-3-(cyclohexylmethoxy)-1-(methylamino)-1-oxobutan-2-yl)-N2-cyclopropyl-N2-methyl-2,6-diazaspiro[3.4]octane-2,8-dicarboxamide I-30 was synthesized according to the procedures outlined for I-294 above using the appropriate commercially available reagents and/or intermediates described elsewhere. LCMS m/z=648.5 [M+H]⁺; ¹H NMR (400 MHz, DMSO-d₆) δ 8.35 (d, J=20.2 Hz, 1H), 8.16 (dd, J=9.0, 3.8 Hz, 1H), 7.88-7.64 (m, 2H), 7.41-7.18 (m, 5H), 5.35 (d, J=4.2 Hz, 2H), 4.27 (dd, J=8.6, 3.4 Hz, 1H), 4.08 (dd, J=19.6, 8.4 Hz, 1H), 4.01-3.91 (m, 2H), 3.88-3.68 (m, 5H), 3.63-3.54 (m, 1H), 3.46 (t, J=6.0 Hz, 2H), 3.23 (t, J=7.6 Hz, 1H), 3.15-3.07 (m, 1H), 2.71 (d, J=2.4 Hz, 3H), 2.58 (dd, J=10.8, 4.6 Hz, 3H), 1.64 (q, J=13.6, 12.4 Hz, 5H), 1.49-1.38 (m, 1H), 1.24-1.08 (m, 3H), 1.03 (dd, J=6.4, 3.2 Hz, 3H), 0.83 (q, J=12.4 Hz, 2H), 0.69 (t, J=6.4 Hz, 2H), 0.64-0.55 (m, 2H).

### Synthesis of N-((2S,3R)-1-((R)-3-aminopiperidin-1-yl)-3-(cyclohexylmethoxy)-1-oxobutan-2-yl)-2-((S)-2,2-dimethylcyclopropane-1-carbonyl)-5-oxa-2-azaspiro[3.4]octane-8-carboxamide I-34

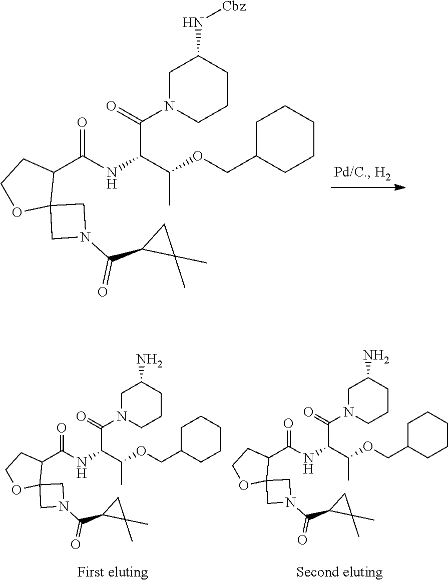

Benzyl ((3R)-1-(O-(cyclohexylmethyl)-N-(2-((S)-2,2-dimethylcyclopropane-1-carbonyl)-5-oxa-2-azaspiro[3.4]octane-8-carbonyl)-L-threonyl)piperidin-3-yl)carbamate was synthesized from 2-((S)-2,2-dimethylcyclopropane-1-carbonyl)-5-oxa-2-azaspiro[3.4]octane-8-carboxylic acid according to the procedures outlined for I-35 using the appropriate commercially available reagents and/or intermediates described elsewhere. LCMS m/z=667.30 [M+H]⁺; ¹H NMR (400 MHz, DMSO-d₆) δ 7.36 (s, 3H), 5.03 (d, J=9.6 Hz, 2H), 4.90-4.77 (m, 1H), 4.39-4.08 (m, 2H), 4.02 (m, 1H), 3.97-3.81 (m, 3H), 3.72 (d, J=5.4 Hz, 2H), 3.62 (d, J=10.0 Hz, 1H), 3.20-3.11 (m, 1H), 2.01 (m, 2H), 1.88 (m, 1H), 1.63 (d, J=13.6 Hz, 5H), 1.49-1.35 (m, 3H), 1.24 (m, 5H), 1.18-0.99 (m, 12H), 0.85 (d, J=6.2 Hz, 3H), 0.68 (m, 1H).

N-((2S,3R)-1-((R)-3-aminopiperidin-1-yl)-3-(cyclohexylmethoxy)-1-oxobutan-2-yl)-2-((S)-2,2-dimethylcyclopropane-1-carbonyl)-5-oxa-2-azaspiro[3.4]octane-8-carboxamide I-34: To a solution of benzyl ((3R)-1-(O-(cyclohexylmethyl)-N-(2-((S)-2,2-dimethylcyclopropane-1-carbonyl)-5-oxa-2-azaspiro[3.4]octane-8-carbonyl)-L-threonyl)piperidin-3-yl)carbamate (70 mg, 0.1 mmol) in MeOH (2 mL) was added 10% Pd/C (15 mg). The reaction was stirred under a H₂ atmosphere for 4 h then the catalyst was removed by filtration through Celite and the filtrate concentrated. The residue obtained was purified by prep-HPLC to afford I-34 (A) (4.3 mg, 8%) as a white solid as the first eluting diastereomer. LCMS m/z=533.5 [M+H]⁺; ¹H NMR (400 MHz, Methanol-d₄) δ 8.49 (s, 1H), 4.93 (s, 1H), 4.70 (d, J=12.4 Hz, 1H), 4.46-4.31 (m, 1H), 4.27-4.16 (m, 1H), 4.07 (d, J=10.8 Hz, 1H), 4.03-3.99 (m, 1H), 3.99-3.92 (m, 1H), 3.88 (t, J=7.2 Hz, 1H), 3.80-3.52 (m, 2H), 3.38-3.33 (m, 1H), 3.29-3.10 (m, 3H), 2.69 (t, J=11.2 Hz, 1H), 2.20 (dq, J=14.8, 7.8, 7.2 Hz, 3H), 1.94-1.45 (m, 10H), 1.41 (m, 1H), 1.28-1.11 (m, 12H), 1.04 (q, J=4.2 Hz, 1H), 0.93 (q, J=11.6 Hz, 2H), 0.78 (m, 1H). Further elution provided I-34 (B) (4.3 mg, 8%) as a white solid. LCMS m/z=533.45 [M+H]⁺; ¹H NMR (400 MHz, Methanol-d₄) δ 8.51 (s, 1H), 4.89 (s, 1H), 4.66 (s, 1H), 4.52 (d, J=9.8 Hz, 1H), 4.30-4.20 (m, 1H), 4.05 (d, J=10.6 Hz, 2H), 3.98-3.82 (m, 3H), 3.74 (q, J=6.4 Hz, 1H), 3.38 (m, J=9.0, 6.4 Hz, 1H), 3.26-3.10 (m, 3H), 2.68 (m, 1H), 2.24-2.13 (m, 3H), 1.85 (s, 1H), 1.73 (dt, J=16.2, 5.6 Hz, 6H), 1.60 (s, 1H), 1.51 (s, 1H), 1.43 (dd, J=8.0, 5.4 Hz, 1H), 1.29-1.21 (m, 3H), 1.19-1.16 (m, 6H), 1.12 (d, J=5.4 Hz, 4H), 1.03 (m, 1H), 0.95 (q, J=11.6 Hz, 2H), 0.77 (m, 1H).

### Synthesis of N-((2S,3R)-3-(cyclohexylmethoxy)-1-oxo-1-(piperidin-1-yl)butan-2-yl)-2-((S)-2,2-dimethylcyclopropane-1-carbonyl)-5-oxa-2-azaspiro[3.4]octane-8-carboxamide I-29

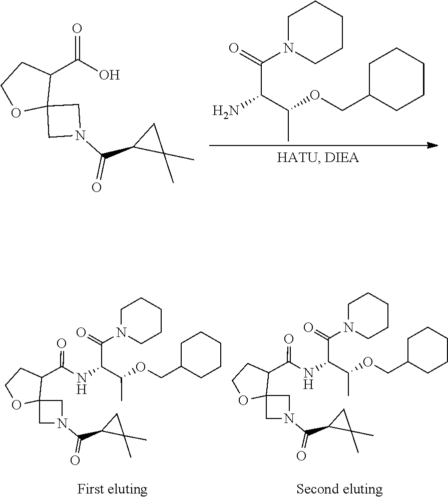

To a solution of 2-((S)-2,2-dimethylcyclopropane-1-carbonyl)-5-oxa-2-azaspiro[3.4]octane-8-carboxylic acid (40 mg, 0.16 mmol) in DCM (2 mL) was added HATU (48 mg, 0.16 mmol) and the reaction was stirred at room temperature for 30 min. (2S,3R)-2-amino-3-(cyclohexylmethoxy)-1-(piperidin-1-yl)butan-1-one (44 mg, 0.16 mmol) and DIPEA (62 mg, 0.48 mmol) were added and stirring continued for 2 h. The solvent was removed under vacuum and the residue obtained was purified by prep-HPLC to afford N-((2S,3R)-3-(cyclohexylmethoxy)-1-oxo-1-(piperidin-1-yl)butan-2-yl)-2-((S)-2,2-dimethylcyclopropane-1-carbonyl)-5-oxa-2-azaspiro[3.4]octane-8-carboxamide I-29 (11 mg, 27%) as a white solid as the first eluting diastereomer. LCMS m/z=518.4 [M+H]⁺; ¹H NMR (400 MHz, DMSO-d₆) δ 8.30 (d, J=8.5 Hz, 1H), 4.96-4.83 (m, 1H), 4.39-4.12 (m, 1H), 4.08-3.81 (m, 3H), 3.76-3.33 (m, 7H), 3.30-3.12 (m, 3H), 2.07-1.97 (m, 2H), 1.71-1.24 (m, 13H), 1.22-0.99 (m, 12H), 0.89-0.78 (m, 3H), 0.71-0.63 (m, 1H). Further elution provided I-29 (11 mg, 27%) as a white solid. LCMS m/z=518.4 [M+H]⁺; ¹H NMR (400 MHz, DMSO-d₆) δ 8.48-8.29 (m, 1H), 4.83 (dd, J=8.8, 6.2 Hz, 1H), 4.26-4.17 (m, 1H), 4.14-3.93 (m, 1H), 3.92-3.84 (m, 2H), 3.76-3.38 (m, 7H), 3.29-3.11 (m, 3H), 2.10-1.95 (m, 2H), 1.70-1.55 (m, 7H), 1.51-1.40 (m, 4H), 1.38-1.24 (m, 2H), 1.20-1.08 (m, 6H), 1.06-1.00 (m, 6H), 0.92-0.81 (m, 3H), 0.68-0.62 (m, 1H).

### Synthesis of [Example 395 and 396]: N-((2S,3R)-1-((S)-3-aminopiperidin-1-yl)-3-(cyclohexylmethoxy)-1-oxobutan-2-yl)-2-((S)-2,2-dimethylcyclopropane-1-carbonyl)-5-oxa-2-azaspiro[3.4]octane-8-carboxamide 1-31

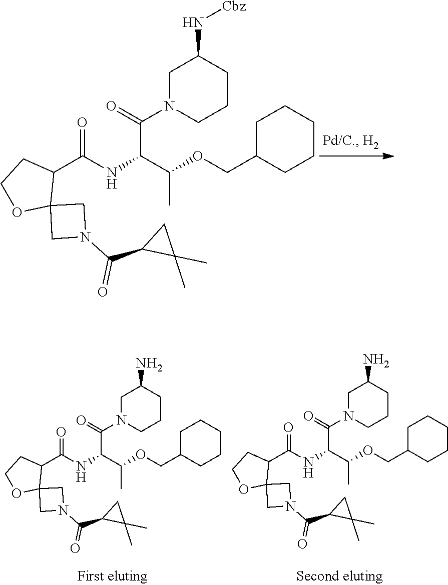

N-((2S,3R)-1-((S)-3-aminopiperidin-1-yl)-3-(cyclohexylmethoxy)-1-oxobutan-2-yl)-2-((S)-2,2-dimethylcyclopropane-1-carbonyl)-5-oxa-2-azaspiro[3.4]octane-8-carboxamide was synthesized according to the procedures outlined for I-34 N-((2S,3R)-1-((R)-3-aminopiperidin-1-yl)-3-(cyclohexylmethoxy)-1-oxobutan-2-yl)-2-((S)-2,2-dimethylcyclopropane-1-carbonyl)-5-oxa-2-azaspiro[3.4]octane-8-carboxamide using the appropriate commercially available reagents and/or intermediates described elsewhere. I-34 (A) was obtained as the first eluting isomer. LCMS m/z=533.4 [M+H]⁺; ¹H NMR (400 MHz, DMSO-d₆) δ 8.41-8.33 (m, 1H), 7.92 (s, 2H), 5.03-4.87 (m, 1H), 4.43-4.27 (m, 1H), 4.21-4.01 (m, 2H), 3.94-3.82 (m, 2H), 3.78-3.69 (m, 2H), 3.66-3.61 (m, 2H), 3.38-3.25 (m, 2H), 3.24-3.13 (m, 2H), 3.09-2.75 (m, 2H), 2.09-1.82 (m, 3H), 1.78-1.58 (m, 5H), 1.57-1.38 (m, 3H), 1.33-1.22 (m, 2H), 1.21-1.11 (m, 4H), 1.10-1.00 (m, 8H), 0.92-0.79 (m, 3H), 0.72-0.64 (m, 1H). Further elution provided I-34 (B) LCMS m/z=533.5 [M+H]⁺; ¹H NMR (400 MHz, DMSO-d₆) δ 8.65-8.32 (m, 1H), 7.91 (s, 2H), 4.94-4.77 (m, 1H), 4.54-4.39 (m, 1H), 4.31-4.13 (m, 2H), 4.02-3.94 (m, 1H), 3.93-3.85 (m, 2H), 3.73 (t, J=8.4 Hz, 2H), 3.65-3.58 (m, 2H), 3.34-3.27 (m, 2H), 3.20-3.11 (m, 1H), 3.03-2.90 (m, 1H), 2.82-2.64 (m, 1H), 2.09-1.97 (m, 3H), 1.87-1.59 (m, 6H), 1.54-1.35 (m, 3H), 1.32-1.18 (m, 2H), 1.17-1.10 (m, 5H), 1.08-0.99 (m, 6H), 0.87 (q, J=10.6, 9.2 Hz, 3H), 0.71-0.64 (m, 1H).

Table I-1: The compounds listed in Table I-1 were synthesized from Intermediate 4 according to the procedures outlined for the synthesis of intermediate 2 and Method 2A under this table using the appropriate commercially available reagents and/or intermediates described above.

| Example | | | |
|---|---|---|---|
| Number | Compound | ¹HNMR | LCMS |
| I-20 | 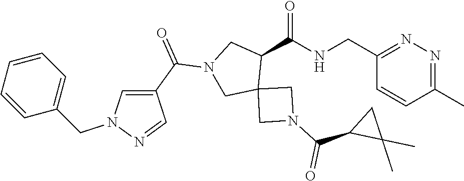 [SMILES: Cc1ccc(CNC(=O)[C@@H]2CN(C(=O)c3cnn(Cc4ccccc4)c3)CC23CN(C(=O)[C@H]2CC2(C)C)C3)nn1] | 1H NMR (400 MHz, DMSO-d₆) δ 8.97-8.93 (m, 1H), 8.36 (d, J = 8.8 Hz, 1H), 7.83 (d, J = 10.4 Hz, 1H), 7.51-7.39 (m, 2H), 7.37-7.25 (m, 5H), 5.35 (s, 2H), 4.65-4.45 (m, 2H), 4.25-4.05 (m, 2H), 3.99-3.64 (m, 6H), 3.25-3.16 (m, 1H), 2.58 (s, 3H), 1.38-1.27 (m, | m/z = 542.4 [M + H]⁺ |
| | | 1H), 1.11-0.98 (m, 6H), | |
| | | 0.85-0.82 (m, 1.5H), | |
| | | 0.68-0.64 (m, 1H). | |
| I-19 | 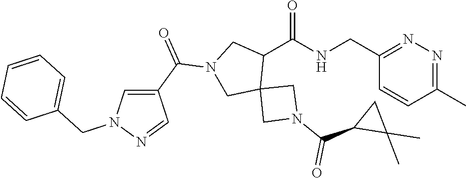 [SMILES: Cc1ccc(CNC(=O)C2CN(C(=O)c3cnn(Cc4ccccc4)c3)CC23CN(C(=O)[C@H]2CC2(C)C)C3)nn1] | 1H NMR (400 MHz, CD3OD) δ 8.22 (d, J = 24.9 Hz, 3H), 7.93 (d, J = 24.2 Hz, 1H), 7.38-7.24 (m, 5H), 5.38 (d, J = 6.1 Hz, 2H), 4.70 (s, 1H), 4.45-3.46 (m, 9H), 2.81 (t, J = 6.5 Hz, 3H), 1.39 (s, 1H), 1.20-1.08 (m, 6H), 1.03 (d, J = 5.0 Hz, 1H), 0.77 (d, J = 7.3 Hz, | m/z = 542.4 [M + H]⁺ |
| | | 1H). | |

### Intermediate 1: tert-butyl (R)-6-benzyl-8-((S)-4-benzyl-2-oxooxazolidine-3-carbonyl)-2,6-diazaspiro[3.4]octane-2-carboxylate and Intermediate 2: tert-butyl (S)-6-benzyl-8-((S)-4-benzyl-2-oxooxazolidine-3-carbonyl)-2,6-diazaspiro[3.4]octane-2-carboxylate

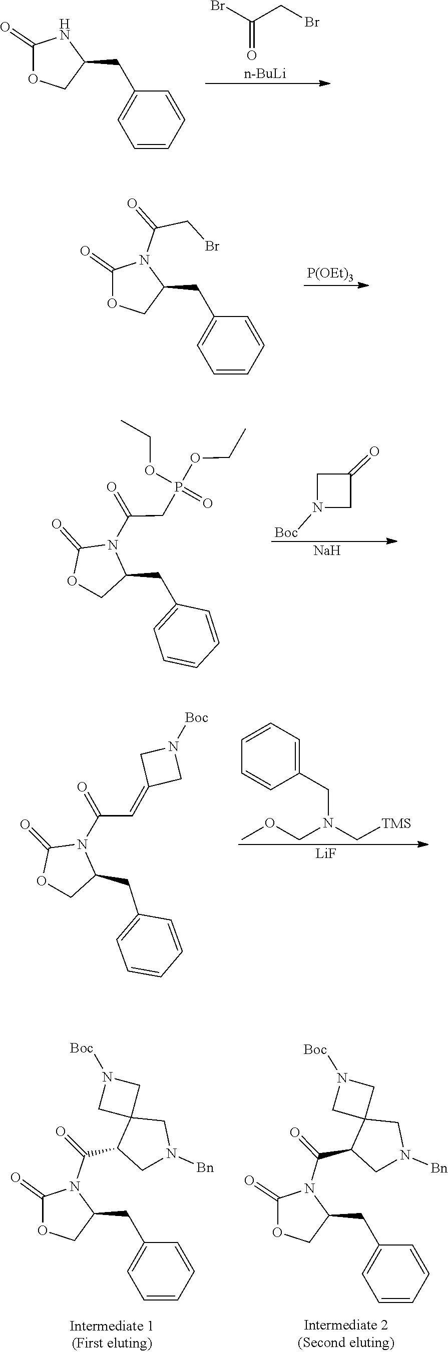

Step 1: To a solution of (S)-4-benzyloxazolidin-2-one (40 g, 225 mmol) in anhydrous THF (400 mL) at −78° C. under a N₂ atmosphere was added n-BuLi (2.5 M in Hexanes, 99 mL, 248 mmol) dropwise. The reaction mixture was stirred at −78° C. for 0.5 h then 2-bromoacetyl bromide (21 mL, 237 mmol) was added. The reaction was allowed to warm to room temperature and stirred for another 2 h. The mixture was diluted with EtOAc (600 mL), washed with water (200 mL×2), dried over Na₂SO₄, filtered and concentrated. The residue obtained was purified by column chromatography on silica gel (eluent: Pet. Ether EtOAc=10:1 to 3:1) to afford (S)-4-benzyl-3-(2-bromoacetyl)oxazolidin-2-one (50 g, 75%) as a yellow oil. ¹H NMR (400 MHz, CDCl₃) δ 7.38-7.26 (m, 3H), 7.24-7.19 (m, 2H), 4.70 (ddt, J=9.6, 7.8, 3.2 Hz, 1H), 4.61-4.48 (m, 2H), 4.30-4.20 (m, 2H), 3.33 (dd, J=13.6, 3.4 Hz, 1H), 2.81 (dd, J=13.4, 9.6 Hz, 1H).

Step 2: A mixture of (S)-4-benzyl-3-(2-bromoacetyl)oxazolidin-2-one (100 g, 335 mmol) in triethyl phosphite (279 g, 1.68 mol) was heated at 50° C. for 18 h. The excess triethyl phosphite was removed under vacuum at 70° C. to afford diethyl (S)-(2-(4-benzyl-2-oxooxazolidin-3-yl)-2-oxoethyl)phosphonate (110 g crude, 92%) as a yellow oil. LCMS m/z=356.0 [M+H]⁺; ¹H NMR (400 MHz, CDCl₃) δ 7.37-7.16 (m, 5H), 4.70 (ddt, J=10.4, 7.0, 3.4 Hz, 1H), 4.26-4.12 (m, 6H), 3.88-3.71 (m, 2H), 3.34 (dd, J=13.4, 3.4 Hz, 1H), 2.75 (dd, J=13.4, 9.8 Hz, 1H), 1.34 (t, J=7.0 Hz, 6H).

Step 3: To a solution of diethyl (S)-(2-(4-benzyl-2-oxooxazolidin-3-yl)-2-oxoethyl)phosphonate (30 g, 84 mmol) in anhydrous THF (300 mL) at 0° C. under N₂ atmosphere was added LiHMDS (1.0 M in THF, 85 mL, 85 mmol) dropwise. The reaction mixture was stirred at 0° C. for 30 min then tert-butyl 3-oxoazetidine-1-carboxylate (21.68 g, 127 mmol) was added. The reaction was allowed to warm to room temperature and stirred for another 1 h. The reaction was diluted with EtOAc (1 L) and the organic layer washed with sat. NH₄Cl (200 mL) and water (200 mL), dried over Na₂SO₄, filtered and concentrated. The residue obtained was purified by column chromatography on silica gel (eluent: Pet. Ether:EtOAc=10:1) to afford tert-butyl (S)-3-(2-(4-benzyl-2-oxooxazolidin-3-yl)-2-oxoethylidene)azetidine-1-carboxylate (17 g, 54%) as a yellow solid. LCMS m/z=373.1 [M+H]⁺; ¹H NMR (400 MHz, DMSO-d₆) δ 7.35-7.29 (m, 2H), 7.28-7.23 (m, 1H), 7.22-7.17 (m, 2H), 7.16-7.11 (m, 1H), 4.85-4.60 (m, 5H), 4.34 (t, J=8.4 Hz, 1H), 4.20 (dd, J=8.8, 2.8 Hz, 1H), 3.04 (dd, J=13.4, 3.2 Hz, 1H), 2.94 (dd, J=13.4, 7.6 Hz, 1H), 1.41 (s, 9H).

Step 4: A mixture of afford tert-butyl (S)-3-(2-(4-benzyl-2-oxooxazolidin-3-yl)-2-oxoethylidene)azetidine-1-carboxylate (10 g, 26.85 mmol), N-benzyl-1-methoxy-N-((trimethylsilyl)methyl)methanamine (8.29 g, 34.91 mmol) and LiF (2.09 g, 80.55 mmol) in acetonitrile (100 mL) was heated at 80° C. for 16 h. After cooling to room temperature, the mixture was diluted with water (200 mL) and extracted with EtOAc (300 mL×2). The combined organic layers were washed with brine, dried over Na₂SO₄, filtered and concentrated. The mixture was purified by column chromatography on silica gel (eluent: Pet. Ether:EtOAc=5:1) to afford tert-butyl (R)-6-benzyl-8-((S)-4-benzyl-2-oxooxazolidine-3-carbonyl)-2,6-diazaspiro[3.4]octane-2-carboxylate (Intermediate 1) (5 g, 37%) as a yellow oil as the first eluting isomer LCMS m/z=506.3 [M+H]⁺; ¹H NMR (400 MHz, DMSO-d₆) δ 7.36-7.31 (m, 4H), 7.30-7.23 (m, 4H), 7.20-7.18 (m, 2H), 4.66 (d, J=7.2 Hz, 1H), 4.33 (t, J=8.4 Hz, 1H), 4.23-4.20 (m, 1H), 4.05 (dd, J=8.0, 5.2 Hz, 1H), 3.90 (d, J=9.0 Hz, 1H), 3.75 (s, 1H), 3.73-3.55 (m, 4H), 3.19-3.09 (m, 1H), 3.00-2.92 (m, 3H), 2.74 (d, J=8.8 Hz, 1H), 2.45 (dd, J=9.6, 5.2 Hz, 1H), 1.35 (s, 9H). Further elution provided tert-butyl (S)-6-benzyl-8-((S)-4-benzyl-2-oxooxazolidine-3-carbonyl)-2,6-diazaspiro[3.4]octane-2-carboxylate (Intermediate 2) (5 g, 37%). LCMS m/z=506.3 [M+H]⁺; ¹H NMR (400 MHz, DMSO-d₆) δ 7.35-7.29 (m, 6H), 7.28-7.22 (m, 4H), 4.67-4.61 (m, 1H), 4.30 (t, J=8.4 Hz, 1H), 4.23 (dd, J=8.2, 6.2 Hz, 1H), 4.19-4.16 (m, 1H), 3.90 (br s, 1H), 3.83 (d, J=9.2 Hz, 1H), 3.68-3.66 (m, 2H), 3.62 (d, J=5.0 Hz, 2H), 3.12-3.05 (m, 1H), 3.02 (d, J=8.8 Hz, 1H), 2.97 (d, J=9.0 Hz, 1H), 2.86 (dd, J=13.4, 8.4 Hz, 1H), 2.65 (d, J=9.0 Hz, 1H), 2.55 (dd, J=9.4, 6.2 Hz, 1H), 1.36 (s, 9H).

### Intermediate 4: (S)-6-(1-benzyl-1H-pyrazole-4-carbonyl)-2-((S)-2,2-dimethylcyclopropane-1-carbonyl)-2,6-diazaspiro[3.4]octane-8-carboxylic acid

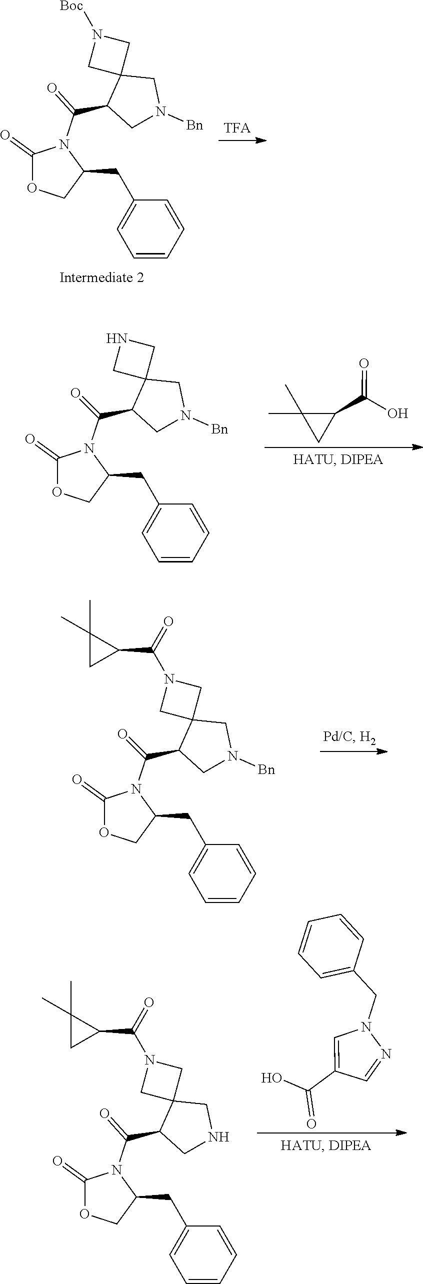

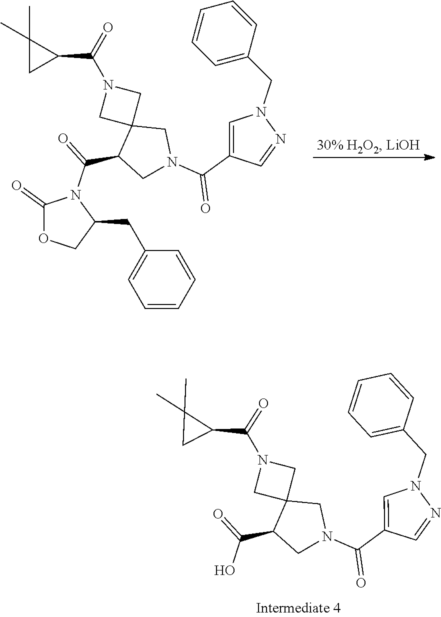

Step 1: To a solution of Intermediate 2 (6.2 g, 12.26 mmol) in DCM (10 mL) was added TFA (5 mL). The reaction mixture was stirred at room temperature for 2 h. The solvent was removed under vacuum to afford crude (S)-4-benzyl-3-((S)-6-benzyl-2,6-diazaspiro[3.4]octane-8-carbonyl)oxazolidin-2-one (5 g, 100%) which was used directly in the next step.

Step 2: To a solution of (S)-2,2-dimethylcyclopropane-1-carboxylic acid (1.55 g, 13.56 mmol) in DCM (50 mL) was added HATU (7.03 g, 18.50 mmol). The mixture was stirred at room temperature for 30 min. (S)-4-benzyl-3-((S)-6-benzyl-2,6-diazaspiro[3.4]octane-8-carbonyl)oxazolidin-2-one (5 g, 12.33 mmol) and DIPEA (6.37 g, 49.32 mmol) were added. The reaction mixture was stirred at room temperature for another 4 h. The mixture was diluted with water (100 mL), and then extracted with DCM (150 mL×2). The combined organic layers were washed with brine, dried over Na₂SO₄, filtered and concentrated. The mixture was purified by column chromatography on silica gel (eluent: Pet. Ether:EtOAc=3:1 to DCM/EtOAc=3/1) to afford (S)-4-benzyl-3-((S)-6-benzyl-2-((S)-2,2-dimethylcyclopropane-1-carbonyl)-2,6-diazaspiro[3.4]octane-8-carbonyl)oxazolidin-2-one (5.2 g, 84%) as a yellow solid. LCMS m/z=502.3 [M+H]⁺; ¹H NMR (400 MHz, DMSO-d₆) δ 7.53-7.36 (m, 5H), 7.35-7.21 (m, 5H), 4.67 (ddt, J=8.2, 5.6, 2.6 Hz, 1H), 4.58-4.46 (m, 1H), 4.37-3.93 (m, 7H), 3.89-3.79 (m, 1H), 3.57-3.40 (m, 2H), 3.10 (dt, J=13.4, 3.6 Hz, 1H), 2.88 (dd, J=13.4, 8.4 Hz, 1H), 1.35 (dd, J=8.0, 5.4 Hz, 1H), 1.13-0.96 (m, 6H), 0.86 (q, J=4.4 Hz, 1H), 0.70 (ddd, J=14.8, 8.0, 3.6 Hz, 1H).

Step 3: To a solution of (S)-4-benzyl-3-((S)-6-benzyl-2-((S)-2,2-dimethylcyclopropane-1-carbonyl)-2,6-diazaspiro[3.4]octane-8-carbonyl)oxazolidin-2-one (1.0 g, 1.99 mmol) in EtOAc (8 mL) was added 10% Pd/C (400 mg). The reaction mixture was stirred under a H₂ atmosphere for 24 h. Conversion was around 50%. The mixture was filtered through celite and concentrated. The residue was redissolved in EtOAc (8 mL) and another batch of 10% Pd/C (400 mg) was added. The reaction was stirred under H₂ atmosphere for another 24 h. The mixture was filtered and concentrated to afford (S)-4-benzyl-3-((S)-2-((S)-2,2-dimethylcyclopropane-1-carbonyl)-2,6-diazaspiro[3.4]octane-8-carbonyl)oxazolidin-2-one (800 mg, 98%) which was used directly in the next step. LCMS m/z=412.2 [M+H]⁺.

Step 4: To a solution of 1-benzyl-1H-pyrazole-4-carboxylic acid (308 mg, 1.52 mmol) in DMF (10 mL) was added HATU (790 mg, 2.08 mmol). The mixture was stirred at room temperature for 30 min. (S)-4-benzyl-3-((S)-2-((S)-2,2-dimethylcyclopropane-1-carbonyl)-2,6-diazaspiro[3.4]octane-8-carbonyl)oxazolidin-2-one (570 mg, 1.39 mmol) and DIPEA (716 mg, 5.54 mmol) were added. The reaction mixture was stirred at room temperature for another 3 h. The mixture was diluted with water (100 mL), extracted with EtOAc (150 mL×2). The combined organic layers were washed with brine, dried over Na₂SO₄, filtered and concentrated. The mixture was purified by column chromatography on silica gel (eluent: DCM/MeOH=30/1) to afford the (S)-4-benzyl-3-((S)-6-(1-benzyl-1H-pyrazole-4-carbonyl)-2-((S)-2,2-dimethylcyclopropane-1-carbonyl)-2,6-diazaspiro[3.4]octane-8-carbonyl)oxazolidin-2-one (410 mg, 50%) as a yellow solid. LCMS m/z=596.3 [M+H]⁺; ¹H NMR (400 MHz, DMSO-d₆) δ 8.44-8.28 (m, 1H), 7.88-7.76 (m, 1H), 7.40-7.18 (m, 10H), 5.36 (d, J=4.4 Hz, 2H), 4.70-4.60 (m, 1H), 4.40-4.23 (m, 4H), 4.21-4.01 (m, 3H), 3.97-3.56 (m, 5H), 3.18-2.84 (m, 3H), 1.42-1.33 (m, 1H), 1.28-1.21 (m, 5H), 1.14-1.02 (m, 7H), 0.86 (d, J=7.2 Hz, 1H), 0.69 (d, J=6.4 Hz, 1H).

Step 5: To a solution of (S)-4-benzyl-3-((S)-6-(1-benzyl-1H-pyrazole-4-carbonyl)-2-((S)-2,2-dimethylcyclopropane-1-carbonyl)-2,6-diazaspiro[3.4]octane-8-carbonyl)oxazolidin-2-one (410 mg, 0.69 mmol) in THF/H₂O (16 mL/2 mL) at 0° C. was added lithium hydroxide monohydrate (58 mg, 1.38 mmol) in H₂O (1 mL) and 30% H₂O₂ (0.18 mL, 1.72 mmol) in H₂O (1 mL). The reaction mixture was stirred at 0° C. for 1 h then diluted with water (20 mL) and extracted with EtOAc (30 mL). The aqueous layer was collected and acidified with 1M HCl to pH ˜2 and extracted with EtOAc (60 mL×3). The combined organic layers were washed with brine, dried over Na₂SO₄, filtered and concentrated to afford crude (S)-6-(1-benzyl-1H-pyrazole-4-carbonyl)-2-((S)-2,2-dimethylcyclopropane-1-carbonyl)-2,6-diazaspiro[3.4]octane-8-carboxylic acid (Intermediate 4) (245 mg, 82%) as a yellow solid. LCMS m/z=437.0 [M+H]⁺; ¹H NMR (400 MHz, DMSO-d₆) δ 12.69 (s, 1H), 8.38-8.28 (m, 1H), 7.88-7.79 (m, 1H), 7.37-7.23 (m, 6H), 5.36 (s, 2H), 4.37-4.24 (m, 1H), 4.17 (s, 1H), 4.11-3.61 (m, 7H), 1.42-1.32 (m, 1H), 1.09 (d, J=23.0 Hz, 6H), 0.90-0.85 (m, 1H), 0.72-0.64 (m, 1H).

### Exemplary Method 2A

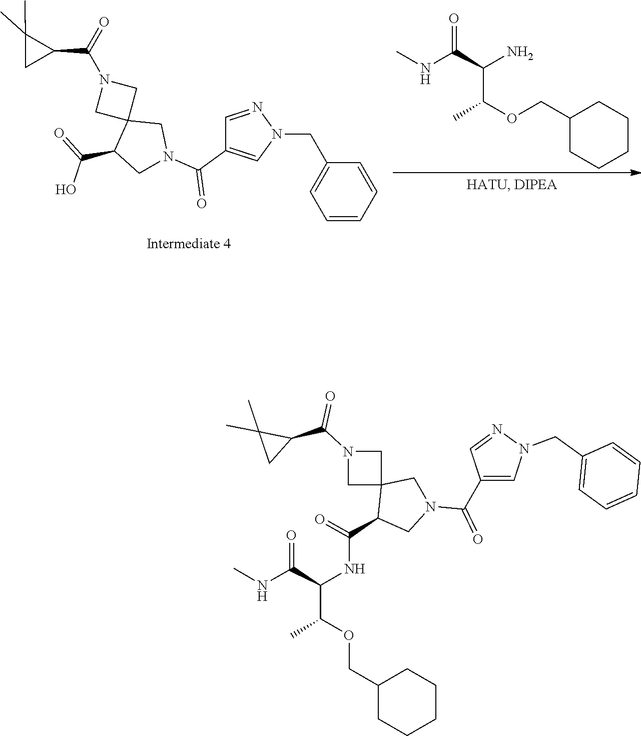 [SMILES: CC1(C)C[C@@H]1C(=O)N1CC2(CN(C(=O)c3cnn(Cc4ccccc4)c3)C[C@H]2C(=O)O)C1 ; CNC(=O)[C@@H](N)[C@@H](C)OCC1CCCCC1 ; CNC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1]

To a solution of (S)-6-(1-benzyl-1H-pyrazole-4-carbonyl)-2-((S)-2,2-dimethylcyclopropane-1-carbonyl)-2,6-diazaspiro[3.4]octane-8-carboxylic acid (Intermediate 4) (1.10 g, 2.52 mmol) in DCM (15 mL) was added HATU (1.44 g, 3.78 mmol) and the reaction mixture stirred at room temperature for 30 min. (2S,3R)-2-amino-3-(cyclohexylmethoxy)-N-methylbutanamide (633 mg, 2.77 mmol) and DIPEA (1.30 g, 10.08 mmol) were added and the reaction stirred for another 3 h. The solvent was removed under vacuum and the residue purified by reverse phase column (51% acetonitrile in water) to afford (S)-6-(1-benzyl-1H-pyrazole-4-carbonyl)-N-((2S,3R)-3-(cyclohexylmethoxy)-1-(methylamino)-1-oxobutan-2-yl)-2-((S)-2,2-dimethylcyclopropane-1-carbonyl)-2,6-diazaspiro[3.4]octane-8-carboxamide (I-123A) (1.15 g, 72%) as a white solid. LCMS m/z=647.5 [M+H]⁺; ¹H NMR (400 MHz, DMSO-d₆) δ 8.41-8.31 (m, 1H), 8.25-8.1 (m, 1H), 7.88-7.68 (m, 2H), 7.4-7.21 (m, 5H), 5.35 (s, 2H), 4.31-4.03 (m, 3H), 4.03-3.58 (m, 7H), 3.55-3.38 (m, 1H), 3.26-3.16 (m, 1H), 3.15-3.05 (m, 1H), 2.63-2.55 (m, 3H), 1.71-1.56 (m, 5H), 1.50-1.26 (m, 2H), 1.22-0.99 (m, 12H), 0.91-0.75 (m, 3H), 0.70-0.63 (m, 1H).

Table I-2 The compounds listed in Table 1-2 were synthesized from Intermediate 2 according to the procedures outlined herein using the appropriate commercially available reagents and/or intermediates described elsewhere.

TABLE I-2

| Example | | | |
|---|---|---|---|
| Number | Compound | ¹HNMR | LCMS |
| I-39 | 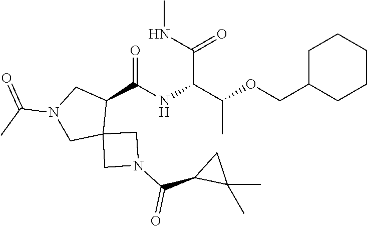 [SMILES: CNC(=O)[C@@H](NC(=O)[C@@H]1CN(C(C)=O)CC12CN(C(=O)[C@H]1CC1(C)C)C2)[C@@H](C)OCC1CCCCC1] | ¹H NMR (400 MHz, DMSO-d₆) δ 8.24-8.10 (m, 1H), 7.84-7.72 (m, 1H), 4.31-3.43 (m, 11H), 3.25-3.19 (m, 1H), 3.14-3.06 (m, 1H), 2.59 (dd, J = 4.6, 2.1 Hz, 3H), 1.98-1.88 (m, 3H), 1.71-1.56 (m, 5H), 1.50- 1.25 (m, 2H), 1.22- 0.99 (m, 12H), 0.89- 0.77 (m, 3H), 0.70-0.63 (m, 1H). | m/z = 505.3 [M + H]⁺ |
| I-36 | 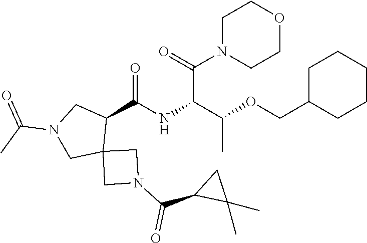 [SMILES: CC(=O)N1C[C@@H](C(=O)N[C@H](C(=O)N2CCOCC2)[C@@H](C)OCC2CCCCC2)C2(C1)CN(C(=O)[C@H]1CC1(C)C)C2] | ¹H NMR (400 MHz, DMSO-d₆) δ 8.42-8.32 (m, 1H), 4.92-4.80 (m, 1H), 4.22-3.65 (m, 5H), 3.65-3.46 (m, 10H), 3.45-3.33 (m, 3H), 3.28- 3.21 (m, 1H), 3.20- 3.13 (m, 1H), 1.96-1.89 (m, 3H), 1.71-1.57 (m, 5H), 1.50-1.38 (m, 1H), 1.38-1.25 (m, 1H), 1.18- 1.03 (m, 11H), 0.91- 0.79 (m, 3H), 0.70-0.64 (m, 1H). | m/z = 561.4 [M + H]⁺ |
| I-38 | 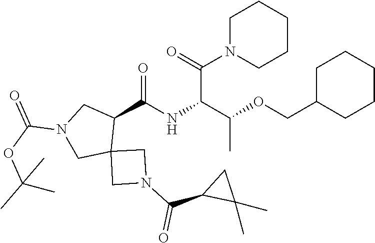 [SMILES: C[C@@H](OCC1CCCCC1)[C@H](NC(=O)[C@@H]1CN(C(=O)OC(C)(C)C)CC12CN(C(=O)[C@H]1CC1(C)C)C2)C(=O)N1CCCCC1] | ¹H NMR (400 MHz, DMSO-d₆) δ 8.31-8.26 (m, 1H), 4.86-4.82 (m, 1H), 4.17-3.97 (m, 2H), 3.91-3.80 (m, 1H), 3.69- 3.56 (m, 3H), 3.49- 3.34 (m, 8H), 3.27-3.21 (m, 1H), 3.17-3.13 (m, 1H), 1.68-1.58 (m, 7H), 1.46-1.44 (m, 4H), 1.39 (s, 9H), 1.36-1.29 (m, 1H), 1.19-1.02 (m, 13H), 0.89-0.84 (m, 3H), 0.68-0.66 (m, 1H). | m/z = 617.5 [M + H]⁺ |
| I-33 | 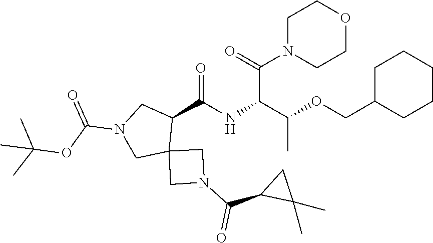 [SMILES: C[C@@H](OCC1CCCCC1)[C@H](NC(=O)[C@@H]1CN(C(=O)OC(C)(C)C)CC12CN(C(=O)[C@H]1CC1(C)C)C2)C(=O)N1CCOCC1] | ¹H NMR (400 MHz, DMSO-d₆) δ 8.39-8.31 (m, 1H), 4.88-4.81 (m, 1H), 4.19-3.78 (m, 3H), 3.71-3.41 (m, 13H), 3.38-3.34 (m, 2H), 3.28- 3.21 (m, 1H), 3.18- 3.12 (m, 1H), 1.71-1.59 (m, 5H), 1.48-1.34 (m, 11H), 1.32-1.23 (m, 1H), 1.16-1.01 (m, 11H), 0.85 (t, J = 4.9 Hz, 3H), 0.69-0.64 (m, 1H). | m/z = 619.4 [M + H]⁺ |

### Synthesis of [Example 294:] (S)—N-((2S,3R)-3-(cyclohexylmethoxy)-1-oxo-1-(piperidin-1-yl)butan-2-yl)-2-((S)-2,2-dimethylcyclopropane-1-carbonyl)-2,6-diazaspiro[3.4]octane-8-carboxamide: (I-37)

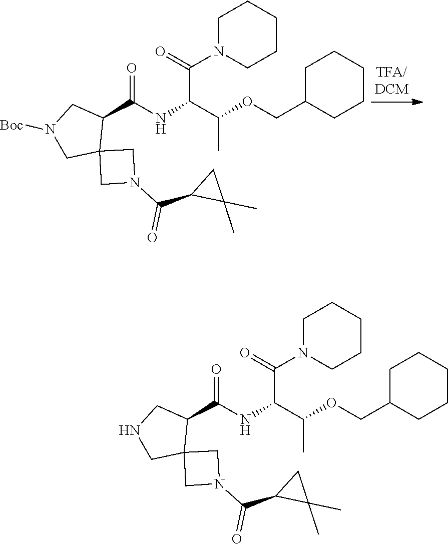 [SMILES: C[C@@H](OCC1CCCCC1)[C@H](NC(=O)[C@@H]1CN(C(=O)OC(C)(C)C)CC12CN(C(=O)[C@H]1CC1(C)C)C2)C(=O)N1CCCCC1 ; C[C@@H](OCC1CCCCC1)[C@H](NC(=O)[C@@H]1CNCC12CN(C(=O)[C@H]1CC1(C)C)C2)C(=O)N1CCCCC1]

(S)—N-((2S,3R)-3-(cyclohexylmethoxy)-1-oxo-1-(piperidin-1-yl)butan-2-yl)-2-((S)-2,2-dimethylcyclopropane-1-carbonyl)-2,6-diazaspiro[3.4]octane-8-carboxamide was synthesized from I-38 according to the procedures reported for (R)-3-((S)-6-benzyl-2,6-diazaspiro[3.4]octane-8-carbonyl)-4-phenyloxazolidin-2-one described in the following paragraph using the appropriate commercially available reagents and/or intermediates described elsewhere. LCMS m/z=517.5 [M+H]⁺; ¹H NMR (400 MHz, DMSO-d₆) δ 8.31-8.28 (m, 1H), 4.89-4.82 (m, 1H), 4.16-3.79 (m, 4H), 3.67-3.36 (m, 6H), 3.28-2.88 (m, 7H), 1.66-0.85 (m, 24H), 0.88-0.81 (m, 3H), 0.68-0.64 (m, 1H).

Synthesis of (R)-3-((S)-6-benzyl-2,6-diazaspiro[3.4]octane-8-carbonyl)-4-phenyloxazolidin-2-one: To a solution of tert-butyl (S)-6-benzyl-8-((R)-2-oxo-4-phenyloxazolidine-3-carbonyl)-2,6-diazaspiro[3.4]octane-2-carboxylate (1.1 g, 2.2 mmol) in DCM (4 mL) was added TFA (2 mL) and the reaction stirred at room temperature for 2 h. The solvent was removed under reduced pressure to afford (R)-3-((S)-6-benzyl-2,6-diazaspiro[3.4]octane-8-carbonyl)-4-phenyloxazolidin-2-one (876 mg, 100%) which was used without purification.

### Synthesis of [Example 295:] (S)—N-((2S,3R)-3-(cyclohexylmethoxy)-1-morpholino-1-oxobutan-2-yl)-2-((S)-2,2-dimethylcyclopropane-1-carbonyl)-2,6-diazaspiro[3.4]octane-8-carboxamide: (I-32)

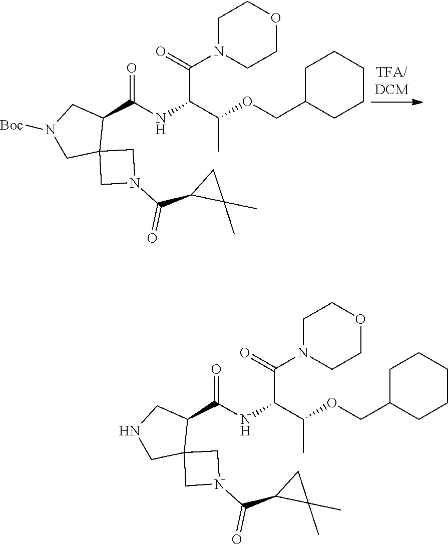 [SMILES: C[C@@H](OCC1CCCCC1)[C@H](NC(=O)[C@@H]1CN(C(=O)OC(C)(C)C)CC12CN(C(=O)[C@H]1CC1(C)C)C2)C(=O)N1CCOCC1 ; C[C@@H](OCC1CCCCC1)[C@H](NC(=O)[C@@H]1CNCC12CN(C(=O)[C@H]1CC1(C)C)C2)C(=O)N1CCOCC1]

(S)—N-((2S,3R)-3-(cyclohexylmethoxy)-1-morpholino-1-oxobutan-2-yl)-2-((S)-2,2-dimethylcyclopropane-1-carbonyl)-2,6-diazaspiro[3.4]octane-8-carboxamide was synthesized from according to the same procedure as I-33 according to the procedures reported for (R)-3-((S)-6-benzyl-2,6-diazaspiro[3.4]octane-8-carbonyl)-4-phenyloxazolidin-2-one described above using the appropriate commercially available reagents and/or intermediates described elsewhere. LCMS m/z=519.40; ¹H NMR (400 MHz, DMSO-d₆) δ 8.61-8.54 (m, 1H), 4.91-4.81 (m, 1H), 4.11-4.08 (m, 1H), 3.93-3.83 (m, 1H), 3.79-3.69 (m, 1H), 3.64-3.41 (m, 11H), 3.30-3.21 (m, 4H), 3.21-3.13 (m, 2H), 1.70-1.57 (m, 5H), 1.50-1.38 (m, 1H), 1.36-1.26 (m, 1H), 1.20-1.03 (m, 12H), 0.91-0.80 (m, 3H), 0.72-0.65 (m, 1H).

### Synthesis of ethyl (S)-2,6-diacetyl-2,6-diazaspiro[3.4]octane-8-carboxylate (I-28)

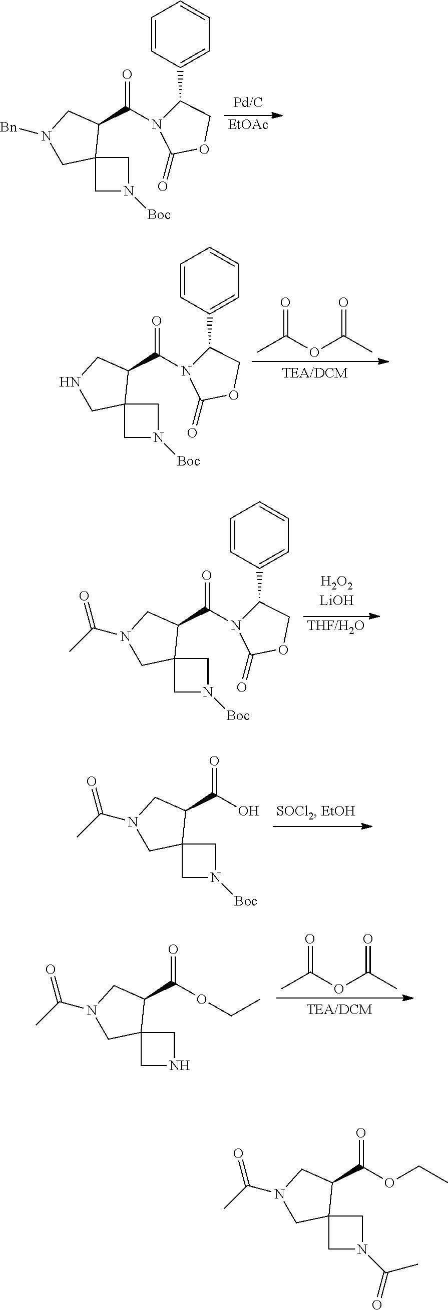

Step 1: tert-butyl (S)-8-((R)-2-oxo-4-phenyloxazolidine-3-carbonyl)-2,6-diazaspiro[3.4]octane-2-carboxylate: To a solution of tert-butyl (S)-6-benzyl-8-((R)-2-oxo-4-phenyloxazolidine-3-carbonyl)-2,6-diazaspiro[3.4]octane-2-carboxylate (1.0 g, 2 mmol) in EtOAc (8 mL) was added 10% Pd/C (400 mg). The reaction was stirred under a H₂ atmosphere for 24 h. 50% conversion was observed so the catalyst was removed by filtration through celite and the filtrate concentrated. The residue was resubjected to the hydrogenation conditions and stirred under a H₂ atmosphere for another 24 h. The catalyst was removed by filtration through Celite and the filtrate concentrated to afford tert-butyl (S)-8-((R)-2-oxo-4-phenyloxazolidine-3-carbonyl)-2,6-diazaspiro[3.4]octane-2-carboxylate (610 mg, 75%). LCMS m/z=402.1 [M+H]⁺.

Step 2: tert-butyl (S)-6-acetyl-8-((R)-2-oxo-4-phenyloxazolidine-3-carbonyl)-2,6-diazaspiro[3.4]octane-2-carboxylate: To a solution of tert-butyl (S)-8-((R)-2-oxo-4-phenyloxazolidine-3-carbonyl)-2,6-diazaspiro[3.4]octane-2-carboxylate (600 mg, 1.5 mmol) in DCM (10 mL) was added triethylamine (303 mg, 3 mmol) and acetic anhydride (766 mg, 7.5 mmol). The reaction mixture was stirred at room temperature for 3 h then was diluted with water (20 mL) and extracted with DCM (50 mL×2). The combined organic layers were washed with brine, dried over Na₂SO₄, filtered and concentrated to afford tert-butyl (S)-6-acetyl-8-((R)-2-oxo-4-phenyloxazolidine-3-carbonyl)-2,6-diazaspiro[3.4]octane-2-carboxylate (600 mg, 90%) as a colourless oil. LCMS m/z=388.1 [M+H-56]+

Step 3: (S)-6-acetyl-2-(tert-butoxycarbonyl)-2,6-diazaspiro[3.4]octane-8-carboxylic acid: To a solution of tert-butyl (S)-6-acetyl-8-((R)-2-oxo-4-phenyloxazolidine-3-carbonyl)-2,6-diazaspiro[3.4]octane-2-carboxylate (600 mg, 1.4 mmol) in a mixture of THF and water (8 mL/1 mL) at 0° C. was added a solution of lithium hydroxide monohydrate (113 mg, 2.7 mmol) and 30% H₂O₂ (385 mg, 3.4 mmol) in water (1 mL). The reaction mixture was stirred at 0° C. for 1 h then was diluted with water (20 mL) and extracted with EtOAc (30 mL). The aqueous layer was collected, acidified with to pH ˜2 1M HCl and extracted with EtOAc (60 mL×3). The combined organic layers were washed with brine, dried over Na₂SO₄, filtered and concentrated to afford crude (S)-6-acetyl-2-(tert-butoxycarbonyl)-2,6-diazaspiro[3.4]octane-8-carboxylic acid (200 mg, 50%) as a white solid which was used directly in the next step. LCMS m/z=243.1 [M+H-56]+.

Step 4: ethyl (S)-6-acetyl-2,6-diazaspiro[3.4]octane-8-carboxylate: To a solution of (S)-6-acetyl-2-(tert-butoxycarbonyl)-2,6-diazaspiro[3.4]octane-8-carboxylic acid (30 mg, 0.1 mmol) in EtOH (3 mL) at 0° C. was added SOCl₂ (0.3 mL) and the mixture stirred at room temperature for 2 h. The reaction was concentrated to afford ethyl (S)-6-acetyl-2,6-diazaspiro[3.4]octane-8-carboxylate (35 mg) as a white solid. LCMS m/z=227.2 [M+H]⁺.

Step 5: ethyl (S)-2,6-diacetyl-2,6-diazaspiro[3.4]octane-8-carboxylate I-28: To a solution of ethyl (S)-6-acetyl-2,6-diazaspiro[3.4]octane-8-carboxylate (40 mg, 0.2 mmol) in DCM (5 mL) was added triethylamine (35 mg, 0.4 mmol) and acetic anhydride (90 mg, 0.9 mmol). The reaction mixture was stirred at room temperature for 3 h then the solvent was removed and the residue obtained was purified by prep-HPLC to afford ethyl (S)-2,6-diacetyl-2,6-diazaspiro[3.4]octane-8-carboxylate (11 mg, 23%) as a white solid. LCMS m/z=269.2 [M+H]⁺; 1H NMR (400 MHz, Chloroform-d) δ 4.32-4.13 (m, 3H), 4.12-3.96 (m, 2H), 3.94-3.74 (m, 3H), 3.73-3.60 (m, 2H), 3.23-3.05 (m, 1H), 2.06 (s, 3H), 1.87 (d, J=7.2 Hz, 3H), 1.33-1.22 (m, 3H).

### Synthesis of (S)-2,6-diacetyl-2,6-diazaspiro[3.4]octane-8-carboxamide 1-22

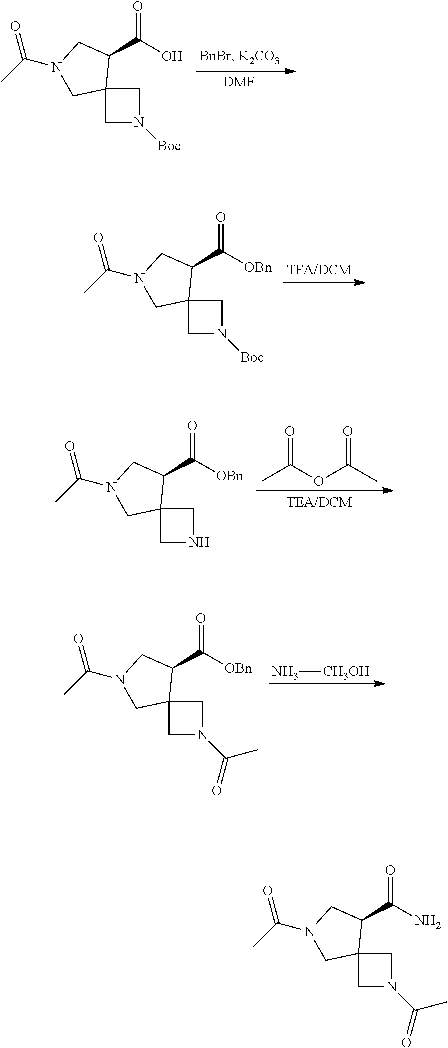

Step 1: 8-benzyl 2-(tert-butyl) (S)-6-acetyl-2,6-diazaspiro[3.4]octane-2,8-dicarboxylate: To a solution of (S)-6-acetyl-2-(tert-butoxycarbonyl)-2,6-diazaspiro[3.4]octane-8-carboxylic acid (20 mg, 0.067 mmol) in DMF (1 mL) was added K₂CO₃ (18.5 mg, 0.134 mmol) and BnBr (23.9 mg, 0.08 mmol). The reaction was stirred at room temperature for 12 h then was diluted with water (10 mL) and extracted with EtOAc (30 mL×2). The combined organic layers were washed with brine, dried over Na₂SO₄, filtered and concentrated. The residue was purified by prep-TLC (eluent: Pet. Ether:EtOAc=3:1) to afford 8-benzyl 2-(tert-butyl) (S)-6-acetyl-2,6-diazaspiro[3.4]octane-2,8-dicarboxylate (7 mg, 27%) as a colorless oil. LCMS m/z=430.15 [M+H]⁺; ¹H NMR (400 MHz, Methanol-d₄) δ 7.45-7.25 (m, 5H), 5.22-5.18 (m, 2H), 4.05 (t, J=8.8 Hz, 1H), 3.94 (d, J=9.2 Hz, 1H), 3.89-3.83 (m, 1H), 3.82-3.75 (m, 3H), 3.72 (dd, J=12.4, 5.8 Hz, 1H), 3.62 (t, J=10.2 Hz, 1H), 3.44-3.33 (m, 1H), 2.03 (d, J=3.8 Hz, 3H), 1.42 (s, 9H).

Step 2: benzyl (S)-6-acetyl-2,6-diazaspiro[3.4]octane-8-carboxylate: To a solution of 8-benzyl 2-(tert-butyl) (S)-6-acetyl-2,6-diazaspiro[3.4]octane-2,8-dicarboxylate (100 mg, 0.3 mmol) in DCM (1 mL) was added TFA (0.5 mL) and the reaction stirred at room temperature for 2 h. The solvent was removed under vacuum to afford (S)-6-acetyl-2,6-diazaspiro[3.4]octane-8-carboxylate (74 mg, 100%) which was used directly in the next step.

Step 3: benzyl (S)-2,6-diacetyl-2,6-diazaspiro[3.4]octane-8-carboxylate: To a solution of (S)-6-acetyl-2,6-diazaspiro[3.4]octane-8-carboxylate (74 mg, 0.3 mmol) in DCM (5 mL) was added triethylamine (53 mg, 0.5 mmol) and acetic anhydride (133 mg, 1.3 mmol). The reaction mixture stirred at room temperature for 3 h then was diluted with water (20 mL) and extracted with DCM (50 mL×2). The combined organic layers were washed with brine, dried over Na₂SO₄, filtered and concentrated. The residue obtained was purified by prep-TLC (DCM:MeOH=20:1) to afford (S)-2,6-diacetyl-2,6-diazaspiro[3.4]octane-8-carboxylate (40 mg, 47%) as a colourless oil. LCMS m/z=331.1 [M+H]⁺.

Step 4: (S)-2,6-diacetyl-2,6-diazaspiro[3.4]octane-8-carboxamide I-22: A solution of (S)-2,6-diacetyl-2,6-diazaspiro[3.4]octane-8-carboxylate (60 mg, 0.2 mmol) in a solution of ammonia in methanol (7 M, 3 mL) was heated at 100° C. in a microwave reactor for 6 h. The solvent was removed and the residue purified by prep-HPLC to afford (S)-2,6-diacetyl-2,6-diazaspiro[3.4]octane-8-carboxamide (8 mg, 19%) as a colourless oil. LCMS m/z=240.1 [M+H]⁺; ¹H NMR (400 MHz, DMSO-d6) δ 7.68-7.57 (m, 1H), 7.19-7.08 (m, 1H), 4.18-4.04 (m, 1H), 4.02-3.79 (m, 2H), 3.76-3.60 (m, 3H), 3.56-3.41 (m, 3H), 3.11-2.96 (m, 2H), 1.92 (d, J=4.4 Hz, 3H), 1.74 (d, J=1.2 Hz, 3H).

### N-((2S,3R)-3-(cyclohexylmethoxy)-1-(methylamino)-1-oxobutan-2-yl)-2-((S)-2,2-dimethylcyclopropane-1-carbonyl)-5-oxa-2-azaspiro[3.4]octane-8-carboxamide (I-35)

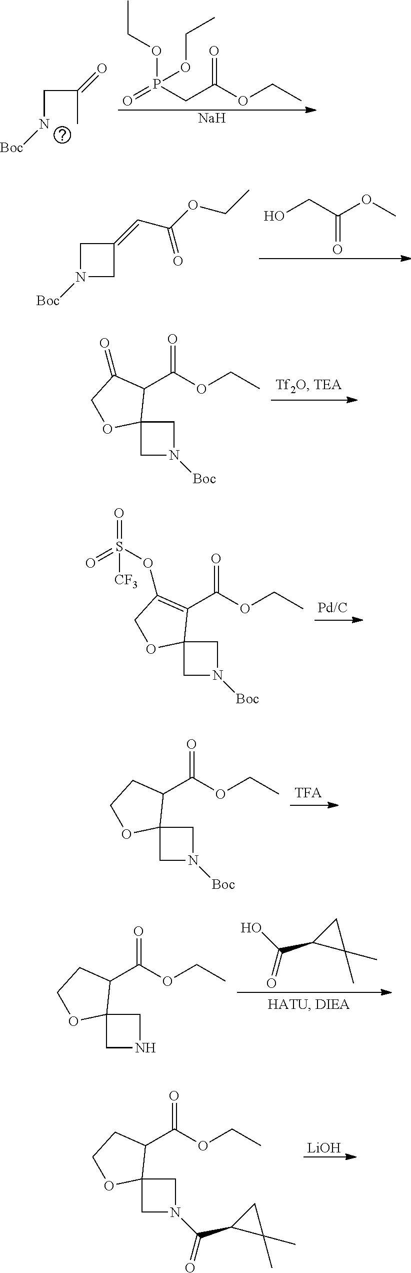

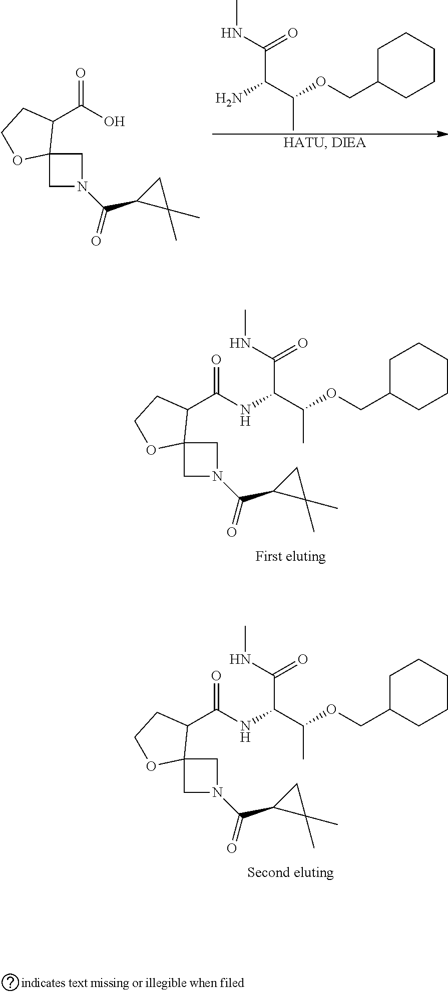

Step 1: tert-butyl 3-(2-ethoxy-2-oxoethylidene)azetidine-1-carboxylate: To a solution of ethyl 2-(diethoxyphosphoryl)acetate (13.1 g, 58.4 mmol) in anhydrous THE (50 mL) at 0° C. was added NaH (2.34 g, 58.4 mmol, 60% dispersion in oil) portionwise over 10 min. The reaction mixture was stirred at room temperature for 30 min then a solution tert-butyl 3-oxoazetidine-1-carboxylate (5 g, 29.2 mmol) in THE (10 mL) was added. The reaction was allowed to warm to room temperature and stirred for another 40 min then was quenched with water (40 mL) and extracted with EtOAc (100 mL×3). The combined organic layers were dried over Na₂SO₄, filtered and concentrated. The residue was purified by column chromatography on silica gel (eluent: Pet. Ether:EtOAc=8:1) to afford tert-butyl 3-(2-ethoxy-2-oxoethylidene)azetidine-1-carboxylate (6.2 g, 88%) as a yellow oil. LCMS m/z=242.1 [M+H]⁺; ¹H NMR (400 MHz, Chloroform-d) δ 5.77-5.73 (m, 1H), 4.83-4.77 (m, 2H), 4.60-4.55 (m, 2H), 4.16 (q, J=7.0 Hz, 2H), 1.44 (s, 9H), 1.26 (t, J=7.0 Hz, 3H).

Step 2: 2-(tert-butyl) 8-ethyl 7-oxo-5-oxa-2-azaspiro[3.4]octane-2,8-dicarboxylate: To a solution of methyl 2-hydroxyacetate (2.79 g, 30.9 mmol) in THF (50 mL) at 0° C. was added NaH (1.24 g, 30.9 mmol, 60% dispersion in oil). The reaction was stirred at room temperature for min then concentrated under vacuum. A solution of tert-butyl 3-(2-ethoxy-2-oxoethylidene)azetidine-1-carboxylate (6.2 g, 25.7 mmol) in DMSO (60 mL) at 0° C. was added to the residue and the reaction stirred at room temperature for 24 h. The reaction was diluted with water and acidified to pH ˜4 with 1 M HCl. The aqueous was extracted with EtOAc (100 mL×3) and the combined organic layers washed with water, dried over Na₂SO₄, filtered and concentrated to afford 2-(tert-butyl) 8-ethyl 7-oxo-5-oxa-2-azaspiro[3.4]octane-2,8-dicarboxylate (4.6 g, 60%). LCMS m/z=298.0 [M−H]⁻; ¹H NMR (400 MHz, Chloroform-d) δ 4.57 (s, 1H), 4.33-3.99 (m, 8H), 1.42-1.41 (m, 911), 1.31 (t, J=7.2 Hz, 3H).

Step 3: 2-(tert-butyl) 8-ethyl 7-(((trifluoromethyl)sulfonyl)oxy)-5-oxa-2-azaspiro[3.4]oct-7-ene-2,8-dicarboxylate: To a solution of 2-(tert-butyl) 8-ethyl 7-oxo-5-oxa-2-azaspiro[3.4]octane-2,8-dicarboxylate (500 mg, 1.67 mmol) and triethylamine (253 mg, 2.5 mmol) in DCM (5 mL) at 0° C. was added Tf₂O (565 mg, 2 mmol). The reaction mixture was stirred for 1 h then was diluted with water, extracted with DCM, dried over Na₂SO₄, filtered and concentrated to afford 2-(tert-butyl) 8-ethyl 7-(((trifluoromethyl)sulfonyl)oxy)-5-oxa-2-azaspiro[3.4]oct-7-ene-2,8-dicarboxylate (700 mg, 97%).

Step 4: 2-(tert-butyl) 8-ethyl 5-oxa-2-azaspiro[3.4]octane-2,8-dicarboxylate: To a solution of 2-(tert-butyl) 8-ethyl 7-(((trifluoromethyl)sulfonyl)oxy)-5-oxa-2-azaspiro[3.4]oct-7-ene-2,8-dicarboxylate (700 mg, 1.62 mmol) in EtOH (7 mL) was added 10% Pd/C (70 mg). The reaction was stirred under a H₂ atmosphere for 6 h then the catalyst was removed by filtration through celite and the filtrate concentrated to afford 2-(tert-butyl) 8-ethyl 5-oxa-2-azaspiro[3.4]octane-2,8-dicarboxylate (450 mg, 97%) which was used directly in the next step. LCMS m/z=571.2 [2M+H]⁺.

Step 5: ethyl 5-oxa-2-azaspiro[3.4]octane-8-carboxylate: To a solution of 2-(tert-butyl) 8-ethyl 5-oxa-2-azaspiro[3.4]octane-2,8-dicarboxylate (450 mg, 1.57 mmol) in DCM (5 mL) was added TFA (2 mL) and the reaction stirred at room temperature for 2 h. The solvent was removed under vacuum to afford crude ethyl 5-oxa-2-azaspiro[3.4]octane-8-carboxylate (290 mg, 100%) which was used directly in the next step. LCMS m/z=186.1 [M+H]⁺

Step 6: ethyl 2-((S)-2,2-dimethylcyclopropane-1-carbonyl)-5-oxa-2-azaspiro[3.4]octane-8-carboxylate: To a solution of (S)-2,2-dimethylcyclopropane-1-carboxylic acid (180 mg, 1.57 mmol) in DCM (4 mL) was added HATU (715 mg, 1.88 mmol). The mixture was stirred at room temperature for 30 min then ethyl 5-oxa-2-azaspiro[3.4]octane-8-carboxylate (290 mg, 1.57 mmol) and DIPEA (810 mg, 6.28 mmol) were added. The reaction mixture was stirred a further 2 h then was diluted with water (30 mL) and extracted with DCM (50 mL×2). The combined organic layers were washed with brine, dried over Na₂SO₄, filtered and concentrated. The mixture was purified by RP-column to afford ethyl 2-((S)-2,2-dimethylcyclopropane-1-carbonyl)-5-oxa-2-azaspiro[3.4]octane-8-carboxylate (330 mg, 75%) as a yellow solid. LCMS m/z=282.1 [M+H]⁺; ¹H NMR (400 MHz, Chloroform-d) δ 4.35-3.83 (m, 8H), 3.11-3.03 (m, 1H), 2.31-2.11 (m, 2H), 1.29-1.07 (m, 11H), 0.73-0.67 (m, 1H).

Step 7: 2-((S)-2,2-dimethylcyclopropane-1-carbonyl)-5-oxa-2-azaspiro[3.4]octane-8-carboxylic acid: To a solution of ethyl 2-((S)-2,2-dimethylcyclopropane-1-carbonyl)-5-oxa-2-azaspiro[3.4]octane-8-carboxylate (330 mg, 1.16 mmol) in a mixture of THF, water and EtOH (8 mL/2 mL/2 mL) was added LiOH (55 mg, 2.3 mmol). The reaction was stirred at room temperature for 2 h then diluted with water (30 mL) and extracted with ether (50 mL). The aqueous layer was collected, acidified to pH ˜2 with 1M HCl and extracted with EtOAc (50 mL×3). The combined organic layers were washed with brine, dried over Na₂SO₄, filtered and concentrated to afford 2-((S)-2,2-dimethylcyclopropane-1-carbonyl)-5-oxa-2-azaspiro[3.4]octane-8-carboxylic acid (230 mg, 79%) as a yellow solid which was used directly in the next step. LCMS m/z=254.1 [M+H]⁺.

Step 8: N-((2S,3R)-3-(cyclohexylmethoxy)-1-(methylamino)-1-oxobutan-2-yl)-2-((S)-2,2-dimethylcyclopropane-1-carbonyl)-5-oxa-2-azaspiro[3.4]octane-8-carboxamide I-35: To a solution of 2-((S)-2,2-dimethylcyclopropane-1-carbonyl)-5-oxa-2-azaspiro[3.4]octane-8-carboxylic acid (50 mg, 0.2 mmol) in DCM (2 mL) was added HATU (112 mg, 0.3 mmol) and the mixture stirred at room temperature for 30 min. (2S,3R)-2-amino-3-(cyclohexylmethoxy)-N-methylbutanamide (50 mg, 0.22 mmol) and DIPEA (103 mg, 0.8 mmol) were added and stirring continued for 3 h. The solvent was removed under vacuum and the residue obtained purified by prep-HPLC to afford 1-35 (A) (5 mg, 5.5%) as a white solid as the first eluting diastereomer. LCMS m/z=464.3 [M+H]⁺; ¹H NMR (400 MHz, DMSO-d₆) δ 8.13 (t, J=9.0 Hz, 1H), 7.81-7.71 (m, 1H), 4.31-4.25 (m, 1H), 4.11-4.01 (m, 3H), 3.89-3.82 (m, 2H), 3.76-3.72 (m, 2H), 3.32-3.27 (m, 1H), 3.26-3.16 (m, 1H), 3.14-3.06 (m, 1H), 2.58 (d, J=4.4 Hz, 3H), 2.11-2.00 (m, 2H), 1.67-1.58 (m, 5H), 1.38-1.24 (m, 1H), 1.18-1.09 (m, 4H), 1.08-0.96 (m, 9H), 0.88-0.75 (m, 3H), 0.70-0.62 (m, 1H). Further elution provided 1-35 (B) (10 mg, 11%) as a white solid. LCMS m/z=464.3 [M+H]⁺; ¹H NMR (400 MHz, DMSO-d₆) δ 8.19-8.12 (m, 1H), 7.78-7.71 (m, 1H), 4.36-4.22 (m, 2H), 4.01-3.84 (m, 3H), 3.77-3.63 (m, 3H), 3.27-3.23 (m, 1H), 3.15-3.08 (m, 1H), 2.57 (d, J=4.6 Hz, 3H), 2.08-1.99 (m, 2H), 1.69-1.60 (m, 5H), 1.46-1.29 (m, 2H), 1.20-1.09 (m, 6H), 1.04-1.00 (m, 6H), 0.91-0.79 (m, 3H), 0.69-0.64 (m, 1H).

### Synthesis of (8S)-2-(tert-butylsulfinyl)-N-((2S,3R)-3-(cyclohexylmethoxy)-1-((S)-3-(methoxymethyl)piperidin-1-yl)-1-oxobutan-2-yl)-6-(thiazole-5-carbonyl)-2,6-diazaspiro[3.4]octane-8-carboxamide (I-17)

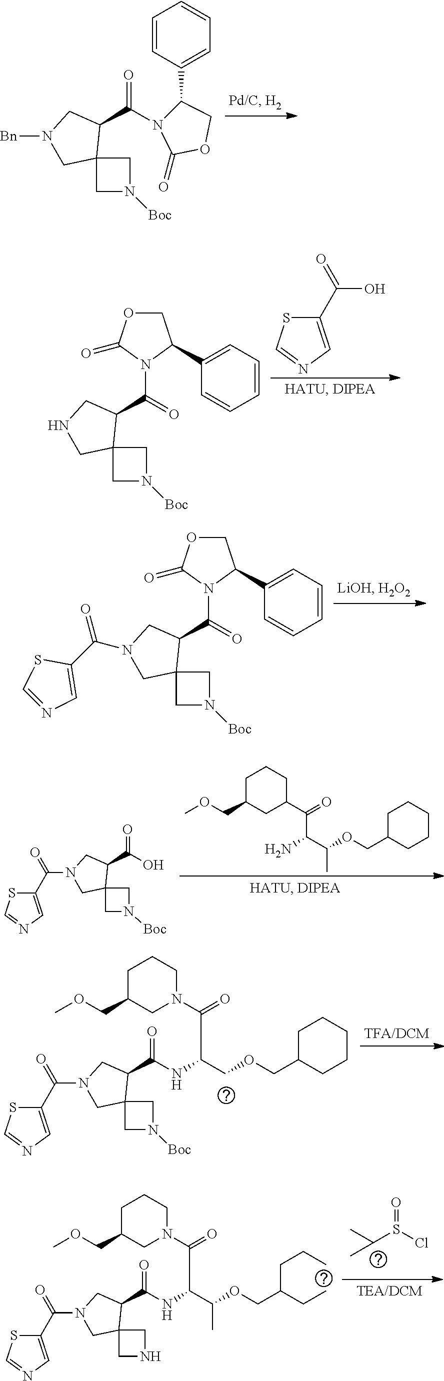

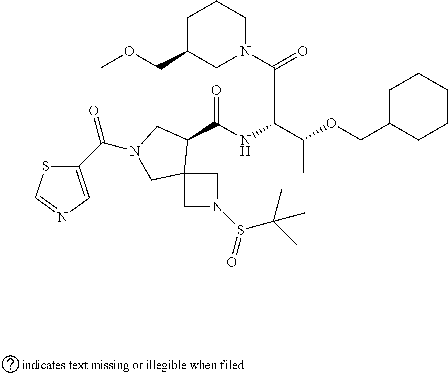 [SMILES: COC[C@H]1CCCN(C(=O)[C@@H](NC(=O)[C@@H]2CN(C(=O)c3cncs3)CC23CN(S(=O)C(C)(C)C)C3)[C@@H](C)OCC2CCCCC2)C1]

Step 1: tert-butyl (S)-8-((R)-2-oxo-4-phenyloxazolidine-3-carbonyl)-2,6-diazaspiro[3.4]octane-2-carboxylate: To a solution of tert-butyl (S)-6-benzyl-8-((R)-2-oxo-4-phenyloxazolidine-3-carbonyl)-2,6-diazaspiro[3.4]octane-2-carboxylate (2.5 g, 5.1 mmol) in EtOAc (20 mL) was added 10% Pd/C (1 g). The reaction was stirred under a H₂ atmosphere for 24 h. 50% conversion was observed, the catalyst was removed by filtration through Celite and the filtrate concentrated. The residue obtained was resubjected to hydrogenation under the same conditions then the catalyst was removed by filtration through Celite and the filtrate concentrated to afford tert-butyl (S)-8-((R)-2-oxo-4-phenyloxazolidine-3-carbonyl)-2,6-diazaspiro[3.4]octane-2-carboxylate (2.1 g) which was used in the next step without purification. LCMS m/z=402.3 [M+H]⁺.

Step 2: tert-butyl (S)-8-((R)-2-oxo-4-phenyloxazolidine-3-carbonyl)-6-(thiazole-5-carbonyl)-2,6-diazaspiro[3.4]octane-2-carboxylate: To a solution of thiazole-5-carboxylic acid (646 mg, 5.0 mmol) in DCM (20 mL) was added HATU (1.9 g, 5.0 mmol) and DIPEA (1.9 g, 15 mmol) and the mixture stirred at room temperature for 30 min. Tert-butyl (S)-6-benzyl-8-((R)-2-oxo-4-phenyloxazolidine-3-carbonyl)-2,6-diazaspiro[3.4]octane-2-carboxylate (2 g, 5.0 mmol) was added and stirring continued for 2 h. The mixture was diluted with water (100 mL) and extracted with DCM (150 mL×3). The combined organic layers were washed with brine, dried over Na₂SO₄, filtered and concentrated. The residue obtained was purified by column chromatography on silica gel (eluent: DCM:MeOH=50:1) to afford tert-butyl (S)-8-((R)-2-oxo-4-phenyloxazolidine-3-carbonyl)-6-(thiazole-5-carbonyl)-2,6-diazaspiro[3.4]octane-2-carboxylate (2 g, 80%) as a yellow oil. LCMS m/z=512.2 [M+H]⁺; ¹H NMR (400 MHz, Chloroform-d) δ 8.89 (s, 1H), 8.11 (s, 1H), 7.39-7.28 (m, 4H), 7.24-7.13 (m, 2H), 5.43-5.38 (m, 1H), 4.80-4.72 (m, 1H), 4.50-4.28 (m, 2H), 3.97-3.62 (m, 7H), 3.18-3.10 (m, 1H), 1.42 (s, 10H).

Step 3: (S)-2-(tert-butoxycarbonyl)-6-(thiazole-5-carbonyl)-2,6-diazaspiro[3.4]octane-8-carboxylic acid: To a solution of tert-butyl (S)-8-((R)-2-oxo-4-phenyloxazolidine-3-carbonyl)-6-(thiazole-5-carbonyl)-2,6-diazaspiro[3.4]octane-2-carboxylate (1.9 g, 3.7 mmol) in a mixture of THE and water (8 mL/1 mL) at 0° C. was added a solution of lithium hydroxide monohydrate (311 mg, 7.4 mmol) and 30% H₂O₂ (1.05 g, 9.3 mmol) in water (1 mL). The reaction mixture was stirred for 1 h then diluted with water (25 mL) and extracted with EtOAc (30 mL). The aqueous layer was collected, acidified to pH ˜2 with 1M HCl and extracted with EtOAc (80 mL×3). The combined organic layers were washed with brine, dried over Na₂SO₄, filtered and concentrated to afford (S)-2-(tert-butoxycarbonyl)-6-(thiazole-5-carbonyl)-2,6-diazaspiro[3.4]octane-8-carboxylic acid (1.3 g, 84%) as a yellow oil. LCMS m/z=312.0 [M+H]⁺; ¹H NMR (400 MHz, Chloroform-d) δ 8.97 (s, 1H), 8.27 (s, 1H), 4.40-4.16 (m, 1H), 4.09-3.57 (m, 7H), 3.29-3.18 (m, 1H), 1.43 (s, 9H).

Step 4: tert-butyl (S)-8-(((2S,3R)-3-(cyclohexylmethoxy)-1-((S)-3-(methoxymethyl)piperidin-1-yl)-1-oxobutan-2-yl)carbamoyl)-6-(thiazole-5-carbonyl)-2,6-diazaspiro[3.4]octane-2-carboxylate: To a solution of (S)-2-(tert-butoxycarbonyl)-6-(thiazole-5-carbonyl)-2,6-diazaspiro[3.4]octane-8-carboxylic acid (200 mg, 0.54 mmol) in DCM (5 mL) was added HATU (205 mg, 0.54 mmol) and DIPEA (279 mg, 2.16 mmol) and the mixture stirred at room temperature for 30 min. (2S,3R)-2-amino-3-(cyclohexylmethoxy)-1-((S)-3-(methoxymethyl)piperidin-1-yl)butan-1-one (176 mg, 0.54 mmol) was added and stirring continued for 2 h. The reaction was diluted with water (50 mL) and extracted with DCM (50 mL×3). The combined organic layers were washed with brine, dried over Na₂SO₄, filtered and concentrated. The residue obtained was purified by prep-TLC (eluent: DCM:MeOH=20:1) to afford tert-butyl (S)-8-(((2S,3R)-3-(cyclohexylmethoxy)-1-((S)-3-(methoxymethyl)piperidin-1-yl)-1-oxobutan-2-yl)carbamoyl)-6-(thiazole-5-carbonyl)-2,6-diazaspiro[3.4]octane-2-carboxylate (250 mg, 68%) as a yellow oil. LCMS m/z=676.3 [M+H]⁺.

Step 5: (S)—N-((2S,3R)-3-(cyclohexylmethoxy)-1-((S)-3-(methoxymethyl)piperidin-1-yl)-1-oxobutan-2-yl)-6-(thiazole-5-carbonyl)-2,6-diazaspiro[3.4]octane-8-carboxamide: To a solution of tert-butyl (S)-8-(((2S,3R)-3-(cyclohexylmethoxy)-1-((S)-3-(methoxymethyl)piperidin-1-yl)-1-oxobutan-2-yl)carbamoyl)-6-(thiazole-5-carbonyl)-2,6-diazaspiro[3.4]octane-2-carboxylate (100 mg, 0.15 mmol) in DCM (2 mL) was added TFA (0.5 mL) and the reaction stirred at room temperature for 1 h. The solvent was removed to afford (S)—N-((2S,3R)-3-(cyclohexylmethoxy)-1-((S)-3-(methoxymethyl)piperidin-1-yl)-1-oxobutan-2-yl)-6-(thiazole-5-carbonyl)-2,6-diazaspiro[3.4]octane-8-carboxamide (85 mg, 100%). LCMS m/z=576.3 [M+H]⁺.

Step 6: (8S)-2-(tert-butylsulfinyl)-N-((2S,3R)-3-(cyclohexylmethoxy)-1-((S)-3-(methoxymethyl)piperidin-1-yl)-1-oxobutan-2-yl)-6-(thiazole-5-carbonyl)-2,6-diazaspiro[3.4]octane-8-carboxamide (I-17): To a solution of (S)—N-((2S,3R)-3-(cyclohexylmethoxy)-1-((S)-3-(methoxymethyl)piperidin-1-yl)-1-oxobutan-2-yl)-6-(thiazole-5-carbonyl)-2,6-diazaspiro[3.4]octane-8-carboxamide (42 mg, 0.07 mmol) and TEA (28 mg, 0.28 mmol) in DCM (2 mL) was added 2-methylpropane-2-sulfinic chloride (10 mg, 0.07 mmol) at 0° C. The reaction was allowed to warm to room temperature and was stirred for 2 h then the solvent was removed under reduced pressure and the residue purified by prep-HPLC to afford (8S)-2-(tert-butylsulfinyl)-N-((2S,3R)-3-(cyclohexylmethoxy)-1-((S)-3-(methoxymethyl)piperidin-1-yl)-1-oxobutan-2-yl)-6-(thiazole-5-carbonyl)-2,6-diazaspiro[3.4]octane-8-carboxamide (15 mg, 31%) as a white solid. LCMS m/z=680.5 [M+H]⁺; ¹H NMR (400 MHz, DMSO-d6) δ 9.24 (d, J=4.9 Hz, 1H), 8.49-8.30 (m, 2H), 4.84 (s, 1H), 4.34-4.18 (m, 1H), 4.12-3.32 (m, 12H), 3.27-3.10 (m, 7H), 3.07-2.74 (m, 1H), 1.72-1.57 (m, 8H), 1.50-1.39 (m, 1H), 1.24-1.13 (m, 4H), 1.10-1.00 (m, 13H), 0.94-0.82 (m, 2H).

Table I-3. The compounds listed in Table I-3 were synthesized from (S)—N-((2S,3R)-3-(cyclohexylmethoxy)-1-((S)-3-(methoxymethyl)piperidin-1-yl)-1-oxobutan-2-yl)-6-(thiazole-5-carbonyl)-2,6-diazaspiro[3.4]octane-8-carboxamide (Step 5 above) according to the procedures outlined for 1-17 using the appropriate commercially available reagents and/or intermediates described elsewhere.

| Number | | | |
|---|---|---|---|
| Example | Compound | ¹HNMR | LCMS |
| I-18 | 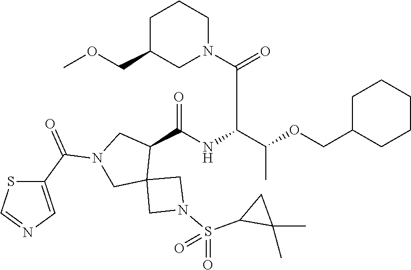 [SMILES: COC[C@H]1CCCN(C(=O)[C@@H](NC(=O)[C@@H]2CN(C(=O)c3cncs3)CC23CN(S(=O)(=O)C2CC2(C)C)C3)[C@@H](C)OCC2CCCCC2)C1] | ¹H NMR (400 MHz, DMSO-d6) δ 9.27-9.23 (m, 1H), 8.58-8.42 (m, 1H), 8.36 (d, J = 25.3 Hz, 1H), 4.84 (s, 1H), 4.33- 4.19 (m, 1H), 4.12-3.74 (m, 8H), 3.68-3.36 (m, 3H), 3.28-3.09 (m, 7H), 3.08-2.77 (m, 1H), 2.67- 2.57 (m, 1H), 1.76- 1.56 (m, 8H), 1.56-0.98 (m, 18H), 0.96-0.77 (m, 2H). | m/z = 707.3 [M + H]⁺ |

### Synthesis of (4-(5-((3,4-dichlorophenyl)difluoromethyl)-1,3,4-oxadiazol-2-yl)-8-oxa-2-azaspiro[4.5]decan-2-yl)(thiazol-5-yl)methanone (I-12)

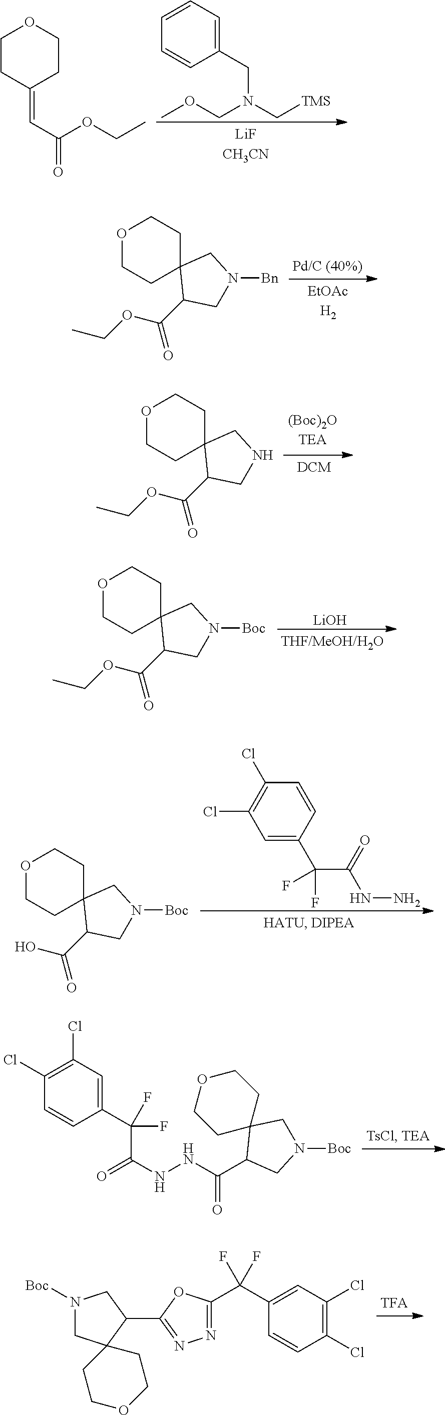

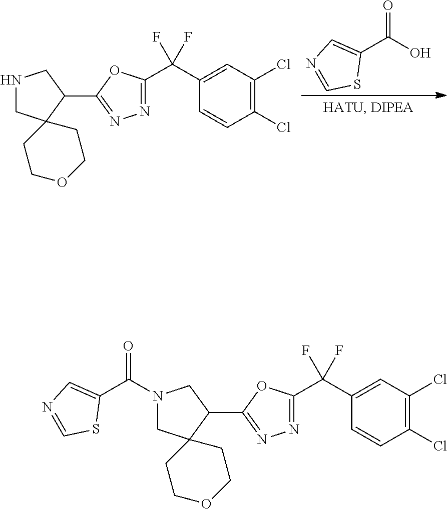

Step 1: ethyl 2-benzyl-8-oxa-2-azaspiro[4.5]decane-4-carboxylate: To a solution of ethyl 2-(tetrahydro-4H-pyran-4-ylidene)acetate (5.0 g, 28.49 mmol) in ACN (50 mL) was added N-benzyl-1-methoxy-N-((trimethylsilyl)methyl)methanamine (11.2 g, 42.74 mmol) and lithium fluoride (2.2 g, 85.78 mmol). The reaction was heated under N₂ atmosphere at 80° C. overnight then was diluted with water (300 mL) and extracted with DCM (100 mL×3). The organic layer was washed with brine, dried over Na₂SO₄, filtered and concentrated. The crude was purified by column chromatography on silica gel (eluent: Pet. Ether:EtOAc=30:1 to 10:1) to afford ethyl 2-benzyl-8-oxa-2-azaspiro[4.5]decane-4-carboxylate (4.4 g, 51%) as yellow oil. LCMS m/z=304.1 [M+H]⁺; ¹H NMR (400 MHz, DMSO-d₆) δ 7.31 (d, J=5.6 Hz, 5H), 4.12-4.01 (m, 2H), 3.64 (dd, J=21.8, 9.1 Hz, 4H), 3.31-3.24 (m, 1H), 2.81 (t, J=8.2 Hz, 1H), 2.75-2.65 (m, 2H), 2.44 (d, J=9.2 Hz, 1H), 1.99 (s, 1H), 1.85-1.75 (m, 1H), 1.51 (d, J=13.2 Hz, 1H), 1.39 (d, J=13.8 Hz, 2H), 1.24-1.14 (m, 4H).

Step 2: ethyl 8-oxa-2-azaspiro[4.5]decane-4-carboxylate: To a solution of ethyl 2-benzyl-8-oxa-2-azaspiro[4.5]decane-4-carboxylate (4.4 g, 14.49 mmol) in EtOAc (35 mL) was added 10% Pd/C (1.76 g). The reaction was heated at 55° C. under a H₂ atmosphere for 48 h then the catalyst was removed by filtration through Celite and the filtrate concentrated to afford ethyl 8-oxa-2-azaspiro[4.5]decane-4-carboxylate (2.6 g, 84%) as colourless oil. LCMS m/z=214.1[M+H]⁺.

Step 3: 2-(tert-butyl) 4-ethyl 8-oxa-2-azaspiro[4.5]decane-2,4-dicarboxylate: To a solution of ethyl 8-oxa-2-azaspiro[4.5]decane-4-carboxylate (2.5 g, 11.72 mmol) in DCM (20 mL) was added triethylamine (3.88 g, 23.44 mmol) and (Boc)₂O (2.4 g, 17.58 mmol) and the reaction stirred at room temperature for 3 h. The reaction was quenched with water (200 mL) and extracted with EtOAc (60 mL×5). The organic layer was washed with brine, dried over Na₂SO₄, filtered and concentrated. The mixture was purified by column chromatography on silica gel (eluent: Pet. Ether:EtOAc=15:1 to 10:1) to afford 2-(tert-butyl) 4-ethyl 8-oxa-2-azaspiro[4.5]decane-2,4-dicarboxylate (2.6 g, 71%) as a yellow oil. LCMS m/z=258.1 [M+H-56]⁺; ¹H NMR (400 MHz, DMSO-d₆) δ 4.10 (t, J=6.2 Hz, 2H), 3.69 (d, J=10.4 Hz, 2H), 3.45 (d, J=16.8 Hz, 5H), 3.23-3.16 (m, 1H), 2.90 (d, J=6.2 Hz, 1H), 1.78 (d, J=13.2 Hz, 1H), 1.46 (s, 1H), 1.40 (s, 9H), 1.34 (s, 2H), 1.20 (t, J=7.2 Hz, 3H).

Step 4: 2-(tert-butoxycarbonyl)-8-oxa-2-azaspiro[4.5]decane-4-carboxylic acid: To a solution of 2-(tert-butyl) 4-ethyl 8-oxa-2-azaspiro[4.5]decane-2,4-dicarboxylate (2.5 g, 7.98 mmol) in a mixture of THF (20 mL), MeOH (5 mL) and water (5 mL) was added lithium hydroxide monohydrate (1.6 g, 39.89 mmol). The reaction was stirred at room temperature for 1 h then was diluted with water (120 mL) and extracted with EtOAc (80 mL×3). The aqueous layer was collected, acidified to pH ˜2 with 1M HCl then extracted with EtOAc (200 mL×6). The combined organic layers were washed with brine, dried over Na₂SO₄, filtered and concentrated to afford 2-(tert-butoxycarbonyl)-8-oxa-2-azaspiro[4.5]decane-4-carboxylic acid (1.8 g, 79%) as a white solid. LCMS m/z=230.0 [M+H-56]⁺; ¹H NMR (400 MHz, DMSO-d6) δ 12.49 (s, 1H), 3.75-3.65 (m, 2H), 3.48-3.39 (m, 5H), 3.16 (t, J=10.6 Hz, 1H), 2.80 (q, J=6.8 Hz, 1H), 1.89-1.74 (m, 1H), 1.46 (d, J=15.8 Hz, 2H), 1.40 (s, 9H), 1.33 (d, J=14.6 Hz, 1H).

Step 5: tert-butyl 4-(2-(2-(3,4-dichlorophenyl)-2,2-difluoroacetyl)hydrazine-1-carbonyl)-8-oxa-2-azaspiro[4.5]decane-2-carboxylate: To a solution of 2-(tert-butoxycarbonyl)-8-oxa-2-azaspiro[4.5]decane-4-carboxylic acid (1.0 g, 3.5 mmol) in DCM (20 mL) was added HATU (1.6 g, 4.21 mmol) 2-(3,4-dichlorophenyl)-2,2-difluoroacetohydrazide (893.8 mg, 3.5 mmol) and DIPEA (1.83 g, 14.02 mmol) and the reaction stirred at room temperature for 2 h. The mixture was diluted with water (100 mL) and extracted with DCM (50 mL×3). The combined organic layers were washed with brine, dried over Na₂SO₄, filtered and concentrated. The mixture was purified by column chromatography on silica gel (eluent: Pet. Ether:EtOAc=70:1 to 30:1) to afford tert-butyl 4-(2-(2-(3,4-dichlorophenyl)-2,2-difluoroacetyl)hydrazine-1-carbonyl)-8-oxa-2-azaspiro[4.5]decane-2-carboxylate (1.3 g, 72%) as a white solid. LCMS m/z=544.1 [M+Na]⁺; ¹H NMR (400 MHz, DMSO-d₆) δ 11.05 (s, 1H), 10.24 (d, J=9.4 Hz, 1H), 7.94-7.85 (m, 2H), 7.64 (d, J=10.8 Hz, 1H), 3.66 (s, 2H), 3.42 (d, J=34.8 Hz, 4H), 3.23 (dd, J=17.8, 8.8 Hz, 2H), 2.78 (s, 1H), 1.58 (t, J=9.2 Hz, 2H), 1.39 (d, J=2.2 Hz, 9H), 1.23 (d, J=3.2 Hz, 1H), 0.85 (d, J=6.6 Hz, 1H).

Step 6: tert-butyl 4-(5-((3,4-dichlorophenyl)difluoromethyl)-1,3,4-oxadiazol-2-yl)-8-oxa-2-azaspiro[4.5]decane-2-carboxylate: To a solution of tert-butyl 4-(2-(2-(3,4-dichlorophenyl)-2,2-difluoroacetyl)hydrazine-1-carbonyl)-8-oxa-2-azaspiro[4.5]decane-2-carboxylate (500 mg, 0.957 mmol) in DCM (10 mL) was added triethylamine (489 mg, 4.79 mmol) and tosyl chloride (921.6 mg, 4.79 mmol). The mixture was stirred at room temperature for 3 h then was diluted with water (60 mL) and extracted with EtOAc (30 mL×4). The organic layers were washed with brine, dried over Na₂SO₄, filtered and concentrated. The mixture was purified by column chromatography on silica gel (eluent: Pet. Ether:EtOAc=10:1 to 1:1) to afford tert-butyl 4-(5-((3,4-dichlorophenyl)difluoromethyl)-1,3,4-oxadiazol-2-yl)-8-oxa-2-azaspiro[4.5]decane-2-carboxylate (340 mg, 70%) as colourless oil. LCMS m/z=448.1 [M+H-56]⁺; ¹H NMR (400 MHz, DMSO-d₆) δ 8.05 (s, 1H), 7.90 (d, J=8.6 Hz, 1H), 7.74 (d, J=8.6 Hz, 1H), 3.73 (s, 4H), 3.67-3.37 (m, 4H), 3.30 (s, 1H), 1.77 (t, J=12.2 Hz, 1H), 1.59 (d, J=13.6 Hz, 1H), 1.41 (d, J=2.4 Hz, 9H), 1.26-1.20 (m, 1H), 1.10 (s, 1H).

Step 7: 4-(5-((3,4-dichlorophenyl)difluoromethyl)-1,3,4-oxadiazol-2-yl)-8-oxa-2-azaspiro[4.5]decane: To a solution of tert-butyl 4-(5-((3,4-dichlorophenyl)difluoromethyl)-1,3,4-oxadiazol-2-yl)-8-oxa-2-azaspiro[4.5]decane-2-carboxylate (80 mg, 0.174 mmol) in DCM (1.5 mL) was added TFA (0.5 mL) and the reaction stirred at room temperature for 1 h. The solvent was removed under vacuum to afford 4-(5-((3,4-dichlorophenyl)difluoromethyl)-1,3,4-oxadiazol-2-yl)-8-oxa-2-azaspiro[4.5]decane (64 mg, 100%) which was used directly in the next step. LCMS m/z=404.1 [M+H]⁺.

Step 8: (4-(5-((3,4-dichlorophenyl)difluoromethyl)-1,3,4-oxadiazol-2-yl)-8-oxa-2-azaspiro[4.5]decan-2-yl)(thiazol-5-yl)methanone I-12: To a solution of thiazole-5-carboxylic acid (23 mg, 0.17 mmol) in DCM (5 mL) was added HATU (79 mg, 0.21 mmol) and the mixture stirred at room temperature for 30 min. 4-(5-((3,4-dichlorophenyl)difluoromethyl)-1,3,4-oxadiazol-2-yl)-8-oxa-2-azaspiro[4.5]decane (64 mg, 0.16 mmol) and DIPEA (82 mg, 0.61 mmol) were added and stirring continued for 2 h. The mixture was diluted with water (60 mL) and extracted with DCM (50 mL×2). The combined organic layers were washed with brine, dried over Na₂SO₄, filtered and concentrated. The mixture was purified by Prep-TLC (eluent: DCM:MeOH=20:1) to afford (4-(5-((3,4-dichlorophenyl)difluoromethyl)-1,3,4-oxadiazol-2-yl)-8-oxa-2-azaspiro[4.5]decan-2-yl)(thiazol-5-yl)methanone (20 mg, 24%) as white solid. LCMS m/z=515.0 [M+H]⁺; ¹H NMR (400 MHz, DMSO-d₆) δ 8.97 (s, 1H), 8.31 (s, 1H), 7.81-7.71 (m, 1H), 7.61 (d, J=8.4 Hz, 1H), 7.51 (d, J=8.6 Hz, 1H), 4.43-3.39 (m, 9H), 2.10-1.92 (m, 1H), 1.67 (s, 1H), 1.38 (s, 2H).

Table 1-4: The compounds listed in Table I-4 were synthesized from 4-(5-((3,4-dichlorophenyl)difluoromethyl)-1,3,4-oxadiazol-2-yl)-8-oxa-2-azaspiro[4.5]decane according to the procedures outlined for 1-12 using the appropriate commercially available reagents and/or intermediates described elsewhere herein.

| Example | | | |
|---|---|---|---|
| Number | Compound | ¹HNMR | LCMS |
| I-10 | 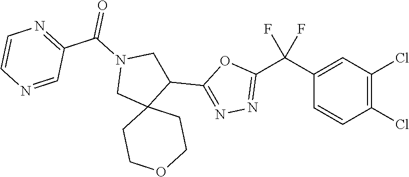 [SMILES: O=C(c1cnccn1)N1CC(c2nnc(C(F)(F)c3ccc(Cl)c(Cl)c3)o2)C2(CCOCC2)C1] | ¹HNMR (400 MHz, Chloroform-d) δ 9.26 (s, 1H), 8.70 (d, J = 11.4 Hz, 1H), 8.57 (d, J = 20.1 Hz, 1H), 7.76 (dd, J = 6.6, 2.1 Hz, 1H), 7.60 (d, J = 8.4 Hz, 1H), 7.50 (d, J = 5.2 Hz, 1H), 4.49 (d, J = 5.6 Hz, 1H), 4.20 (dd, J = 41.4, 10.3 Hz, 2H), 4.07- 3.74 (m, 3H), 3.63-3.41 (m, 3H), 2.09-1.93 (m, 1H), 1.70 (s, 1H), 1.38 (d, J = 21.6 Hz, 2H) | m/z = 532.1 [M + H]⁺ |
| I-7 | 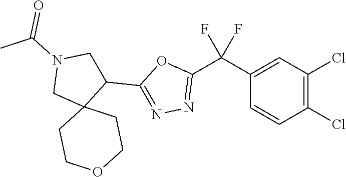 [SMILES: CC(=O)N1CC(c2nnc(C(F)(F)c3ccc(Cl)c(Cl)c3)o2)C2(CCOCC2)C1] | ¹HNMR (400 MHz, Chloroform-d) δ 7.76 (s, 1H), 7.64-7.58 (m, 1H), 7.52 (d, J = 7.0 Hz, 1H), 4.11-3.70 (m, 5H), 3.60- 3.44 (m, 4H), 2.12 (d, J = 5.8 Hz, 3H), 2.05-1.90 (m, 1H), 1.41-1.21 (m, 3H). | m/z = 446.1 [M + H]⁺ |
| I-1 | 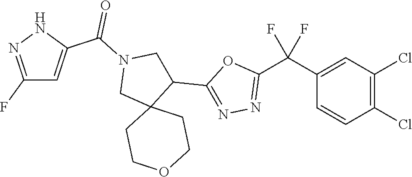 [SMILES: O=C(c1cc(F)n[nH]1)N1CC(c2nnc(C(F)(F)c3ccc(Cl)c(Cl)c3)o2)C2(CCOCC2)C1] | ¹HNMR (400 MHz, DMSO-d6) δ 8.04 (d, J = 4.6 Hz, 1H), 7.92-7.86 (m, 1H), 7.76-7.71 (m, 1H), 6.58 (dd, J = 75.0, 6.0 Hz, 1H), 4.21 (dd, J = 17.0, 6.7 Hz, 1H), 4.03 (d, J = 9.0 Hz, 1H), 3.89 (d, J = 10.6 Hz, 1H), 3.77 (d, J = 32.9 Hz, 2H), 3.70- 3.39 (m, 4H), 1.82 (d, J = 23.0 Hz, 1H), 1.66 (d, J = 13.6 Hz, 1H), 1.25-1.18 (m, 2H). | m/z = 515.0 [M + H]⁺ |

### Synthesis of 4-(1-(O-(cyclohexylmethyl)-N-(2-(thiazole-5-carbonyl)-8-oxa-2-azaspiro[4.5]decane-4-carbonyl)-L-threonyl)piperidin-4-yl)benzoic acid (I-4)

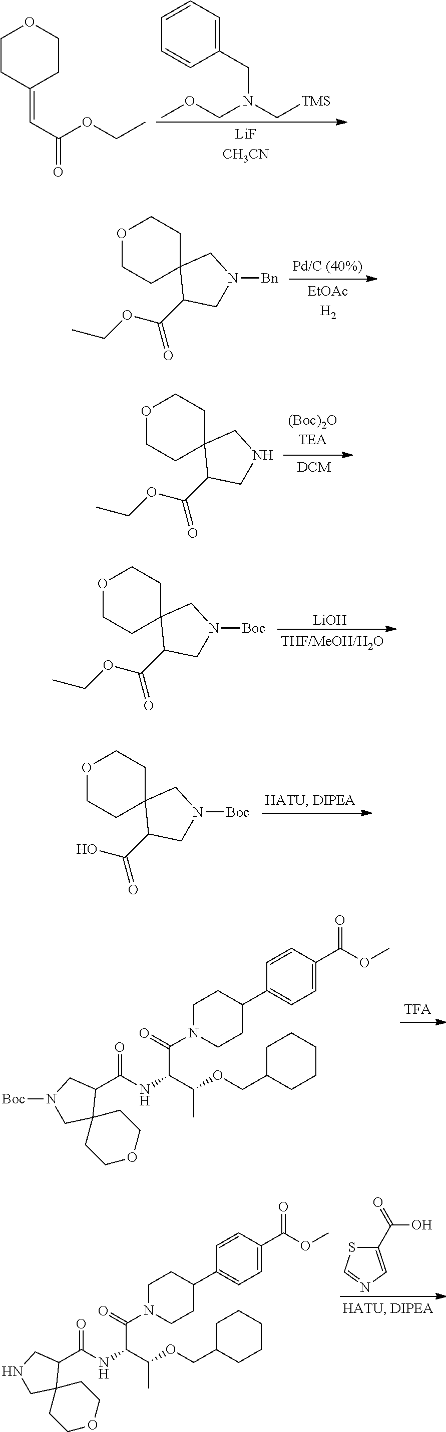

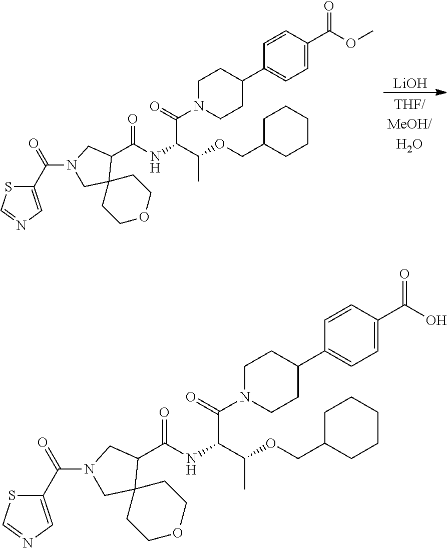 [SMILES: COC(=O)c1ccc(C2CCN(C(=O)[C@@H](NC(=O)C3CN(C(=O)c4cncs4)CC34CCOCC4)[C@@H](C)OCC3CCCCC3)CC2)cc1 ; "LiOH" ; C[C@@H](OCC1CCCCC1)[C@H](NC(=O)C1CN(C(=O)c2cncs2)CC12CCOCC2)C(=O)N1CCC(c2ccc(C(=O)O)cc2)CC1]

Step 1: ethyl 2-benzyl-8-oxa-2-azaspiro[4.5]decane-4-carboxylate: To a solution of ethyl 2-(tetrahydro-4H-pyran-4-ylidene)acetate (5.0 g, 28.49 mmol) in ACN (50 mL) was added N-benzyl-1-methoxy-N-((trimethylsilyl)methyl)methanamine (11.2 g, 42.74 mmol) and lithium fluoride (2.2 g, 85.78 mmol). The reaction was heated under N₂ atmosphere at 80° C. overnight then was diluted with water (300 mL) and extracted with DCM (100 mL×3). The organic layers were washed with brine, dried over Na₂SO₄, filtered and concentrated. The residue obtained was purified by column chromatography on silica gel (eluent: Pet. Ether:EtOAc=30:1 to 10:1) to afford ethyl 2-benzyl-8-oxa-2-azaspiro[4.5]decane-4-carboxylate (4.4 g, 51%) as yellow oil. LCMS m/z=304.1 [M+H]⁺; ¹H NMR (400 MHz, DMSO-d₆) δ 7.31 (d, J=5.6 Hz, 5H), 4.12-4.01 (m, 2H), 3.64 (dd, J=21.8, 9.1 Hz, 4H), 3.31-3.24 (m, 1H), 2.81 (t, J=8.2 Hz, 1H), 2.75-2.65 (m, 2H), 2.44 (d, J=9.2 Hz, 1H), 1.99 (s, 1H), 1.85-1.75 (m, 1H), 1.51 (d, J=13.2 Hz, 1H), 1.39 (d, J=13.8 Hz, 2H), 1.24-1.14 (m, 4H).

Step 2: ethyl 8-oxa-2-azaspiro[4.5]decane-4-carboxylate: To a solution of ethyl 2-benzyl-8-oxa-2-azaspiro[4.5]decane-4-carboxylate (4.4 g, 14.49 mmol) in EtOAc (35 mL) was added 10% Pd/C (1.76 g). The reaction was heated at 55° C. under a H₂ atmosphere for 48 h. The catalyst was removed by filtration through Celite and the filtrate concentrated to afford ethyl 8-oxa-2-azaspiro[4.5]decane-4-carboxylate (2.6 g, 84%) as colourless oil. LCMS m/z=214.1[M+H]⁺.

Step 3: 2-(tert-butyl) 4-ethyl 8-oxa-2-azaspiro[4.5]decane-2,4-dicarboxylate: To a solution of ethyl 8-oxa-2-azaspiro[4.5]decane-4-carboxylate (2.5 g, 11.72 mmol) in DCM (20 mL) was added triethylamine (3.88 g, 23.44 mmol) and (Boc)₂O (2.4 g, 17.58 mmol) and the reaction stirred at room temperature for 3 h. The reaction was quenched with water (200 mL) and extracted with EtOAc (60 mL×5). The organic layers were washed with brine, dried over Na₂SO₄, filtered and concentrated. The mixture was purified by column chromatography on silica gel (eluent: Pet. Ether:EtOAc=15:1 to 10:1) to afford 2-(tert-butyl) 4-ethyl 8-oxa-2-azaspiro[4.5]decane-2,4-dicarboxylate (2.6 g, 71%) as yellow oil. LCMS m/z=258.1 [M-56+H]⁺; ¹H NMR (400 MHz, DMSO-d₆) δ 4.10 (t, J=6.2 Hz, 2H), 3.69 (d, J=10.4 Hz, 2H), 3.45 (d, J=16.8 Hz, 5H), 3.23-3.16 (m, 1H), 2.90 (d, J=6.2 Hz, 1H), 1.78 (d, J=13.2 Hz, 1H), 1.46 (s, 1H), 1.40 (s, 9H), 1.34 (s, 2H), 1.20 (t, J=7.2 Hz, 3H).

Step 4: 2-(tert-butoxycarbonyl)-8-oxa-2-azaspiro[4.5]decane-4-carboxylic acid: To a solution of 2-(tert-butyl) 4-ethyl 8-oxa-2-azaspiro[4.5]decane-2,4-dicarboxylate (2.5 g, 7.98 mmol) in a mixture of THE (20 mL), MeOH (5 mL) and water (5 mL) was added lithium hydroxide monohydrate (1.6 g, 39.89 mmol) and the reaction stirred at room temperature for 1 h. The reaction was diluted with water (120 mL) and extracted with EtOAc (80 mL×3). The aqueous layer was collected, acidified to pH ˜2 with 1M HCl then extracted with EtOAc (200 mL×6). The combined organic layers were washed with brine, dried over Na₂SO₄, filtered and concentrated to afford 2-(tert-butoxycarbonyl)-8-oxa-2-azaspiro[4.5]decane-4-carboxylic acid (1.8 g, 79%) as a white solid. LCMS m/z=230.0 [M+H-56]⁺; ¹H NMR (400 MHz, DMSO-d₆) δ 12.49 (s, 1H), 3.75-3.65 (m, 2H), 3.48-3.39 (m, 5H), 3.16 (t, J=10.6 Hz, 1H), 2.80 (q, J=6.8 Hz, 1H), 1.89-1.74 (m, 1H), 1.46 (d, J=15.8 Hz, 2H), 1.40 (s, 9H), 1.33 (d, J=14.6 Hz, 1H).

Step 5: tert-butyl 4-(((2S,3R)-3-(cyclohexylmethoxy)-1-(4-(4-(methoxycarbonyl)phenyl)piperidin-1-yl)-1-oxobutan-2-yl)carbamoyl)-8-oxa-2-azaspiro[4.5]decane-2-carboxylate: To a solution of 2-(tert-butoxycarbonyl)-8-oxa-2-azaspiro[4.5]decane-4-carboxylic acid (328 mg 1.15 mmol) in DCM (4 mL) was added HATU (365 mg, 0.96 mmol) and the mixture stirred at room temperature for 30 min. 4-(1-(0-(cyclohexylmethyl)-L-threonyl)piperidin-4-yl)benzoate (400 mg, 0.96 mmol) and DIPEA (593 mg, 4.6 mmol) were added and stirring continued for 2 h. The mixture was diluted with water (100 mL) and extracted with DCM (50 mL×3). The combined organic layers were washed with brine, dried over Na₂SO₄, filtered and concentrated. The mixture was purified by RP column (78% MeOH in H₂O) to afford tert-butyl 4-(((2S,3R)-3-(cyclohexylmethoxy)-1-(4-(4-(methoxycarbonyl)phenyl)piperidin-1-yl)-1-oxobutan-2-yl)carbamoyl)-8-oxa-2-azaspiro[4.5]decane-2-carboxylate (440 mg, 74%) as white solid. LCMS m/z=584.2 [M+H-100]⁺; ¹HNMR (400 MHz, DMSO-d₆) δ 8.32-8.18 (m, 1H), 7.89 (s, 2H), 7.36 (d, J=18.5 Hz, 2H), 4.87 (d, J=8.1 Hz, 1H), 4.52 (d, J=15.1 Hz, 1H), 4.33-4.15 (m, 1H), 3.83 (s, 3H), 3.80-3.55 (m, 4H), 3.37 (s, 4H), 3.22 (s, 4H), 2.92 (s, 2H), 2.67 (s, 1H), 1.85 (d, J=14.9 Hz, 3H), 1.71-1.49 (m, 9H), 1.39 (s, 9H), 1.36-1.22 (m, 3H), 1.18-1.01 (m, 5H), 0.87 (d, J=10.3 Hz, 2H).

Step 6: 4-(1-(O-(cyclohexylmethyl)-N-(8-oxa-2-azaspiro[4.5]decane-4-carbonyl)-L-threonyl)piperidin-4-yl)benzoate: To a solution of tert-butyl 4-(((2S,3R)-3-(cyclohexylmethoxy)-1-(4-(4-(methoxycarbonyl)phenyl)piperidin-1-yl)-1-oxobutan-2-yl)carbamoyl)-8-oxa-2-azaspiro[4.5]decane-2-carboxylate (200 mg, 0.29 mmol) in DCM (3 mL) was added TFA (1 mL) and the reaction stirred at room temperature for 1 h. The mixture was concentrated to afford methyl 4-(1-(O-(cyclohexylmethyl)-N-(8-oxa-2-azaspiro[4.5]decane-4-carbonyl)-L-threonyl)piperidin-4-yl)benzoate (170 mg, 100%) as white solid which was used directly in the next step. LCMS m/z=584.2 [M+H]⁺.

Step 7: methyl 4-(1-(O-(cyclohexylmethyl)-N-(2-(thiazole-5-carbonyl)-8-oxa-2-azaspiro[4.5]decane-4-carbonyl)-L-threonyl)piperidin-4-yl)benzoate: To a solution of thiazole-5-carboxylic acid (41 mg 0.32 mmol) in DCM (3 mL) was added HATU (110 mg, 0.29 mmol) and the mixture stirred at room temperature for 30 min. 4-(1-(O-(cyclohexylmethyl)-N-(8-oxa-2-azaspiro[4.5]decane-4-carbonyl)-L-threonyl)piperidin-4-yl)benzoate (170 mg, 0.29 mmol) and DIPEA (150 mg, 1.16 mmol) were added and stirring continued for 2 h. The mixture was diluted with water (30 mL) and extracted with DCM (50 mL×3). The combined organic layers were washed with brine, dried over Na₂SO₄, filtered and concentrated. The mixture was purified by RP column (80% MeOH in H₂O) to afford methyl 4-(1-(O-(cyclohexylmethyl)-N-(2-(thiazole-5-carbonyl)-8-oxa-2-azaspiro[4.5]decane-4-carbonyl)-L-threonyl)piperidin-4-yl)benzoate (120 mg, 60%) as white solid. LCMS: LCMS m/z=695.2 [M+H]⁺; ¹HNMR (400 MHz, DMSO-d6) δ 9.27-9.19 (m, 1H), 8.50-8.32 (m, 2H), 7.94-7.82 (m, 2H), 7.42-7.29 (m, 2H), 4.89 (d, J=8.0 Hz, 1H), 4.62-4.49 (m, 1H), 4.32-4.00 (m, 2H), 3.84 (s, 3H), 3.81-3.70 (m, 3H), 3.69-3.59 (m, 3H), 3.54 (d, J=12.6 Hz, 2H), 3.44 (s, 1H), 3.15 (d, J=8.6 Hz, 2H), 2.96-2.88 (m, 1H), 2.71-2.63 (m, 1H), 1.91-1.80 (m, 2H), 1.71-1.54 (m, 8H), 1.48-1.34 (m, 3H), 1.25 (d, J=9.0 Hz, 2H), 1.18-1.02 (m, 6H), 0.96-0.74 (m, 2H).

Step 8: 4-(1-(O-(cyclohexylmethyl)-N-(2-(thiazole-5-carbonyl)-8-oxa-2-azaspiro[4.5]decane-4-carbonyl)-L-threonyl)piperidin-4-yl)benzoic acid I-4: To a solution of methyl 4-(1-(O-(cyclohexylmethyl)-N-(2-(thiazole-5-carbonyl)-8-oxa-2-azaspiro[4.5]decane-4-carbonyl)-L-threonyl)piperidin-4-yl)benzoate (100 mg, 0.14 mmol) in a mixture of THF (2 mL), EtOH (1 mL) and water (1 mL) was added lithium hydroxide monohydrate (25 mg, 0.57 mmol). The reaction was stirred at room temperature for 1 h then was diluted with water (20 mL) and extracted with EtOAc (20 mL). The aqueous layer was collected, acidified to pH ˜2 with 1M HCl then extracted with EtOAc (30 mL×6). The combined organic layers were washed with brine, dried over Na₂SO₄, filtered and concentrated. The residue obtained was purified by prep-HPLC to afford 4-(1-(O-(cyclohexylmethyl)-N-(2-(thiazole-5-carbonyl)-8-oxa-2-azaspiro[4.5]decane-4-carbonyl)-L-threonyl)piperidin-4-yl)benzoic acid as the first eluting diastereomer: (A) (15.4 mg, 15.8%) as white solid. LCMS: 681.2 [M+H]⁺; ¹HNMR (400 MHz, Chloroform-d) δ 8.91 (s, 1H), 8.29 (s, 1H), 8.05 (d, J=7.4 Hz, 2H), 7.30 (d, J=7.6 Hz, 2H), 7.16 (t, J=9.4 Hz, 1H), 5.08 (d, J=13.4 Hz, 1H), 4.81 (d, J=14.2 Hz, 1H), 4.19 (d, J=11.4 Hz, 1H), 4.04-3.94 (m, 2H), 3.93-3.61 (m, 5H), 3.55 (d, J=10.2 Hz, 2H), 3.34 (s, 1H), 3.22 (d, J=21.4 Hz, 2H), 2.86 (s, 2H), 2.74 (d, J=22.0 Hz, 1H), 2.02 (d, J=12.4 Hz, 4H), 1.77-1.62 (m, 8H), 1.52 (d, J=14.8 Hz, 3H), 1.25 (s, 1H), 1.15 (s, 4H), 0.95-0.84 (m, 2H). Further elution provided (B) (15.8 mg, 16.2%) as white solid. LCMS: 681.2 [M+H]⁺; ¹HNMR (400 MHz, Chloroform-d) δ 8.97-8.88 (m, 1H), 8.35-8.23 (m, 1H), 8.09-7.98 (m, 2H), 7.37-7.26 (m, 4H), 5.17-5.06 (m, 1H), 4.86-4.76 (m, 1H), 4.28-4.13 (m, 1H), 4.06-3.82 (m, 4H), 3.77-3.50 (m, 4H), 3.38-3.15 (m, 3H), 2.92-2.81 (m, 2H), 2.79-2.68 (m, 1H), 2.04-1.87 (m, 3H), 1.82-1.46 (m, 12H), 1.28-1.10 (m, 7H), 0.95-0.84 (m, 2H).

### Synthesis of (8-(5-((3,4-dichlorophenyl)difluoromethyl)-1,3,4-oxadiazol-2-yl)-6-azaspiro[3.4]octan-6-yl)(thiazol-5-yl)methanone: (I-11)

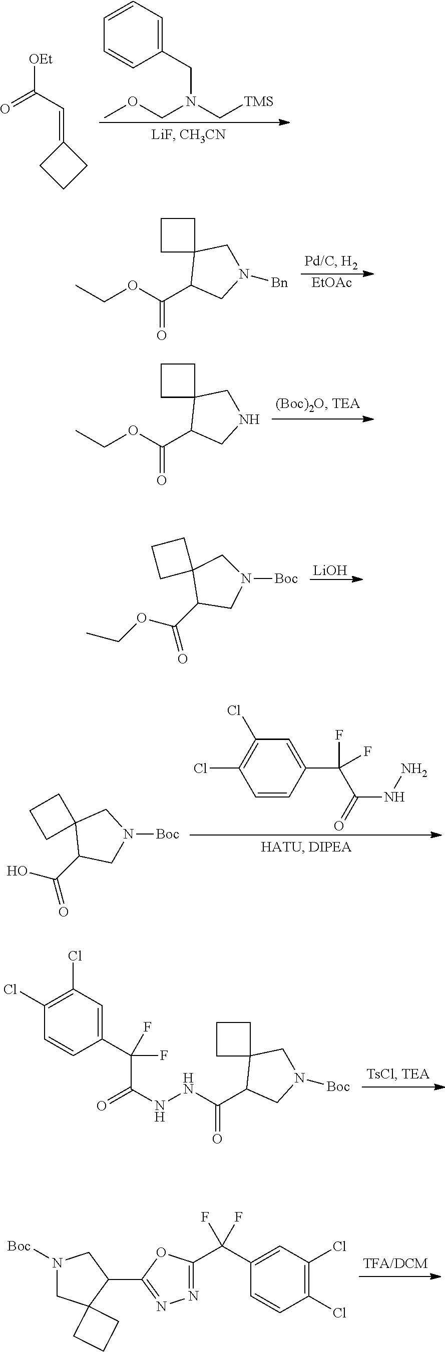

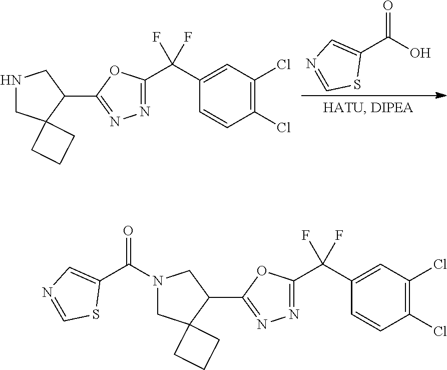

Step 1: ethyl 6-benzyl-6-azaspiro[3.4]octane-8-carboxylate: To a solution of ethyl 2-cyclobutylideneacetate (2 g, 14.28 mmol) and LiF (1.3 g, 51.40 mmol) in CH₃CN (4 mL) was added N-benzyl-1-methoxy-N-((trimethylsilyl)methyl)methanamine (4.1 g, 17.13 mmol). The reaction mixture was heated under a N₂ atmosphere at 80° C. overnight. The mixture was diluted with water (50 mL) and extracted with EtOAc (120 mL×2). The combined organic layers were washed with brine, dried over Na₂SO₄, filtered and concentrated. The residue obtained was purified by prep-TLC (eluent: Pet. Ether:EtOAc=10:1) to afford ethyl 6-benzyl-6-azaspiro[3.4]octane-8-carboxylate (1.5 g, 39%) as a colorless oil. LCMS m/z=274.2 [M+H]⁺; ¹H NMR (400 MHz, DMSO-d6) δ 7.29 (s, 4H), 7.24-7.21 (m, 1H), 4.09 (dd, J=12.6, 7.1 Hz, 2H), 3.61-3.53 (m, 3H), 2.82-2.73 (m, 3H), 2.64 (d, J=2.4 Hz, 1H), 2.11-2.00 (m, 1H), 1.96-1.87 (m, 2H), 1.73 (s, 3H), 1.20 (s, 3H).

Step 2: ethyl 6-azaspiro[3.4]octane-8-carboxylate: To a solution of ethyl 6-benzyl-6-azaspiro[3.4]octane-8-carboxylate (1.5 g, 5.50 mmol) in EtOAc (8 mL) was added 40% Pd/C (600 mg). The mixture was heated at 40° C. under a H₂ atmosphere for 2 days then the catalyst was removed by filtration through celite and the filtrate concentrated to afford ethyl 6-azaspiro[3.4]octane-8-carboxylate (682 mg, 68%) which was used without further purification. LCMS m/z=184.2 [M+H]⁺; ¹H NMR (400 MHz, DMSO-d6) δ 4.09 (dd, J=10.8, 7.2 Hz, 2H), 2.99 (d, J=8.2 Hz, 1H), 2.84 (d, J=10.6 Hz, 2H), 2.75-2.62 (m, 1H), 2.53 (d, J=4.6 Hz, 1H), 2.09-1.99 (m, 1H), 1.80 (d, J=22.8 Hz, 6H), 1.21 (s, 3H).

Step 3: 6-(tert-butyl) 8-ethyl 6-azaspiro[3.4]octane-6,8-dicarboxylate: To a solution of ethyl 6-azaspiro[3.4]octane-8-carboxylate (480 mg, 2.62 mmol) and TEA (530 mg, 5.24 mmol) in DCM (3 mL) was added (Boc)₂O (686 mg, 3.14 mmol) and the mixture stirred at room temperature for 4 h. The reaction was quenched with water (30 mL) and extracted with DCM (100 mL×2). The combined organic layers were washed with brine, dried over Na₂SO₄, filtered and concentrated. The residue obtained was purified by column chromatography on silica gel (eluent: Pet. Ether:EtOAc=10:1 to 5:1) to afford 6-(tert-butyl) 8-ethyl 6-azaspiro[3.4]octane-6,8-dicarboxylate (713 mg, 40%) as a yellow oil. LCMS m/z=284.2 [M+H]⁺; ¹H NMR (400 MHz, DMSO-d6) δ 4.20-3.97 (m, 2H), 3.44-3.33 (m, 4H), 2.98-2.90 (m, 1H), 2.24-1.98 (m, 2H), 1.88 (s, 2H), 1.79 (s, 2H), 1.39 (d, J=1.8 Hz, 9H), 1.21 (t, J=7.2 Hz, 3H).

Step 4: 6-(tert-butoxycarbonyl)-6-azaspiro[3.4]octane-8-carboxylic acid: To a solution of 6-(tert-butyl) 8-ethyl 6-azaspiro[3.4]octane-6,8-dicarboxylate (400 mg, 1.41 mmol) in a mixture of THE (2 mL), EtOH (0.5 mL) and water (0.5 mL) was added LiOH (85 mg, 3.53 mmol). The reaction mixture was stirred at room temperature for 3 h then diluted with water (30 mL) and extracted with EtOAc (80 mL). The aqueous layer was collected and acidified to pH˜2 with 4M HCl then extracted with EtOAc (100 mL×2). The combined organic layers were washed with brine, dried over Na₂SO₄, filtered and concentrated to afford 6-(tert-butoxycarbonyl)-6-azaspiro[3.4]octane-8-carboxylic acid (174 mg, 49%) as a yellow oil. LCMS m/z=254.2 [M−H]⁻; ¹H NMR (400 MHz, DMSO-d6) δ 4.03 (d, J=7.2 Hz, 1H), 3.37 (d, J=4.4 Hz, 1H), 3.33-3.27 (m, 3H), 2.84 (dd, J=7.8, 3.2 Hz, 1H), 2.15-1.98 (m, 3H), 1.89-1.79 (m, 3H), 1.39 (s, 9H).

Step 5: tert-butyl 8-(2-(2-(3,4-dichlorophenyl)-2,2-difluoroacetyl)hydrazine-1-carbonyl)-6-azaspiro[3.4]octane-6-carboxylate: To a solution of 6-(tert-butoxycarbonyl)-6-azaspiro[3.4]octane-8-carboxylic acid (170 mg, 0.67 mmol) in DCM (5 mL) was added HATU (253 mg, 0.67 mmol) and the mixture stirred at room temperature for 30 min. 2-(3,4-dichlorophenyl)-2,2-difluoroacetohydrazide (170 mg, 0.67 mmol) and DIPEA (258 mg, 1.99 mmol) were then added and the reaction stirred for another 2 h. The mixture was diluted with water (25 mL) and extracted with DCM (80 mL×2). The combined organic layers were washed with brine, dried over Na₂SO₄, filtered and concentrated. The residue obtained was purified by prep-TLC (eluent: Pet. Ether:EtOAc=1:1) to afford tert-butyl 8-(2-(2-(3,4-dichlorophenyl)-2,2-difluoroacetyl)hydrazine-1-carbonyl)-6-azaspiro[3.4]octane-6-carboxylate (250 mg, 76%) as a white solid. LCMS m/z=490.1 [M−H]⁻; ¹H NMR (400 MHz, DMSO-d6) δ 7.90 (d, J=2.2 Hz, 2H), 7.64 (dd, J=8.4, 2.1 Hz, 1H), 3.34 (s, 3H), 3.16 (d, J=1.6 Hz, 2H), 2.83 (s, 1H), 2.14 (s, 1H), 1.99 (s, 1H), 1.85 (t, J=6.4 Hz, 4H), 1.38 (d, J=2.8 Hz, 9H).

Step 6: tert-butyl 8-(5-((3,4-dichlorophenyl)difluoromethyl)-1,3,4-oxadiazol-2-yl)-6-azaspiro[3.4]octane-6-carboxylate: To a solution of tert-butyl 8-(2-(2-(3,4-dichlorophenyl)-2,2-difluoroacetyl)hydrazine-1-carbonyl)-6-azaspiro[3.4]octane-6-carboxylate (250 mg, 0.51 mmol) and TEA (258 mg, 2.55 mmol) in DCM (3 mL) was added TsCl (291 mg, 1.53 mmol). The mixture was stirred at room temperature for 1 h then was diluted with water (30 mL) and extracted with DCM (100 mL×2). The combined organic layers were washed with brine, dried over Na₂SO₄, filtered and concentrated. The residue was purified by prep-TLC (eluent: Pet. Ether:EtOAc=3:1) to afford tert-butyl 8-(5-((3,4-dichlorophenyl)difluoromethyl)-1,3,4-oxadiazol-2-yl)-6-azaspiro[3.4]octane-6-carboxylate (243 mg, 100%) as a yellow oil. LCMS m/z=418.2 [M+H-56]⁺; ¹H NMR (400 MHz, DMSO-d6) δ 8.04 (d, J=2.2 Hz, 1H), 7.90 (d, J=8.4 Hz, 1H), 7.75-7.71 (m, 1H), 3.76 (d, J=5.0 Hz, 1H), 3.64 (d, J=4.6 Hz, 2H), 3.48-3.35 (m, 2H), 2.13 (d, J=7.4 Hz, 1H), 1.99 (s, 1H), 1.84 (dd, J=13.0, 5.6 Hz, 3H), 1.68 (s, 1H), 1.40 (d, J=2.4 Hz, 9H).

Step 7: 2-((3,4-dichlorophenyl)difluoromethyl)-5-(6-azaspiro[3.4]octan-8-yl)-1,3,4-oxadiazole: To a solution of tert-butyl 8-(5-((3,4-dichlorophenyl)difluoromethyl)-1,3,4-oxadiazol-2-yl)-6-azaspiro[3.4]octane-6-carboxylate (100 mg, 0.21 mmol) in DCM (2 mL) was added TFA (1 mL) and the reaction stirred at room temperature for 1 h. The solvent was removed under vacuum to afford 2-((3,4-dichlorophenyl)difluoromethyl)-5-(6-azaspiro[3.4]octan-8-yl)-1,3,4-oxadiazole (79 mg) which was used directly in the next step. (TFA salt) LCMS m/z=374.1 [M+H]⁺.

Step 8: (8-(5-((3,4-dichlorophenyl)difluoromethyl)-1,3,4-oxadiazol-2-yl)-6-azaspiro[3.4]octan-6-yl)(thiazol-5-yl)methanone (I-11): To a solution of thiazole-5-carboxylic acid (27 mg, 0.21 mmol) in DCM (2 mL) was added HATU (89 mg, 0.23 mmol) and the mixture stirred at room temperature for 30 min. 2-((3,4-dichlorophenyl)difluoromethyl)-5-(6-azaspiro[3.4]octan-8-yl)-1,3,4-oxadiazole (79 mg, 0.21 mmol) and DIPEA (82 mg, 0.64 mmol) were added and the reaction stirred for another 2 h. The mixture was diluted with water (25 mL) and extracted with DCM (50 mL×2). The combined organic layers were washed with brine, dried over Na₂SO₄, filtered and concentrated. The residue obtained was purified by prep-HPLC to afford (8-(5-((3,4-dichlorophenyl)difluoromethyl)-1,3,4-oxadiazol-2-yl)-6-azaspiro[3.4]octan-6-yl)(thiazol-5-yl)methanone (45 mg, 45%) as a white solid. LCMS m/z=485.1 [M+H]⁺; ¹H NMR (400 MHz, DMSO-d6) δ 9.26 (d, J=7.2 Hz, 1H), 8.39 (d, J=13.6 Hz, 1H), 8.03 (dd, J=10.2, 2.2 Hz, 1H), 7.89 (d, J=8.4 Hz, 1H), 7.74 (dd, J=8.6, 2.2 Hz, 1H), 4.17 (d, J=6.2 Hz, 1H), 4.03-3.87 (m, 3H), 3.78-3.64 (m, 1H), 2.18 (d, J=8.6 Hz, 1H), 2.11-2.00 (m, 1H), 1.90 (d, J=6.2 Hz, 3H), 1.72 (d, J=12.0 Hz, 1H).

### Synthesis of (4-(5-((3,4-dichlorophenyl)difluoromethyl)-1,3,4-oxadiazol-2-yl)-8,8-dioxido-8-thia-2-azaspiro[4.5]decan-2-yl)(thiazol-5-yl)methanone I-8

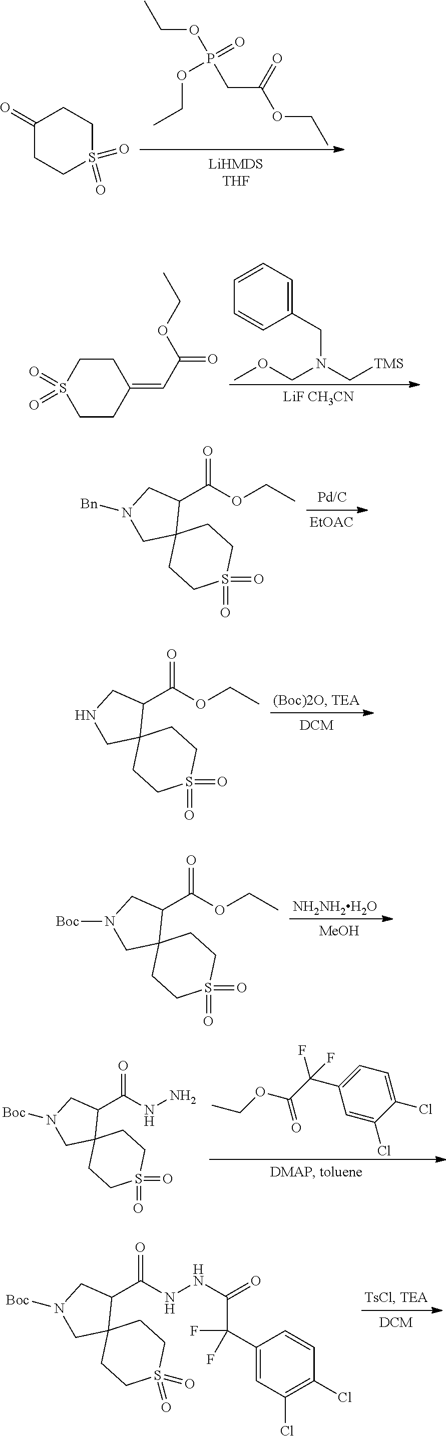

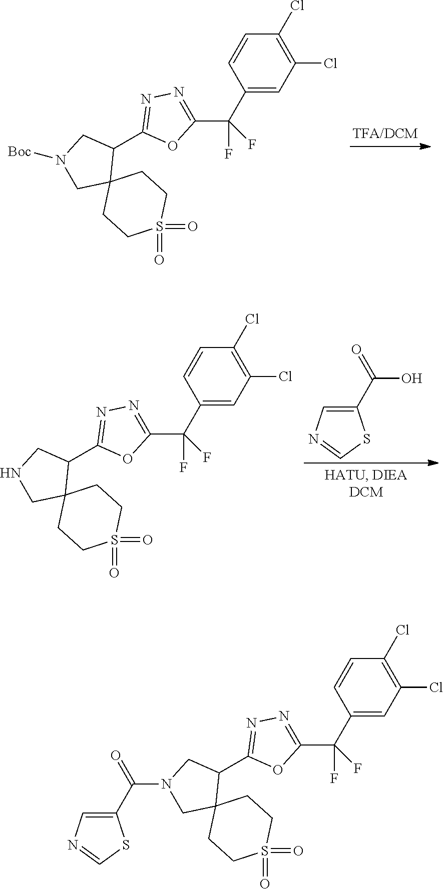 [SMILES: CC(C)(C)OC(=O)N1CC(c2nnc(C(F)(F)c3ccc(Cl)c(Cl)c3)o2)C2(CCS(=O)(=O)CC2)C1 ; O=S1(=O)CCC2(CC1)CNCC2c1nnc(C(F)(F)c2ccc(Cl)c(Cl)c2)o1 ; O=C(O)c1cncs1 ; O=C(c1cncs1)N1CC(c2nnc(C(F)(F)c3ccc(Cl)c(Cl)c3)o2)C2(CCS(=O)(=O)CC2)C1]

Step 1: ethyl 2-(1,1-dioxidotetrahydro-4H-thiopyran-4-ylidene)acetate: To a solution of ethyl 2-(diethoxyphosphoryl)acetate (1.5 g, 6.7 mmol) in anhydrous THE (10 mL) at 0° C. under N₂ atmosphere was added LiHMIDS (1.0 M in THF, 1.2 g, 7.4 mmol) dropwise. The reaction mixture was stirred at 0° C. for 30 min then tetrahydro-4H-thiopyran-4-one 1,1-dioxide (1 g, 6.7 mmol) was added. The reaction was warmed to room temperature and stirred for 1.5 h. The reaction was diluted with EtOAc (200 mL) and the organic layer washed with sat.NH₄Cl (100 mL), dried over Na₂SO₄, filtered and concentrated. The residue was purified by column chromatography on silica gel (eluent: Pet. Ether:EtOAc=4:1) to afford ethyl 2-(1,1-dioxidotetrahydro-4H-thiopyran-4-ylidene)acetate (570 mg, 41%) as a white solid. LCMS m/z=219.1 [M+H]⁺; ¹H NMR (400 MHz, DMSO-d₆) δ 5.91 (s, 1H), 4.14-4.06 (m, 2H), 3.27-3.18 (m, 6H), 2.75-2.68 (m, 2H), 1.23-1.17 (m, 3H).

Step 2: ethyl 2-benzyl-8-thia-2-azaspiro[4.5]decane-4-carboxylate 8,8-dioxide: A mixture of ethyl 2-(1,1-dioxidotetrahydro-4H-thiopyran-4-ylidene)acetate (2.2 g, 10.1 mmol), N-benzyl-1-methoxy-N-((trimethylsilyl)methyl)methanamine (6 g, 25.2 mmol) and LiF (0.78 g, 0.03 mmol) in acetonitrile (20 mL) was heated at 80° C. for 16 h. After cooling to room temperature, the mixture was diluted with water (100 mL) and extracted with EtOAc (100 mL×3). The combined organic layers were washed with brine, dried over Na₂SO₄, filtered and concentrated. The mixture was purified by column chromatography on silica gel (eluent: Pet. Ether:EtOAc=3:1) to afford ethyl 2-benzyl-8-thia-2-azaspiro[4.5]decane-4-carboxylate 8,8-dioxide (2.7 g, 77%) as a yellow oil. LCMS m/z=352.1 [M+H]⁺; H NMR (400 MHz, DMSO-d₆) δ 7.34-7.29 (m, 4H), 7.26-7.21 (m, 1H), 4.18-4.02 (m, 2H), 3.67-3.57 (m, 2H), 3.19-3.16 (m, 1H), 3.13-3.00 (m, 3H), 2.82-2.63 (m, 5H), 2.34-2.25 (m, 1H), 2.04-1.90 (m, 2H), 1.82-1.73 (m, 1H), 1.23-1.18 (m, 3H).

Step 3: ethyl 8-thia-2-azaspiro[4.5]decane-4-carboxylate 8,8-dioxide: To a solution of ethyl 2-benzyl-8-thia-2-azaspiro[4.5]decane-4-carboxylate 8,8-dioxide (200 mg, 0.6 mmol) in EtOAc (2 mL) was added 10% Pd/C (80 mg). The reaction mixture was stirred under a H₂ atmosphere for 24 h. The mixture was filtered and concentrated to afford ethyl 8-thia-2-azaspiro[4.5]decane-4-carboxylate 8,8-dioxide (162 mg, 100%) which was used directly in the next step. LCMS m/z=262.1 [M+H]⁺; ¹H NMR (400 MHz, DMSO-d₆) δ 4.19-3.99 (m, 2H), 3.16 (s, 1H), 3.13-2.89 (m, 6H), 2.75-2.61 (m, 2H), 2.32-2.19 (m, 1H), 1.98-1.65 (m, 4H), 1.24-1.16 (m, 3H).

Step 4: 2-(tert-butyl) 4-ethyl 8-thia-2-azaspiro[4.5]decane-2,4-dicarboxylate 8,8-dioxide: To a solution of ethyl 8-thia-2-azaspiro[4.5]decane-4-carboxylate 8,8-dioxide (160 mg, 0.6 mmol) in DCM (2 mL) was added TEA (124 mg, 1.2 mmol) and (Boc)₂O (200 mg, 0.9 mmol). The reaction mixture was stirred at room temperature for 1.5 h then was diluted with water (20 mL), extracted with DCM (30 mL×3). The combined organic layers were washed with brine, dried over Na₂SO₄, filtered and purified by column chromatography on silica gel (eluent: DCM:MeOH=70:1) to afford 2-(tert-butyl) 4-ethyl 8-thia-2-azaspiro[4.5]decane-2,4-dicarboxylate 8,8-dioxide (150 mg, 68%) as a colorless oil. LCMS m/z=306 [M+H-56]⁺; ¹H NMR (400 MHz, DMSO-d₆) δ 4.21-4.03 (m, 3H), 3.58-3.45 (m, 3H), 3.16 (d, J=5.1 Hz, 2H), 3.13-3.09 (m, 2H), 2.32 (d, J=6.9 Hz, 1H), 2.07-1.90 (m, 2H), 1.88-1.79 (m, 1H), 1.75-1.60 (m, 1H), 1.40 (s, 9H), 1.23-1.18 (m, 3H).

Step 5: tert-butyl 4-(hydrazinecarbonyl)-8-thia-2-azaspiro[4.5]decane-2-carboxylate 8,8-dioxide: To a solution of 2-(tert-butyl) 4-ethyl 8-thia-2-azaspiro[4.5]decane-2,4-dicarboxylate 8,8-dioxide (50 mg, 0.1 mmol) in MeOH (0.5 mL) was added N₂H₄·H₂O (98%, 3 drops). The reaction mixture was heated at 80° C. for 24 h then was diluted with water (10 mL) and extracted with EtOAc (10 mL×3). The combined organic layers were washed with brine, dried over Na₂SO₄, filtered and concentrated to afford tert-butyl 4-(hydrazinecarbonyl)-8-thia-2-azaspiro[4.5]decane-2-carboxylate 8,8-dioxide (150 mg, 88%) as a colorless oil. LCMS m/z=292.2 [M+H-56]⁺; ¹H NMR (400 MHz, DMSO-d₆) δ 4.31-4.26 (m, 1H), 3.48-3.40 (m, 1H), 3.32-3.21 (m, 3H), 3.11-2.99 (m, 4H), 2.75-2.69 (m, 1H), 2.01-1.82 (m, 4H), 1.41-1.38 (m, 9H).

Step 6: tert-butyl 4-(2-(2-(3,4-dichlorophenyl)-2,2-difluoroacetyl)hydrazine-1-carbonyl)-8-thia-2-azaspiro[4.5]decane-2-carboxylate 8,8-dioxide: To a solution of ethyl 2-(3,4-dichlorophenyl)-2,2-difluoroacetate (130 mg, 0.4 mmol) in toluene (2 mL) was added tert-butyl 4-(hydrazinecarbonyl)-8-thia-2-azaspiro[4.5]decane-2-carboxylate 8,8-dioxide (167 mg, 0.4 mmol) and DMAP (30 mg, 0.2 mmol). The reaction mixture was heated at 100° C. for 24 h then was diluted with water (10 mL) and extracted with EtOAc (20 mL×3). The combined organic layers were washed with brine, dried over Na₂SO₄, filtered and and purified by RP-column to afford tert-butyl 4-(2-(2-(3,4-dichlorophenyl)-2,2-difluoroacetyl)hydrazine-1-carbonyl)-8-thia-2-azaspiro[4.5]decane-2-carboxylate 8,8-dioxide (100 mg, 36%) as a colorless oil. LCMS m/z=469.9 [M+H-100]⁺; ¹H NMR (400 MHz, DMSO-d₆) δ 7.91-7.85 (m, 2H), 7.66-7.61 (m, 1H), 3.57-3.41 (m, 2H), 3.30-3.28 (m, 2H), 3.18-3.15 (m, 1H), 3.14-3.07 (m, 2H), 3.06-2.98 (m, 1H), 2.93-2.88 (m, 1H), 2.08-1.86 (m, 4H), 1.42-1.37 (m, 9H).

Step 7: tert-butyl 4-(5-((3,4-dichlorophenyl)difluoromethyl)-1,3,4-oxadiazol-2-yl)-8-thia-2-azaspiro[4.5]decane-2-carboxylate 8,8-dioxide: To a solution of tert-butyl 4-(2-(2-(3,4-dichlorophenyl)-2,2-difluoroacetyl)hydrazine-1-carbonyl)-8-thia-2-azaspiro[4.5]decane-2-carboxylate 8,8-dioxide (90 mg, 0.1 mmol) in DCM (1 mL) was added TEA (80 mg, 0.7 mmol) and TsCl (90 mg, 0.4 mmol). The reaction mixture was stirred at room temperature for 2 h then was concentrated and the residue obtained purified by RP-column to afford tert-butyl 4-(5-((3,4-dichlorophenyl)difluoromethyl)-1,3,4-oxadiazol-2-yl)-8-thia-2-azaspiro[4.5]decane-2-carboxylate 8,8-dioxide (40 mg, 46%) as a white solid. LCMS m/z=469.9 [M+H-56]⁺; ¹H NMR (400 MHz, DMSO-d₆) δ 8.06-8.03 (m, 1H), 7.92-7.88 (m, 1H), 7.74-7.70 (m, 1H), 3.97-3.91 (m, 1H), 3.79-3.71 (m, 2H), 3.60-3.54 (m, 1H), 3.38-3.33 (m, 1H), 3.21-3.10 (m, 3H), 3.01-2.92 (m, 1H), 2.36-2.17 (m, 2H), 2.14-2.05 (m, 1H), 1.83-1.74 (m, 1H), 1.43-1.40 (m, 9H).

Step 8: 4-(5-((3,4-dichlorophenyl)difluoromethyl)-1,3,4-oxadiazol-2-yl)-8-thia-2-azaspiro[4.5]decane 8,8-dioxide: To a solution of tert-butyl 4-(5-((3,4-dichlorophenyl)difluoromethyl)-1,3,4-oxadiazol-2-yl)-8-thia-2-azaspiro[4.5]decane-2-carboxylate 8,8-dioxide (40 mg, 0.07 mmol) in DCM (0.8 mL) was added TFA (0.2 mL). The reaction mixture was stirred at room temperature for 40 min. The solvent was removed under vacuum to afford crude 4-(5-((3,4-dichlorophenyl)difluoromethyl)-1,3,4-oxadiazol-2-yl)-8-thia-2-azaspiro[4.5]decane 8,8-dioxide (33 mg, 100%) which was used directly in the next step. LCMS m/z=451.9 [M+H]⁺.

Step 9: (4-(5-((3,4-dichlorophenyl)difluoromethyl)-1,3,4-oxadiazol-2-yl)-8,8-dioxido-8-thia-2-azaspiro[4.5]decan-2-yl)(thiazol-5-yl)methanone: (I-8): To a solution of thiazole-5-carboxylic acid (6 mg, 0.04 mmol) in DCM (0.5 mL) was added HATU (19 mg, 0.05 mmol) and DIPEA (24 mg, 0.05 mmol) and the mixture stirred at room temperature for 30 min. 4-(5-((3,4-dichlorophenyl)difluoromethyl)-1,3,4-oxadiazol-2-yl)-8-thia-2-azaspiro[4.5]decane 8,8-dioxide (21 mg, 0.04 mmol) was added and stirring continued for 2 h. The mixture was diluted with water (10 mL) and extracted with DCM (30 mL×3). The combined organic layers were washed with brine, dried over Na₂SO₄, filtered and concentrated. The mixture was purified by prep-TLC (eluent: DCM:MeOH=10:1) to afford (4-(5-((3,4-dichlorophenyl)difluoromethyl)-1,3,4-oxadiazol-2-yl)-8,8-dioxido-8-thia-2-azaspiro[4.5]decan-2-yl)(thiazol-5-yl)methanone (10.3 mg, 39% yield) as a white solid. LCMS m/z=563.1 [M+H]⁻; ¹H NMR (400 MHz, Chloroform-d) δ 8.97 (s, 1H), 8.29 (s, 1H), 7.78 (s, 1H), 7.65-7.60 (m, 1H), 7.52-7.48 (m, 1H), 4.43-4.03 (m, 3H), 3.81-3.67 (m, 2H), 3.19-2.98 (m, 4H), 2.69-2.46 (m, 1H), 2.26-2.14 (m, 1H), 2.03-1.92 (m, 1H), 1.87-1.78 (m, 1H).

### Synthesis of 8-(5-((3,4-dichlorophenyl)difluoromethyl)-1,3,4-oxadiazol-2-yl)-2-((S)-2,2-dimethylcyclopropane-1-carbonyl)-6-(thiazol-5-ylmethyl)-2,6-diazaspiro[3.4]octan-5-one: I-6

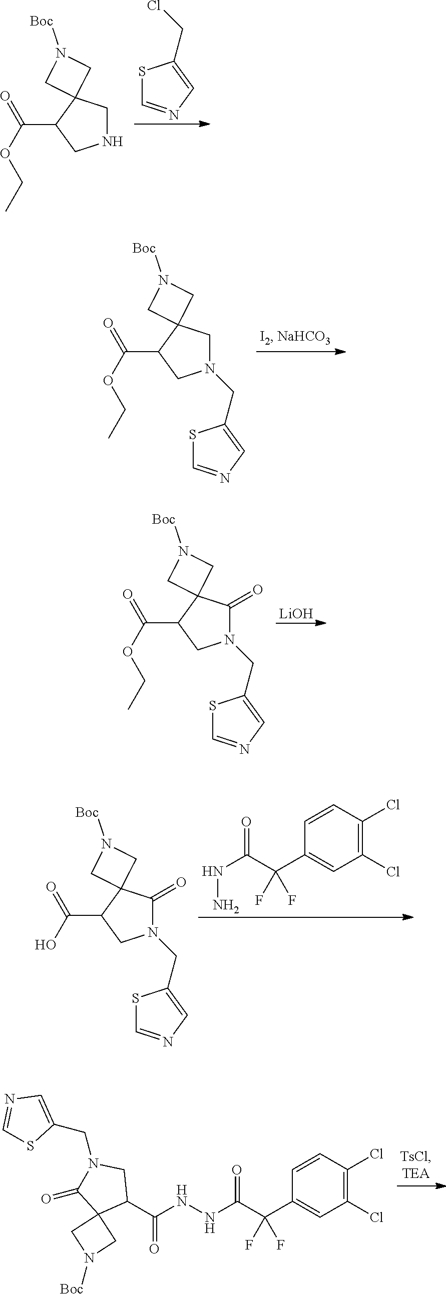

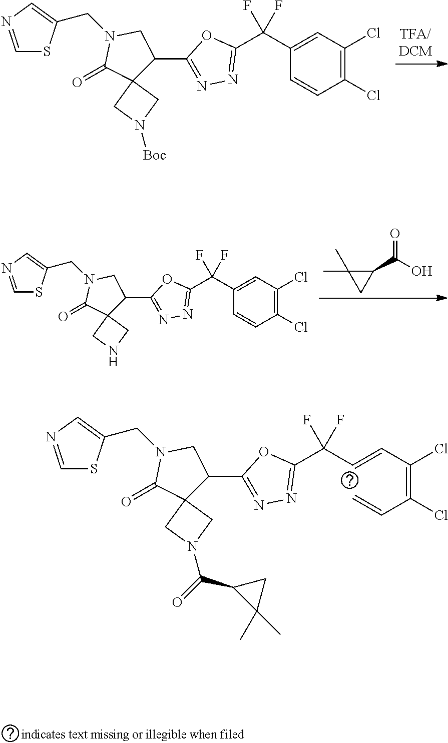

Step 1: 2-(tert-butyl) 8-ethyl 6-(thiazol-5-ylmethyl)-2,6-diazaspiro[3.4]octane-2,8-dicarboxylate): To a solution of 2-(tert-butyl) 8-ethyl 2,6-diazaspiro[3.4]octane-2,8-dicarboxylate (836 mg, 2.94 mmol) in MeCN (15 mL) was added Cs₂CO₃ (2.87 g, 8.82 mmol) and 5-(chloromethyl)thiazole (500 mg, 2.94 mmol). The reaction was heated at 40° C. for 4 h then was diluted with water (40 mL) and extracted with EtOAc (100 mL×3). The combined organic layers were washed with brine, dried over Na₂SO₄, filtered and concentrated. The residue was purified by prep-TLC (eluent: DCM:MeOH=30:1) to afford 2-(tert-butyl) 8-ethyl 6-(thiazol-5-ylmethyl)-2,6-diazaspiro[3.4]octane-2,8-dicarboxylate (800 mg, 71%) as yellow oil. LCMS m/z=382.2 [M+H]⁺; ¹H NNR (400 MHz, Chloroform-d) δ 8.75 (s, 1H), 7.71 (s, 1H), 4.18 (m, 2H), 3.97-3.78 (m, 5H), 3.70 (d, J 9.4 Hz, 1H), 2.91-3.09 (m, 3H), 2.88-2.72 (m, 2H), 1.41 (s, 9H), 1.28 (t, J=7.2 Hz, 3H).

Step 2: 2-(tert-butyl) 8-ethyl 5-oxo-6-(thiazol-5-ylmethyl)-2,6-diazaspiro[3.4]octane-2,8-dicarboxylate: To a solution of 2-(tert-butyl) 8-ethyl 6-(thiazol-5-ylmethyl)-2,6-diazaspiro[3.4]octane-2,8-di carboxylate (800 mg, 2.099 mmol) in a mixture of TH-T and water (15 mL/6 mL) was added iodine (5.33 g, 20.99 mmol) and sodium bicarbonate (1.76 g, 20.99 mmol). The mixture was stirred at room temperature overnight then was diluted with saturated sodium thiosulfate solution (50 mL) and extracted with DCM (100 mL×3). The combined organic layers were washed with brine, dried over Na₂SO₄, filtered and concentrated. The residue was purified by prep-TLC (eluent: DCM:MeOH=30:1) to afford 2-(tert-butyl) 8-ethyl 5-oxo-6-(thiazol-5-ylmethyl)-2,6-diazaspiro[3.4]octane-2,8-dicarboxylate (674 mg, 81%) as a yellow oil. LCMS m/z=395.2 [M+H]⁺; ¹H NIMR (400 MHz, DMSO-d₆) δ 9.04 (s, 1H), 7.83 (s, 1H), 5.75 (s, 1H), 4.79-4.54 (m, 2H), 4.21-4.03 (m, 2H), 3.97-3.65 (m, 4H), 3.54 (dd, J=7.6, 5.6 Hz, 1H), 3.45 (t, J=9.0 Hz, 1H), 1.37 (d, J=8.2 Hz, 9H), 1.16 (t, J=7.2 Hz, 3H).

Step 3: 2-(tert-butoxycarbonyl)-5-oxo-6-(thiazol-5-ylmethyl)-2,6-diazaspiro[3.4]octane-8-carboxylic acid: To a solution of 2-(tert-butyl) 8-ethyl 5-oxo-6-(thiazol-5-ylmethyl)-2,6-diazaspiro[3.4]octane-2,8-dicarboxylate (670 mg, 1.69 mmol) in a mixture of THF, MeOH and water (4 mL/1 mL/1 mL) was added LiOH (7 mg, 5.08 mmol) and the reaction stirred at room temperature for 2 h then diluted with water (50 mL) and extracted with EtOAc (50 mL). The aqueous layer was collected, acidified to pH ˜5 with 1M HCl and extracted with EtOAc (100 mL×3). The combined organic layers were washed with brine, dried over Na₂SO₄, filtered and concentrated to afford 2-(tert-butoxycarbonyl)-5-oxo-6-(thiazol-5-ylmethyl)-2,6-diazaspiro[3.4]octane-8-carboxylic acid (510 mg, 81%) as a white solid which was used directly in the next step. LCMS m/z=367.1 [M+H]⁺; ¹H NMR (400 MHz, DMSO-d6) δ 9.03 (s, 1H), 7.82 (s, 1H), 4.73-4.58 (m, 2H), 3.84 (d, J=46.2 Hz, 4H), 3.40-3.36 (m, 3H), 1.38 (s, 9H).

Step 4: tert-butyl 8-(2-(2-(3,4-dichlorophenyl)-2,2-difluoroacetyl)hydrazine-1-carbonyl)-5-oxo-6-(thiazol-5-ylmethyl)-2,6-diazaspiro[3.4]octane-2-carboxylate: To a solution of 2-(tert-butoxycarbonyl)-5-oxo-6-(thiazol-5-ylmethyl)-2,6-diazaspiro[3.4]octane-8-carboxylic acid (510 mg, 1.39 mmol) in DCM (10 mL) was added HATU (528 mg, 1.39 mmol) and DIPEA (539 mg, 4.16 mmol) and the mixture stirred at room temperature for 30 min. 2-(3,4-dichlorophenyl)-2,2-difluoroacetohydrazide (354 mg, 1.39 mmol) was added and stirring continued for 2 h. The reaction was diluted with water (100 mL) and extracted with DCM (100 mL×3). The combined organic layers were washed with brine, dried over Na₂SO₄, filtered and concentrated. The mixture was purified by prep-TLC (eluent: DCM:MeOH=10:1) to afford tert-butyl 8-(2-(2-(3,4-dichlorophenyl)-2,2-difluoroacetyl)hydrazine-1-carbonyl)-5-oxo-6-(thiazol-5-ylmethyl)-2,6-diazaspiro[3.4]octane-2-carboxylate (500 mg, 59%) as a yellow oil. LCMS m/z=548.0 [M+H]⁺.

Step 5: tert-butyl 8-(5-((3,4-dichlorophenyl)difluoromethyl)-1,3,4-oxadiazol-2-yl)-5-oxo-6-(thiazol-5-ylmethyl)-2,6-diazaspiro[3.4]octane-2-carboxylate: To a solution of tert-butyl 8-(2-(2-(3,4-dichlorophenyl)-2,2-difluoroacetyl)hydrazine-1-carbonyl)-5-oxo-6-(thiazol-5-ylmethyl)-2,6-diazaspiro[3.4]octane-2-carboxylate (500 mg, 0.829 mmol) in DCM (10 mL) was added TEA (419 mg, 4.145 mmol) and TsCl (474 mg, 2.487 mmol). The reaction was stirred at room temperature for 2 h then was diluted with water (100 mL) and extracted with EtOAc (150 mL×3). The combined organic layers were washed with brine, dried over Na₂SO₄, filtered and concentrated. The residue was purified by RP-column (48% MeCN in water) to afford tert-butyl 8-(5-((3,4-dichlorophenyl)difluoromethyl)-1,3,4-oxadiazol-2-yl)-5-oxo-6-(thiazol-5-ylmethyl)-2,6-diazaspiro[3.4]octane-2-carboxylate (130 g, 27%) as a yellow oil. LCMS m/z=529.9 [M+H]⁺; ¹H NMR (400 MHz, Methanol-d₄) δ 8.96 (s, 1H), 7.90-7.80 (m, 2H), 7.74 (d, J=8.6 Hz, 1H), 7.60 (dd, J=8.6, 2.2 Hz, 1H), 4.82-4.69 (m, 2H), 4.26 (dd, J=7.4, 5.6 Hz, 1H), 4.16 (s, 1H), 4.09 (d, J=7.8 Hz, 1H), 4.01 (d, J=9.2 Hz, 1H), 3.80 (dd, J=10.4, 7.6 Hz, 1H), 3.68 (dd, J=10.6, 5.4 Hz, 2H), 1.43 (s, 9H).

Step 6: 8-(5-((3,4-dichlorophenyl)difluoromethyl)-1,3,4-oxadiazol-2-yl)-6-(thiazol-5-ylmethyl)-2,6-diazaspiro[3.4]octan-5-one: To a solution of tert-butyl 8-(5-((3,4-dichlorophenyl)difluoromethyl)-1,3,4-oxadiazol-2-yl)-5-oxo-6-(thiazol-5-ylmethyl)-2,6-diazaspiro[3.4]octane-2-carboxylate (70 mg, 0.12 mmol) in DCM (2 mL) was added TFA (1.5 mL) and the reaction stirred at room temperature for 1 h. The solvent was removed under reduced pressure to afford 8-(5-((3,4-dichlorophenyl)difluoromethyl)-1,3,4-oxadiazol-2-yl)-6-(thiazol-5-ylmethyl)-2,6-diazaspiro[3.4]octan-5-one (58 mg, 100%) which was used directly in the next step. LCMS m/z=486.0 [M+H]⁺.

Step 7: 8-(5-((3,4-dichlorophenyl)difluoromethyl)-1,3,4-oxadiazol-2-yl)-2-((S)-2,2-dimethylcyclopropane-1-carbonyl)-6-(thiazol-5-ylmethyl)-2,6-diazaspiro[3.4]octan-5-on: To a solution of (S)-2,2-dimethylcyclopropane-1-carboxylic acid (13 mg, 0.113 mmol) in DCM (2 mL) was added HATU (43 mg, 0.113 mmol) and DIPEA (44 mg, 0.34 mmol) and the mixture stirred at room temperature for 30 min. 8-(5-((3,4-dichlorophenyl)difluoromethyl)-1,3,4-oxadiazol-2-yl)-6-(thiazol-5-ylmethyl)-2,6-diazaspiro[3.4]octan-5-one (55 mg, 0.113 mmol) was added and stirring continued for 2 h. The mixture was diluted with water (20 mL) and extracted with DCM (30 mL×3). The combined organic layers were washed with brine, dried over Na₂SO₄, filtered and concentrated. The residue was purified by prep-HPLC to afford 8-(5-((3,4-dichlorophenyl)difluoromethyl)-1,3,4-oxadiazol-2-yl)-2-((S)-2,2-dimethylcyclopropane-1-carbonyl)-6-(thiazol-5-ylmethyl)-2,6-diazaspiro[3.4]octan-5-one (25 mg, 25%) as a white solid. LCMS m/z=582.0 [M+H]⁻; ¹H NMR (400 MHz, Methanol-d₄) δ 8.97 (d, J=3.8 Hz, 1H), 7.86 (d, J=7.0 Hz, 2H), 7.75 (m, 1H), 7.61 (d, J=10.0 Hz, 1H), 4.88 (d, J=6.2 Hz, 1H), 4.78 (td, J=9.6, 4.8 Hz, 2H), 4.47-4.36 (m, 1H), 4.29 (d, J=9.6 Hz, 1H), 4.11 (m, 2H), 3.84 (dd, J=10.6, 7.2 Hz, 1H), 3.79-3.64 (m, 1H), 1.47-1.27 (m, 1H), 1.22-1.00 (m, 7H), 0.83-0.73 (m, 1H).

### Synthesis of (S)-6-((1-benzyl-1H-pyrazol-4-yl)methyl)-N-((2S,3R)-3-(cyclohexylmethoxy)-1-oxo-1-(4-(thiazol-2-yl)piperidin-1-yl)butan-2-yl)-2-((S)-2,2-dimethylcyclopropane-1-carbonyl)-5-oxo-2,6-diazaspiro[3.4]octane-8-carboxamide (I-5)

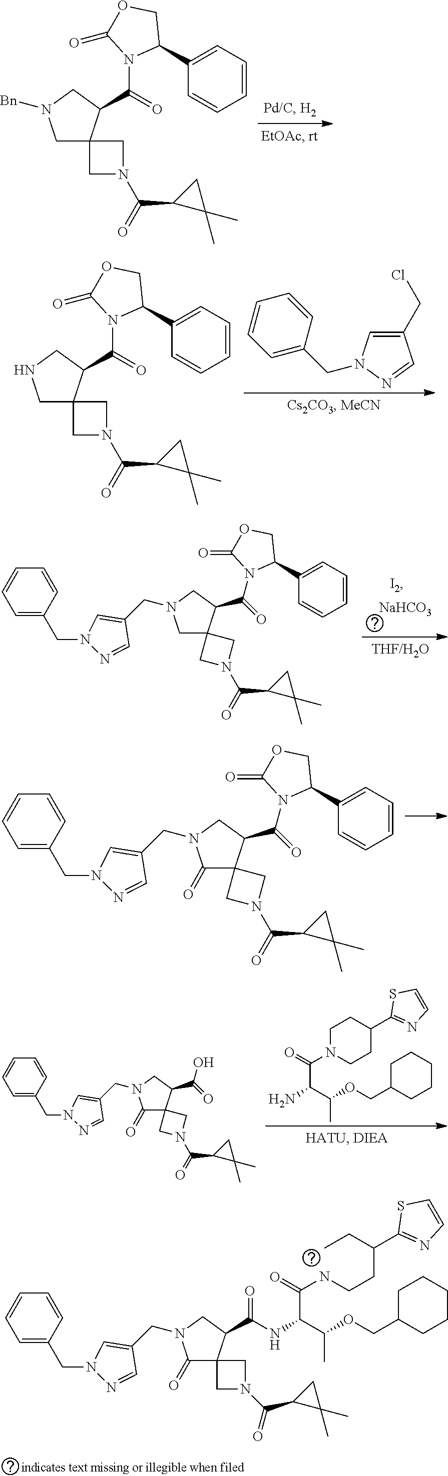

Step 1: (R)-3-((S)-2-((S)-2,2-dimethylcyclopropane-1-carbonyl)-2,6-diazaspiro[3.4]octane-8-carbonyl)-4-phenyloxazolidin-2-one: To a solution of (R)-3-((S)-6-benzyl-2-((S)-2,2-dimethylcyclopropane-1-carbonyl)-2,6-diazaspiro[3.4]octane-8-carbonyl)-4-phenyloxazolidin-2-one (1.0 g, 2.05 mmol) in EtOAc (8 mL) was added 10% Pd/C (300 mg). The reaction mixture was stirred under a H₂ atmosphere for 24 h then the catalyst was removed by filtration through Celite and the filtrate concentrated to afford (R)-3-((S)-2-((S)-2,2-dimethylcyclopropane-1-carbonyl)-2,6-diazaspiro[3.4]octane-8-carbonyl)-4-phenyloxazolidin-2-one (700 g, 86%) which was used directly in the next step. LCMS m/z=398.1 [M+H]⁺.

Step 2: (R)-3-((S)-6-((1-benzyl-1H-pyrazol-4-yl)methyl)-2-((S)-2,2-dimethylcyclopropane-1-carbonyl)-2,6-diazaspiro[3.4]octane-8-carbonyl)-4-phenyloxazolidin-2-one: To a solution of (R)-3-((S)-2-((S)-2,2-dimethylcyclopropane-1-carbonyl)-2,6-diazaspiro[3.4]octane-8-carbonyl)-4-phenyloxazolidin-2-one (1.0 g, 2.51 mmol) in MeCN (10 mL) was added 1-benzyl-4-(chloromethyl)-1H-pyrazole (520 mg, 2.51 mmol) and Cs₂CO₃ (2.45 g, 7.53 mmol). The reaction was heated at 40° C. for 4 h then was diluted with water (30 mL) and extracted with EtOAc (50 mL×2). The combined organic layers were washed with brine, dried over Na₂SO₄, filtered and concentrated. The residue was purified by prep-TLC (DCM:MeOH=15:1) to afford (R)-3-((S)-6-((1-benzyl-1H-pyrazol-4-yl)methyl)-2-((S)-2,2-dimethylcyclopropane-1-carbonyl)-2,6-diazaspiro[3.4]octane-8-carbonyl)-4-phenyloxazolidin-2-one (300 mg, 21%) as a yellow oil. LCMS m/z=568.2 [M+H]⁺; ¹H NMR (400 MHz, DMSO-d6) δ 7.66 (s, 1H), 7.38-7.24 (m, 9H), 7.19-7.14 (m, 2H), 5.48 (m, 1H), 5.27 (s, 2H), 4.73 (t, J=8.6 Hz, 1H), 4.35-4.23 (m, 1H), 4.15 (m, 2H), 4.10-3.99 (m, 1H), 3.85 (dd, J=9.2, 5.9 Hz, 1H), 3.75 (d, J=9.2 Hz, 1H), 3.62 (dd, J=14.8, 9.6 Hz, 1H), 3.41 (d, J=4.2 Hz, 2H), 3.21 (m, 1H), 2.93 (d, J=8.9 Hz, 1H), 2.33 (m, 1H), 1.35 (m, 1H), 1.12-0.99 (m, 6H), 0.85 (q, J=5.0 Hz, 1H), 0.66 (dd, J=8.0, 3.8 Hz, 1H).

Step 3: (R)-3-((S)-6-((1-benzyl-1H-pyrazol-4-yl)methyl)-2-((S)-2,2-dimethylcyclopropane-1-carbonyl)-5-oxo-2,6-diazaspiro[3.4]octane-8-carbonyl)-4-phenyloxazolidin-2-one: To a solution of (R)-3-((S)-6-((1-benzyl-1H-pyrazol-4-yl)methyl)-2-((S)-2,2-dimethylcyclopropane-1-carbonyl)-2,6-diazaspiro[3.4]octane-8-carbonyl)-4-phenyloxazolidin-2-one (450 mg, 0.79 mmol) in a mixture of THF and water (10 mL/5 mL) was added iodine (1.50 g, 5.94 mmol) and sodium bicarbonate (500 mg, 5.949 mmol). The mixture was stirred at room temperature overnight then was diluted with saturated sodium thiosulfate solution (50 mL) and extracted with DCM (100 mL×3). The combined organic layers were washed with brine, dried over Na₂SO₄, filtered and concentrated. The residue was purified by prep-TLC (eluent: DCM:MeOH=15:1) to afford (R)-3-((S)-6-((1-benzyl-1H-pyrazol-4-yl)methyl)-2-((S)-2,2-dimethylcyclopropane-1-carbonyl)-5-oxo-2,6-diazaspiro[3.4]octane-8-carbonyl)-4-phenyloxazolidin-2-one (45 mg, 10%) as a yellow oil. ¹H NMR (400 MHz, Methanol-d₄) δ 7.54-7.44 (m, 1H), 7.40-7.17 (m, 10H), 7.14 (d, J=7.2 Hz, 1H), 5.48 (q, J=8.4, 7.8 Hz, 1H), 5.30 (s, 1H), 5.18 (q, J=15.2 Hz, 1H), 4.79-4.74 (m, 1H), 4.56 (s, 1H), 4.50-4.19 (m, 5H), 4.17-4.01 (m, 2H), 3.92 (m, 1H), 3.54-3.45 (m, 1H), 1.29 (s, 3H), 1.18-1.11 (m, 6H).

Step 4: (S)-6-((1-benzyl-1H-pyrazol-4-yl)methyl)-2-((S)-2,2-dimethylcyclopropane-1-carbonyl)-5-oxo-2,6-diazaspiro[3.4]octane-8-carboxylic acid: To a solution of (R)-3-((S)-6-((1-benzyl-1H-pyrazol-4-yl)methyl)-2-((S)-2,2-dimethylcyclopropane-1-carbonyl)-5-oxo-2,6-diazaspiro[3.4]octane-8-carbonyl)-4-phenyloxazolidin-2-one (45 mg, 0.077 mmol) in a mixture of THF and water (1.5 mL/0.5 mL) at 0° C. was added a solution of lithium hydroxide monohydrate (4 mg, 0.155 mmol) and 30% H₂O₂ (17 mg, 0.155 mmol) in water (0.2 mL). The reaction was stirred at 0° C. for 2 h then was diluted with water (10 mL) and extracted with EtOAc (30 mL). The aqueous layer was collected, acidified to pH ˜3 with 1M HCl and extracted with EtOAc (50 mL×3). The combined organic layers were washed with brine, dried over Na₂SO₄, filtered and concentrated to afford (S)-6-((1-benzyl-1H-pyrazol-4-yl)methyl)-2-((S)-2,2-dimethylcyclopropane-1-carbonyl)-5-oxo-2,6-diazaspiro[3.4]octane-8-carboxylic acid (17 mg, 51%) as a colorless oil which was used directly in the next step. LCMS m/z=437.2 [M+H]⁺; ¹H NMR (400 MHz, Chloroform-d) δ 7.90-7.79 (m, 1H), 7.42-7.29 (m, 4H), 7.25-7.17 (m, 2H), 5.29 (d, J=9.6 Hz, 2H), 4.56 (s, 1H), 4.20-3.84 (m, 4H), 3.75-3.57 (m, 3H), 3.47 (t, J=6.6 Hz, 1H), 1.58 (dd, J=8.6, 6.4 Hz, 1H), 1.26 (s, 6H), 0.92 (t, J=7.4 Hz, 2H).

Step 5: (S)-6-((1-benzyl-1H-pyrazol-4-yl)methyl)-N-((2S,3R)-3-(cyclohexylmethoxy)-1-oxo-1-(4-(thiazol-2-yl)piperidin-1-yl)butan-2-yl)-2-((S)-2,2-dimethylcyclopropane-1-carbonyl)-5-oxo-2,6-diazaspiro[3.4]octane-8-carboxamide (I-5): To a solution of (S)-6-((1-benzyl-1H-pyrazol-4-yl)methyl)-2-((S)-2,2-dimethylcyclopropane-1-carbonyl)-5-oxo-2,6-diazaspiro[3.4]octane-8-carboxylic acid (12 mg, 0.028 mmol) in DCM (1 mL) was added HATU (10 mg, 0.028 mmol) and DIPEA (10 mg, 0.084 mmol) and the mixture stirred at room temperature for 30 min. Methyl (2S,3R)-2-amino-3-(cyclohexylmethoxy)-1-(4-(thiazol-2-yl)piperidin-1-yl)butan-1-one (12 mg, 0.028 mmol) was added and stirring continued for 1.5 h. The mixture was diluted with water (20 mL) and extracted with EtOAc (30 mL×3). The combined organic layers were washed with brine, dried over Na₂SO₄, filtered and concentrated. The residue obtained was purified by prep-HPLC to afford (S)-6-((1-benzyl-1H-pyrazol-4-yl)methyl)-N-((2S,3R)-3-(cyclohexylmethoxy)-1-oxo-1-(4-(thiazol-2-yl)piperidin-1-yl)butan-2-yl)-2-((S)-2,2-dimethylcyclopropane-1-carbonyl)-5-oxo-2,6-diazaspiro[3.4]octane-8-carboxamide (7 mg, 31%) as a white solid. LCMS m/z=784.4 [M+H]⁺; ¹H NMR (400 MHz, Chloroform-d) δ 7.83 (s, 2H), 7.75 (s, 1H), 7.35 (d, J=7.6 Hz, 3H), 7.29-7.22 (m, 2H), 6.90 (d, J=7.6 Hz, 1H), 5.31 (s, 2H), 5.00 (s, 1H), 4.64 (s, 1H), 4.07 (dd, J=70.2, 52.6 Hz, 10H), 3.64 (s, 1H), 3.25 (d, J=51.4 Hz, 3H), 2.92 (d, J=28.8 Hz, 2H), 2.27-2.19 (m, 2H), 1.93-1.76 (m, 7H), 1.39-1.26 (m, 6H), 1.14 (d, J=7.4 Hz, 9H), 0.82 (d, J=56.6 Hz, 3H).

### Synthesis of 8-(tert-butyl) 4-ethyl 2,8-diazaspiro[4.5]decane-4,8-dicarboxylate

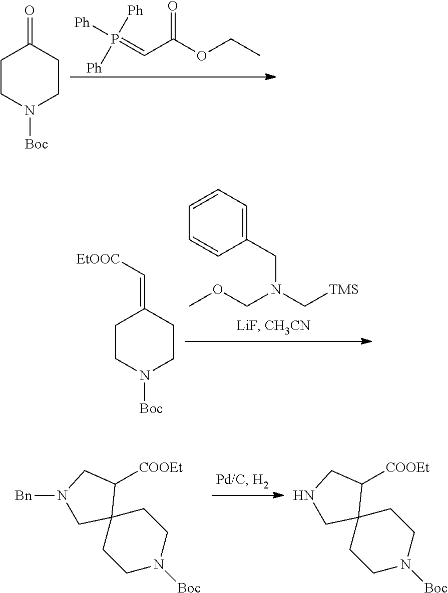

Step 1: 2-(2-(L-prolyl)-6-(thiazole-5-carbonyl)-2,6-diazaspiro[3.4]octan-8-yl)-5-((3,4-dichlorophenyl)difluoromethyl)-1,3,4-oxadiazole: A solution of tert-butyl 4-oxopiperidine-1-carboxylate (5.0 g, 25.09 mmol) and ethyl 2-(triphenyl-λ5-phosphanylidene)acetate (9.6 g, 27.60 mmol) in toluene (50 mL) was heated at reflux under a N₂ atmosphere for 18 h. The reaction was concentrated and the residue obtained purified by column chromatography on silica gel (eluent: Pet ether:EtOAc=20:1) to afford 2-(2-(L-prolyl)-6-(thiazole-5-carbonyl)-2,6-diazaspiro[3.4]octan-8-yl)-5-((3,4-dichlorophenyl)difluoromethyl)-1,3,4-oxadiazole (6.2 g, quant) as a white solid. ¹H NMR (400 MHz, CDCl₃) δ 5.71 (s, 1H), 4.15 (q, J=7.2 Hz, 2H), 3.53-3.43 (m, 4H), 2.93 (t, J=5.9 Hz, 2H), 2.27 (t, J=5.8 Hz, 2H), 1.47 (s, 9H), 1.28 (t, J=7.2 Hz, 3H).

Step 2: 8-(tert-butyl) 4-ethyl 2-benzyl-2,8-diazaspiro[4.5]decane-4,8-dicarboxylate: To a solution of 2-(2-(L-prolyl)-6-(thiazole-5-carbonyl)-2,6-diazaspiro[3.4]octan-8-yl)-5-((3,4-dichlorophenyl)difluoromethyl)-1,3,4-oxadiazole (6.2 g, 24.29 mmol) in MeCN (40 mL) was added N-benzyl-1-methoxy-N-((trimethylsilyl)methyl)methanamine (8.6 g, 36.44 mmol) and the mixture stirred at room temperature for 30 min. LiF (1.9 g, 72.87 mmol) was added and the reaction heated at reflux overnight. The mixture was diluted with EtOAc (400 mL), washed with water (100 mL×2), dried over Na₂SO₄, filtered and concentrated. The residue was purified by column chromatography on silica gel (eluent: Pet ether:EtOAc=5:1) to afford 8-(tert-butyl) 4-ethyl 2-benzyl-2,8-diazaspiro[4.5]decane-4,8-dicarboxylate (6.0 g, 61%) as a yellow oil. LCMS m/z=403.1 [M+H]⁺; ¹H NMR (400 MHz, CDCl₃) δ 7.36-7.27 (m, 4H), 7.25-7.20 (m, 1H), 4.16-4.09 (m, 2H), 3.84 (s, 2H), 3.70-3.61 (m, 2H), 3.04-2.65 (m, 6H), 2.35 (d, J=9.2 Hz, 1H), 1.87-1.71 (m, 2H), 1.70-1.50 (m, 2H), 1.44 (s, 9H), 1.26 (t, J=7.0 Hz, 3H).

Step 3: 8-(tert-butyl) 4-ethyl 2,8-diazaspiro[4.5]decane-4,8-dicarboxylate: To a solution of 8-(tert-butyl) 4-ethyl 2-benzyl-2,8-diazaspiro[4.5]decane-4,8-dicarboxylate (2.0 g, 4.97 mmol) in EtOAc (20 mL) was added 10% Pd/C (1.2 g). The reaction was stirred under a H₂ atmosphere for 24 h then the catalyst was removed by filtration trough Celite and the filtrate concentrated to afford 8-(tert-butyl) 4-ethyl 2,8-diazaspiro[4.5]decane-4,8-dicarboxylate (1.38 g, 92%) as a gray oil. LCMS m/z=313.2 [M+H]⁺; ¹H NMR (400 MHz, CDCl₃) δ 4.19-4.08 (m, 2H), 3.83-3.67 (m, 2H), 3.30-3.21 (m, 1H), 3.18-2.88 (m, 4H), 2.82 (d, J=11.2 Hz, 1H), 2.64-2.55 (m, 1H), 2.02 (s, 1H), 1.83-1.70 (m, 1H), 1.54-1.47 (m, 2H), 1.46-1.42 (m, 9H), 1.26 (t, J=7.1 Hz, 3H).

### Synthesis of (4-(5-((3,4-dichlorophenyl)difluoromethyl)-1,3,4-oxadiazol-2-yl)-2-(thiazole-5-carbonyl)-2,8-diazaspiro[4.5]decan-8-yl)((R)-2,2-difluorocyclopropyl)methanone (I-2)

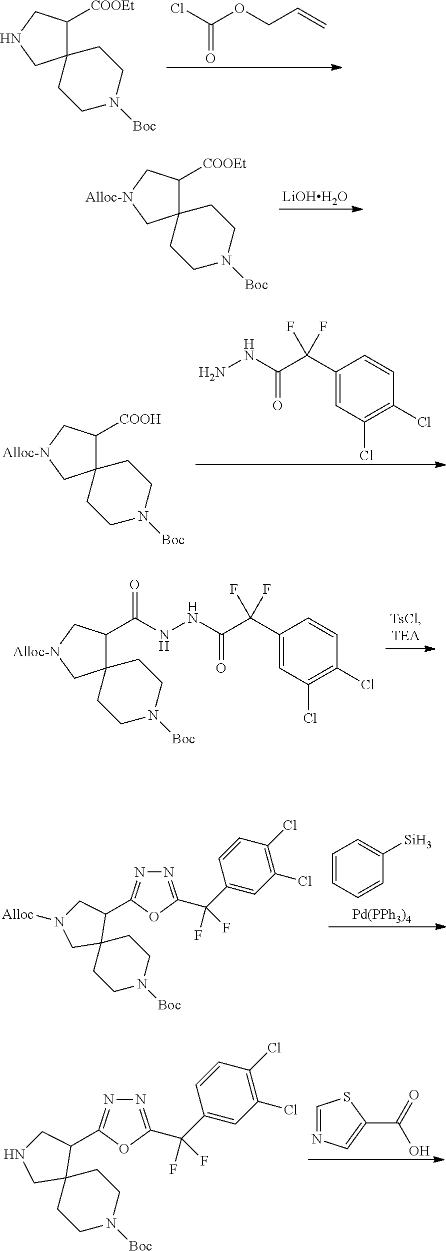

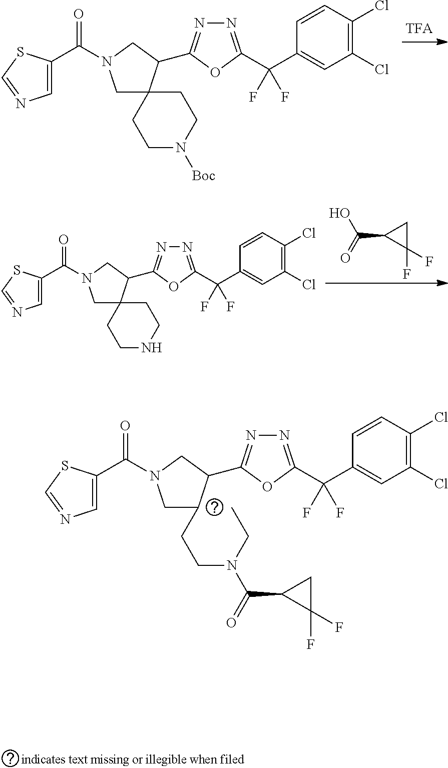

Step 1: 2-allyl 8-(tert-butyl) 4-ethyl 2,8-diazaspiro[4.5]decane-2,4,8-tricarboxylate: To a solution of 8-(tert-butyl) 4-ethyl 2,8-diazaspiro[4.5]decane-4,8-dicarboxylate (1.13 g, 3.62 mmol) and NaHCO₃ (304 mg, 3.62 mmol) in toluene (10 mL) was added allyl chloroformate (436 mg, 3.62 mmol). The reaction mixture was stirred at room temperature under a N₂ atmosphere overnight then was diluted with EtOAc (50 mL), washed with water (50 mL×2), dried over Na₂SO₄, filtered and concentrated. The residue obtained was purified by column chromatography on silica gel (eluent: Pet ether:EtOAc=5:1) to afford 2-allyl 8-(tert-butyl) 4-ethyl 2,8-diazaspiro[4.5]decane-2,4,8-tricarboxylate (1.2 g, 86%) as a yellow oil. LCMS m/z=419.4 [M+Na]; ¹H NMR (400 MHz, CDCl₃) δ 6.00-5.87 (m, 1H), 5.37-5.12 (m, 2H), 4.59 (d, 2H), 4.24-4.09 (m, 2H), 3.95-3.48 (m, 5H), 3.36-3.27 (m, 1H), 3.09-2.87 (m, 2H), 2.84-2.71 (m, 1H), 1.85-1.72 (m, 2H), 1.53-1.42 (m, 2H), 1.44 (s, 9H), 1.26 (t, J=7.2 Hz, 3H).

Step 2: 2-((allyloxy)carbonyl)-8-(tert-butoxycarbonyl)-2,8-diazaspiro[4.5]decane-4-carboxylic acid backspace backspace: To a solution of 2-allyl 8-(tert-butyl) 4-ethyl 2,8-diazaspiro[4.5]decane-2,4,8-tricarboxylate (1.2 g, 3.03 mmol) in a mixture of THF and water (10 mL/2 mL) at 0° C. was added LiOH·H₂O (254 mg, 6.06 mmol). The reaction was stirred at room temperature for 2 h then was diluted with water (20 mL), acidified with 1M HCl to pH˜2 and then extracted with EtOAc (50 mL×3). The combined organic layers were washed with brine, dried over Na₂SO₄, filtered and concentrated to afford 2-((allyloxy)carbonyl)-8-(tert-butoxycarbonyl)-2,8-diazaspiro[4.5]decane-4-carboxylic acid (1.0 g, 91%) as a white solid. LCMS M z=268.7 [M-99]⁺; ¹H NMR (400 MHz, CDCl₃) δ 6.02-5.84 (m, 1H), 5.36-5.17 (m, 2H), 4.65-4.55 (m, 2H), 3.98-3.52 (m, 5H), 3.33 (d, J=10.8, 3.2 Hz, 1H), 3.11-2.78 (m, 3H), 1.96-1.75 (m, 1H), 1.68-1.48 (m, 4H), 1.45 (s, 9H).

Step 3: 2-allyl 8-(tert-butyl) 4-(2-(2-(3,4-dichlorophenyl)-2,2-difluoroacetyl)hydrazine-1-carbonyl)-2,8-diazaspiro[4.5]decane-2,8-dicarboxylate: To a solution of 2-((allyloxy)carbonyl)-8-(tert-butoxycarbonyl)-2,8-diazaspiro[4.5]decane-4-carboxylic acid (950 mg, 2.58 mmol) in DMF (10 mL) was added EDCI (743 mg, 3.87 mmol), HOBt (523 mg, 3.87 mmol), 2-(3,4-dichlorophenyl)-2,2-difluoroacetohydrazide (790 mg, 3.09 mmol) and DIPEA (1.3 g, 10.32 mmol) and the reaction mixture stirred at room temperature overnight. The mixture was diluted with water (60 mL) and extracted with EtOAc (50 mL×2). The combined organic layers were washed with water, brine, dried over Na₂SO₄, filtered and concentrated. The residue obtained was purified by RP-column to afford 2-allyl 8-(tert-butyl) 4-(2-(2-(3,4-dichlorophenyl)-2,2-difluoroacetyl)hydrazine-1-carbonyl)-2,8-diazaspiro[4.5]decane-2,8-dicarboxylate (1.1 g, 69%) as a white solid. LCMS m/z=505.2 [M-99]⁺; ¹H NMR (400 MHz, CDCl₃) δ 9.24 (s, 1H), 7.73 (s, 1H), 7.58-7.43 (m, 2H), 5.99-5.84 (m, 1H), 5.34-5.14 (m, 2H), 4.64-4.49 (m, 2H), 3.90-3.30 (m, 7H), 3.16-2.80 (m, 2H), 2.79-2.63 (m, 1H), 1.71-1.45 (m, 4H), 1.41 (s, 9H).

Step 4: 2-allyl 8-(tert-butyl) 4-(5-((3,4-dichlorophenyl)difluoromethyl)-1,3,4-oxadiazol-2-yl)-2,8-diazaspiro[4.5]decane-2,8-dicarboxylate: To a solution of 2-allyl 8-(tert-butyl) 4-(2-(2-(3,4-dichlorophenyl)-2,2-difluoroacetyl)hydrazine-1-carbonyl)-2,8-diazaspiro[4.5]decane-2,8-dicarboxylate (1.0 g, 1.65 mmol) in DCM (10 mL) was added TEA (0.5 g, 4.96 mmol) and TsCl (944 mg, 4.96 mmol). The reaction was stirred at room temperature overnight then was diluted with water (50 mL) and extracted with DCM (30 mL×2). The combined organic layers were washed with brine, dried over Na₂SO₄, filtered and concentrated. The residue obtained was purified by column chromatography on silica gel (eluent: DCM:MeOH=60:1) to afford 2-allyl 8-(tert-butyl) 4-(5-((3,4-dichlorophenyl)difluoromethyl)-1,3,4-oxadiazol-2-yl)-2,8-diazaspiro[4.5]decane-2,8-dicarboxylate (1.0 g, 82%) as a white solid. LCMS m/z=487.1 [M-99]⁺; ¹H NMR (400 MHz, CDCl₃) δ 7.78-7.74 (m, 1H), 7.60 (d, J=8.4 Hz, 1H), 7.50 (d, J=8.6 Hz, 1H), 6.04-5.86 (m, 1H), 5.38-5.15 (m, 2H), 4.70-4.51 (m, 2H), 4.04-3.59 (m, 5H), 3.52-3.35 (m, 2H), 3.07-2.83 (m, 2H), 1.91-1.74 (m, 1H), 1.64-1.58 (m, 1H), 1.50-1.45 (m, 1H), 1.43 (s, 9H), 1.38-1.31 (m, 1H).

Step 5: tert-butyl 4-(5-((3,4-dichlorophenyl)difluoromethyl)-1,3,4-oxadiazol-2-yl)-2,8-diazaspiro[4.5]decane-8-carboxylate: To a solution of 2-allyl 8-(tert-butyl) 4-(5-((3,4-dichlorophenyl)difluoromethyl)-1,3,4-oxadiazol-2-yl)-2,8-diazaspiro[4.5]decane-2,8-dicarboxylate (0.9 g, 1.53 mmol) in DCM (10 mL) was added phenylsilane (498 mg, 4.60 mmol) and Pd(PPh₃)₄ (173 mg, 0.15 mmol). The reaction was stirred at room temperature under a N₂ atmosphere for 2 h then was diluted with water (100 mL) and extracted with DCM (30 mL×2). The combined organic layers were dried over Na₂SO₄, filtered and concentrated. The residue was purified by RP-column to afford tert-butyl 4-(5-((3,4-dichlorophenyl)difluoromethyl)-1,3,4-oxadiazol-2-yl)-2,8-diazaspiro[4.5]decane-8-carboxylate (90 mg, 12% yield) as a yellow oil. LCMS m/z=503.1 [M+H]⁺; ¹H NMR (400 MHz, CDCl₃) δ 7.76 (s, 1H), 7.61 (d, J=8.4 Hz, 1H), 7.52-7.48 (m, 1H), 3.98-3.85 (m, 1H), 3.81-3.65 (m, 1H), 3.51 (d, J=7.2 Hz, 2H), 3.32 (t, 1H), 3.38-2.81 (m, 4H), 1.88-1.75 (m, 2H), 1.71-1.62 (m, 1H), 1.44 (s, 9H) 1.38-1.30 (m, 1H), 1.14-1.01 (m, 1H).

Step 6: tert-butyl 4-(5-((3,4-dichlorophenyl)difluoromethyl)-1,3,4-oxadiazol-2-yl)-2-(thiazole-5-carbonyl)-2,8-diazaspiro[4.5]decane-8-carboxylate: To a solution of thiazole-5-carboxylic acid (25 mg, 0.19 mmol) in DCM (1 mL) was added EDCI (46 mg, 0.24 mmol) and HOBt (33 mg, 0.24 mmol), tert-butyl 4-(5-((3,4-dichlorophenyl)difluoromethyl)-1,3,4-oxadiazol-2-yl)-2,8-diazaspiro[4.5]decane-8-carboxylate (80 mg, 0.16 mmol) and DIPEA (83 mg, 0.64 mmol) and the reaction mixture stirred at room temperature overnight. The mixture was diluted with water (10 mL) and extracted with EtOAc (20 mL×2). The combined organic layers were washed with brine, dried over Na₂SO₄, filtered and concentrated. The residue obtained was purified by column chromatography on silica gel (eluent: DCM:MeOH=80:1) to afford tert-butyl 4-(5-((3,4-dichlorophenyl)difluoromethyl)-1,3,4-oxadiazol-2-yl)-2-(thiazole-5-carbonyl)-2,8-diazaspiro[4.5]decane-8-carboxylate (80 mg, 73%) as a white solid. LCMS m/z=558.0 [M-55]⁺; ¹H NMR (400 MHz, CD₃OD) δ 9.15 (s, 1H), 8.39 (d, J=11.4 Hz, 1H), 7.93-7.84 (m, 1H), 7.73 (d, J=8.4 Hz, 1H), 7.65-7.60 (m, 1H), 4.45-4.32 (m, 1H), 4.17 (d, 1H), 4.04-3.77 (m, 4H), 3.73-3.64 (m, 1H), 3.20-3.00 (m, 2H), 1.89-1.73 (m, 2H), 1.45-1.41 (m, 9H), 1.33-1.24 (m, 1H), 1.21-1.12 (m, 1H).

Step 7: (4-(5-((3,4-dichlorophenyl)difluoromethyl)-1,3,4-oxadiazol-2-yl)-2,8-diazaspiro[4.5]decan-2-yl)(thiazol-5-yl)methanone: To a solution of tert-butyl 4-(5-((3,4-dichlorophenyl)difluoromethyl)-1,3,4-oxadiazol-2-yl)-2-(thiazole-5-carbonyl)-2,8-diazaspiro[4.5]decane-8-carboxylate (100 mg, 0.16 mmol) in DCM (1 mL) was added TFA (0.5 mL) and the reaction stirred at room temperature for 1 h. The solvent was removed under vacuum to afford (4-(5-((3,4-dichlorophenyl)difluoromethyl)-1,3,4-oxadiazol-2-yl)-2,8-diazaspiro[4.5]decan-2-yl)(thiazol-5-yl)methanone (82 mg, quant.) as a grey oil which was used directly in the next step. LCMS m/z=514.1 [M-55]⁺; ¹H NMR (400 MHz, CD₃OD) δ 9.22-9.14 (m, 1H), 8.41 (d, J=18.6 Hz, 2H), 7.94-7.87 (m, 1H), 7.75 (d, J=8.4 Hz, 1H), 7.70-7.59 (m, 1H), 4.49-4.33 (m, 1H), 4.23-3.74 (m, 4H), 3.53-3.37 (m, 1H), 3.26-3.09 (m, 2H), 2.26-2.00 (m, 2H), 1.82 (t, 1H), 1.65-1.50 (m, 1H).

Step 8: (4-(5-((3,4-dichlorophenyl)difluoromethyl)-1,3,4-oxadiazol-2-yl)-2-(thiazole-5-carbonyl)-2,8-diazaspiro[4.5]decan-8-yl)((R)-2,2-difluorocyclopropyl)methanone: To a solution of (R)-2,2-difluorocyclopropane-1-carboxylic acid (20 mg, 0.16 mmol) in DCM (1 mL) was added HATU (91 mg, 0.24 mmol) and DIPEA (62 mg, 0.48 mmol) and the mixture stirred for min. (4-(5-((3,4-dichlorophenyl)difluoromethyl)-1,3,4-oxadiazol-2-yl)-2,8-diazaspiro[4.5]decan-2-yl)(thiazol-5-yl)methanone (82 mg, 0.16 mmol) was added and stirring continued for 2 h. The mixture was diluted with water (60 mL) and extracted with DCM (30 mL×2). The combined organic layers were washed with brine, dried over Na₂SO₄, filtered and concentrated. The residue obtained was purified by RP-column to afford (4-(5-((3,4-dichlorophenyl)difluoromethyl)-1,3,4-oxadiazol-2-yl)-2-(thiazole-5-carbonyl)-2,8-diazaspiro[4.5]decan-8-yl)((R)-2,2-difluorocyclopropyl)methanone (50 mg, 51%) as a yellow solid. LCMS m/z=618.1 [M+H]⁺; ¹H NMR (400 MHz, CD₃OD) δ 9.18 (s, 1H), 8.46-8.37 (m, 1H), 7.95-7.83 (m, 1H), 7.74 (d, J=8.4 Hz, 1H), 7.67-7.57 (m, 1H), 4.43-3.71 (m, 7H), 3.60-3.38 (m, 1H), 3.28-2.80 (m, 2H), 1.99-1.10 (m, 6H).

### Synthesis of tert-butyl 4-(5-((3,4-dichlorophenyl)difluoromethyl)-1,3,4-oxadiazol-2-yl)-2-(thiazole-5-carbonyl)-2,8-diazaspiro[4.5]decane-8-carboxylate (I-14)

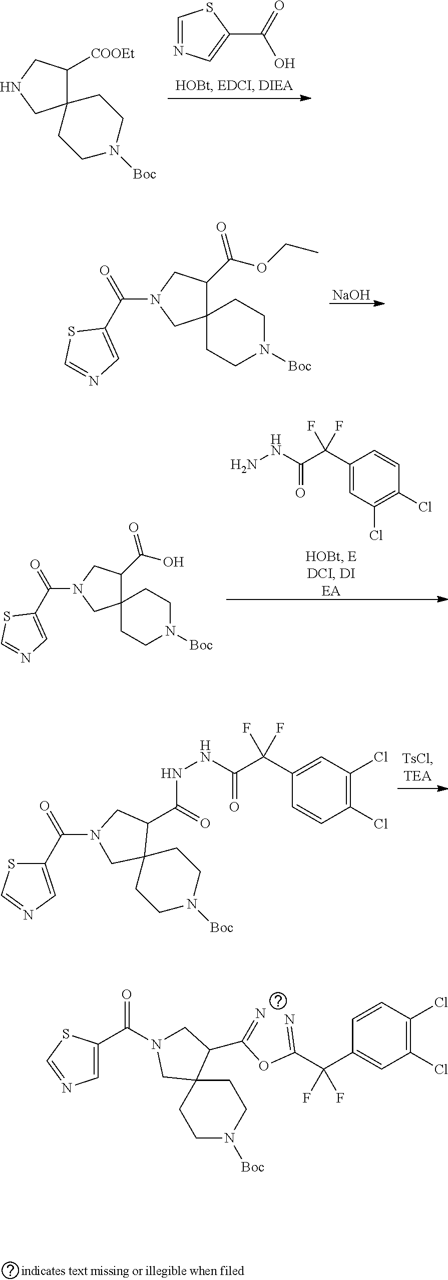

Step 1: 8-(tert-butyl) 4-ethyl 2-(thiazole-5-carbonyl)-2,8-diazaspiro[4.5]decane-4,8-dicarboxylate: To a solution of thiazole-5-carboxylic acid (496 mg, 3.84 mmol) in DCM (10 mL) was added EDCI (920 mg, 4.80 mmol), HOBt (649 mg, 4.80 mmol), 8-(tert-butyl) 4-ethyl 2,8-diazaspiro[4.5]decane-4,8-dicarboxylate (1.0 g, 3.20 mmol) and DIPEA (1.7 g, 12.80 mmol). The reaction was stirred at room temperature overnight then was diluted with water (60 mL) and extracted with EtOAc (50 mL×2). The combined organic layers were washed with brine, dried over Na₂SO₄, filtered and concentrated. The residue obtained was purified by column chromatography on silica gel (DCM/MeOH=80/1) to afford 8-(tert-butyl) 4-ethyl 2-(thiazole-5-carbonyl)-2,8-diazaspiro[4.5]decane-4,8-dicarboxylate (870 mg, 64%) as a white solid. LCMS m/z=446.2 [M+Na]⁺; ¹H NMR (400 MHz, CDCl3) δ 8.91 (s, 1H), 8.25 (d, J=12.2 Hz, 1H), 4.26-3.55 (m, 8H), 3.19-2.90 (m, 3H), 1.64-1.59 (m, 4H), 1.47-1.43 (m, 9H), 1.31-1.27 (m, 3H).

Step 2: 8-(tert-butoxycarbonyl)-2-(thiazole-5-carbonyl)-2,8-diazaspiro[4.5]decane-4-carboxylic acid: To a solution of 8-(tert-butyl) 4-ethyl 2-(thiazole-5-carbonyl)-2,8-diazaspiro[4.5]decane-4,8-dicarboxylate (100 mg, 0.24 mmol) in a mixture of MeOH and water at 0° C. was added a solution of NaOH (189 mg, 0.47 mmol) in water (0.5 mL). The reaction was stirred at room temperature for 2 h then was diluted with water (20 mL) and extracted with EtOAc (20 mL). The aqueous layer was collected, acidified with 1M HCl to pH ˜2 and extracted with EtOAc (20 mL×3). The combined organic layers were washed with brine, dried over Na₂SO₄, filtered and concentrated to afford 8-(tert-butoxycarbonyl)-2-(thiazole-5-carbonyl)-2,8-diazaspiro[4.5]decane-4-carboxylic acid (40 mg, 43%) as a white solid which was used directly in the next step. LCMS m/z=418.2 [M+Na]⁺; ¹H NMR (400 MHz, CDCl3) δ 8.94 (s, 1H), 8.28 (d, J=12.6 Hz, 1H), 5.30 (s, 1H), 4.20-3.87 (m, 5H), 3.81-3.53 (m, 3H), 3.21-2.90 (m, 4H), 1.32-1.28 (m, 9H).

Step 3: tert-butyl 4-(2-(2-(3,4-dichlorophenyl)-2,2-difluoroacetyl)hydrazine-1-carbonyl)-2-(thiazole-5-carbonyl)-2,8-diazaspiro[4.5]decane-8-carboxylate: To a solution of 2-(3,4-dichlorophenyl)-2,2-difluoroacetohydrazide (40 mg, 0.10 mmol) in DCM (0.5 mL) was added EDCI (29 mg, 0.15 mmol), HOBt (20 mg, 0.15 mmol), 8-(tert-butoxycarbonyl)-2-(thiazole-5-carbonyl)-2,8-diazaspiro[4.5]decane-4-carboxylic acid (31 mg, 0.12 mmol) and DIPEA (52 mg, 0.40 mmol) and the reaction stirred at room temperature overnight. The mixture was diluted with water (10 mL) and extracted with EtOAc (10 mL×2). The combined organic layers were washed with brine, dried over Na₂SO₄, filtered and concentrated. The residue obtained was purified by prep-TLC (DCM/MeOH=15/1) to afford tert-butyl 4-(2-(2-(3,4-dichlorophenyl)-2,2-difluoroacetyl)hydrazine-1-carbonyl)-2-(thiazole-5-carbonyl)-2,8-diazaspiro[4.5]decane-8-carboxylate (40 mg, 63%) as a white solid. LCMS m/z=575.9 [M-56]⁺;

Step 4: tert-butyl 4-(5-((3,4-dichlorophenyl)difluoromethyl)-1,3,4-oxadiazol-2-yl)-2-(thiazole-5-carbonyl)-2,8-diazaspiro[4.5]decane-8-carboxylate: To a solution of tert-butyl 4-(2-(2-(3,4-dichlorophenyl)-2,2-difluoroacetyl)hydrazine-1-carbonyl)-2-(thiazole-5-carbonyl)-2,8-diazaspiro[4.5]decane-8-carboxylate (290 mg, 0.46 mmol) in DCM (3 mL) was added TEA (232 mg, 2.29 mmol) and TsCl (437 mg, 2.29 mmol) and the reaction was stirred at room temperature for 2 hours. The mixture was diluted with water (50 mL) and extracted with EtOAc (20 mL×3). The combined organic layers were washed with brine, dried over Na₂SO₄, filtered and concentrated. The residue was purified by prep-TLC (DCM/MeOH=15/1) to afford tert-butyl 4-(5-((3,4-dichlorophenyl)difluoromethyl)-1,3,4-oxadiazol-2-yl)-2-(thiazole-5-carbonyl)-2,8-diazaspiro[4.5]decane-8-carboxylate (150 mg, 53%) as a white solid. LCMS m/z=558.0 [M-55]⁺; ¹H NMR (400 MHz, CD₃OD) δ 9.15 (s, 1H), 8.39 (d, J=11.2 Hz, 1H), 7.88 (d, 1H), 7.73 (d, J=8.5 Hz, 1H), 7.65-7.60 (m, 1H), 4.45-4.32 (m, 1H), 4.17 (d, 1H), 4.04-3.77 (m, 4H), 3.73-3.64 (m, 1H), 3.20-3.00 (m, 2H), 1.89-1.73 (m, 2H), 1.45-1.41 (m, 10H), 1.21-1.12 (m, 1H).

### Synthesis of (4-(5-((3,4-dichlorophenyl)difluoromethyl)-1,3,4-oxadiazol-2-yl)-2-(thiazole-5-carbonyl)-2,8-diazaspiro[4.5]decan-8-yl)((S)-2,2-dimethylcyclopropyl)methanone (I-15)

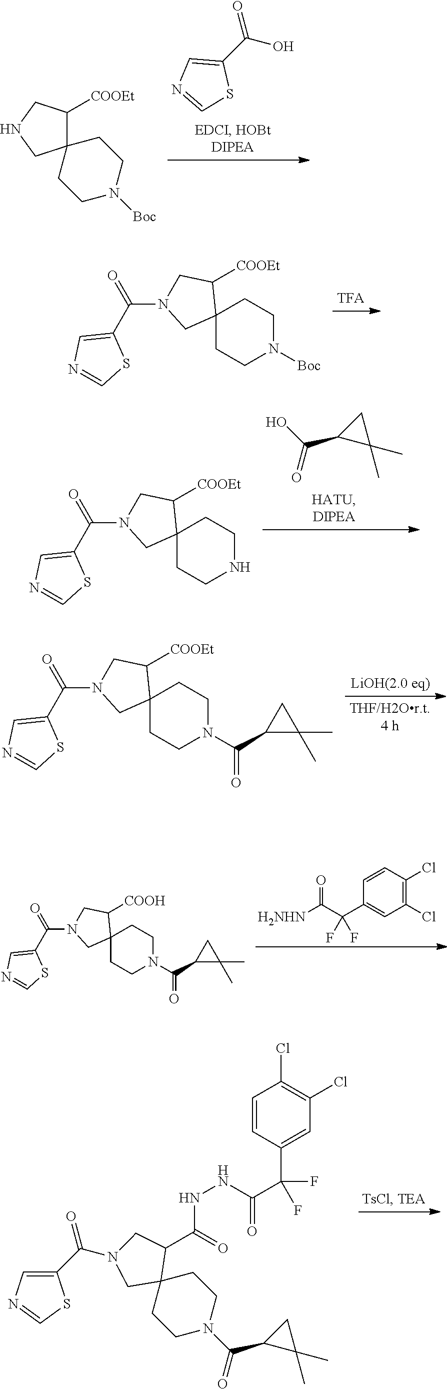

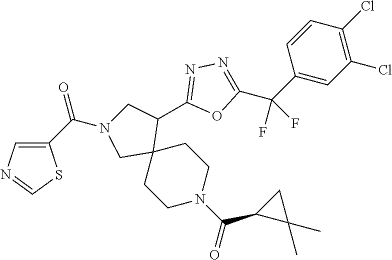 [SMILES: CC1(C)C[C@@H]1C(=O)N1CCC2(CC1)CN(C(=O)c1cncs1)CC2c1nnc(C(F)(F)c2ccc(Cl)c(Cl)c2)o1]

Step 1: 8-(tert-butyl) 4-ethyl 2-(thiazole-5-carbonyl)-2,8-diazaspiro[4.5]decane-4,8-dicarboxylate: To a solution of thiazole-5-carboxylic acid (39 mg, 0.30 mmol) in DMF (2.0 mL) was added 8-(tert-butyl) 4-ethyl 2,8-diazaspiro[4.5]decane-4,8-dicarboxylate (78 mg, 0.25 mmol), EDCI (73 mg, 0.38 mmol), HOBt (51 mg, 0.38 mmol) and DIPEA (98 mg, 0.75 mmol) and the reaction was stirred overnight. The mixture was diluted with water (10 mL) and extracted with EtOAc (20 mL×2). The combined organic layers were washed with brine, dried over Na₂SO₄, filtered and concentrated. The residue obtained was purified by prep-TLC (eluent: Pet. DCM:methanol=12:1) to afford 8-(tert-butyl) 4-ethyl 2-(thiazole-5-carbonyl)-2,8-diazaspiro[4.5]decane-4,8-dicarboxylate (33 mg, 31%) as a colorless oil. LCMS m/z=426.2 [M+Na]⁺; ¹H NMR (400 MHz, CDCl3) δ 8.89 (s, 1H), 8.23 (d, J=12.2 Hz, 1H), 4.29-4.05 (m, 3H), 4.02-3.83 (m, 4H), 3.76-3.53 (m, 2H), 3.21-2.88 (m, 3H), 2.01-1.69 (m, 3H), 1.44 (d, J=5.4 Hz, 9H), 1.27 (t, J=7.2 Hz, 3H).

Step 2: ethyl 2-(thiazole-5-carbonyl)-2,8-diazaspiro[4.5]decane-4-carboxylate: To a solution of 8-(tert-butyl) 4-ethyl 2-(thiazole-5-carbonyl)-2,8-diazaspiro[4.5]decane-4,8-dicarboxylate (288 mg, 0.68 mmol) in DCM (3 mL) was added TFA (3.0 mL) and the reaction stirred at room temperature for 2 h. The solvent was removed under reduced pressure to afford ethyl 2-(thiazole-5-carbonyl)-2,8-diazaspiro[4.5]decane-4-carboxylate (220 mg, quant.) as a grey oil which was used directly in the next step

Step 3: ethyl 8-((S)-2,2-dimethylcyclopropane-1-carbonyl)-2-(thiazole-5-carbonyl)-2,8-diazaspiro[4.5]decane-4-carboxylate: To a solution of (S)-2,2-dimethylcyclopropane-1-carboxylic acid (93 mg, 0.82 mmol) in DCM (3.0 mL) was added HATU (388 mg, 1.02 mmol) and DIPEA (352 mg, 2.72 mmol) and the mixture stirred at room temperature for 30 min. Ethyl 2-(thiazole-5-carbonyl)-2,8-diazaspiro[4.5]decane-4-carboxylate (220 mg, 0.68 mmol) was added and stirring continued for 2 h. The mixture was diluted with water (100 mL) and extracted with DCM (20 mL×2). The combined organic layers were washed with brine, dried over Na₂SO₄, filtered and concentrated. The residue obtained was purified by column chromatography on silica gel (eluent: DCM/MeOH=75/1) to afford ethyl 8-((S)-2,2-dimethylcyclopropane-1-carbonyl)-2-(thiazole-5-carbonyl)-2,8-diazaspiro[4.5]decane-4-carboxylat (200 mg, 70%) as a colorless oil. LCMS m/z=420.2 [M+H]⁺; ¹H NMR (400 MHz, CDCl₃) δ 8.92 (s, 1H), 8.26 (d, J=12.6 Hz, 1H), 4.32-3.51 (m, 8H), 3.44-2.94 (m, 3H), 1.62-1.46 (m, 4H), 1.38-1.25 (m, 4H), 1.22-1.10 (m, 4H), 1.07-0.96 (m, 3H), 0.77-0.66 (m, 1H).

Step 4: 8-((S)-2,2-dimethylcyclopropane-1-carbonyl)-2-(thiazole-5-carbonyl)-2,8-diazaspiro[4.5]decane-4-carboxylic acid: To a solution of 8-(tert-butyl) 4-ethyl 2-(thiazole-5-carbonyl)-2,8-diazaspiro[4.5]decane-4,8-dicarboxylate (200 mg, 0.48 mmol) in a mixture of THF and water (4 mL/1 mL) was added LiOH·H₂O (38 mg, 0.96 mmol) and the reaction stirred at room temperature for 2 h. The mixture was diluted with water (10 mL) and extracted with EtOAc (10 mL). The aqueous layer was collected, acidified with 1M HCl to pH ˜2 and extracted with EtOAc (20 mL×3). The combined organic layers were washed with brine, dried over Na₂SO₄, filtered and concentrated to afford ethyl 2-(thiazole-5-carbonyl)-2,8-diazaspiro[4.5]decane-4-carboxylate (130 mg, 69%) as a colorless oil which was used directly in the next step. LCMS m/z=392.3 [M+H]⁺; ¹H NMR (400 MHz, CD₃OD) δ 9.16 (s, 1H), 8.42-8.36 (m, 1H), 4.35-3.76 (m, 5H), 3.65-3.38 (m, 1H), 3.22-3.00 (m, 1H), 2.20-1.88 (m, 1H), 1.86-1.53 (m, 5H), 1.25-1.18 (m, 4H), 1.10-1.00 (m, 4H), 0.79-0.67 (m, 1H).

Step 5: N′-(2-(3,4-dichlorophenyl)-2,2-difluoroacetyl)-8-((S)-2,2-dimethylcyclopropane-1-carbonyl)-2-(thiazole-5-carbonyl)-2,8-diazaspiro[4.5]decane-4-carbohydrazide: To a solution of 8-((S)-2,2-dimethylcyclopropane-1-carbonyl)-2-(thiazole-5-carbonyl)-2,8-diazaspiro[4.5]decane-4-carboxylic acid (30 mg, 0.077 mmol) in DMF (1 mL) was added EDCI (30 mg, 0.154 mmol), HOBt (21 mg, 0.154 mmol), 2-(3,4-dichlorophenyl)-2,2-difluoroacetohydrazide (23 mg, 0.077 mmol) and DIPEA (40 mg, 0.308 mmol). The reaction was stirred at room temperature for 6 h then the solvent was removed and the residue obtained purified by RP-column to afford N′-(2-(3,4-dichlorophenyl)-2,2-difluoroacetyl)-8-((S)-2,2-dimethylcyclopropane-1-carbonyl)-2-(thiazole-5-carbonyl)-2,8-diazaspiro[4.5]decane-4-carbohydrazide (40 mg, 83%) as a colorless solid. LCMS m/z=628.2 [M+H]⁺.

Step 6: (4-(5-((3,4-dichlorophenyl)difluoromethyl)-1,3,4-oxadiazol-2-yl)-2-(thiazole-5-carbonyl)-2,8-diazaspiro[4.5]decan-8-yl)((S)-2,2-dimethylcyclopropyl)methanone (I-15): To a solution of N′-(2-(3,4-dichlorophenyl)-2,2-difluoroacetyl)-8-((S)-2,2-dimethylcyclopropane-1-carbonyl)-2-(thiazole-5-carbonyl)-2,8-diazaspiro[4.5]decane-4-carbohydrazide (50 mg, 0.08 mmol) in DCM (2.0 mL) was added TEA (40 mg, 0.40 mmol) and TsCl (76 mg, 0.40 mmol) and the reaction stirred at room temperature overnight. The mixture was diluted with water and extracted with EtOAc (30 mL×3). The combine organic layers were washed with brine, dried over Na₂SO₄, filtered and concentrated. The residue obtained was purified by prep-TLC (DCM/MeOH=15/1) and column chromatography on silica gel (eluent: DCM/MeOH=10/1) to afford (4-(5-((3,4-dichlorophenyl)difluoromethyl)-1,3,4-oxadiazol-2-yl)-2-(thiazole-5-carbonyl)-2,8-diazaspiro[4.5]decan-8-yl)((S)-2,2-dimethylcyclopropyl)methanone (36 mg, 73%) as a white solid. LCMS m/z=610.2 [M+H]⁺; ¹H NMR (400 MHz, CD₃OD) δ 9.17 (d, J=3.7 Hz, 1H), 8.47-8.37 (m, 1H), 7.94-7.85 (m, 1H), 7.78-7.72 (m, 1H), 7.68-7.57 (m, 1H), 4.56-3.69 (m, 7H), 3.53-3.37 (m, 1H), 3.28-2.99 (m, 1H), 2.06-1.76 (m, 2H), 1.75-1.43 (m, 2H), 1.24-0.95 (m, 7H), 0.94-0.81 (m, 1H), 0.80-0.63 (m, 1H).

### Synthesis of 8-(5-((3,4-dichlorophenyl)difluoromethyl)-1,3,4-oxadiazol-2-yl)-6-(thiazole-5-carbonyl)-6-azaspiro[3.4]octane-2-carboxylic acid and I-16

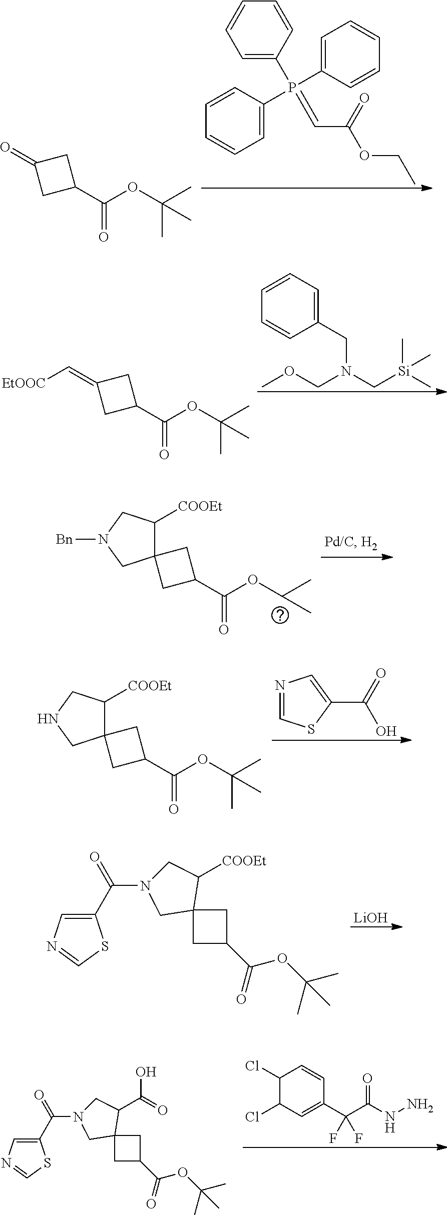

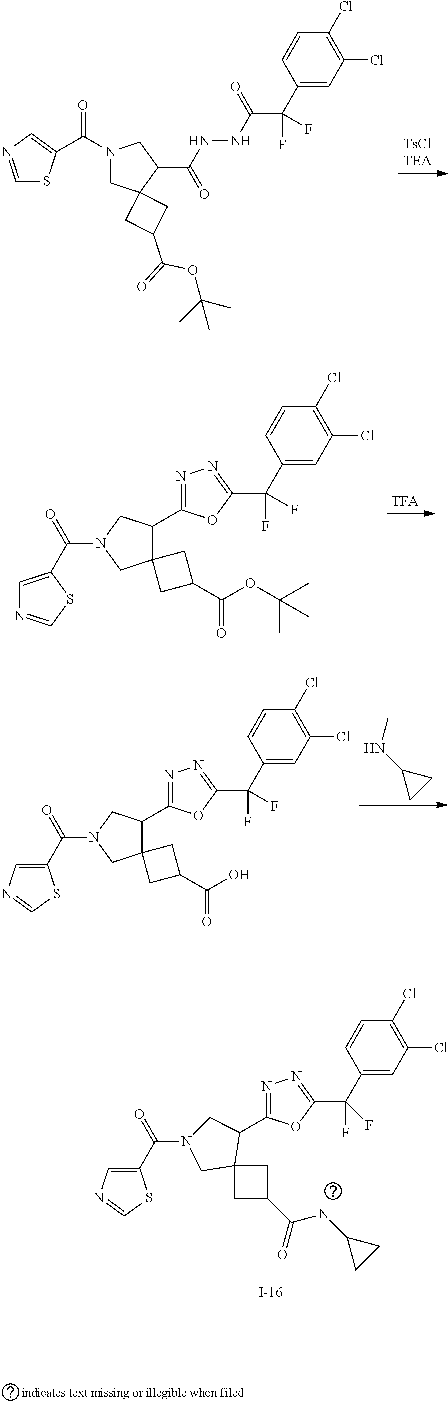

Step 1: tert-butyl 3-(2-ethoxy-2-oxoethylidene)cyclobutane-1-carboxylate: To a solution of tert-butyl 3-oxocyclobutane-1-carboxylate (7.7 g, 45.24 mmol) in toluene (30 mL) was added ethyl 2-(triphenyl-15-phosphanylidene)acetate (23.6 g, 67.86 mmol) and the reaction heated at 55° C. overnight. The solvent was removed under vacuum and the residue obtained purified by column chromatography on silica gel (eluent: Pet.ether:EtOAc=60:1) to afford tert-butyl 3-(2-ethoxy-2-oxoethylidene)cyclobutane-1-carboxylate (7.68 g, 71%) as colorless oil. LCMS m/z=263.20 [M+Na]⁺; ¹H NMR (400 MHz, DMSO-d6) δ 5.65 (p, J=2.2 Hz, 1H), 4.06 (q, J=7.2 Hz, 2H), 3.30-2.88 (m, 5H), 1.41 (s, 9H), 1.19 (t, J=7.2 Hz, 3H).

Step 2: 2-(tert-butyl) 8-ethyl 6-benzyl-6-azaspiro[3.4]octane-2,8-dicarboxylate: To a solution of tert-butyl 3-(2-ethoxy-2-oxoethylidene)cyclobutane-1-carboxylate (7.7 g, 31.96 mmol) and N-benzyl-1-methoxy-N-((trimethylsilyl)methyl)methanamine (11.3 g, 47.9 mmol) in CH₃CN (28 mL) was added LiF (2.5 g, 95.8 mmol) and the reaction heated at 90° C. for 2 days. The mixture was diluted with water (130 mL) and extracted with EtOAc (100 mL×3). The combined organic layers were washed with saturated aqueous CuSO₄ and 2M K₂CO₃, dried over Na₂SO₄, filtered and concentrated. The mixture was purified by column chromatography on silica gel (eluent: Pet. Ether:EtOAc=8:1) to afford 2-(tert-butyl) 8-ethyl 6-benzyl-6-azaspiro[3.4]octane-2,8-dicarboxylate (10.3 g, 87%) as a yellow oil. LCMS m/z=374.25 [M+H]⁺; ¹H NMR (400 MHz, DMSO-d₆) δ 7.39-7.16 (m, 5H), 4.22-3.99 (m, 2H), 3.69-3.48 (m, 2H), 2.98-2.52 (m, 5H), 2.49-1.84 (m, 5H), 1.35 (d, J=18.8 Hz, 9H), 1.26-1.17 (m, 3H).

Step 3: 2-(tert-butyl) 8-ethyl 6-azaspiro[3.4]octane-2,8-dicarboxylate: To a solution of 2-(tert-butyl) 8-ethyl 6-benzyl-6-azaspiro[3.4]octane-2,8-dicarboxylate (5.0 g, 13.39 mmol) in EtOAc (40 mL) was added 10% Pd/C (2 g) and the reaction heated at 50° C. under a H₂ atmosphere for 24 h. The catalyst was removed by filtration through Celite and th filtrate concentrated to afford 2-(tert-butyl) 8-ethyl 6-azaspiro[3.4]octane-2,8-dicarboxylate (3.5 g, 94%) which was used directly in the next step. LCMS m/z=284.20 [M+H]⁺; ¹H NMR (400 MHz, Chloroform-d) δ 4.23-4.09 (m, 2H), 3.23-3.01 (m, 2H), 2.98-2.84 (m, 2H), 2.85-2.70 (m, 1H), 2.38-2.21 (m, 3H), 2.21-1.96 (m, 3H), 1.43 (d, J=3.2 Hz, 9H), 1.34-1.22 (m, 3H).

Step 4: 2-(tert-butyl) 8-ethyl 6-(thiazole-5-carbonyl)-6-azaspiro[3.4]octane-2,8-dicarboxylate: To a solution of thiazole-5-carboxylic acid (250 mg, 1.9 mmol) in DMF (15 mL) was added HATU (1.0 g, 2.7 mmol) and DIPEA (684 mg, 5.3 mmol) and the mixture stirred at room temperature for 30 min. 2-(tert-butyl) 8-ethyl 6-azaspiro[3.4]octane-2,8-dicarboxylate (500 mg, 1.8 mmol) was added and the reaction was stirred overnight. The mixture was diluted with water (30 mL) and extracted with EtOAc (30 mL×3). The combined organic layers were washed with brine, dried over Na₂SO₄, filtered and concentrated. The residue obtained was purified by prep-TLC (DCM:MeOH=15:1) to afford 2-(tert-butyl) 8-ethyl 6-(thiazole-5-carbonyl)-6-azaspiro[3.4]octane-2,8-dicarboxylate (393 mg, 57%) as a yellow oil. LCMS m/z=417.15 [M+H]⁺; ¹H NMR (400 MHz, Chloroform-d) δ 8.90 (d, J=2.8 Hz, 1H), 8.24 (d, J=7.8 Hz, 1H), 4.34-4.09 (m, 2H), 4.08-3.64 (m, 4H), 3.12-2.94 (m, 2H), 2.55-2.09 (m, 4H), 1.53-1.37 (m, 9H), 1.35-1.26 (m, 3H).

Step 5: 2-(tert-butoxycarbonyl)-6-(thiazole-5-carbonyl)-6-azaspiro[3.4]octane-8-carboxylic acid: To a solution of 2-(tert-butyl) 8-ethyl 6-(thiazole-5-carbonyl)-6-azaspiro[3.4]octane-2,8-dicarboxylate (390 mg, 0.99 mmol) in a mixture of THF (8 mL) and water (2 mL) was added LiOH·H₂O (83 mg, 1.98 mmol) and the reaction stirred at room temperature overnight. The mixture was diluted with water (20 mL) and extracted with EtOAc (20 mL×2). The aqueous phase was collected, acidified with 1M HCl to pH ˜2 and extracted with EtOAc (50 mL×3). The combined organic layers were washed with brine, dried over Na₂SO₄ filtered and concentrated to afford 2-(tert-butoxycarbonyl)-6-(thiazole-5-carbonyl)-6-azaspiro[3.4]octane-8-carboxylic acid (96 mg, 27%) as a white solid. LCMS m/z=367.15 [M+H]⁺.

Step 6: tert-butyl 8-(2-(2-(3,4-dichlorophenyl)-2,2-difluoroacetyl)hydrazine-1-carbonyl)-6-(thiazole-5-carbonyl)-6-azaspiro[3.4]octane-2-carboxylate: To a solution of 2-(3,4-dichlorophenyl)-2,2-difluoroacetohydrazide (96 mg, 0.26 mmol) in DMF (8 mL) was added 2-(tert-butoxycarbonyl)-6-(thiazole-5-carbonyl)-6-azaspiro[3.4]octane-8-carboxylic acid (73 mg, 0.29 mmol), EDCI (75 mg, 0.39 mmol), HOBt (53 mg, 0.39 mmol) and DIPEA (101 mg, 0.79 mmol) and the reaction stirred at room temperature for 2 h. The mixture was diluted with water (20 mL) and extracted with EtOAc (20 mL×3). The combined organic layers were washed with brine, dried over Na₂SO₄, filtered and concentrated. The residue obtained was purified by prep-TLC (DCM:MeOH=15:1) to afford tert-butyl 8-(2-(2-(3,4-dichlorophenyl)-2,2-difluoroacetyl)hydrazine-1-carbonyl)-6-(thiazole-5-carbonyl)-6-azaspiro[3.4]octane-2-carboxylate (99 mg, 63%) as colorless oil. LCMS m/z=603.15 [M+H]⁺; ¹H NMR (400 MHz, Chloroform-d) δ 8.96-8.85 (m, 1H), 8.24 (d, J=12.8 Hz, 1H), 7.74 (s, 1H), 7.63-7.37 (m, 2H), 4.13-3.61 (m, 4H), 3.27-2.91 (m, 2H), 2.65-1.96 (m, 4H), 1.50-1.34 (m, 9H).

Step 7: tert-butyl 8-(5-((3,4-dichlorophenyl)difluoromethyl)-1,3,4-oxadiazol-2-yl)-6-(thiazole-5-carbonyl)-6-azaspiro[3.4]octane-2-carboxylate: To a solution of tert-butyl 8-(2-(2-(3,4-dichlorophenyl)-2,2-difluoroacetyl)hydrazine-1-carbonyl)-6-(thiazole-5-carbonyl)-6-azaspiro[3.4]octane-2-carboxylate (90 mg, 0.15 mmol) in DCM (1 mL) was added TEA (45 mg, 0.45 mmol) and TsCl (85 mg, 0.45 mmol) and the reaction stirred at room temperature for 2 h. The mixture was diluted with H₂O (20 mL), extracted with DCM. The combined organic layers were washed with brine, dried over Na₂SO₄, filtered and concentrated. The residue was purified by column chromatography on silica gel (eluent: DCM:MeOH=30:1) to afford tert-butyl 8-(5-((3,4-dichlorophenyl)difluoromethyl)-1,3,4-oxadiazol-2-yl)-6-(thiazole-5-carbonyl)-6-azaspiro[3.4]octane-2-carboxylate (58 mg, 67%) as a yellow oil. LCMS m/z=585.10 [M+H]⁺; ¹H NMR (400 MHz, Methanol-d₄) δ 9.17 (d, J=6.6 Hz, 1H), 8.36 (s, 1H), 7.95-7.87 (m, 1H), 7.77-7.71 (m, 1H), 7.66 (s, 1H), 4.27 (d, J=5.6 Hz, 1H), 4.20-3.76 (m, 5H), 1.47 (s, 4H), 1.43-1.37 (m, 9H).

Step 8: 8-(5-((3,4-dichlorophenyl)difluoromethyl)-1,3,4-oxadiazol-2-yl)-6-(thiazole-5-carbonyl)-6-azaspiro[3.4]octane-2-carboxylic acid: To a solution of tert-butyl 8-(5-((3,4-dichlorophenyl)difluoromethyl)-1,3,4-oxadiazol-2-yl)-6-(thiazole-5-carbonyl)-6-azaspiro[3.4]octane-2-carboxylate (100 mg, 0.17 mmol) in DCM (2 mL) was added TFA (1 mL) and the reaction stirred at room temperature for 2 h. The solvent was removed under vacuum to afford 8-(5-((3,4-dichlorophenyl)difluoromethyl)-1,3,4-oxadiazol-2-yl)-6-(thiazole-5-carbonyl)-6-azaspiro[3.4]octane-2-carboxylic acid (90 mg, 100%) which was used directly in the next step. LCMS m/z=529.10 [M+H]⁺.

### Synthesis of rac-(R)—N-cyclopropyl-8-(5-((3,4-dichlorophenyl)difluoromethyl)-1,3,4-oxadiazol-2-yl)-N-methyl-6-(thiazole-5-carbonyl)-6-azaspiro[3.4]octane-2-carboxamide I-16

A solution of 8-(5-((3,4-dichlorophenyl)difluoromethyl)-1,3,4-oxadiazol-2-yl)-6-(thiazole-5-carbonyl)-6-azaspiro[3.4]octane-2-carboxylic acid (90 mg, 0.17 mmol) and HATU (97 mg, 0.26 mmol) in DMF (5 mL) was stirred at room temperature for 30 min. N-methylcyclopropanamine (13 mg, 0.18 mmol) was added and stirring continued overnight. The mixture was diluted with water (30 mL) and extracted with EtOAc (20 mL×3). The combined organic layers were washed with brine, dried over Na₂SO₄, filtered and concentrated. The mixture was was purified by prep-HPLC to afford rac-(R)—N-cyclopropyl-8-(5-((3,4-dichlorophenyl)difluoromethyl)-1,3,4-oxadiazol-2-yl)-N-methyl-6-(thiazole-5-carbonyl)-6-azaspiro[3.4]octane-2-carboxamide (19 mg, 19%) as a white solid. LCMS m/z=582.15 [M+H]⁺; ¹H NMR (400 MHz, Chloroform-d) δ 8.92 (s, 1H), 8.28 (s, 1H), 7.79 (d, J=19.6 Hz, 1H), 7.63-7.48 (m, 2H), 4.32-3.69 (m, 6H), 2.89 (s, 3H), 2.64-2.22 (m, 5H), 0.92-0.51 (m, 4H).

Table I-5: The compounds listed in Table I-5 were synthesized from 8-(5-((3,4-dichlorophenyl)difluoromethyl)-1,3,4-oxadiazol-2-yl)-6-(thiazole-5-carbonyl)-6-azaspiro[3.4]octane-2-carboxylic acid (see step 8 above) according to the procedures outlined for I-16 using the appropriate commercially available reagents and/or intermediates described above.

| Example | | | |
|---|---|---|---|
| Number | Compound | ¹HNMR | LCMS |
| I-3 | 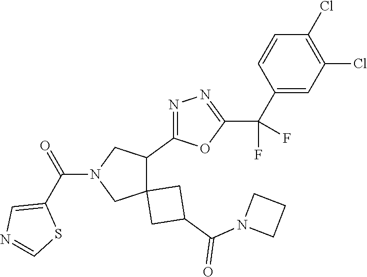 [SMILES: O=C(c1cncs1)N1CC(c2nnc(C(F)(F)c3ccc(Cl)c(Cl)c3)o2)C2(CC(C(=O)N3CCC3)C2)C1] | ¹H NMR (400 MHz, Methanol-d₄) δ 9.16 (d, J = 6.8 Hz, 1H), 8.42-8.31 (m, 1H), 7.97-7.86 (m, 1H), 7.74 (d, J = 8.4 Hz, 1H), 7.69-7.57 (m, 1H), 4.37-3.72 (m, 9H), 3.24-2.82 (m, 1H), 2.53-2.01 (m, 6H). | m/z = 568.1 [M + H]⁺ |

### Synthesis of 8-(5-((3,4-dichlorophenyl)difluoromethyl)-1,3,4-oxadiazol-2-yl)-6-(thiazole-5-carbonyl)-6-azaspiro[3.4]octane-2-carboxamide I-13

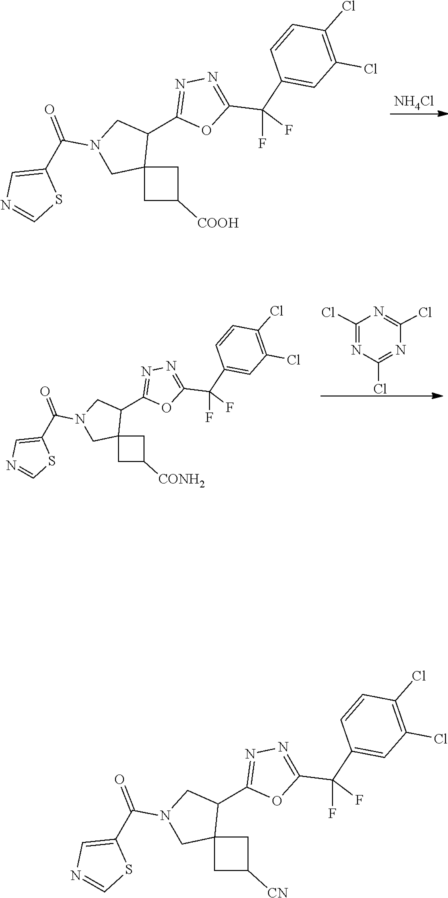

Step 1: 8-(5-((3,4-dichlorophenyl)difluoromethyl)-1,3,4-oxadiazol-2-yl)-6-(thiazole-5-carbonyl)-6-azaspiro[3.4]octane-2-carboxamide: To a solution of 8-(5-((3,4-dichlorophenyl)difluoromethyl)-1,3,4-oxadiazol-2-yl)-6-(thiazole-5-carbonyl)-6-azaspiro[3.4]octane-2-carboxylic acid (90.5 mg, 0.171 mmol) in DMF (5 mL) was added HATU (97.59 mg, 0.256 mmol) and DIPEA (66.34 mg, 0.513 mmol) and the mixture stirred at room temperature for 30 min. Ammonium chloride (27.34 mg, 0.513 mmol) was added and stirring continued for 2 h. The mixture was diluted with water (10 mL) and extracted with EtOAc (30 mL×3). The combined organic layers were washed with brine, dried over Na₂SO₄, filtered and concentrated. The mixture was purified by RP-column (ACN:water=1:3) to afford 8-(5-((3,4-dichlorophenyl)difluoromethyl)-1,3,4-oxadiazol-2-yl)-6-(thiazole-5-carbonyl)-6-azaspiro[3.4]octane-2-carboxamide (50.0 mg, 53%) as a yellow solid. LCMS m/z=528.0[M+H]⁺; ¹H NMR (400 MHz, DMSO-d₆) δ 9.36-9.21 (m, 1H), 8.49-8.31 (m, 1H), 8.04 (dd, J=11.8, 2.2 Hz, 1H), 7.88 (d, J=8.4 Hz, 1H), 7.80-7.62 (m, 1H), 7.28-7.13 (m, 1H), 6.86-6.63 (m, 1H), 4.30-4.11 (m, 1H), 4.10-3.82 (m, 3H), 3.81-3.58 (m, 1H), 2.88-2.72 (m, 1H), 2.41-2.23 (m, 2H), 2.18-2.03 (m, 2H).

Step 2: 8-(5-((3,4-dichlorophenyl)difluoromethyl)-1,3,4-oxadiazol-2-yl)-6-(thiazole-5-carbonyl)-6-azaspiro[3.4]octane-2-carbonitrile: To a solution of 8-(5-((3,4-dichlorophenyl)difluoromethyl)-1,3,4-oxadiazol-2-yl)-6-(thiazole-5-carbonyl)-6-azaspiro[3.4]octane-2-carboxamide (40.0 mg, 0.076 mmol) in DMF (1 mL) at 0° C. under N₂ was added 2,4,6-trichloro-1,3,5-triazine (15.4 mg, 0.083 mmol). The resulting mixture was stirred 3 hours then was diluted with water (100 mL) and extracted with EtOAc three times. The combined organic layers were washed with water, brine, dried over Na₂SO₄, filtered and concentrated. The residue was purified by prep-TLC (eluent: DCM:MeOH=15:1) to afford 8-(5-((3,4-dichlorophenyl)difluoromethyl)-1,3,4-oxadiazol-2-yl)-6-(thiazole-5-carbonyl)-6-azaspiro[3.4]octane-2-carbonitrile (13.0 mg, 34%) as a white solid. LCMS m/z=510.0 [M+H]⁺; ¹H NMR (400 MHz, CD₃OD) δ 9.21-9.10 (m, 1H), 8.40-8.29 (m, 1H), 7.94-7.83 (m, 1H), 7.74 (d, J=8.4 Hz, 1H), 7.68-7.59 (m, 1H), 4.34-3.80 (m, 5H), 3.28-3.24 (m, 1H), 2.79-2.39 (m, 4H).

### Synthesis of (2R,3S)-2-acetamido-3-(cyclohexylmethoxy)-N-(2-((S)-2,2-dimethylcyclopropane-1-carbonyl)-6-(thiazole-5-carbonyl)-2,6-diazaspiro[3.4]octan-8-yl)butanamide (I-24)

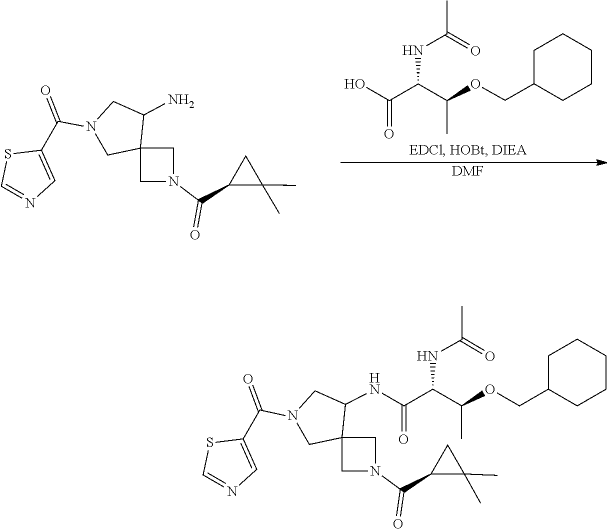

(2R,3S)-2-acetamido-3-(cyclohexylmethoxy)-N-(2-((S)-2,2-dimethylcyclopropane-1-carbonyl)-6-(thiazole-5-carbonyl)-2,6-diazaspiro[3.4]octan-8-yl)butanamide was synthesized from (8-amino-2-((S)-2,2-dimethylcyclopropane-1-carbonyl)-2,6-diazaspiro[3.4]octan-6-yl)(thiazol-5-yl)methanone according to the method outlined in (2R,3S)-2-acetamido-N-(6-(1-benzyl-1H-pyrazole-4-carbonyl)-2-((S)-2,2-dimethylcyclopropane-1-carbonyl)-2,6-diazaspiro[3.4]octan-8-yl)-3-(cyclohexylmethoxy)butanamide (I-25). LCMS m/z=574.4 [M+H]⁺; ¹H NMR (400 MHz, CD₃OD) δ 9.17 (s, 1H), 8.37 (dd, J=24.4, 5.6 Hz, 1H), 4.79-4.69 (m, 1H), 4.50-3.59 (m, 10H), 3.43-3.32 (m, 1H), 3.23-3.12 (m, 1H), 2.03-1.99 (m, 3H), 1.74-1.64 (m, 5H), 1.60-1.35 (m, 2H), 1.28-1.02 (m, 13H), 0.98-0.75 (m, 3H).

### Synthesis of (2R,3S)-2-acetamido-N-(6-(1-benzyl-1H-pyrazole-4-carbonyl)-2-((S)-2,2-dimethylcyclopropane-1-carbonyl)-2,6-diazaspiro[3.4]octan-8-yl)-3-(cyclohexylmethoxy)butanamide I-25

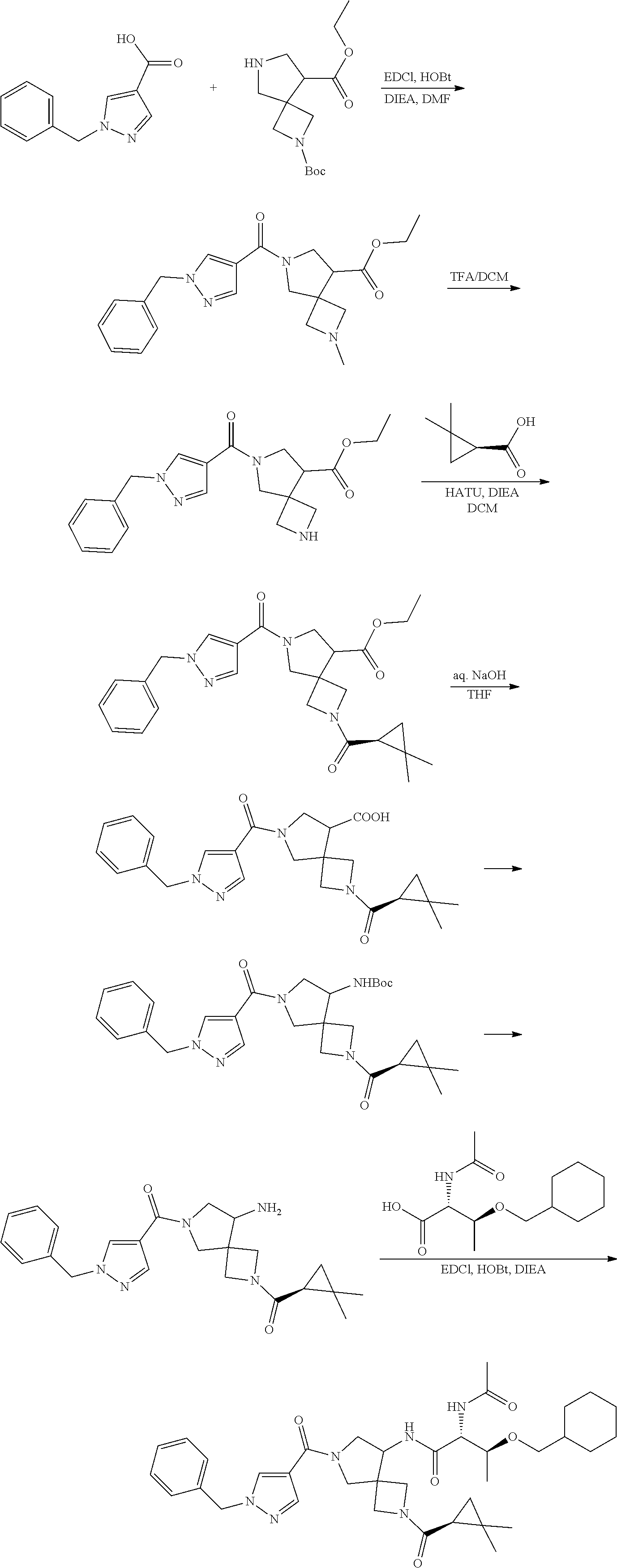

Step 1: 2-(tert-butyl) 8-ethyl 6-(1-benzyl-1H-pyrazole-4-carbonyl)-2,6-diazaspiro[3.4]octane-2,8-dicarboxylate A mixture of 2-(tert-butyl) 8-ethyl 2,6-diazaspiro[3.4]octane-2,8-dicarboxylate (22 g, 77.5 mmol), 1-benzyl-1H-pyrazole-4-carboxylic acid (17.2 g, 85.2 mmol), EDCI (22 g, 116 mmol), HOBt (12.5 g, 93 mmol) and DIEA (30 g, 232 mmol) in DMF (150 mL) was stirred at room temperature overnight. Water was added and the aqueous extracted with EtOAc three times. The combined organic layers were washed with water and brine, dried over Na₂SO₄, filtered and concentrated. The residue obtained was purified by silica gel column (3.3% MeOH/DCM) to afford 2-(tert-butyl) 8-ethyl 6-(1-benzyl-1H-pyrazole-4-carbonyl)-2,6-diazaspiro[3.4]octane-2,8-dicarboxylate (19 g, 53%) as a colorless oil. LCMS m/z=469.2 [M+H]⁺

Step 2: ethyl 6-(1-benzyl-1H-pyrazole-4-carbonyl)-2,6-diazaspiro[3.4]octane-8-carboxylate TFA (30 mL) was added to a solution of 2-(tert-butyl) 8-ethyl 6-(1-benzyl-1H-pyrazole-4-carbonyl)-2,6-diazaspiro[3.4]octane-2,8-dicarboxylate (19 g, 40.6 mmol) in DCM (30 mL) and the reaction stirred at room temperature for 4 h. The solvent was removed and the crude was used in next step directly. LCMS m/z=369.1 [M+H]⁺

Step 3: ethyl 6-(1-benzyl-1H-pyrazole-4-carbonyl)-2-((S)-2,2-dimethylcyclopropane-1-carbonyl)-2,6-diazaspiro[3.4]octane-8-carboxylate A solution of (S)-2,2-dimethylcyclopropane-1-carboxylic acid (5.1 g, 44.8 mmol), HATU (23 g, 61 mmol) and DIEA (13.8 g, 122 mmol) in DCM (50 mL) was stirred at room temperature for 30 min. Ethyl 6-(1-benzyl-1H-pyrazole-4-carbonyl)-2,6-diazaspiro[3.4]octane-8-carboxylate (15 g, 40.7 mmol) was added and the reaction stirred at room temperature overnight. The solvent was removed and the crude was purified by silica gel column (3.3% MeOH/DCM) to afford ethyl 6-(1-benzyl-1H-pyrazole-4-carbonyl)-2-((S)-2,2-dimethylcyclopropane-1-carbonyl)-2,6-diazaspiro[3.4]octane-8-carboxylate (12 g, 63%) as yellow oil. LCMS m/z=465.4 [M+H]⁺

Step 4: 6-(1-benzyl-1H-pyrazole-4-carbonyl)-2-((S)-2,2-dimethylcyclopropane-1-carbonyl)-2,6-diazaspiro[3.4]octane-8-carboxylic acid To a solution of ethyl 6-(1-benzyl-1H-pyrazole-4-carbonyl)-2-((S)-2,2-dimethylcyclopropane-1-carbonyl)-2,6-diazaspiro[3.4]octane-8-carboxylate (12 g, 25.8 mmol) in MeOH (20 mL) was added aq. NaOH (10%, 15 mL) and the resulting mixture stirred at room temperature for 4 hours. The pH was adjusted to ˜1 with 1M HCl and the aqueous was extracted with EtOAc three times. The combined organic layers were washed with water and brine, dried over Na₂SO₄, filtered and concentrated. The solvent was removed to afford 6-(1-benzyl-1H-pyrazole-4-carbonyl)-2-((S)-2,2-dimethylcyclopropane-1-carbonyl)-2,6-diazaspiro[3.4]octane-8-carboxylic acid (8 g, 71%) as a white solid which was used in next step directly. LCMS m/z=437.3 [M+H]⁺

Step 5: Tert-butyl (6-(1-benzyl-1H-pyrazole-4-carbonyl)-2-((S)-2,2-dimethylcyclopropane-1-carbonyl)-2,6-diazaspiro[3.4]octan-8-yl)carbamate A mixture of 6-(1-benzyl-1H-pyrazole-4-carbonyl)-2-((S)-2,2-dimethylcyclopropane-1-carbonyl)-2,6-diazaspiro[3.4]octane-8-carboxylic acid (200 mg, 0.46 mmol), DPPA (126 mg, 0.46 mmol), (Boc)₂O (298 mg, 1.37 mmol) and TEA (138 mg, 1.37 mmol) in t-BuOH (40.0 mL) was heated at reflux overnight. The mixture was diluted with water (30 mL) and extracted with EtOAc (60 mL×2). The combined organic layers were washed with brine, dried over Na₂SO₄, filtered and concentrated. The residue obtained was purified by prep-TLC (DCM/MeOH=15/1) to afford tert-butyl (2-((S)-2,2-dimethylcyclopropane-1-carbonyl)-6-(thiazole-5-carbonyl)-2,6-diazaspiro[3.4]octan-8-yl)carbamate (86 mg, 37%) as a white solid. LCMS m/z=508.2 [M+H]⁺.

Step 6: (8-amino-2-((S)-2,2-dimethylcyclopropane-1-carbonyl)-2,6-diazaspiro[3.4]octan-6-yl)(1-benzyl-1H-pyrazol-4-yl)methanone hydrochloride To a solution of Tert-butyl (6-(1-benzyl-1H-pyrazole-4-carbonyl)-2-((S)-2,2-dimethylcyclopropane-1-carbonyl)-2,6-diazaspiro[3.4]octan-8-yl)carbamate (80 mg, 0.16 mmol) in MeOH (4 mL) was added a solution of HCl in dixoane (4M, 2 mL). The reaction was stirred for 2 h, then the solvent was removed under reduced pressure to afford (8-amino-2-((S)-2,2-dimethylcyclopropane-1-carbonyl)-2,6-diazaspiro[3.4]octan-6-yl)(1-benzyl-1H-pyrazol-4-yl)methanone hydrochloride (100 mg, quant.). LCMS m/z=408.1 [M+H]⁺.

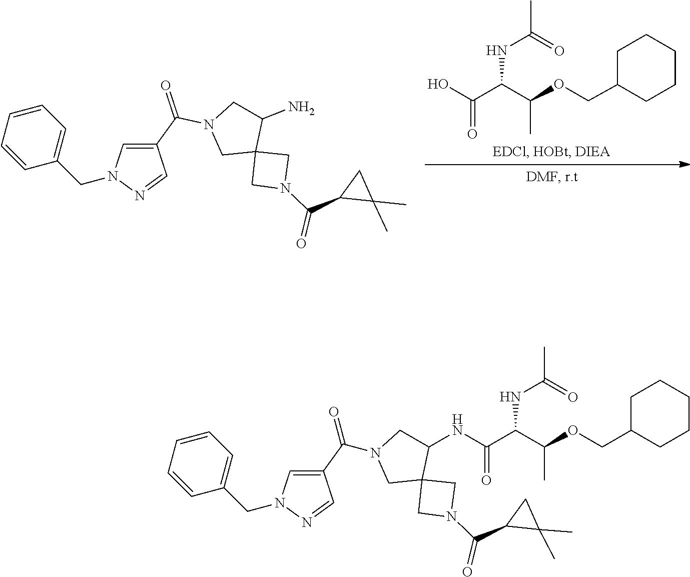 [SMILES: CC1(C)C[C@@H]1C(=O)N1CC2(CN(C(=O)c3cnn(Cc4ccccc4)c3)CC2N)C1 ; CC(=O)N[C@@H](C(=O)O)[C@H](C)OCC1CCCCC1 ; CC(=O)N[C@@H](C(=O)NC1CN(C(=O)c2cnn(Cc3ccccc3)c2)CC12CN(C(=O)[C@H]1CC1(C)C)C2)[C@H](C)OCC1CCCCC1]

Step 7: To a solution of N-acetyl-O-(cyclohexylmethyl)-D-threonine (25 mg, 0.098 mmol) in DMF (2 mL) was added (8-amino-2-((S)-2,2-dimethylcyclopropane-1-carbonyl)-2,6-diazaspiro[3.4]octan-6-yl)(1-benzyl-1H-pyrazol-4-yl)methanone (40 mg, 0.098 mmol), EDCI (28 mg, 0.147 mmol), HOBt (20 mg, 0.147 mmol) and DIPEA (50 mg, 0.392 mmol). The resulting mixture was stirred at room temperature for 14 h then was diluted with water (20 mL) and extracted with EtOAc (40 mL×2). The combined organic layers were washed with brine, dried over Na₂SO₄, filtered and concentrated. The residue was purified by prep-HPLC to afford (2R,3S)-2-acetamido-N-(6-(1-benzyl-1H-pyrazole-4-carbonyl)-2-((S)-2,2-dimethylcyclopropane-1-carbonyl)-2,6-diazaspiro[3.4]octan-8-yl)-3-(cyclohexylmethoxy)butanamide (12 mg, 18%) as a white solid. LCMS m/z=647.4 [M+H]⁺; ¹H NMR (400 MHz, CD₃OD) δ 8.29-8.15 (m, 1H), 7.98-7.83 (m, 1H), 7.38-7.24 (m, 5H), 5.41-5.35 (m, 2H), 4.75-4.63 (m, 1H), 4.52-3.91 (m, 6H), 3.91-3.50 (m, 4H), 3.39-3.33 (m, 1H), 3.23-3.09 (m, 1H), 2.06-1.93 (m, 3H), 1.78-1.57 (m, SH), 1.54-1.37 (m, 2H), 1.25-0.94 (m, 14H), 0.93-0.72 (m, 3H).

### Synthesis of 2-((S)-2,2-dimethylcyclopropane-1-carbonyl)-6-(thiazole-5-carbonyl)-2,6-diazaspiro[3.4]octane-8-carbonitrile (I-27)

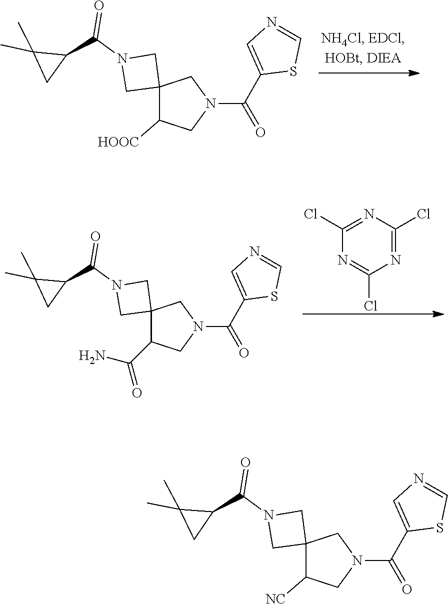 [SMILES: CC1(C)C[C@@H]1C(=O)N1CC2(CN(C(=O)c3cncs3)CC2C(=O)O)C1 ; CC1(C)C[C@@H]1C(=O)N1CC2(CN(C(=O)c3cncs3)CC2C(N)=O)C1 ; Clc1nc(Cl)nc(Cl)n1 ; CC1(C)C[C@@H]1C(=O)N1CC2(CN(C(=O)c3cncs3)CC2C#N)C1]

Step 1: 2-((S)-2,2-dimethylcyclopropane-1-carbonyl)-6-(thiazole-5-carbonyl)-2,6-diazaspiro[3.4]octane-8-carboxamide: To a solution of 2-((S)-2,2-dimethylcyclopropane-1-carbonyl)-6-(thiazole-5-carbonyl)-2,6-diazaspiro[3.4]octane-8-carboxylic acid (10.0 g, 27.5 mmol) in DMF (100 mL) was added NH₄Cl (4.42 g, 82.6 mmol), EDCI (7.91 g, 41.3 mmol), HOBt (5.58 g, 41.3 mmol) and DTPEA (10.7 g, 82.6 mmol). The resulting mixture was stirred at room temperature for 48 h then the solvent was removed under reduced pressure and the residue obtained purified by RP-column (24% MeCN in water) to afford 2-((S)-2,2-dimethylcyclopropane-1-carbonyl)-6-(thiazole-5-carbonyl)-2,6-diazaspiro[3.4]octane-8-carboxamide (9.8 g, 98%) as a light yellow solid. LCMS m/z=363.1[M+H]⁺; ¹H NMR (400 MHz, DMSO-d6) δ 9.25 (s, 1H), 8.37 (dd, J=13.2, 2.8 Hz, 1H), 7.70 (d, J=9.6 Hz, 1H), 7.20 (s, 1H), 4.40-3.60 (m, 8H), 3.24-3.02 (m, 1H), 1.41-1.27 (m, 1H), 1.15-1.01 (m, 6H), 0.89-0.82 (m, 1H), 0.71-0.62 (m, 1H).

Step 2: 2-((S)-2,2-dimethylcyclopropane-1-carbonyl)-6-(thiazole-5-carbonyl)-2,6-diazaspiro[3.4]octane-8-carbonitrile: To a solution of 2-((S)-2,2-dimethylcyclopropane-1-carbonyl)-6-(thiazole-5-carbonyl)-2,6-diazaspiro[3.4]octane-8-carboxamide (8.8 g, 24.3 mmol) in DMF (80 mL) was added 2,4,6-trichloro-1,3,5-triazine (4.92 g, 26.7 mmol). The reaction was stirred at 0° C. for 2 h then was diluted with water and extracted with EtOAc (200 mL×3). The combined organic layers were washed with water, brine, dried over Na₂SO₄ filtered and concentrated. The residue was purified by column chromatography on silica gel (eluent: DCM:MeOH=20:1) to afford 2-((S)-2,2-dimethylcyclopropane-1-carbonyl)-6-(thiazole-5-carbonyl)-2,6-diazaspiro[3.4]octane-8-carbonitrile (2.5 g, 30%) as a white solid. LCMS m/z=345.1 [M+H]⁺; ¹H NMR (400 MHz, DMSO-d6) δ 9.28 (s, 1H), 8.47-8.34 (m, 1H), 4.39-3.70 (m, 9H), 1.47-1.32 (m, 1H), 1.16-1.04 (m, 6H), 0.91-0.84 (m, 1H), 0.74-0.65 (m, 1H).

### Synthesis of 6-(1-benzyl-1H-pyrazole-4-carbonyl)-2-((S)-2,2-dimethylcyclopropane-1-carbonyl)-2,6-diazaspiro[3.4]octane-8-carbonitrile (I-40)

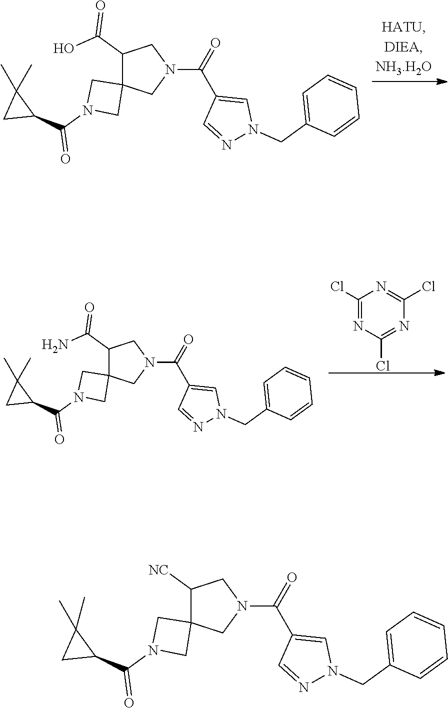 [SMILES: CC1(C)C[C@@H]1C(=O)N1CC2(CN(C(=O)c3cnn(Cc4ccccc4)c3)CC2C(=O)O)C1 ; CC1(C)C[C@@H]1C(=O)N1CC2(CN(C(=O)c3cnn(Cc4ccccc4)c3)CC2C(N)=O)C1 ; Clc1nc(Cl)nc(Cl)n1 ; CC1(C)C[C@@H]1C(=O)N1CC2(CN(C(=O)c3cnn(Cc4ccccc4)c3)CC2C#N)C1]

Step 1: 6-(1-benzyl-1H-pyrazole-4-carbonyl)-2-((S)-2,2-dimethylcyclopropane-1-carbonyl)-2,6-diazaspiro[3.4]octane-8-carboxamide: To a solution of 6-(1-benzyl-1H-pyrazole-4-carbonyl)-2-((S)-2,2-dimethylcyclopropane-1-carbonyl)-2,6-diazaspiro[3.4]octane-8-carboxylic acid (250 mg, 0.57 mmol) in DMF (5 mL) was added HATU (327 mg, 0.86 mmol) and DIPEA (222 mg, 1.72 mmol). The mixture was stirred at room temperature for 30 min then poured into aq. NH₄OH (25 mL). The reaction was stirred for another 2 hours then the solvent was removed under reduced pressure and the residue obtained purified by reverse-phase column to afford 6-(1-benzyl-1H-pyrazole-4-carbonyl)-2-((S)-2,2-dimethylcyclopropane-1-carbonyl)-2,6-diazaspiro[3.4]octane-8-carboxamide (120 mg, 48%) as a white solid. LCMS m/z=436.3 [M+H]⁺.

Step 2: 6-(1-benzyl-1H-pyrazole-4-carbonyl)-2-((S)-2,2-dimethylcyclopropane-1-carbonyl)-2,6-diazaspiro[3.4]octane-8-carbonitrile (I-40): To a solution of 6-(1-benzyl-1H-pyrazole-4-carbonyl)-2-((S)-2,2-dimethylcyclopropane-1-carbonyl)-2,6-diazaspiro[3.4]octane-8-carboxamide (50 mg, 0.11 mmol) in DMF (2 mL) at 0° C. was added 2,4,6-trichloro-1,3,5-triazine (21.2 mg, 0.11 mmol). The reaction was stirred for 2 h then water was added and the aqueous extracted with EtOAc. The combined organic layers were washed with water, brine and dried over Na₂SO₄. The residue obtained was purified by prep-TLC (6% MeOH/DCM) to afford 6-(1-benzyl-1H-pyrazole-4-carbonyl)-2-((S)-2,2-dimethylcyclopropane-1-carbonyl)-2,6-diazaspiro[3.4]octane-8-carbonitrile (25 mg, 52%) as a white solid. LCMS m/z=418.3 [M+H]⁺; ¹HNMR (400 MHz, CD₃OD) δ 8.23 (d, J=12.8 Hz, 1H), 7.93 (d, J=12.8 Hz, 1H), 7.43-7.21 (m, 5H), 5.38 (s, 2H), 4.58-3.65 (m, 9H), 1.51-1.40 (m, 1H), 1.22-1.04 (m, 7H), 0.85-0.76 (m, 1H).

### Synthesis of N-((2S,3R)-3-(cyclohexylmethoxy)-1-oxo-1-(piperidin-1-yl)butan-2-yl)-2-((S)-2,2-dimethylcyclopropane-1-carbonyl)-6-thia-2-azaspiro[3.4]octane-8-carboxamide 6,6-dioxide (I-26)

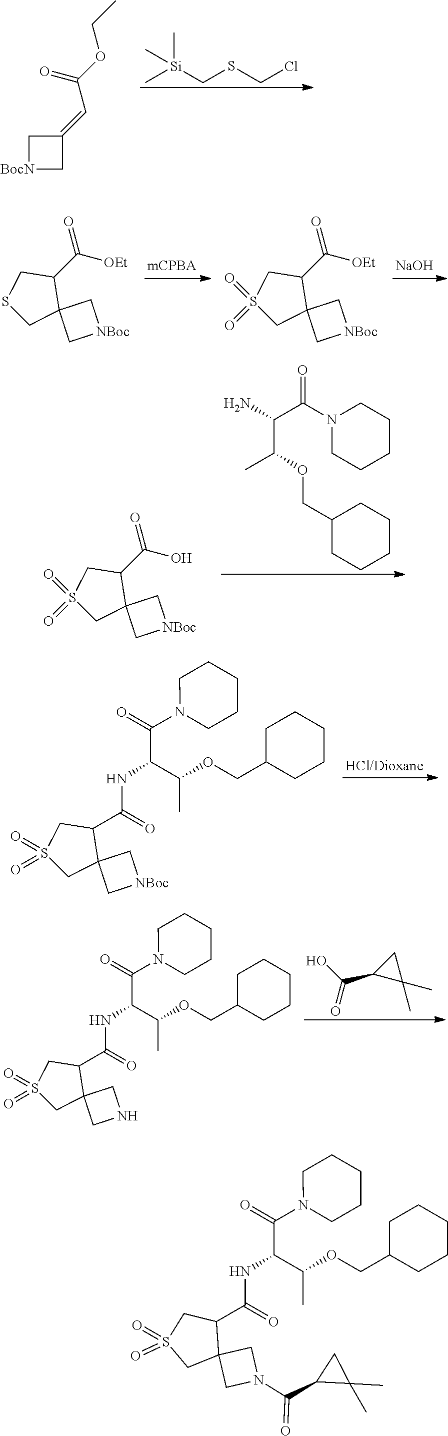

Step 1: 2-(tert-butyl) 8-ethyl 6-thia-2-azaspiro[3.4]octane-2,8-dicarboxylate: To a solution of tert-butyl 3-(2-ethoxy-2-oxoethylidene)azetidine-1-carboxylate (200 mg, 0.83 mmol) in ACN (10 mL) was added (((chloromethyl)thio)methyl)trimethylsilane (378 mg, 0.24 mmol) and CsF (756 Ng, 4.97 mol). The mixture was heated at 80° C. for 12 h then was diluted with water (10 mL) and extracted with EtOAc (20 mL×2). The combined organic layers were washed with water and dried over Na₂SO₄. The residue obtained was purified by column chromatography on silica gel (eluted with Pet ether:EtOAc=10:1) to afford 2-(tert-butyl) 8-ethyl 6-thia-2-azaspiro[3.4]octane-2,8-dicarboxylate (167 mg, 67%) as a colorless oil. LCMS m/z=246.0 [M-55]⁺.

Step 2: 2-(tert-butyl) 8-ethyl 6-thia-2-azaspiro[3.4]octane-2,8-dicarboxylate 6,6-dioxide: To a solution of 2-(tert-butyl) 8-ethyl 6-thia-2-azaspiro[3.4]octane-2,8-dicarboxylate (117 mg, 0.388 mmol) in DCM (1 mL) was added m-CPBA (266 mg, 1.55 mmol). The reaction was stirred at room temperature for 1 hour then diluted with water (10 mL) and extracted with DCM (10 mL×2). The combined organic layers was washed with water, dried over Na₂SO₄, filtered and concentrated under reduced pressure. The residue obtained was purified by column chromatography on silica gel (eluted with Pet ether:EtOAc=4:1) to afford 2-(tert-butyl) 8-ethyl 6-thia-2-azaspiro[3.4]octane-2,8-dicarboxylate 6,6-dioxide (63 mg, 53%) as a yellow oil. LCMS m/z=278.0 [M-55]⁺.

Step 3: 2-(tert-butoxycarbonyl)-6-thia-2-azaspiro[3.4]octane-8-carboxylic acid 6,6-dioxide: To a solution of 2-(tert-butyl) 8-ethyl 6-thia-2-azaspiro[3.4]octane-2,8-dicarboxylate 6,6-dioxide (64 mg, 0.19 mmol) in MeOH (1 mL) was added NaOH (10% in water, 1 mL) and the reaction stirred at room temperature for 12 h. The reaction was diluted with water (20 mL), acidified with 1M HCl to pH˜2, then extracted with EtOAc (20 mL×3). The combined organic layers were washed with brine, dried over Na₂SO₄, filtered and concentrated to afford 2-(tert-butoxycarbonyl)-6-thia-2-azaspiro[3.4]octane-8-carboxylic acid 6,6-dioxide (45 mg, 77%) as a yellow solid.

Step 4: tert-butyl 8-(((2S,3R)-3-(cyclohexylmethoxy)-1-oxo-1-(piperidin-1-yl)butan-2-yl)carbamoyl)-6-thia-2-azaspiro[3.4]octane-2-carboxylate 6,6-dioxide: To a solution of 2-(tert-butoxycarbonyl)-6-thia-2-azaspiro[3.4]octane-8-carboxylic acid 6,6-dioxide (37 mg, 0.12 mmol) in DMA (2 mL) were added (2S,3R)-2-amino-3-(cyclohexylmethoxy)-1-(piperidin-1-yl)butan-1-one hydrochloride (51 mg, 0.18 mmol), EDCI (35 mg, 0.18 mmol), HOBt (20 mg, 0.15 mmol) and DIEA (31 mg, 0.24 mmol) and the reaction was stirred at room temperature overnight. The reaction mixture was treated with water (20 mL) and extracted with EtOAc (20 mL×2). The combined organic layers was washed with water (20 mL×3), dried over Na₂SO₄, filtered and concentrated. The residue obtained was purified by column chromatography on silica gel (eluted with DCM/MeOH=20/1) to afford the tert-butyl 8-(((2S,3R)-3-(cyclohexylmethoxy)-1-oxo-1-(piperidin-1-yl)butan-2-yl)carbamoyl)-6-thia-2-azaspiro[3.4]octane-2-carboxylate 6,6-dioxide (37 mg, 53%) as a colorless oil. LCMS m/z=568.3 [M−H]⁻.

Step 5: N-((2S,3R)-3-(cyclohexylmethoxy)-1-oxo-1-(piperidin-1-yl)butan-2-yl)-6-thia-2-azaspiro[3.4]octane-8-carboxamide 6,6-dioxide hydrochloride: To a solution of tert-butyl 8-(((2S,3R)-3-(cyclohexylmethoxy)-1-oxo-1-(piperidin-1-yl)butan-2-yl)carbamoyl)-6-thia-2-azaspiro[3.4]octane-2-carboxylate 6,6-dioxide (35 mg, 0.06 mmol) in DCM (1 mL) was added a solution of HCl in Dioxane (4 M, 1 mL). The reaction was stirred at room temperature for 4 h then the solvent removed under reduced pressure to afford N-((2S,3R)-3-(cyclohexylmethoxy)-1-oxo-1-(piperidin-1-yl)butan-2-yl)-6-thia-2-azaspiro[3.4]octane-8-carboxamide 6,6-dioxide hydrochloride (39 mg, quant.) as a white solid.

Step 6: N-((2S,3R)-3-(cyclohexylmethoxy)-1-oxo-1-(piperidin-1-yl)butan-2-yl)-2-((S)-2,2-dimethylcyclopropane-1-carbonyl)-6-thia-2-azaspiro[3.4]octane-8-carboxamide 6,6-dioxide (I-26): To a solution of N-((2S,3R)-3-(cyclohexylmethoxy)-1-oxo-1-(piperidin-1-yl)butan-2-yl)-6-thia-2-azaspiro[3.4]octane-8-carboxamide 6,6-dioxide hydrochloride (35 mg, 0.07 mmol) in DMA (2 mL) was added (S)-2,2-dimethylcyclopropane-1-carboxylic acid (13 mg, 0.11 mmol), EDCI (21 mg, 0.11 mmol), HOBt (12 mg, 0.09 mmol) and DIEA (19 mg, 0.15 mmol) and the mixture stirred at room temperature overnight. The reaction was quenched with water (10 mL) and extracted with EtOAc (20 mL×2). The combined organic layers were washed with water (20 mL×3), dried over Na₂SO₄, filtered and concentrated under reduced pressure. The residue obtained was purified by column chromatography (eluted with DCM:MeOH=20:1) to afford N-((2S,3R)-3-(cyclohexylmethoxy)-1-oxo-1-(piperidin-1-yl)butan-2-yl)-2-((S)-2,2-dimethylcyclopropane-1-carbonyl)-6-thia-2-azaspiro[3.4]octane-8-carboxamide 6,6-dioxide (15 mg, 36%) as a oil. LCMS m/z=566.3[M+H]⁺; ¹H NMR (400 MHz, Methanol-d₄) δ 4.99-4.91 (m, 1H), 4.64-3.84 (m, 4H), 3.79-3.64 (m, 3H), 3.61-3.51 (m, 3H), 3.46-3.35 (m, 3H), 3.27-3.14 (m, 1H), 1.78-1.48 (m, 11H), 1.40-1.26 (m, 5H), 1.22-1.09 (m, 10H), 1.06-1.00 (m, 1H), 1.00-0.85 (m, 3H), 0.81-0.74 (m, 1H).

### Synthesis of N-((2S,3R)-3-(cyclohexylmethoxy)-1-oxo-1-(piperidin-1-yl)butan-2-yl)-2-((S)-2,2-dimethylcyclopropane-1-carbonyl)-6-thia-2-azaspiro[3.4]octane-8-carboxamide I-23

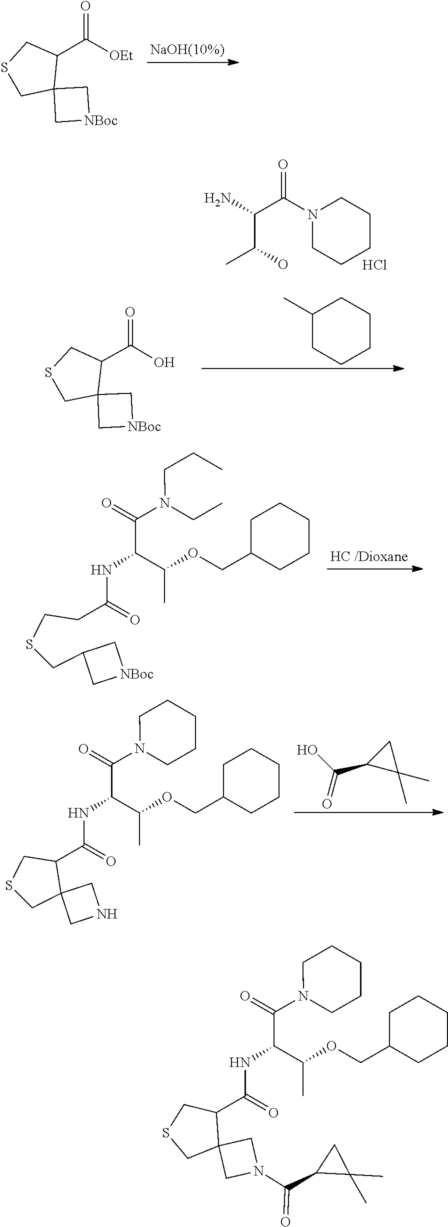

Step 1: 2-(tert-butoxycarbonyl)-6-thia-2-azaspiro[3.4]octane-8-carboxylic acid: To a solution of methyl 2-(tert-butyl) 8-ethyl 6-thia-2-azaspiro[3.4]octane-2,8-dicarboxylate (50 mg, 0.17 mmol) in MeOH (1 mL) was added NaOH (10% in water, 1 mL) and the reaction stirred at room temperature for 12 h. The reaction was diluted with water (20 mL), acidified with IN HCl to pH˜2 and extracted with EtOAc (20 mL×3). The combined organic layers were washed with brine, dried over Na₂SO₄, filtered and concentrated to afford 2-(tert-butoxycarbonyl)-6-thia-2-azaspiro[3.4]octane-8-carboxylic acid (45 mg, quant.) as a colorless oil.

Step 2: tert-butyl 8-(((2S,3R)-3-(cyclohexylmethoxy)-1-oxo-1-(piperidin-1-yl)butan-2-yl)carbamoyl)-6-thia-2-azaspiro[3.4]octane-2-carboxylate: To a solution of 2-(tert-butoxycarbonyl)-6-thia-2-azaspiro[3.4]octane-8-carboxylic acid (45 mg, 0.16 mmol) in DMF (2 mL) was added (2S,3R)-2-amino-3-(cyclohexylmethoxy)-1-(piperidin-1-yl)butan-1-one hydrochloride (70 mg, 0.25 mmol), EDCI (47 mg, 0.25 mmol), HOBt (27 mg, 0.20 mmol) and DIEA (43 mg, 0.33 mmol) and the reaction stirred at room temperature overnight. The reaction was quenched with water (10 mL) and extracted with EtOAc (50 mL×2). The combined organic layers were washed with water (10 mL×3), dried over Na₂SO₄, filtered and concentrated under reduced pressure. The residue obtained was purified by prep-TLC (eluted with DCM/MeOH=20/1) to afford tert-butyl 8-(((2S,3R)-3-(cyclohexylmethoxy)-1-oxo-1-(piperidin-1-yl)butan-2-yl)carbamoyl)-6-thia-2-azaspiro[3.4]octane-2-carboxylate (56 mg, 63%) as a colorless oil. LCMS m/z=579.2 [M+H]⁺.

Step 3: N-((2S,3R)-3-(cyclohexylmethoxy)-1-oxo-1-(piperidin-1-yl)butan-2-yl)-6-thia-2-azaspiro[3.4]octane-8-carboxamide hydrochloride: To a solution of tert-butyl 8-(((2S,3R)-3-(cyclohexylmethoxy)-1-oxo-1-(piperidin-1-yl)butan-2-yl)carbamoyl)-6-thia-2-azaspiro[3.4]octane-2-carboxylate (36 mg, 0.07 mmol) in DCM (1 mL) was added a solution of HCl in dioxane (4 M, 1 mL). The mixture was stirred at room temperature for 4 h then the solvent was removed under reduced pressure to afford N-((2S,3R)-3-(cyclohexylmethoxy)-1-oxo-1-(piperidin-1-yl)butan-2-yl)-6-thia-2-azaspiro[3.4]octane-8-carboxamide hydrochloride (67 mg, quant.) as a white solid.

Step 4: N-((2S,3R)-3-(cyclohexylmethoxy)-1-oxo-1-(piperidin-1-yl)butan-2-yl)-2-((S)-2,2-dimethylcyclopropane-1-carbonyl)-6-thia-2-azaspiro[3.4]octane-8-carboxamide (I-23): To a solution of N-((2S,3R)-3-(cyclohexylmethoxy)-1-oxo-1-(piperidin-1-yl)butan-2-yl)-6-thia-2-azaspiro[3.4]octane-8-carboxamide hydrochloride (60 mg, 0.14 mmol) in DMA (2 mL) was added (S)-2,2-dimethylcyclopropane-1-carboxylic acid (23 mg, 0.21 mmol), EDCI (39 mg, 0.21 mmol), HOBt (22 mg, 0.16 mmol) and DIEA (35 mg, 0.27 mmol) and the reaction stirred overnight. The reaction mixture was diluted with water (10 mL) and and extracted with EtOAc (20 mL×3). The combined organic layers were washed with water (10 mL×3), dried over Na₂SO₄, filtered and concentrated. The residue obtained was purified by prep-HPLC to afford N-((2S,3R)-3-(cyclohexylmethoxy)-1-oxo-1-(piperidin-1-yl)butan-2-yl)-2-((S)-2,2-dimethylcyclopropane-1-carbonyl)-6-thia-2-azaspiro[3.4]octane-8-carboxamide (5 mg, 7%) as a oil. LCMS m/z=534.4 [M+H]⁺; ¹H NMR (400 MHz, Methanol-d₄) δ 4.98-4.91 (m, 1H), 4.58-3.70 (m, 5H), 3.65-3.48 (m, 4H), 3.41-3.33 (m, 1H), 3.29-3.16 (m, 3H), 3.11-2.99 (m, 3H), 1.79-1.57 (m, 10H), 1.45-1.33 (m, 2H), 1.23-1.08 (m, 11H), 1.04-0.87 (m, 5H), 0.80-0.73 (m, 1H).

### Building Blocks:

### Synthesis of 1-benzyl-4-(chloromethyl)-1H-pyrazole

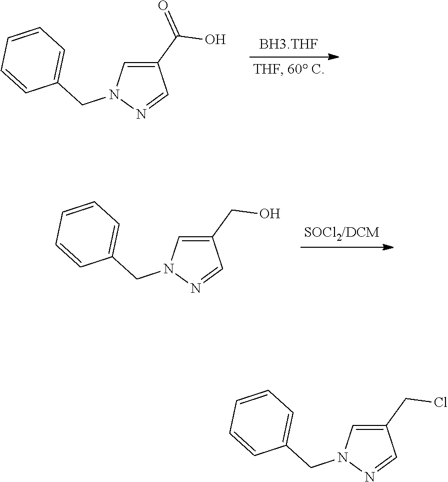 [SMILES: O=C(O)c1cnn(Cc2ccccc2)c1 ; OCc1cnn(Cc2ccccc2)c1 ; ClCc1cnn(Cc2ccccc2)c1]

Step 1: (1-benzyl-1H-pyrazol-4-yl)methanol: To a solution of 1-benzyl-1H-pyrazole-4-carboxylic acid (1.5 g, 7.43 mmol) in THF (12 mL) was added BH₃THF (22 mL, 22 mmol). The reaction was heated at 60° C. for 4 h then quenched with saturated ammonium chloride solution (30 mL) and extracted with EtOAc (50 mL×3). The combined organic layers were washed with brine, dried over Na₂SO₄, filtered and concentrated. The residue was purified by RP column (30% CH₃CN in water) to afford (1-benzyl-1H-pyrazol-4-yl)methanol (411 mg, 30%) as a colorless oil. LCMS m/z=189.2 [M+H]⁺; ¹H NMR (400 MHz, DMSO-d₆) δ 7.67 (s, 1H), 7.39-7.25 (m, 4H), 7.24-7.19 (m, 2H), 5.27 (s, 2H), 4.81 (t, J=5.6 Hz, 1H), 4.34 (d, J=5.4 Hz, 2H).

Step 2: 1-benzyl-4-(chloromethyl)-1H-pyrazole: To a solution of (1-benzyl-1H-pyrazol-4-yl)methanol (490 mg, 2.44 mmol) in DCM (6 mL) was added SOCl₂ (0.7 mL, 9.77 mmol). The reaction mixture was stirred at room temperature for 2 h then the solvent was removed under vacuum to afford crude 1-benzyl-4-(chloromethyl)-1H-pyrazole (505 mg, 94%) which was used directly in the next step. ¹H NMR (400 MHz, DMSO-d₆) δ 7.92 (s, 1H), 7.53 (s, 1H), 7.36-7.26 (m, 3H), 7.25-7.20 (m, 2H), 5.30 (s, 2H), 4.69 (s, 2H).

### Synthesis of methyl O-(cyclohexylmethyl)-L-threoninate

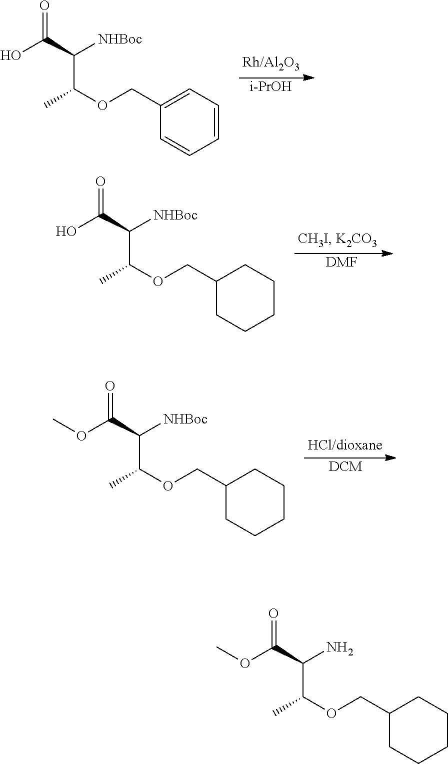

Step 1: N-(tert-butoxycarbonyl)-O-(cyclohexylmethyl)-L-threonine: To a solution of O-benzyl-N-(tert-butoxycarbonyl)-L-threonine (2 g, 6.47 mmol) in i-PrOH (12 mL) was added rhodium on Al₂O₃ (600 mg). The reaction was stirred under a H₂ atmosphere for 48 h then the catalyst was removed by filtration through Celite and the filtrate concentrated to afford crude N-(tert-butoxycarbonyl)-O-(cyclohexylmethyl)-L-threonine (1.92 g, 94%) as a colorless oil. LCMS m/z=260.2 [M+H-56]⁺.

Step 2: methyl N-(tert-butoxycarbonyl)-O-(cyclohexylmethyl)-L-threoninate: To a solution of N-(tert-butoxycarbonyl)-O-(cyclohexylmethyl)-L-threonine (1.92 g, 6.1 mmol) in DMF (12 mL) was added K₂CO₃ (1.68 g, 12.2 mmol) and CH₃I (0.76 mL, 12.2 mmol). The reaction mixture was stirred at room temperature for 4 h then was diluted with water (30 mL) and extracted with EtOAc (50 mL×2). The combined organic layers were washed with brine, dried over Na₂SO₄, filtered and concentrated. The residue was purified by column chromatography on silica gel (eluent: Pet. Ether:EtOAc=6:1) to afford methyl N-(tert-butoxycarbonyl)-O-(cyclohexylmethyl)-L-threoninate (1.7 g, 58%) as a yellow oil. LCMS m/z=352.2 [M+Na]⁺; ¹H NMR (400 MHz, DMSO-d₆) δ 6.53 (d, J=9.0 Hz, 1H), 4.12 (dd, J=9.0, 3.8 Hz, 1H), 3.79 (m, 1H), 3.63 (s, 3H), 3.25 (dd, 1=9.0, 6.2 Hz, 1H), 3.04 (dd, 1=9.0, 6.6 Hz, 1H), 1.63 (m, 5H), 1.39 (s, 9H), 1.27-1.09 (m, 4H), 1.06 (d, J=6.4 Hz, 3H), 0.84 (m, 2H).

Step 3: methyl O-(cyclohexylmethyl)-L-threoninate: To a solution of methyl N-(tert-butoxycarbonyl)-O-(cyclohexylmethyl)-L-threoninate (1.3 g, 4.07 mmol) in DCM (6 mL) was added a solution of HCl in dioxane (4M, 3 mL). The reaction mixture was stirred at room temperature for 2 h then the solvent was removed under vacuum to afford methyl 0-(cyclohexylmethyl)-L-threoninate (932 mg, 100%) which was used directly in the next step. LCMS m/z=230.2 [M+H]⁺.

### Synthesis of (2S,3R)-2-amino-3-(cyclohexylmethoxy)-1-(piperidin-1-yl)butan-1-one hydrochloride

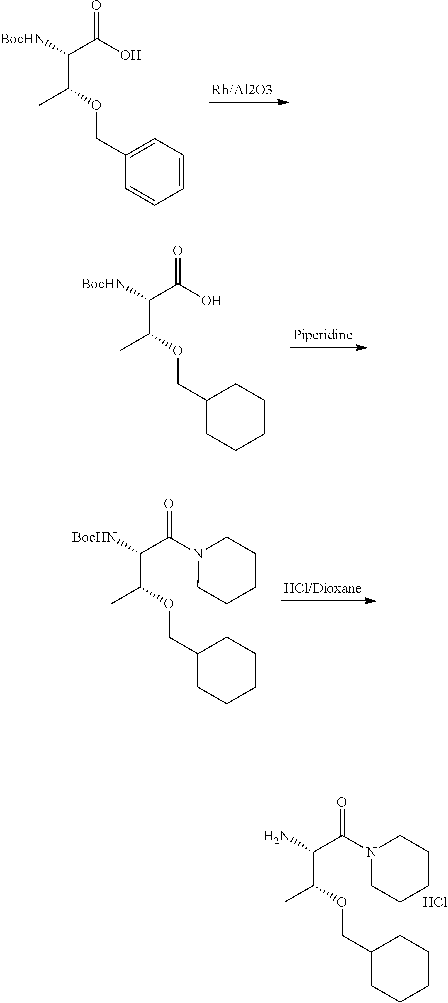 [SMILES: C[C@@H](OCc1ccccc1)[C@H](NC(=O)OC(C)(C)C)C(=O)O ; C[C@@H](OCC1CCCCC1)[C@H](NC(=O)OC(C)(C)C)C(=O)O ; C[C@@H](OCC1CCCCC1)[C@H](NC(=O)OC(C)(C)C)C(=O)N1CCCCC1 ; C[C@@H](OCC1CCCCC1)[C@H](N)C(=O)N1CCCCC1 ; Cl]

Step 1: N-(tert-butoxycarbonyl)-O-(cyclohexylmethyl)-L-threonine: To a solution of O-benzyl-N-(tert-butoxycarbonyl)-L-threonine (3 g, 9.7 mmol) in iPrOH (30 mL) was added Rh/Al₂O₃ (300 mg). The mixture was stirred at room temperature under H₂ overnight then the catalyst was removed by filtration through Celite and the filtrate concentrate to afford N-(tert-butoxycarbonyl)-O-(cyclohexylmethyl)-L-threonine (1.7 g, 56%) as a colorless solid. LCMS m/z=260.0 [M-55]⁺.

Step 2: tert-butyl ((2S,3R)-3-(cyclohexylmethoxy)-1-oxo-1-(piperidin-1-yl)butan-2-yl)carbamate: To a solution of N-(tert-butoxycarbonyl)-O-(cyclohexylmethyl)-L-threonine (300 mg, 0.95 mmol) in DMA (3 mL) were added piperidine (120 mg, 1.43 mmol), EDCI (274 mg, 1.43 mmol), HOBt (154 mg, 1.14 mmol) and DIEA (246 mg, 1.9 mmol) and the reaction stirred at room temperature overnight. The reaction mixture was treated with water (10 mL) and extracted with EtOAc (20 mL×2). The combined organic layers were washed with water (20 mL×3), dried over Na₂SO₄, filtered and concentrated. The residue obtained was purified by RP-column to afford tert-butyl ((2S,3R)-3-(cyclohexylmethoxy)-1-oxo-1-(piperidin-1-yl)butan-2-yl)carbamate (332 mg, 91%) as a white solid. LCMS m/z=383.3 [M+H]⁺.

Step 3: (2S,3R)-2-amino-3-(cyclohexylmethoxy)-1-(piperidin-1-yl)butan-1-one hydrochloride: To a solution of tert-butyl ((2S,3R)-3-(cyclohexylmethoxy)-1-oxo-1-(piperidin-1-yl)butan-2-yl)carbamate (300 mg, 0.78 mmol) in DCM (3 mL) was added a solution of HCl in dioxane (4 M, 3 mL). The mixture was stirred at room temperature for 4 h then the solvent was removed in vacuo to afford (2S,3R)-2-amino-3-(cyclohexylmethoxy)-1-(piperidin-1-yl)butan-1-one hydrochloride (230 mg, 90%) as a white solid. LCMS m/z=283.2 [M+H]⁺.

### 2-amino-2-(5-cyclohexyl-1,3,4-oxadiazol-2-yl)-N-methylacetamide

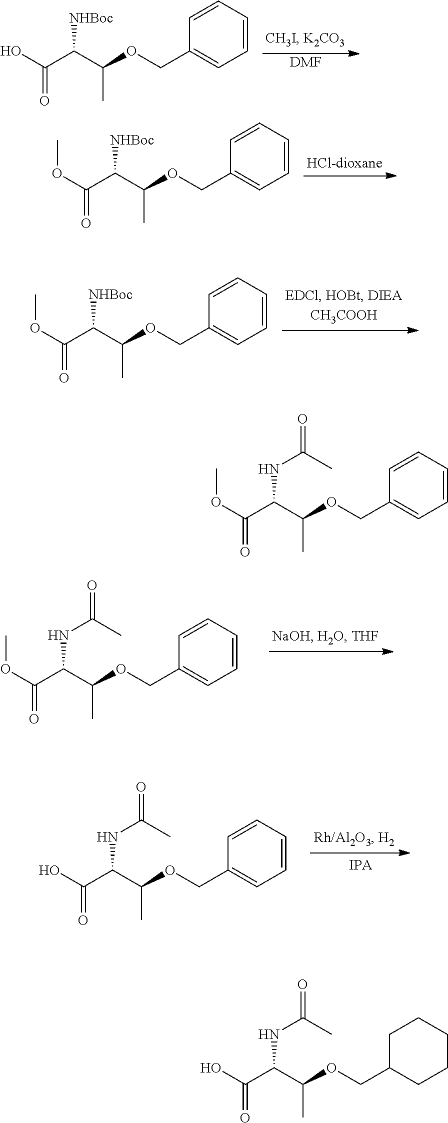

Step 1: methyl O-benzyl-N-(tert-butoxycarbonyl)-D-threoninate To a solution of O-benzyl-N-(tert-butoxycarbonyl)-D-threonine (1.0 g, 3.23 mmol) in DMF (10 mL) was added CH₃I (688 mg, 4.85 mmol) and K₂CO₃ (893 mg, 6.46 mmol). The reaction was stirred in sealed tube at room temperature overnight. The mixture was diluted with water (50 mL) and extracted with EtOAc (100 mL×2). The combined organic layers were washed with brine, dried over Na₂SO₄, filtered and concentrated. The residue obtained was purified by column chromatography on silica gel (eluent: Pet. Ether:EtOAc=5:1) to afford methyl O-benzyl-N-(tert-butoxycarbonyl)-D-threoninate (810 mg, 77%) as a yellow oil. LCMS m/z=268.0 [M-Boc+H]⁺; ¹H NMR (400 MHz, DMSO-d6) δ 6.56-6.41 (m, 5H), 4.02 (s, 1H), 3.76 (d, J=11.6 Hz, 1H), 3.57 (d, J=11.6 Hz, 1H), 3.43 (d, J=3.2 Hz, 1H), 3.32-3.22 (m, 1H), 2.84 (s, 3H), 0.64 (s, 9H), 0.41 (d, J=6.4 Hz, 3H).

Step 2: methyl O-benzyl-D-threoninate hydrochloride To a solution of methyl O-benzyl-N-(tert-butoxycarbonyl)-D-threoninate (800 mg, 2.47 mmol) in MeOH (1 mL) was added a solution of HCl in dixoane (4M, 1 mL). The resulting mixture was stirred for 3 h then the solvent was removed under reduced pressure to afford methyl O-benzyl-D-threoninate hydrochloride (552 mg, quant). LCMS m/z=224.0 [M+H]⁺.

Step 3: methyl N-acetyl-O-benzyl-D-threoninate To a solution of methyl O-benzyl-D-threoninate hydrochloride (552 mg, 2.48 mmol) in DMF (5 mL) was added acetic acid (223 mg, 3.72 mmol), EDCI (713 mg, 3.72 mmol), HOBt (502 mg, 3.72 mmol) and DIPEA (958 mg, 7.44 mmol) and the reaction stirred at room temperature for 14 h. The mixture was diluted with water (30 mL) and extracted with EtOAc (80 mL×2). The combined organic layers were washed with brine, dried over Na₂SO₄, filtered and concentrated. The residue obtained was purified by column chromatography on silica gel (eluent: Pet. Ether:EtOAc=5:1) to afford methyl N-acetyl-O-benzyl-D-threoninate (580 mg, 28%) as a yellow oil. LCMS m/z=265.1 [M+H]⁺.

Step 4: tert-butyl (1-(5-cyclohexyl-1,3,4-oxadiazol-2-yl)-2-(methylamino)-2-oxoethyl)carbamate To a solution of methyl N-acetyl-O-benzyl-D-threoninate (580 mg, 2.19 mmol) in MeOH (3 mL) was added aqueous NaOH (1 M, 2 mL). The resulting mixture was stirred at room temperature for 3 h, then was diluted with water (30 mL) and extracted with EtOAc (50 mL). The aqueous layer was collected, acidified with 1M HCl to pH ˜2 and extracted with EtOAc (100 mL×3). The combined organic layers were washed with brine, dried over Na₂SO₄, filtered and concentrated to afford tert-butyl (1-(5-cyclohexyl-1,3,4-oxadiazol-2-yl)-2-(methylamino)-2-oxoethyl)carbamate (500 mg, 90%) as a yellow oil. LCMS m/z=252.1 [M+H]⁺.

Step 5: 2-amino-2-(5-cyclohexyl-1,3,4-oxadiazol-2-yl)-N-methylacetamide To a solution of tert-butyl (1-(5-cyclohexyl-1,3,4-oxadiazol-2-yl)-2-(methylamino)-2-oxoethyl)carbamate (500 mg, 1.99 mol) in MeOH (3 mL) was added Rh/Al₂O₃ (50 mg). The resulting mixture was stirred under an atmosphere of H₂ overnight. The catalyst was removed by filtration through Celite and the filtrate concentrated to afford 2-amino-2-(5-cyclohexyl-1,3,4-oxadiazol-2-yl)-N-methylacetamide (400 mg, 78%) as a yellow oil. LCMS m/z=257.9 [M+H]⁺.

### Synthesis of 4-(1-(O-(cyclohexylmethyl)-N-(8-((S)-2,2-dimethylcyclopropane-1-carbonyl)-2-(thiazole-5-carbonyl)-2,8-diazaspiro[4.5]decane-4-carbonyl)-L-threonyl)piperidin-4-yl)benzoic acid I-41

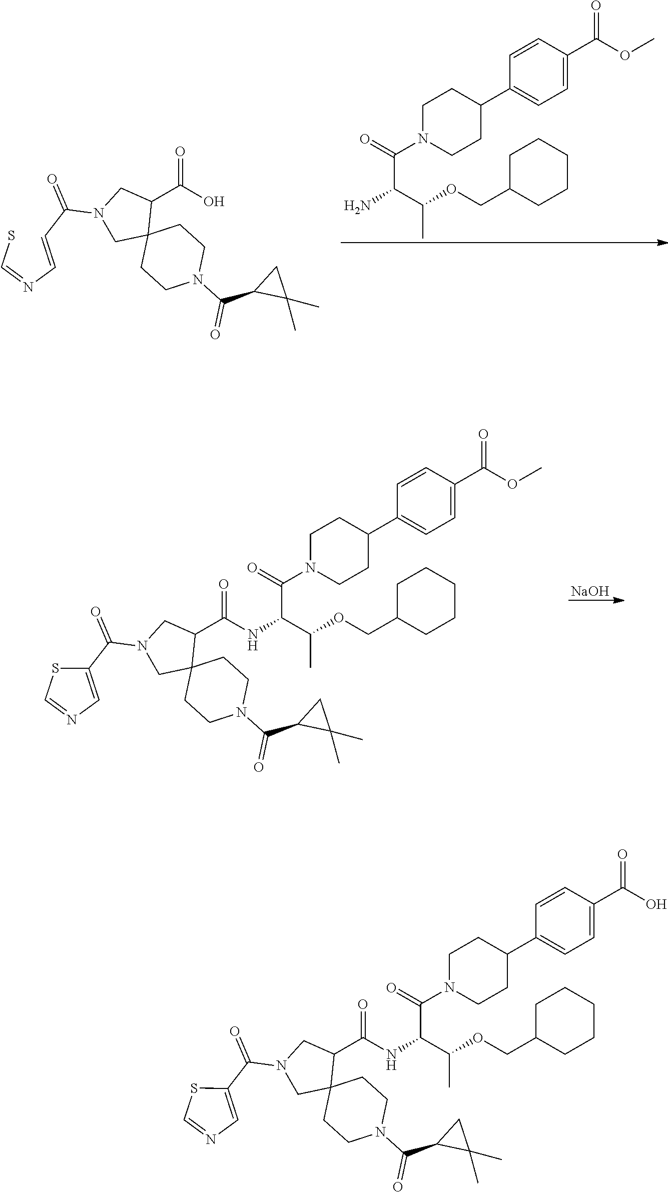

Step 1: methyl 4-(1-(O-(cyclohexylmethyl)-N-(8-((S)-2,2-dimethylcyclopropane-1-carbonyl)-2-(thiazole-5-carbonyl)-2,8-diazaspiro[4.5]decane-4-carbonyl)-L-threonyl)piperidin-4-yl)benzoate: To a solution of 8-((S)-2,2-dimethylcyclopropane-1-carbonyl)-2-(thiazole-5-carbonyl)-2,8-diazaspiro[4.5]decane-4-carboxylic acid (100 mg, 0.26 mmol) in DCM (1 mL) was added HATU (99 mg, 0.26 mmol) and DIPEA (101 mg, 0.78 mmol) and the mixture stirred at room temperature for 30 min. Methyl 4-(1-(O-(cyclohexylmethyl)-L-threonyl)piperidin-4-yl)benzoate (106 mg, 0.26 mmol) was added and stirring continued for 2 h. The mixture was diluted with water (20 mL) and extracted with DCM (30 mL×2). The combined organic layers were washed with brine, dried over Na₂SO₄, filtered and concentrated. The residue obtained was purified by prep-TLC to afford methyl 4-(1-(O-(cyclohexylmethyl)-N-(8-((S)-2,2-dimethylcyclopropane-1-carbonyl)-2-(thiazole-5-carbonyl)-2,8-diazaspiro[4.5]decane-4-carbonyl)-L-threonyl)piperidin-4-yl)benzoate (120 mg, 58%) as a yellow oil. LCMS m/z=790.3 [M+H]⁺; ¹H NMR (400 MHz, CD₃OD) δ 9.15 (s, 1H), 8.42-8.34 (m, 1H), 7.99-7.90 (m, 2H), 7.36 (d, J=7.8 Hz, 2H), 5.07-4.92 (m, 1H), 4.74-4.58 (m, 1H), 4.41-3.37 (m, 12H), 3.28-3.08 (m, 3H), 3.03-2.70 (m, 2H), 2.08-1.45 (m, 14H), 1.44-1.12 (m, 10H), 1.11-0.83 (m, 8H), 0.72 (s, 1H).

Step 2: 4-(1-(O-(cyclohexylmethyl)-N-(8-((S)-2,2-dimethylcyclopropane-1-carbonyl)-2-(thiazole-5-carbonyl)-2,8-diazaspiro[4.5]decane-4-carbonyl)-L-threonyl)piperidin-4-yl)benzoic acid: To a solution of methyl 4-(1-(O-(cyclohexylmethyl)-N-(8-((S)-2,2-dimethylcyclopropane-1-carbonyl)-2-(thiazole-5-carbonyl)-2,8-diazaspiro[4.5]decane-4-carbonyl)-L-threonyl)piperidin-4-yl)benzoate (100 mg, 0.13 mmol) in THF (1.0 mL) was added 10% NaOH (0.1 mL) and the reaction stirred at room temperature for 2 h. The mixture was diluted with water (10 mL) and extracted with EtOAc (15 mL). The aqueous layer was collected, acidified with 1M HCl to pH ˜2 and extracted with EtOAc (30 mL×3). The combined organic layers were washed with brine, dried over Na₂SO₄, filtered and concentrated. The residue was purified by RP-column to afford 4-(1-(O-(cyclohexylmethyl)-N-(8-((S)-2,2-dimethylcyclopropane-1-carbonyl)-2-(thiazole-5-carbonyl)-2,8-diazaspiro[4.5]decane-4-carbonyl)-L-threonyl)piperidin-4-yl)benzoic acid (20 mg, 20%) as a white solid. LCMS m/z=776.2 [M+H]⁺; ¹H NMR (400 MHz, CD₃OD) δ 9.16 (s, 1H), 8.44-8.33 (m, 1H), 8.01-7.90 (m, 2H), 7.35 (d, 2H), 5.07-4.94 (m, 1H), 4.73-4.62 (m, 1H), 4.39-3.70 (m, 8H), 3.67-3.35 (m, 3H), 3.29-3.09 (m, 3H), 3.02-2.90 (m, 1H), 2.86-2.72 (m, 1H), 2.03-1.51 (m, 16H), 1.24 (s, 8H), 1.10-0.90 (m, 6H), 0.72 (s, 1H).

### Synthesis of (S)-6-acetyl-N-((2S,3R)-3-(cyclohexylmethoxy)-1-morpholino-1-oxobutan-2-yl)-2-((S)-2,2-dimethylcyclopropane-1-carbonyl)-2,6-diazaspiro[3.4]octane-8-carboxamide I-36

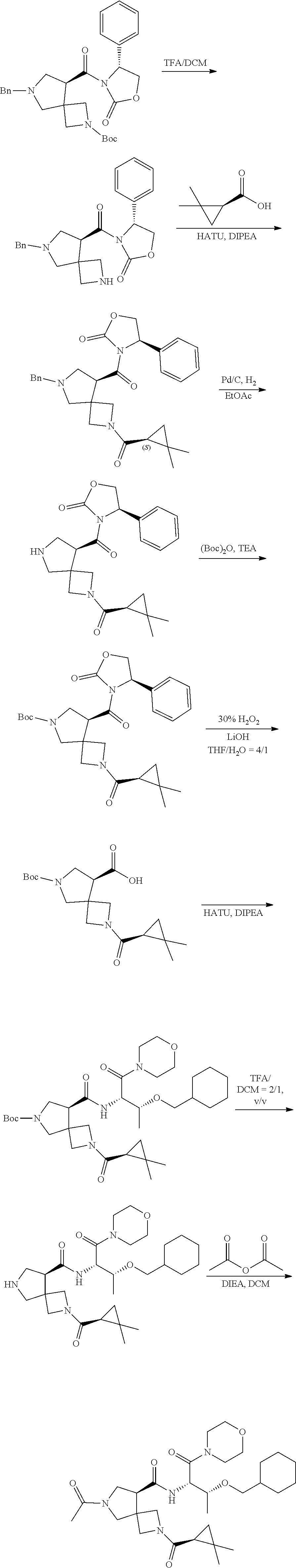

Step 1: (R)-3-((S)-6-benzyl-2,6-diazaspiro[3.4]octane-8-carbonyl)-4-phenyloxazolidin-2-one: To a solution of tert-butyl (S)-6-benzyl-8-((R)-2-oxo-4-phenyloxazolidine-3-carbonyl)-2,6-diazaspiro[3.4]octane-2-carboxylate (Intermediate 11) (1.1 g, 2.2 mmol) in DCM (4 mL) was added TFA (2 mL) and the reaction stirred at room temperature for 2 h. The solvent was removed under reduced pressure to afford (R)-3-((S)-6-benzyl-2,6-diazaspiro[3.4]octane-8-carbonyl)-4-phenyloxazolidin-2-one (876 mg, 100%) which was used without purification.

Step 2: (R)-3-((S)-6-benzyl-2-((S)-2,2-dimethylcyclopropane-1-carbonyl)-2,6-diazaspiro[3.4]octane-8-carbonyl)-4-phenyloxazolidin-2-one: To a solution of (S)-2,2-dimethylcyclopropane-1-carboxylic acid (274 mg, 2.4 mmol) in DCM (10 mL) was added HATU (1.3 g, 3.3 mmol) and the mixture was stirred at room temperature for 30 min. (S)-4-benzyl-3-((S)-6-benzyl-2,6-diazaspiro[3.4]octane-8-carbonyl)oxazolidin-2-one (876 mg, 2.2 mmol) and DIPEA (1.1 g, 8.8 mmol) were added and stirring continued for another 4 h. The mixture was diluted with water (20 mL) and extracted with DCM (50 mL×2). The combined organic layers were washed with brine, dried over Na₂SO₄, filtered and concentrated. The mixture was purified by column chromatography on silica gel (eluent: Pet. Ether:EtOAc=3:1 to DCM/EtOAc=3:1) to afford (R)-3-((S)-6-benzyl-2-((S)-2,2-dimethylcyclopropane-1-carbonyl)-2,6-diazaspiro[3.4]octane-8-carbonyl)-4-phenyloxazolidin-2-one (900 mg, 82%) as a yellow solid. LCMS m/z=488.1 [M+H]⁺; ¹H NMR (400 MHz, DMSO-d6) δ 7.44-7.24 (m, 10H), 5.53-5.46 (m, 1H), 4.78-4.71 (m, 1H), 4.50-3.48 (m, 9H), 3.21-2.53 (m, 3H), 1.39-1.32 (m, 1H), 1.28-1.22 (m, 2H), 1.10 (d, J=2.8 Hz, 3H), 1.03 (d, J=24.4 Hz, 3H), 0.87-0.82 (m, 1H), 0.71-0.64 (m, 1H).

Step 3: (R)-3-((S)-2-((S)-2,2-dimethylcyclopropane-1-carbonyl)-2,6-diazaspiro[3.4]octane-8-carbonyl)-4-phenyloxazolidin-2-one: To a solution of (S)-4-benzyl-3-((S)-6-benzyl-2-((S)-2,2-dimethylcyclopropane-1-carbonyl)-2,6-diazaspiro[3.4]octane-8-carbonyl)oxazolidin-2-one (1.0 g, 2.1 mmol) in EtOAc (8 mL) was added 10% Pd/C (400 mg). The reaction was stirred under a H₂ atmosphere for 24 h, conversion was around 50%. The catalyst was removed and the mixture redissolved in EtOAc (8 mL) and resubjected to hydrogenation under the same conditions for a further 24 hrs. The catalyst was removed by filtration through celite and the filtrate concentrated to afford (R)-3-((S)-2-((S)-2,2-dimethylcyclopropane-1-carbonyl)-2,6-diazaspiro[3.4]octane-8-carbonyl)-4-phenyloxazolidin-2-one (800 mg, 98%) which was used directly in the next step. LCMS m/z=398.1 [M+H]⁺.

Step 4: tert-butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate: To a solution of (R)-3-((S)-2-((S)-2,2-dimethylcyclopropane-1-carbonyl)-2,6-diazaspiro[3.4]octane-8-carbonyl)-4-phenyloxazolidin-2-one (800 mg, 2.028 mmol) in DCM (10 mL) were added (Boc)₂O (438 mg, 2.208 mmol) and TEA (407 mg, 4.056 mmol). The reaction mixture was stirred at room temperature for 2 h then was concentrated in vacuo and the residue obtained purified by RP-column (38% ACN in water) to afford tert-butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate (450 mg, 45%) as a white solid. LCMS m/z=498.4 [M+H]⁺; ¹H NMR (400 MHz, DMSO-d6) δ 7.37-7.23 (m, 5H), 5.46-5.45 (m, 1H), 4.78-4.73 (m, 1H), 4.18-4.15 (m, 3H), 4.02-3.91 (m, 1H), 3.82-3.65 (m, 2H), 3.62-3.52 (m, 3H), 3.41-3.37 (m, 1H), 1.40 (s, 1H), 1.34-1.33 (m, 8H), 1.11-1.03 (m, 7H), 0.87-0.84 (m, 1H), 0.69-0.67 (m, 1H).

Step 5: (S)-6-(tert-butoxycarbonyl)-2-((S)-2,2-dimethylcyclopropane-1-carbonyl)-2,6-diazaspiro[3.4]octane-8-carboxylic acid To a solution of tert-butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate (490 mg, 0.985 mmol) in a mixture of THE and H₂O (4 mL/1 mL) at 0° C. was added a solution of lithium hydroxide monohydrate (103 mg, 2.46 mmol) and 30% H₂O₂ (67 mg, 1.97 mmol) in water (1 mL). The reaction mixture was stirred at 0° C. for 1 h then was diluted with water (200 mL) and extracted with EtOAc (20 mL). The aqueous layer was collected, acidified to pH ˜2 with 1M HCl and extracted with EtOAc (20 mL×3). The combined organic layers were washed with brine, dried over Na₂SO₄, filtered and concentrated to afford crude (S)-6-(tert-butoxycarbonyl)-2-((S)-2,2-dimethylcyclopropane-1-carbonyl)-2,6-diazaspiro[3.4]octane-8-carboxylic acid (285 mg, 82%) as a white solid which was used directly in the next step. LCMS m/z=353 [M+H]⁺; ¹H NMR (400 MHz, DMSO-d₆) δ 12.76 (s, 1H), 4.15-3.70 (m, 4H), 3.59-3.42 (m, 4H), 3.22-3.14 (m, 1H), 1.40 (s, 9H), 1.36-1.33 (m, 1H), 1.11 (d, J=3.6 Hz, 3H), 1.04 (d, J=8 Hz, 3H), 0.87-0.85 (m, 1H), 0.70-0.64 (m, 1H).

Step 6: tert-butyl (S)-8-(((2S,3R)-3-(cyclohexylmethoxy)-1-morpholino-1-oxobutan-2-yl)carbamoyl)-2-((S)-2,2-dimethylcyclopropane-1-carbonyl)-2,6-diazaspiro[3.4]octane-6-carboxylate: To a solution of (S)-6-(tert-butoxycarbonyl)-2-((S)-2,2-dimethylcyclopropane-1-carbonyl)-2,6-diazaspiro[3.4]octane-8-carboxylic acid (91 mg, 0.14 mmol) in DCM (3 mL) was added HATU (118 mg, 0.15 mmol) and DIPEA (146 mg, 0.57 mmol) and the mixture stirred at room temperature for 30 min. (2S,3R)-2-amino-3-(cyclohexylmethoxy)-1-morpholinobutan-1-one (100 mg, 0.14 mmol) was added and stirring continued for 2 h. The solvent was removed under vacuum and the residue obtained was purified by prep-HPLC to afford tert-butyl (S)-8-(((2S,3R)-3-(cyclohexylmethoxy)-1-morpholino-1-oxobutan-2-yl)carbamoyl)-2-((S)-2,2-dimethylcyclopropane-1-carbonyl)-2,6-diazaspiro[3.4]octane-6-carboxylate (6.9 mg, 4%) as a yellow solid. LCMS m/z=619.5 [M+H]⁻; ¹H NMR (400 MHz, DMSO-d6) δ 8.39-8.31 (m, 1H), 4.88-4.81 (m, 1H), 4.19-3.78 (m, 3H), 3.71-3.41 (m, 13H), 3.38-3.34 (m, 2H), 3.28-3.21 (m, 1H), 3.18-3.12 (m, 1H), 1.71-1.59 (m, 5H), 1.48-1.34 (m, 11H), 1.32-1.23 (m, 1H), 1.16-1.01 (m, 11H), 0.85 (t, J=4.9 Hz, 3H), 0.69-0.64 (m, 1H).

Step 7: (S)—N-((2S,3R)-3-(cyclohexylmethoxy)-1-morpholino-1-oxobutan-2-yl)-2-((S)-2,2-dimethylcyclopropane-1-carbonyl)-2,6-diazaspiro[3.4]octane-8-carboxamide I-32 To a solution of tert-butyl (S)-8-(((2S,3R)-3-(cyclohexylmethoxy)-1-morpholino-1-oxobutan-2-yl)carbamoyl)-2-((S)-2,2-dimethylcyclopropane-1-carbonyl)-2,6-diazaspiro[3.4]octane-6-carboxylate (30 mg, 0.07 mmol) in DCM (0.3 mL) was added TFA (0.6 mL) and the reaction stirred at room temperature for 1 h. The residue obtained was purified by prep-HPLC to afford (S)—N-((2S,3R)-3-(cyclohexylmethoxy)-1-morpholino-1-oxobutan-2-yl)-2-((S)-2,2-dimethylcyclopropane-1-carbonyl)-2,6-diazaspiro[3.4]octane-8-carboxamide (13.5 mg, 54%) as a yellow solid. LCMS m/z=519.4 [M+H]⁺; ¹H NMR (400 MHz, DMSO-d6) δ 8.61-8.54 (m, 1H), 4.91-4.81 (m, 1H), 4.11-4.08 (m, 1H), 3.93-3.83 (m, 1H), 3.79-3.69 (m, 1H), 3.64-3.41 (m, 11H), 3.30-3.21 (m, 4H), 3.21-3.13 (m, 2H), 1.70-1.57 (m, SH), 1.50-1.38 (m, 1H), 1.36-1.26 (m, 1H), 1.20-1.03 (m, 12H), 0.91-0.80 (m, 3H), 0.72-0.65 (m, 1H).

Step 8: (S)-6-acetyl-N-((2S,3R)-3-(cyclohexylmethoxy)-1-morpholino-1-oxobutan-2-yl)-2-((S)-2,2-dimethylcyclopropane-1-carbonyl)-2,6-diazaspiro[3.4]octane-8-carboxamide I-36: To a solution of (S)—N-((2S,3R)-3-(cyclohexylmethoxy)-1-morpholino-1-oxobutan-2-yl)-2-((S)-2,2-dimethylcyclopropane-1-carbonyl)-2,6-diazaspiro[3.4]octane-8-carboxamide (27 mg, 0.05 mmol) in DCM (1 mL) was added DIPEA (26 mg, 0.2 mmol) and acetic anhydride (15 mg, 0.2 mmol). The reaction mixture was stirred at room temperature for 2 h then was concentrated in vacuo and the residue obtained purified by prep-HPLC to afford (S)-6-acetyl-N-((2S,3R)-3-(cyclohexylmethoxy)-1-morpholino-1-oxobutan-2-yl)-2-((S)-2,2-dimethylcyclopropane-1-carbonyl)-2,6-diazaspiro[3.4]octane-8-carboxamide (15.6 mg, 54%) as a white solid. LCMS m/z=561.4[M+H]⁺; ¹H NMR (400 MHz, DMSO-d6) δ 8.42-8.32 (m, 1H), 4.92-4.80 (m, 1H), 4.22-3.65 (m, 5H), 3.65-3.46 (m, 10H), 3.45-3.33 (m, 3H), 3.28-3.21 (m, 1H), 3.20-3.13 (m, 1H), 1.96-1.89 (m, 3H), 1.71-1.57 (m, 5H), 1.50-1.38 (m, 1H), 1.38-1.25 (m, 1H), 1.20-1.03 (m, 12H), 0.91-0.79 (m, 3H), 0.70-0.64 (m, 1H).

The following compound I-9 of Table I-6 was prepared according to the procedure of I-14

TABLE I-6

| Example | | | |
|---|---|---|---|
| Number | Compound | ¹HNMR | LCMS |
| I-9 | 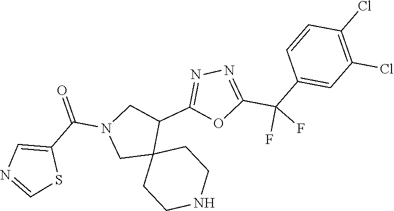 [SMILES: O=C(c1cncs1)N1CC(c2nnc(C(F)(F)c3ccc(Cl)c(Cl)c3)o2)C2(CCNCC2)C1] | ¹H NMR (400 MHz, CD₃OD)δ 9.18 (d, J = 4.9 Hz, 1H), 8.39 (s, 1H), 7.94-7.87 (m, 1H), 7.75 (d, J = 8.5 Hz, 1H), 7.70- 7.59 (m, 1H), 4.49- 4.33 (m, 1H), 4.23- 3.74 (m, 4H), 3.42 (s, 1H), 3.33-3.09 (m, 3H), 2.26-2.00 (m, 2H), 1.87-1.72 (m, 1H), 1.65-1.50 (m, 1H). | m/z = 514.1 [M + H]⁺ |

### Example A1: CDK2/Cyclin E1 Caliper Assay

Inhibition of CDK2/Cyclin E1 activity in the presence of compounds of the present disclosure was evaluated using a Caliper LabChip® EZ Reader mobility shift assay. In the assay, CDK2/Cyclin E1 (Eurofins, 14-475) catalyzed the phosphorylation of a fluorescently tagged peptide 5-FAM-QSPKKG-CONH2 (PerkinElmer, FL Peptide 18) which induced a difference in capillary electrophoresis mobility. The peptide substrate and product were measured, and the conversion ratio was used to determine the inhibition (as % activity and IC50 values) of CDK2/Cyclin E1. Reactions contained 50 mM HEPES pH 7.5, 10 mM MgCl2, 1 mM EDTA, 2 mM DTT, 0.01% Brij35, 0.5 mg/mL BSA, 0.1% DMSO, 2.5 nM CDK2/Cyclin E1, 100 μM ATP, and 1.5 μM fluorescent peptide substrate.

Dose titrations of inhibitors in 100% DMSO were combined with 3.25 nM CDK2/Cyclin E1 and 130 μM of ATP in reaction buffer. The mixtures were incubated for 30 minutes before the addition of fluorescent peptide substrate to initiate the kinase reaction. The final conditions were 2.5 nM CDK2/Cyclin E1, 100 μM ATP, and 1.5 μM fluorescent peptide. The reactions were stopped after 100 minutes with the addition of EDTA (400 mM final EDTA concentration). The stopped reactions were analyzed on a Caliper LabChip® EZ Reader II. The conversion ratios were normalized to yield % activity, plotted against compound concentration, and fit to a four-parameter equation to determine the IC50 for each compound.

The results of the Caliper Assay are reported in Table 3, below. Compounds with an IC₅₀ less than or equal to 0.01 μM are designated as “A”. Compounds with an IC₅₀ greater than 0.01 μM and less than or equal to 0.1 μM are designated as “B”. Compounds with an IC₅₀ greater than 0.1 μM and less than or equal to 1.0 μM are designated as “C”. Compounds with an IC₅₀ greater than 1.0 μM and less than or equal to 10.0 μM are designated as “D”. Compounds with an IC₅₀ greater than 10.0 μM are designated as “E”.

### Example A2: ADPGLO (CDK2/E1-37C)

Inhibition of CDK2/Cyclin E1 activity by the presence of small molecules was evaluated using ADP-Glo Luminescent Kinase Assay (Promega). Activated CDK2/Cyclin E1 was incubated with its substrate Histone H1 (SignalChem H10-54N) in the kinase reaction buffer (100 μM ATP, 50 mM HEPES pH 7.5, 10 mM MgCl2, 1 mM EDTA, 2 mM DTT, 0.01% Brij35, 0.5 mg/mL BSA). Luminescence was recorded with an Envision plate reader (PerkinElmer).

Dose titrations of inhibitors in 100% DMSO were combined with 0.36 nM CDK2/Cyclin E1 in reaction buffer. The mixtures were incubated for 60 minutes at 37° C. before the addition of ATP and Histone H1 substrate to initiate the kinase reaction. The final conditions were 0.18 nM CDK2/Cyclin E1, 100 μM ATP, and 1.5 μM Histone H1. The reactions were incubated at 37° C. for 90 minutes before being stopped with the addition of ADP-Glo reagent. This mixture was incubated at room temperature for 60 minutes before Kinase Detection Solution is added to generate luminescence. The stopped reactions were analyzed on an Envision plate reader. The conversion ratios were normalized to yield % activity, plotted against compound concentration, and fit to a four-parameter equation to determine the IC₅₀ for each compound.

The results of the ADPGLO assay are reported in Table 3, below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 μM and less than or equal to 10.0 μM are designated as “C”. Compounds with an IC₅₀ greater than 10.0 μM are designated as “D”. Compounds with an IC₅₀ greater than 100.0 μM are designated as “E”.

### Example A3: IncuCyte® Cell Proliferation Assay

IncuCyte® assay was used to measure the effect of disclosed compounds on cell proliferation. Fluorescent microscopy images of cells were taken immediately after compound treatment and 72 hours later. Image analysis software was used to obtain cell counts as a function of compound concentration. Kuramochi cells labeled with mApple-H2B were seeded on 384-well assay-ready plates. Plates were placed in an IncuCyte® (Sartorius) and scanned at 0 and 72 hours. IncuCyte® software was used to count the number of fluorescent nuclei in each well. The fold change in cell count from 0 to 72 hours in wells treated with increasing compounds concentrations (10 pts, 1/2 log dilution, 20 μM top concentration) was normalized to DMSO control wells. The normalized cell counts were fit with dose response curves and a GI50 was calculated.

The results of the IncuCyte® cell proliferation assay are reported in Table 3, below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 μM and less than or equal to 20.0 μM are designated as “C”. Compounds with an IC₅₀ greater than 20.0 μM are designated as “D”.

TABLE 3
Assay Results

| | Caliper Assay | ADPGLO | IncuCyte |
|---|---|---|---|
| | (CDK2/CCNE1): | (CDK2/E1-37C): | Kuramochi RB-WT: |
| Cmpd # | IC50 (μM) | IC50 (μM) | IC50 (mM) |
| I-1 | E | | D |
| I-2 | E | | D |
| I-3 | E | | D |
| I-4 1^{st} | | D | D |
| I-4 2^{nd} | | D | D |
| I-5 | | A | A |
| I-6 | E | E | D |
| I-7 | E | | D |
| I-8 | E | | D |
| I-9 | E | | D |
| I-10 | E | | D |
| I-11 | E | | D |
| I-12 | E | | D |
| I-13 | E | | D |
| I-14 | E | | C |
| I-15 | E | | D |
| I-16 | E | | D |
| I-17 | D | | D |
| I-18 | D | | |
| I-19 | D | | D |
| I-20 | E | | D |
| I-21 | C | | C |
| I-22 | E | | D |
| I-23 | E | | D |
| I-24 | D | | D |
| I-25 | C | | D |
| I-26 | E | | D |
| I-27 | E | | D |
| I-28 | E | | D |
| I-29 1^{st} | E | | D |
| I-29 2^{nd} | E | | D |
| I-30 | C | | D |
| I-31 1^{st} | E | | D |
| I-31 2^{nd} | E | | D |
| I-32 | E | | D |
| I-33 | D | | D |
| I-34 1^{st} | E | | D |
| I-34 2^{nd} | E | | D |
| I-35 1^{st} | E | | D |
| I-35 2^{nd} | E | | D |
| I-36 | E | | D |
| I-37 | E | | D |
| I-38 | D | | D |
| I-39 | E | | D |
| I-40 | E | | |
| I-41 | | A | D |

1. A compound according to Formula I: 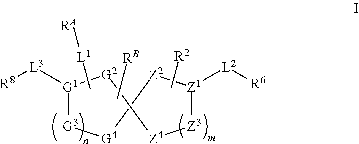
or a pharmaceutically acceptable salt thereof, wherein:
G¹ is N or CH;
each G², G³, and G⁴ is independently optionally substituted methylene, —O—, —S—, —N(R)—, —C(O)—, —S(O)—, or —S(O)₂—;
Z¹ is N or CH; and
each Z², Z³ and Z⁴ is independently optionally substituted methylene, —O—, —S—, —N(R)—, —C(O)—, —S(O)—, or —S(O)₂—;
R^{A} is an amide isostere, 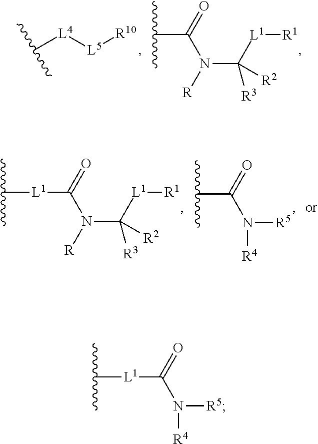
R^{B} is a hydrogen, an optionally substituted C₁₋₆ aliphatic group, or a halogen;
each L¹ is independently a covalent bond or a saturated or unsaturated, straight or branched, optionally substituted bivalent C₁₋₆ hydrocarbon chain, wherein 0-4 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
each of R² and R³ is independently hydrogen, an optionally substituted C₁₋₆ aliphatic group, —C₁₋₆ alkylene-OR, —C₁₋₃ alkylene-O—C₁₋₃ alkylene-R, —C(O)OR, or —C(O)NR₂; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R⁵ is hydrogen; or:
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R⁶ is hydrogen, —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁷ or R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, -L⁴-Cy, or Cy;
L³ is a covalent bond, a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or, -Cy-, —NRC(O)NR—;
each L⁴ is independently a covalent bond, a saturated or unsaturated, straight or branched, optionally substituted bivalent C₁₋₄ hydrocarbon chain, wherein 0-2 methylene units of L⁴ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
L⁵ is a covalent bond or a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy-, or —NRC(O)NR—;
R¹ is hydrogen, a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, -L⁴-Cy, or Cy;
R¹⁰ is hydrogen, —CN, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹;
each Cy is independently an optionally substituted cyclic group which may be bivalent selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), an 8-10 membered bicyclic aromatic carbocyclic ring, a 7-12 membered saturated or partially unsaturated bicyclic heterocyclic ring that is optionally bridged bicyclic (having 1-4 heteroatoms independently selected from nitrogen, oxygen, and sulfur), an 8-10 membered bicyclic heteroaromatic ring (having 1-5 heteroatoms independently selected from nitrogen, oxygen, and sulfur), and a 5-6 membered monocyclic heteroaromatic ring (having 1-4 heteroatoms independently selected from nitrogen, oxygen, and sulfur);
R^{z} is hydrogen, —CN, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of -L⁴-R⁹ or R⁹; and
each R is independently hydrogen, halogen or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
an R group of R^{A} and one of Z¹, Z², Z³, Z⁴, G¹, G², G³, or G4 are taken together with their intervening atoms to form an optionally substituted 3-7 membered heterocyclic ring; and/or
an R group of R^{A} and one of R¹, R², or R³ are taken together with their intervening atoms to form a 5-7 membered heterocyclic ring; and/or
two R groups on the same atom are taken together with their intervening atoms to form an optionally substituted 3-7 membered saturated, partially unsaturated, or heteroaryl ring (having 0-3 heteroatoms independently selected from nitrogen, oxygen, and sulfur); or the two R groups on the same atom are taken together with their intervening atoms to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
m is 0, 1, or 2; and
n is 0, 1, or 2.

2. (canceled)

3. The compound of claim 1, wherein R^{A} is

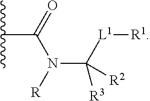

4. (canceled)

5. The compound of claim 1, wherein R^{A} is

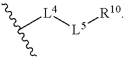

6-12. (canceled)

13. The compound of claim 1, wherein L¹ is

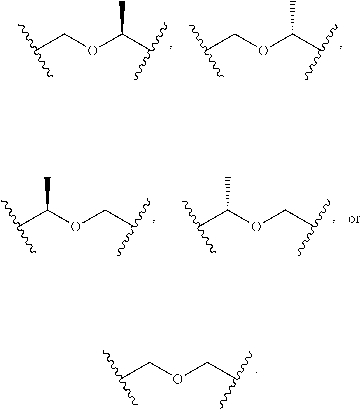

14. The compound of claim 1, wherein L¹ is 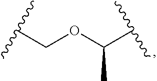 [SMILES: *CO[C@@H](*)C]
wherein the [symbol] on the left of 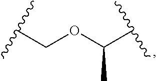 [SMILES: *CO[C@@H](*)C]
is attached to R¹.

15. The compound of claim 1, wherein R¹ is

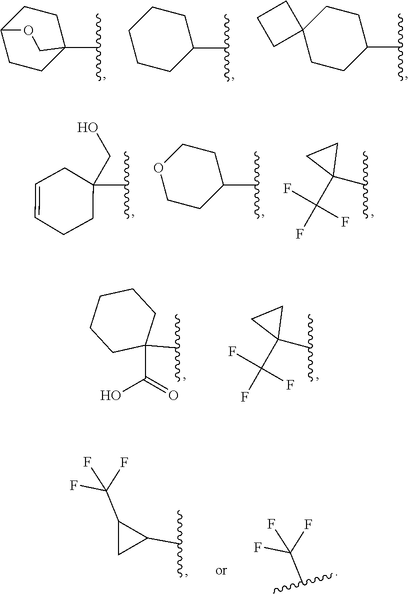

16. The compound of claim 1, wherein R¹

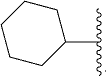 [SMILES: *C1CCCCC1]

17-25. (canceled)

26. The compound of claim 1, wherein L⁴ is a substituent of Table L4-a.

27. The compound of claim 1, wherein L⁴ is an optionally substituted 5-membered heteroaryl.

28. The compound of claim 1, wherein L⁴ is

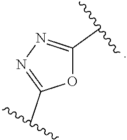 [SMILES: *c1nnc(*)o1]

29. The compound of claim 1, wherein L⁴ (as read from left to right 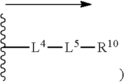
is —C(CH₃)H—O—CH₂—, —CH₂—O—C(CH₃)H—, —CH₂OCH₂—, —CH₂—NH—CH₂—, —CH₂—N(CH₃)—CH₂—, —C(O)NH—S(O)₂—, —CH₂—S(O)₂—CH₂—, —NHC(O)—, —CH₂O—, or —OCH₂.

30. The compound of claim 1, wherein R¹⁰ is a substituent of Table R10-a or Table R10-b.

31. The compound of claim 1, wherein R¹⁰ is optionally substituted phenyl.

32. The compound of claim 1, wherein R¹⁰ is optionally substituted cyclohexyl.

33-57. (canceled)

58. A method of treating a disease or disorder associated with CDK2 activity in a patient comprising administering to the patient in need thereof a compound of claim 1, wherein the disease or disorder associated with CDK2 activity is a cancer selected from the group consisting of breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

59-66. (canceled)
